(19) United States
(12) Patent Application Publication
(10) Pub. No.: US 2023/0303719 A1
(43) Pub. Date: Sep. 28, 2023

(54) HUMANIZED 3E10 ANTIBODIES, VARIANTS, AND ANTIGEN BINDING FRAGMENTS THEREOF

(71) Applicants: YALE UNIVERSITY, New Haven, CT (US); GENNAO BIO, INC., Pennington, NJ (US)

(72) Inventors: Elias Quijano, New Haven, CT (US); Peter Glazer, New Haven, CT (US); Stephen Squinto, New York, NY (US); Dale Ludwig, Rockaway, NJ (US)

(21) Appl. No.: 18/177,571

(22) Filed: Mar. 2, 2023

Related U.S. Application Data
(60) Provisional application No. 63/316,338, filed on Mar. 3, 2022.

(51) Int. Cl. C07K 16/44; A61K 47/68; A61K 39/395; A61P 35/02; A61K 38/17; A61P 1/18

(52) U.S. Cl. CPC C07K 16/44; A61K 47/6843; A61K 39/39583; A61P 35/02; A61K 38/1793; A61P 1/18; A61K 2039/505

(57) ABSTRACT

The disclosure provides humanized 3E10 antibodies and antigen binding fragments thereof. Compositions and methods of using the humanized 3E10 antibodies and antigen binding fragments thereof to deliver cargo are also disclosed.

### CROSS REFERENCE TO RELATED APPLICATION

This application claims priority to U.S. Provisional Application No. 63/316,338 filed Mar. 3, 2022, the disclosure of which is herein incorporated by reference in its entirety.

### TECHNICAL FIELD

The present disclosure relates to humanized 3E10 antibodies and antigen binding fragments thereof, e.g., for delivering therapeutic cargo into cells.

### BRIEF DESCRIPTION OF THE SEQUENCE LISTING

This submission is accompanied by a “Sequence Listing XML” containing SEQ ID NOs: 1-161 created on Mar. 1, 2023, 169 KB, in accordance with 37 CFR §§ 1.831 through 1.835, submitted as an XML file, via the USPTO patent electronic filing system. 37 CFR § 1.835(a)(1).

### BACKGROUND

Among the polynucleotide-based cancer therapies, many different strategies have evolved. For instance, immunostimulant polynucleotides, such as pattern recognition receptors, have been used to agonize mediators of proinflammatory cytokines in various cancer immunotherapies. Gene-regulating polynucleotides, e.g., siRNA, miRNA, ASO, etc., have been used to silence targeted genes, regulating signaling pathways involved in cancer progression. Polynucleotides encoding therapeutic proteins, e.g., mRNA or plasmids encoding antigens or cancer immunotherapeutic proteins have been used therapeutically. Functional nucleic acids, such as aptamers, have been used similarly to antibody-based cancer therapies, e.g., by binding and blocking key oncology targets, such as PD-1. Gene editing polynucleotides have also been used to silence expression of cancer mediators. For a review of these various polynucleotide-based cancer therapies. See, for example, Zhou S. et at., Medicine in Drug Discovery, 2020. 6:100023 and Hager et al., Cells. 2020. 9(9):2061, the contents of which are incorporated by reference herein in their entirety.

Although these polynucleotide-based therapies have shown some success in preclinical studies, they have fallen short of expectations in clinical trials when evaluated for therapeutic efficacy. See, for example, Lopes et al., Cancer DNA vaccines: Current preclinical and clinical developments and future perspectives. J. Exp. Clin. Cancer Res. 2019. 38, 146; Dome et al., Therapeutic Cancer Vaccination with ex vivo RNA-Transfected Dendritic Cells-An Update. Pharmaceutics. 2020. 12, 92, the contents of which are incorporated by reference herein in their entirety. One obstacle is that nucleic acids do not readily cross the cell membrane. Furthermore, nucleic acids are readily degraded by extracellular nucleases present in skin, tissues, and blood. Kowalski P S et al., Mol Ther., 27(4):710-28 (2019), the content of which is incorporated by reference herein in its entirety.

The murine anti-DNA antibody 3E10 is known to penetrate cells and at least partially localizes to the nucleus of the cell. See, for example, Weisbart R. H. et al. 1998. J. Autoimmun.; 11:539-546, the contents of which is incorporated by reference herein in its entirety. As such, it has been suggested that 3E10 and derivatives thereof may serve as a targeting agent for the delivery of therapeutic agents in vivo. However, murine antibodies are immunogenic when administered in humans, particularly during chronic administration. Several techniques for reducing the immunogenicity of non-human antibodies by humanization are known in the art. However, not all humanized antibodies retain the advantageous features of their parental non-human antibody.

### SUMMARY

Given the background above, there is a need in the art for humanized 3E10 antibodies and antigen binding fragments thereof. Such humanized 3E10 antibodies would facilitate improved methods for delivering therapeutic cargo, such as therapeutic polynucleotides, polypeptide, and chemical agents into targeted cells. Polynucleotide-based therapies, for example, present a promising path for treating diseases because of their versatility to encode any polypeptide, the availability of highly reproducible manufacturing methods, the ability to make simple and precise adjustments to polynucleotide sequences, their inexpensive nature, their ability to specifically target and/or edit any genetic sequence, etc. However, the delivery of polynucleotide therapeutics to specific tissues in vivo has posed many challenges, including the rapid degradation of foreign nucleic acids in the body and immunogenicity caused by common delivery vehicles, such as liposomes and viral vectors. See, for example, Zhou et al., Medicine in Drug Discovery, 6 (2020) 100023 and Dahlman et al., Nature Nanotechnol. 9(8):648-655 (2014), the contents of which are incorporated by reference herein in their entirety.

Advantageously, the present disclosure provides humanized 3E10 antibodies and antigen binding fragments thereof that retain core 3E10 properties, such as ENT2-based cell penetrating activity and nucleic acid binding activity. Accordingly, as described herein, the present disclosure provides humanized 3E10 antibodies and antigen binding fragments thereof, pharmaceutical compositions, and methods for treating various medical disorders using the same.

In some embodiments, a humanized 3E10 antibody or antigen binding fragment thereof described herein includes a light chain variable domain (3E10-VL) comprising an amino acid sequence that is at least 90% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90).

In some embodiments, the light chain variable domain (3E10-VL) comprises one or more amino acid residues selected from proline (Pro) at position 15, threonine (Thr) at position 22, tyrosine (Tyr) at position 49, Thr at position 74, asparagine (Asn) at position 76, alanine (Ala) at position 80, Asn at position 81, Thr at position 83, Asn at position 85, and valine (Val) at position 104, numbered according to Kabat numbering. In some embodiments, the 3E10-VL includes a set of CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

Similarly, in some embodiments, a humanized 3E10 antibody or antigen binding fragment thereof described herein includes a heavy chain variable domain (3E10-VH (SEQ ID NO:2)) comprising an amino acid sequence that is at least 90% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In some embodiments, the heavy chain variable domain (3E10-VH) comprises one or more amino acid residues selected from glutamine (Gln) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, numbered according to Kabat numbering. In some embodiments, the 3E10-VH includes a set of CDRs collectively having no more than 6 amino acid substitutions relative to the set of CDRs having the amino acid sequences of 3E10-VH-CDR1 D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein comprises a heavy chain CDR1 with an aspartic acid at position 31, numbered according to Kabat numbering.

In some embodiments, a humanized 3E10 antibody or antigen binding fragment thereof described herein comprises a lysine at position 72 in the 3E10-VL, numbered according to Kabat numbering, which supports nucleic acid binding affinity. A mutation in this amino acid residue to tyrosine abrogates high affinity binding to DNA substrate. Furthermore, arginine and lysine at position 37 and 38, respectively, in the 3E10-VH appear to be beneficial for nucleic acid binding. Mutation of these residues to leucine and arginine, respectively, reduces the ability of the antibody to bind DNA substrate at high affinity.

In some embodiments, the present disclosure provides compositions and methods for improved delivery of nucleic acids into cells, e.g., that is not reliant upon a separate delivery vehicle such as a liposome, viral vector, etc. In some embodiments, the present disclosure provides compositions and methods for improved delivery of nucleic acids into cells. In some embodiments, the compositions include (i) a humanized 3E10 antibody or antigen binding fragment thereof with nucleic acid binding activity and (ii) a nucleic acid cargo, for example, a therapeutic polynucleotide, a nucleic acid encoding a polypeptide, a functional nucleic acid, a nucleic acid encoding a functional nucleic acid, or a combination thereof. In some embodiments, elements (i) and (ii) are non-covalently associated to form a complex. In some embodiments, elements (i) and (ii) are covalently associated. In various embodiments, the nucleic acid comprises DNA (single stranded or double stranded), RNA, PNA, or other modified nucleic acids.

In some embodiments, the present disclosure provides humanized 3E10 antibodies or antigen binding fragments thereof covalently conjugated to a therapeutic agent. In some embodiments, the therapeutic agent is a therapeutic nucleic acid. In some embodiments, the therapeutic agent is a chemical agent, in some embodiments, the therapeutic agent is a therapeutic protein or polypeptide. In some embodiments, these conjugates are used in a method for delivering the therapeutic agent into a cell, e.g., without the need for a separate delivery vehicle such as a liposome, viral vector, etc. In some embodiments, the conjugate is a fusion protein in which a polypeptide of the 3E10 antibody or antigen binding fragment thereof and the therapeutic polypeptide are encoded and translated from the same open reading frame.

Methods of delivering a cargo into cells, e.g., a nucleic acid, chemical agent, or therapeutic protein or polypeptide, by contacting the cells with an effective amount of the complexes and complexes described herein are also provided. The contacting can occur in vitro, ex vivo, or in vivo. In some embodiments, an effective amount of ex vivo treated cells is administered to a subject in need thereof, e.g., in an effective amount to treat one or more symptoms of a disease or disorder.

In some embodiments, the contacting occurs in vivo following administration to a subject in need thereof. The subject can have a disease or disorder, such as a genetic disorder or cancer. The compositions can be administered to the subject, for example by injection or infusion, in an effective amount to reduce one or more symptoms of the disease or disorder in the subject.

Applications of the compositions and methods are also provided, and include, but are not limited to, gene therapy and T cell or CAR T cell manufacture, formation, and/or therapy.

### BRIEF DESCRIPTION OF THE DRAWINGS

FIG. 1 (SEQ ID NO:1-12) illustrates amino acid sequences for the parent 3E10 monoclonal antibody.

FIGS. 2A (SEQ ID NO:13-25), 2B, and 2C illustrate amino acid sequences for the D31N variant (FIG. 2A (SEQ ID NO:13-25)), other CDR variants (FIG. 2B), and additionally contemplated CDR variants (FIG. 2C) of the 3E10 monoclonal antibody, in accordance with some embodiments of the present disclosure.

FIG. 3 illustrates example charge-conserved CDR variants of the 3E10 monoclonal antibody, in accordance with various embodiments of the present disclosure.

FIG. 4 illustrates example CDR variants containing a combination of amino acid substitutions, charged-conserved amino acid substitutions, and rationally designed amino acid substitutions of the 3E10 monoclonal antibody, in accordance with various embodiments of the present disclosure.

FIG. 5 illustrates amino acid sequences of humanized 3E10 variable heavy (3E10-VH) domains, in accordance with various embodiments of the present disclosure.

FIG. 6 illustrates amino acid sequences of mature humanized 3E10 heavy chains (3E10-HC), lacking a signal peptide, in accordance with various embodiments of the present disclosure.

FIG. 7 illustrates amino acid sequences of humanized 3E10 heavy chains (3E10-HC), in accordance with various embodiments of the present disclosure.

FIG. 8 illustrates amino acid sequences of humanized 3E10 variable light (3E10-VL) domains, in accordance with various embodiments of the present disclosure.

FIG. 9 illustrates amino acid sequences of mature humanized 3E10 light chains (3E10-LC), lacking a signal peptide, in accordance with various embodiments of the present disclosure.

FIG. 10 illustrates amino acid sequences of humanized 3E10 light chains (3E10-LC), in accordance with various embodiments of the present disclosure.

FIGS. 11A and 11B illustrate electrostatic surface potential renderings of a molecular model of a 3E10-scFv construct, revealing a putative Nucleic Acid Binding pocket (NAB1). FIG. 11A additionally shows predicted structural and electrostatic potential changes induced by amino acid substitutions at residue HC CDR1 residue 31. FIG. 11B is an illustration of molecular modeling of 3E10-scFv (Pymol) with NAB1 amino acid residues highlighted by punctate dots.

FIGS. 12A, 12B, 12C, 12D, and 12E collectively show results of nucleic acid binding characterization of various humanized 3E10 constructs, as described in Example 1, in accordance with some implementations of the present disclosure.

FIG. 13 illustrate histograms collectively showing a 4-day time course of the type-1 IFN response in THP-1 monocytes PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in Example 3.

FIG. 14 illustrate histograms collectively showing a 4-day time course of the type-1 IFN response in THP-1 monocytes PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in Example 3.

FIG. 15 illustrate histograms collectively showing a 4-day time course of the type-1 IFN response in THP-1 monocytes PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in Example 4.

FIG. 16 illustrate histograms collectively showing a 4-day time course of the type-1 IFN response in THP-1 monocytes PBS (control), the 3p-hpRNA RIG-I agonist alone (1 ug/well), increasing amounts of humanized 3E10 antibody alone, and non-humanized 3E10 antibody/3p-hpRNA (1 ug 3p-hpRNA/well), as indicated in Example 3.

FIG. 17 illustrate histograms collectively showing humanized 3E10 antibody uptake (+/−) dipyridamole, in tumor, liver, kidney, spleen, quadriceps, and gastrocnemius in tumor bearing mice (CT-26 colorectal cancer model).

FIG. 18 illustrates the experimental schema for measuring the biodistribution of 3E10-D31N IgG4 Variants in a Pancreatic ductal adenocarcinoma (PDAC) murine model.

FIGS. 19A, 19B, 19C, 19D, and 19E illustrates binding kinetics and affinity measurements for 3E10-D31N and 3E10-D31N IgG4 Fc variants, as indicated in Example 8.

FIG. 20A illustrates chimeric 3E10-D31N delivery of GFP mRNA in an MDA-MB-231 murine model.

FIG. 20B illustrates a comparability study testing chimeric 3E10-D31N delivery of GFP mRNA and humanized 3E10 antibody construct (V66) delivery of GFP mRNA in a KPC syngeneic tumor model.

FIG. 21 illustrates tumor and normal tissue expression for targeted functional delivery of GFP mRNA payload.

FIG. 22 illustrates comparability of chimeric 3E10 D31N and humanized 3E10 antibodies (V66) in a B16 tumor model measuring tumor volumes days post-implantation of antibody:3p-hpRNA complex.

FIGS. 23A, 23B, 23C, 23D, 23E, 23F, 23G, 23H, 23I, 23J, 23K, 23L, 23M, 23N, 230, 23P, and 23Q collectively illustrate serum and tissue pharmacokinetic profiles of a humanized 3E10(D31N) antibody V66, as indicated in each figure, from a single dose, dose escalation, pharmacokinetic study performed in C57Bl/6 mice, as described in Example 12.

FIGS. 24A, 24B, 24C, and 24D collectively provide summary analysis and statistics for the serum and tissue pharmacokinetic study of the humanized 3E10(D31) antibody V66, as described in Example 12.

### DETAILED DESCRIPTION

In various aspects and embodiments, the present disclosure provides humanized 3E10 antibodies and antigen binding fragments thereof, as well as methods for delivering cargo, e.g., polynucleotides, polypeptides, or chemical agents, into cells using the same. The humanized 3E10 antibodies and antigen binding fragments thereof described herein can penetrate cells, assisting in the delivery of cargo, e.g., polynucleotides, polypeptides, or chemical agents, across the plasma membrane and into cell cytoplasm and/or nuclei without the need for a separate delivery vehicle. Advantageously, because the 3E10 antibodies and antigen binding fragments described herein have been humanized, they will be less immunogenic when administered to humans as compared to the murine and chimeric 3E10 parental antibodies. Further, the humanized 3E10 antibodies and antigen binding fragments described herein retain key 3E10 properties, such as sequence non-specific nucleic acid binding and antigen mediated cellular penetration. Moreover, as reported in the Examples, various combinations of the humanized 3E10 variable heavy and variable light domains have different nucleic acid binding affinities, allowing for better control of nucleic acid binding and release in vivo.

### Definitions

The terminology used in the present disclosure is for the purpose of describing particular embodiments only and is not intended to be limiting of the invention. As used in the description of the invention and the attached claims, the singular forms “a”, “an” and “the” are intended to include the plural forms as well, unless the context clearly indicates otherwise. It will also be understood that the term “and/or” as used herein refers to and encompasses any and all possible combinations of one or more of the associated listed items. Unless the context requires otherwise, it will be further understood that the terms “includes,” “comprising,” or any variation thereof, when used in this specification, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%.

The term “and/or” as used herein refers to and encompasses any and all possible combinations of one or more of the associated listed items.

Unless the context requires otherwise, the terms “includes,” “comprising,” or any variations thereof, specify the presence of stated features, integers, steps, operations, elements, and/or components, but do not preclude the presence or addition of one or more other features, integers, steps, operations, elements, components, and/or groups thereof. Furthermore, to the extent that the terms “including,” “includes,” “having,” “has,” “with,” or variants thereof are used in either the detailed description and/or the claims, such terms are intended to be inclusive in a manner similar to the term “comprising.”

Recitation of ranges of values herein are merely intended to serve as a shorthand method of referring individually to each separate value falling within the range, unless otherwise indicated herein, and each separate value is incorporated into the specification as if it were individually recited herein.

As used herein, the term “subject” means any individual who is the target of administration. The subject can be a vertebrate, for example, a mammal. Thus, the subject can be a human. The term does not denote a particular age or sex.

As used herein, the term “pharmaceutically effective amount” means that the amount of the composition used is of sufficient quantity to ameliorate one or more causes or symptoms of a disease or disorder. Such amelioration only requires a reduction or alteration, not necessarily elimination. The precise dosage will vary according to a variety of factors such as subject-dependent variables (e.g., age, immune system health, etc.), the disease or disorder being treated, as well as the route of administration and the pharmacokinetics of the agent being administered.

As used herein, the term “carrier” or “excipient” refers to an organic or inorganic ingredient, natural or synthetic inactive ingredient in a formulation, with which one or more active ingredients are combined. The carrier or excipient would naturally be selected to minimize degradation of the active ingredient or to minimize adverse side effects in the subject, as would be well known to one of skill in the art.

As used herein, the term “treat” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. As used herein, the term “inhibit” or “reduce” means to decrease an activity, response, condition, disease, or other biological parameter. This can include, but is not limited to, the complete ablation of the activity, response, condition, or disease. This may also include, for example, a statistically significant reduction in the activity, response, condition, or disease as compared to the native or control level.

In the present disclosure, the term “CNS cancer” or “cancer of the central nervous system” refers to abnormal growth of cells from any tissue of the central nervous system, including the brain, spinal cord, meninges, or hematopoietic tissue of the primary CNS of a subject. Non-limited examples of CNS cancers include neuroepithelial cancers (such as gliomas, mature neuron cancers, primitive neuroectodermal tumors, and primitive brain cancers), meningeal cancers, and primary central nervous system hematopoietic cancers.

### Antibodies and Variants Thereof

As used herein, an “antigen binding domain” or “ABD” refers to a set of six Complementary Determining Regions (CDRs) that, when present as part of a polypeptide sequence or sequences, specifically binds a target antigen as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 for the heavy chain and vlCDR1, vlCDR2 and vlCDR3 for the light. The CDRs are present in the variable heavy and variable light domains, respectively, and together form an Fv region. Various CDR numbering schemes are known, as outlined in the table and related discussion below. Thus, in some cases, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, including optional domain linkers on each side, depending on the format used. In general, the C-terminus of the scFv domain is attached to the N-terminus of the hinge in the second monomer.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 2003. 27(1):55-77.

Comparisons of CDR numbering

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

For all amino acid positions discussed in the disclosure relating to antibodies, the amino acid position numbering is according to the EU index. The EU index or EU index as in Kabat or EU numbering scheme refers to the numbering of the EU antibody. Kabat et al. collected numerous primary sequences of the variable regions of heavy chains and light chains. Based on the degree of conservation of the sequences, they classified individual primary sequences into the CDR and the framework and made a list thereof. See, SEQUENCES OF IMMUNOLOGICAL INTEREST, 5th edition, NIH publication, No. 91-3242, E. A. Kabat et al.; Edelman et al., 1969, Proc Natl Acad Sci USA 63:78-85, the contents of which are incorporated by reference herein in their entirety. The modification can be an addition, deletion, or substitution.

As used herein, the terms “antibody variant” or “variant antibody” refer to an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2, IgG3, or IgG4, as further described herein.

In some embodiments, a parent polypeptide, e.g., an Fc parent polypeptide, is a human wild type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105, the contents of which are incorporated by reference herein in their entirety, can be employed. The protein variant sequence herein will preferably possess at least about 75% identity with a parent protein sequence, or at least about 80% identity with a parent protein sequence, and most preferably at least about 90% identity, more preferably at least about 95%, or at least about 98%, or at least about 99% sequence identity.

In some embodiments, the protein variant sequence herein has at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% sequence identity with a parent protein sequence.

As used herein, an “isotype” refers to any of the subclasses of immunoglobulins defined by the chemical and antigenic characteristics of their constant regions. It should be understood that therapeutic antibodies can also comprise hybrids of isotypes and/or subclasses.

As used herein, a “Fab” or “Fab region” refers to a polypeptide that comprises the VH, CH1, VL, and CL immunoglobulin domains, generally on two different polypeptide chains (e.g., VH-CH1 on one chain and VL-CL on the other). Fab may refer to this region in isolation, or this region in the context of an antibody of the disclosure. In the context of a Fab, the Fab comprises an Fv region in addition to the CH1 and CL domains.

As used herein, an “Fv” or “Fv fragment” or “Fv region” refers to a polypeptide that comprises the VL and VH domains of an ABD. Fv regions can be formatted as both Fabs (as discussed above, generally two different polypeptides that also include the constant regions as outlined above) and scFvs, where the v and vh domains are combined (generally with a linker as discussed herein) to form an scFv.

As used herein, a “single chain Fv” or “scFv” refers to a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the present disclosure, particularly outlined in the figures, the order of the vh and vl domain is indicated in the name, e.g., H.X_L.Y which, from N- to C-terminal, is vh-linker-vl, and L.Y_H.X is vl-linker-vh.

As used herein, an “Fc” or “Fc region” or “Fc domain” refers to a polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.). Human IgG Fc domains are of particular use in the present disclosure, and can be the Fc domain from human IgG1, IgG2 or IgG4.

As used herein, a “variant Fc domain” contains amino acid modifications as compared to a parental Fc domain. Thus, a “variant human IgG1 Fc domain” is one that contains amino acid modifications (generally amino acid substitutions, although in the case of ablation variants, amino acid deletions are included) as compared to the human IgG1 Fc domain. In general, variant Fc domains have at least about 80, about 85, about 90, about 95, about 97, about 98 or about 99 percent identity to the corresponding parental human IgG Fc domain (using the identity algorithms discussed below, with one embodiment utilizing the BLAST algorithm as is known in the art, using default parameters). Alternatively, the variant Fc domains can have from 1 to about 20 (e.g., 1, 2, 3, 4, 5, 6, 7, 8, 9, 10, 11, 12, 13, 14, 15, 16, 17, 18, 19 or 20) amino acid modifications as compared to the parental Fc domain. Additionally, as discussed herein, the variant Fc domains herein still retain the ability to form a dimer with another Fc domain as measured using known techniques as described herein, such as non-denaturing gel electrophoresis.

As used herein, the term “heavy chain constant region” refers to the CH1-hinge-CH2-CH3 portion of an antibody (or fragments thereof), excluding the variable heavy domain; in EU numbering of human IgG1 this is amino acids 118-447 By “heavy chain constant region fragment” herein is meant a heavy chain constant region that contains fewer amino acids from either or both of the N- and C-termini but still retains the ability to form a dimer with another heavy chain constant region.

As used herein, the terms “variable region” or “variable domain” refer to the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

As used herein, the term “full-length heavy chain” refers to the entire heavy chain of an antibody, inclusive of the signal peptide (S), having the structure S-VH-CH1-hinge-CH2-CH3.

As used herein, the term “mature heavy chain” refers to the portion of the heavy chain of the antibody that excludes the signal peptide, having the structure VH-CH1-hinge-CH2-CH3.

As used herein, the term “full-length light chain” refers to the entire light chain of an antibody, inclusive of the signal peptide (S), having the structure S-VL-CL.

As used herein, the term “mature light chain” refers to the portion of the light chain of the antibody that excludes the signal peptide, having the structure S-VL-CL.

As used herein, the terms “IgG subclass modification” or “isotype modification” refers to an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

As used herein, the term “non-naturally occurring modification” refers to an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

The antibodies and antigen-binding fragments thereof of the disclosure are recombinant antibodies that have been engineered to have the various properties described herein and are generally isolated prior to use. As used herein, the term “isolated”, when used to describe the various polypeptides described herein, refers to a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogenous host cells, and they can be isolated as well.

As used herein, a “3E10 antibody” refers to an antibody with a set of heavy chain CDRs (VH CDR1, VH CDR2, and VH CDR3), identified according to the Kabat system, comprising amino acid sequences that vary from SEQ ID NOS: 58, 59, and 60 by no more than two amino acids each, respectively, a set of light chain CDRs (VL CDR1, VL CDR2, and VL CRD3) comprising amino acid sequences that vary from SEQ ID NOS: 61, 62, and 63 by no more than two amino acids each, respectively, and that binds nucleic acids. As described herein, the 3E10 antigen is a polynucleotide.

As used herein, the term “cell-penetrating” refers to an antibody or antigen binding fragment thereof that can penetrate a cell, e.g., a mammalian cell, without the aid of an exogeneous transport vehicle, such as a liposome, or a conjugated cell-penetrating peptide. With respect to 3E10 antibodies and antigen binding fragments thereof, the cell-penetrating antibody or antigen binding fragment thereof can penetrate a cell expressing an ENT2 receptor on its cell surface in the presence of nucleic acids, e.g., non-covalently bound and/or conjugated to the 3E10 antibody or antigen binding fragment thereof, resulting in internalization of the 3E10 antibodies and antigen binding fragments thereof. In some embodiments, the cell-penetrating 3E10 antibody or antigen binding fragment thereof is conjugated to a functional molecule, e.g., a chemical agent, polynucleotide, or polypeptide.

By “variant protein” or “protein variant” or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 99.5% identical to the parent protein, using the alignment programs described below, such as BLAST. Although amino acid sequence modifications to a 3E10 antibody or antigen binding fragment thereof as described herein may produce a protein and/or polypeptide that is referred to as a variant 3E10 antibody or antigen binding fragment thereof, such variants still fall within the classification of a 3E10 antibody or antigen binding fragment thereof as long as they maintain the CDR sequence and cell penetrating activity requirements of a 3E10 antibody or antigen binding fragment thereof.

Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol., 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see the webpage located at URL blast.ncbi.nlm.nih.gov/Blast.cgi. When using any of the aforementioned algorithms, the default parameters (for Window length, gap penalty, etc.) are used. Unless specifically stated otherwise, sequence identity is determined using the BLAST algorithm, using default parameters

### 3E10 Antibodies, Variants, and Fragments Thereof

In embodiments, the present disclosure relates to humanized 3E10 antibodies and antigen binding fragments thereof and use of the same for delivering therapeutic agents for the treatment of various diseases. As is discussed herein, humanized 3E10 antibodies that find use in the methods and compositions of the disclosure can incorporate an array of amino acid substitutions in any portion of the antibody. Further, the antigen binding fragments of the humanized 3E10 antibodies can take on many forms including, but not limited to, the example formats described herein.

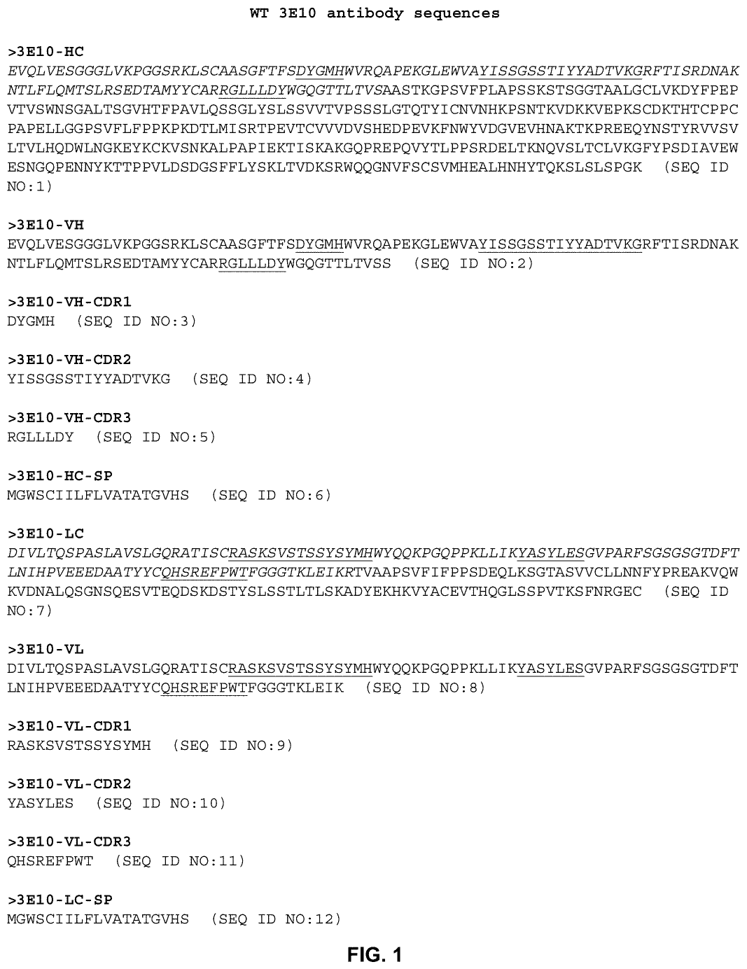

Amino acid sequences of 3E10 monoclonal antibodies and binding fragments thereof are known in the art. Example sequences of 3E10 heavy and light chains are provided below and shown in various figures. Where present, single underlining indicates CDR regions identified according to the Kabat system, italics indicates the variable domains, and double underlining indicates the signal peptide. The murine version of the 3E10 antibody is described in Zack, et al., Immunology and Cell Biology, 72:513-520 (1994). The heavy and light chains of the murine 3E10, as well as the variable regions and CDRs are shown in FIG. 1 (SEQ ID NO:1-12).

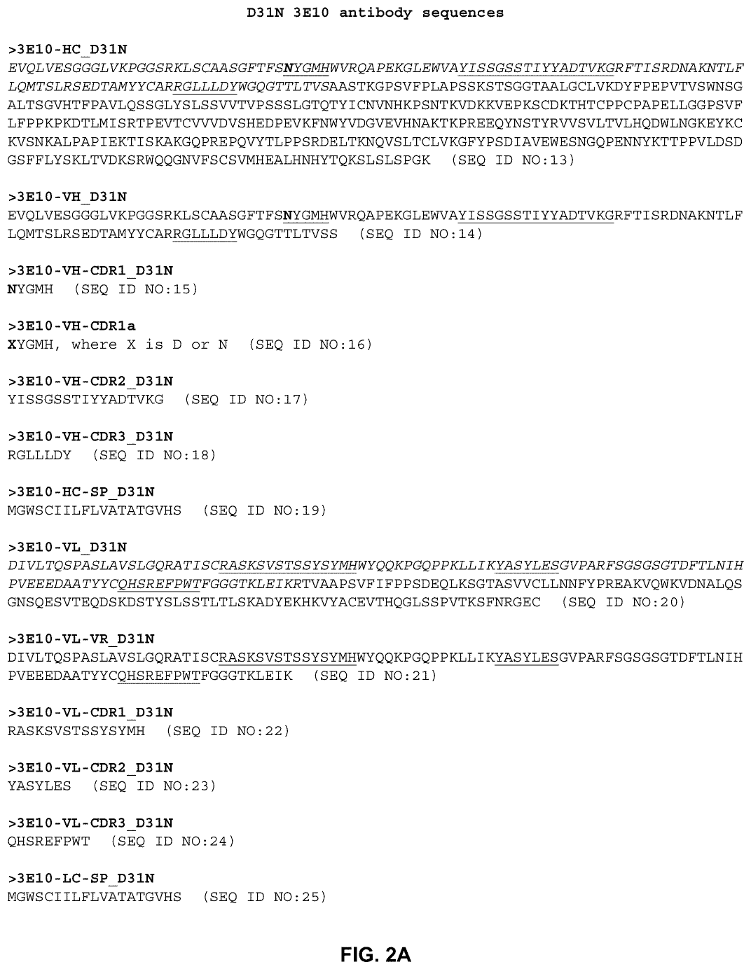

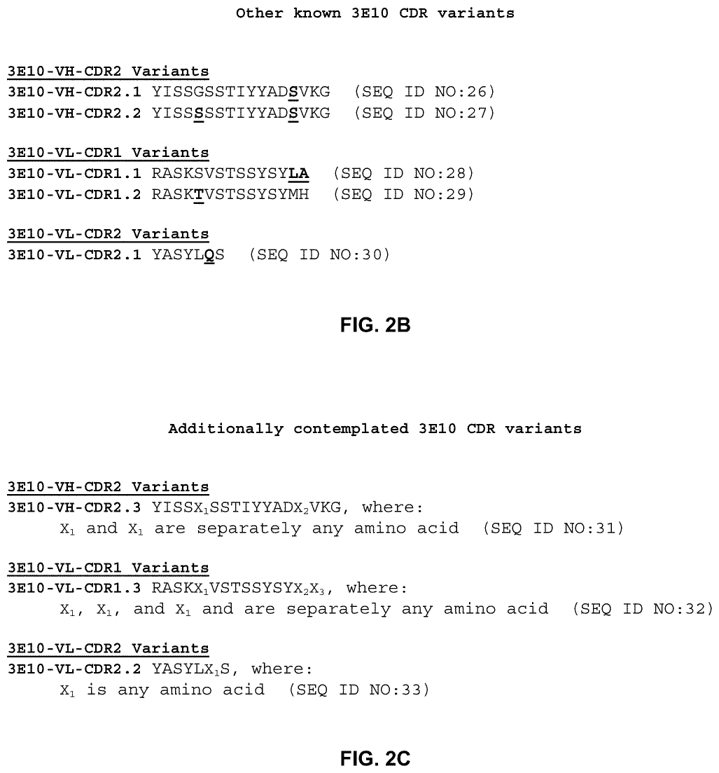

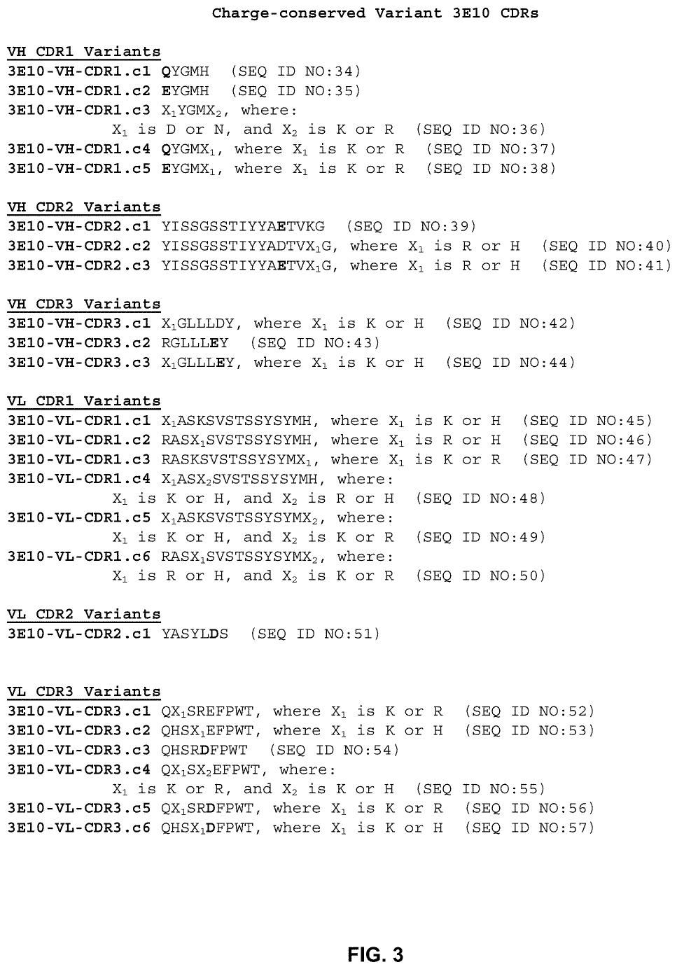

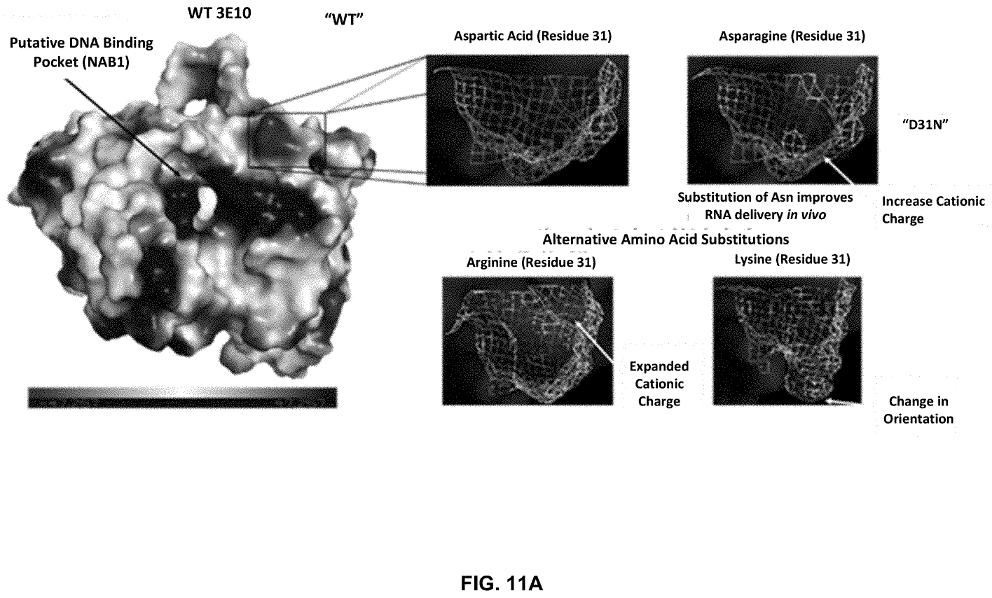

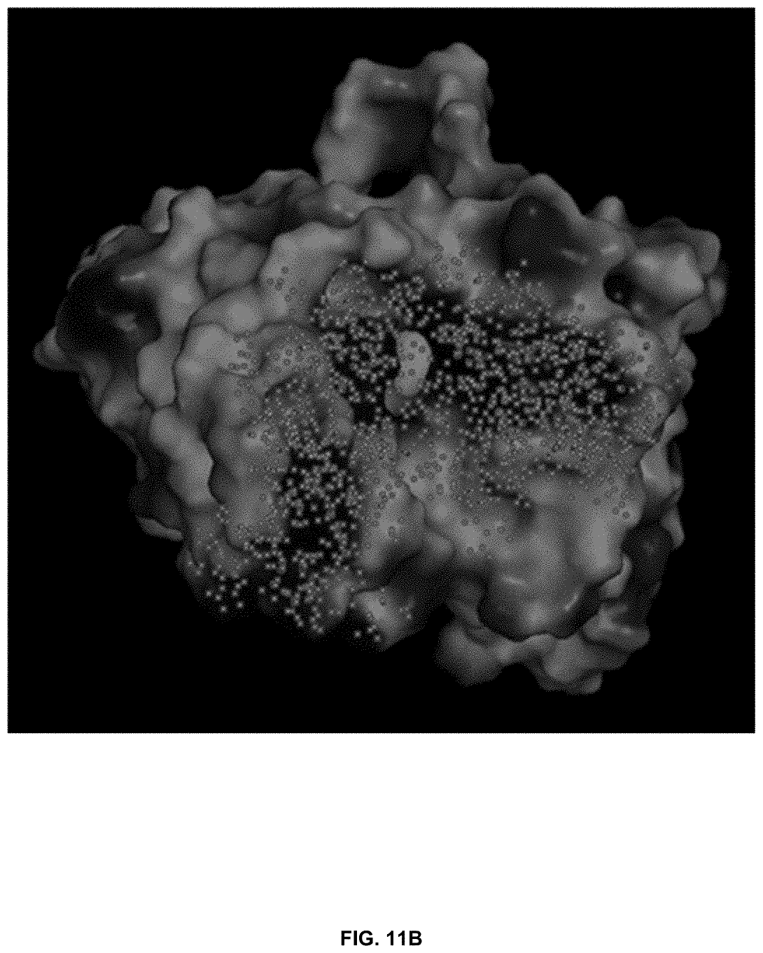

Amino acid variants of the 3E10 antibody are also known in the art, for example, as described in Zack, et al., J. Immunol., 157(5):2082-8 (1996). For example, amino acid position 31, in CDR1 of the heavy chain variable region of 3E10, influences nucleic acid binding and the antibody's ability to penetrate nuclei. Substitution of the ‘wild type’ (e.g., relative to the original murine antibody) aspartic acid by asparagine (the ‘D31N’ mutation) improves nucleic acid binding and nuclei penetration of the antibody, relative to the ‘wild type’ murine antibody. See, for example, Zack, et al., Immunology and Cell Biology, 72:513-520 (1994); Weisbart, et al., J. Autoimmun., 11, 539-546 (1998); and Weisbart, Int. J. Oncol., 25, 1867-1873 (2004). Sequences for the murine 3E10 with the D31N substitution as shown in FIG. 2. Accordingly, in some embodiments, the humanized 3E10 antibodies and binding fragments thereof disclosed herein include the D31N substitution. In other embodiments, other amino acids are substituted at position 31 in the humanized 3E10 antibodies and binding fragments thereof disclosed herein. For example, as modeled in FIG. 11A, D31R or D31K substitutions are incorporated in some embodiments of the present disclosure.

Other 3E10 light chain sequences are known in the art. See, for example, Zack, et al., J. Immunol., 15; 154(4):1987-94 (1995); GenBank: L16981.1—Mouse Ig rearranged L-chain gene, partial cds; GenBank: AAA65681.1—immunoglobulin light chain, partial [Mus musculus])

Traditional antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Human light chains are classified as kappa and lambda light chains. The present disclosure is directed to antibodies that generally are based on the IgG class, which has several subclasses, including, but not limited to IgG1, IgG2, IgG3, and IgG4. In general, IgG1, IgG2 and IgG4 are used more frequently than IgG3. It should be noted that IgG1 has different allotypes with polymorphisms at 356 (D or E) and 358 (L or M).

The light chain generally comprises two domains, the variable light domain (containing the light chain CDRs and together with the variable heavy domains forming the Fv region), and a constant light chain region (often referred to as CL or Ck). The heavy chain comprises a variable heavy domain and a constant domain, which includes a CHI-optional hinge-Fc domain comprising a CH2-CH3.

The hypervariable region of an antibody generally encompasses amino acid residues from about amino acid residues 24-34 (LCDR1; “L” denotes light chain), 50-56 (LCDR2) and 89-97 (LCDR3) in the light chain variable region and around about 31-35B (HCDR1; “H” denotes heavy chain), 50-65 (HCDR2), and 95-102 (HCDR3) in the heavy chain variable region; Kabat et al., SEQUENCES OF PROTEINS OF IMMUNOLOGICAL INTEREST, 5th Ed. Public Health Service, National Institutes of Health, Bethesda, Md. (1991) and/or those residues forming a hypervariable loop (e.g. residues 26-32 (LCDR1), 50-52 (LCDR2) and 91-96 (LCDR3) in the light chain variable region and 26-32 (HCDR1), 53-55 (HCDR2) and 96-101 (HCDR3) in the heavy chain variable region; Chothia and Lesk J. Mol. Biol. 1987. 196:901-917. Specific CDRs useful for the compositions and methods described herein are described below.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is described in Lafranc et al., Dev. Comp. Immunol. 2003. 27(1):55-77 (2003).

Throughout the present disclosure, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The present disclosure provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences. The CDRs contribute to the formation of the antigen-binding, or more specifically, epitope binding site of antibodies. “Epitope” refers to a determinant that interacts with a specific antigen binding site in the variable region of an antibody molecule known as a paratope.

Epitopes are groupings of molecules such as nucleic acids, amino acids, or sugar side chains and usually have specific structural characteristics, as well as specific charge characteristics. A single antigen may have more than one epitope. The antibodies described herein bind to nucleic acid epitopes in a partially sequence-independent manner. That is, while the antibodies described herein bind to some polynucleotide structures and sequences with greater affinity than other nucleic acid structures and sequences, they have some general affinity for polynucleotides.

The “Fc domain” of the heavy chain includes the —CH2-CH3 domain, and optionally a hinge domain (—H-CH2-CH3). For IgG, the Fc domain comprises immunoglobulin domains CH2 and CH3 (Cy2 and Cy3) and the lower hinge region between CHI (Cy1) and CH2 (Cy2). Although the boundaries of the Fc region may vary, the human IgG heavy chain Fc region is usually defined to include residues C226 or P230 to its carboxyl-terminus, wherein the numbering is according to the EU index as in Kabat. Accordingly, “CH” domains in the context of IgG are as follows: “CHI” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. Thus, the “Fc domain” includes the —CH2-CH3 domain, and optionally a hinge domain (hinge-CH2-CH3). In the embodiments herein, when a scFv is attached to an Fc domain, it is generally the C-terminus of the scFv construct that is attached to all or part of the hinge of the Fc domain; for example, it is generally attached to the sequence EPKS which is the beginning of the hinge. In some embodiments, as is more fully described below, amino acid modifications are made to the Fc region, for example to alter binding to one or more FcyR receptors or to the FcRn receptor, and to enable heterodimer formation and purification, as outlined herein.

Another part of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CHI domain ends at EU position 215, and the IgG CH2 domain begins at residue EU position 231. Thus, for IgG the antibody hinge is herein defined to include positions 216 (E216 in IgG1) to 230 (p230 in IgG1), wherein the numbering is according to the EU index as in Kabat. In some cases, a “hinge fragment” is used, which contains fewer amino acids at either or both of the N- and C-termini of the hinge domain.

An scFv comprises a variable heavy chain, an scFv linker, and a variable light domain. In most of the constructs and sequences outlined herein, the C-terminus of the variable heavy chain is attached to the N-terminus of the scFv linker, the C-terminus of which is attached to the N-terminus of a variable light chain (N-vh-linker-vl-C) although that can be switched (N-vl-linker-vh-C).

Thus, the present disclosure relates to different antibody domains. As described herein and known in the art, the heterodimeric antibodies described in certain embodiments of the disclosure comprise different domains within the heavy and light chains, which can be overlapping as well. These domains include, but are not limited to, the Fc domain, the CHI domain, the CH2 domain, the CH3 domain, the hinge domain, the heavy constant domain (CH1-hinge-Fc domain or CH-hinge-CH2-CH3), the variable heavy domain, the variable light domain, the light constant domain, Fab domains and scFv domains.

In certain embodiments, the antibodies of the disclosure comprise a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence, e.g., that of the 3E10 antibody. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene. In certain cases, the humanized antibody may display no more than 5, or even no more than 4, 3, 2, or 1 amino acid difference from the amino acid sequence encoded by the germline immunoglobulin gene.

In one embodiment, the parent antibody has been affinity matured, as is known in the art. Structure-based methods may be employed for humanization and affinity maturation, for example as described in U.S. application Ser. No. 11/004,590, the contents of which are incorporated by reference herein in their entirety. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16): 10678-10684; Rosok et al., 1996, J. Biol. Chem. 271 (37): 22611-22618; Rader et al., 1998, Proc. Natl. Acad. Sci. USA 95: 8910-8915; Krauss et al., 2003, Protein Engineering 16(10):753-759, all of which are incorporated herein by reference. Other humanization methods may involve the grafting of only parts of the CDRs, including but not limited to methods described in U.S. application Ser. No. 09/810,510; Tan et al., 2002, J. Immunol. 169:1119-1125; De Pascalis et al., 2002, J. Immunol. 169:3076-3084, the contents of which are incorporated by reference herein in their entirety.

In some embodiments, the present disclosure relates to the use of antigen binding domains (ABDs) that bind to nucleic acids, and specifically that bind to therapeutic polynucleotides used to a disease, e.g., cancer, derived from a 3E10 antibody. The amino acid sequence of the heavy and light chains of the parent 3E10 antibody are shown in FIG. 1 (SEQ ID NO:1-12). Accordingly, in some embodiments, the compositions described herein include a 3E10 antibody or antigen-binding fragment thereof.

As used herein an “antigen binding fragment” of a humanized 3E10 antibody include, but are not limited to, fragments, variants, and fusion proteins, such as scFv, di-scFv, tr-scFv, and other single chain variable fragments, with nucleic acid binding properties.

A humanized 3E10 antibody or antigen binding fragment thereof is capable of being transported into the cytoplasm and/or nucleus of the cells without the aid of a carrier or conjugate. For example, the monoclonal antibody 3E10 and active fragments thereof that are transported in vivo to the nucleus of mammalian cells without cytotoxic effect are disclosed in U.S. Pat. Nos. 4,812,397 and 7,189,396 to Richard Weisbart.

In some embodiments, a humanized 3E10 antibody or antigen binding fragment thereof binds and/or inhibits Rad51. See, e.g., Turchick, et al., Nucleic Acids Res., 45(20): 11782-11799 (2017), WO 2020/047344, and WO 2020/047353, each of which is specifically incorporated by reference herein, in its entirety.

Humanized 3E10 antibodies and antigen binding fragments thereof that can be used in the compositions and methods include whole immunoglobulin (e.g., an intact antibody) of any class, fragments thereof, and synthetic proteins containing at least nucleic acid binding and ENT2 mediated cell internalization that are the hallmark of a 3E10 antibody. Antigen-binding activity is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in close proximity by the FR regions and, with the CDRs from the other chain, contribute to the formation of the antigen binding site of antibodies.

### Humanized 3E10 Antibodies and Antigen Binding Fragments Thereof

Generally, a humanized antibody is the result of a process in which the sequence of a parental antibody from a non-human species is modified to increase the overall similarity of the parental antibody to human antibodies, while retaining antigen binding activity of the parental antibody. Generally, the process involves identifying a human antibody, sometimes referred to as a scaffold antibody, and then either (i) replacing amino acids in the parent (non-human) antibody with equivalent amino acids from the scaffold (human) antibody, e.g., framework amino acids having little to no effect on antigen binding or (ii) replacing amino acids in the scaffold (human) antibody with equivalent amino acids from the parent (non-human) antibody, e.g., CDRs and other amino acids with significant effects on antigen binding. Various methods for humanization are known in the art, including framework-homology-based humanization, germline humanization, complementary determining regions (CDR)-homology-based humanization, and specificity determining residues (SDR) grafting. For a review of these methods see, for example, Safdari Y. et al., Biotechnology and Genetic Engineering Reviews, 29:2, 175-86 (2013).

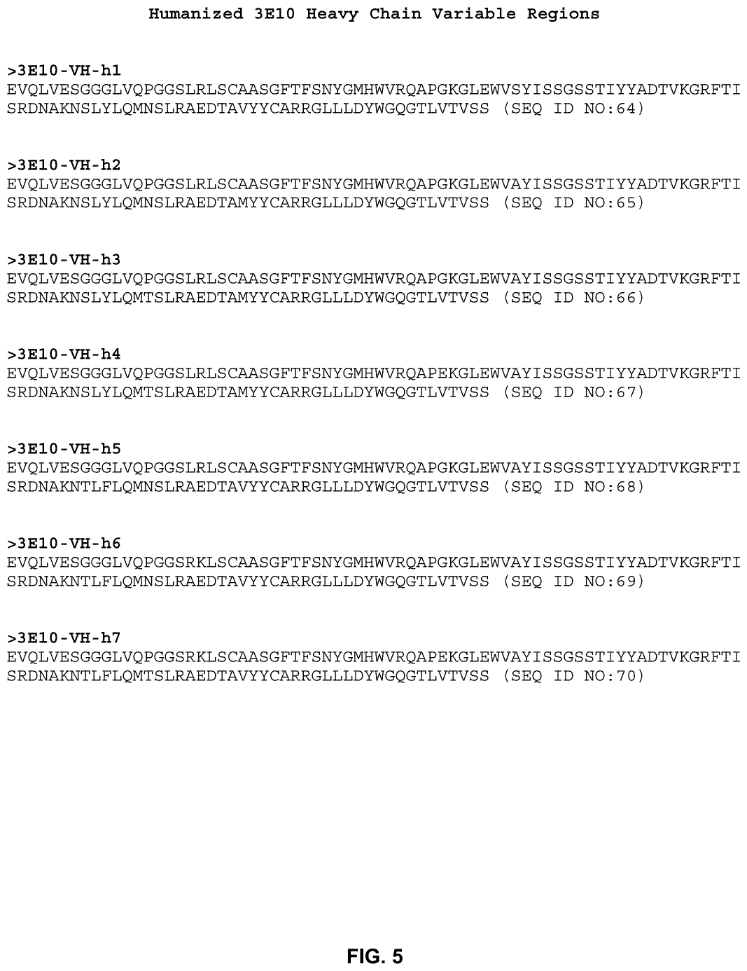

As described in the Examples, seven humanized 3E10 variable light domains and six humanized 3E10 variable heavy domains were generated, the sequences of which are shown in FIGS. 5 (heavy chain variable regions), 6 (heavy chain without signal sequence), 7 (heavy chain with signal peptide), 8 (light chain variable regions), 9 (light chain without signal sequence), and 10 (light chain with signal peptide). These variable light and variable heavy domains can be combined in any of the possible 42 combinations (each of the seven variable light domains with each of the variable heavy domains) to form humanized 3E10 antibodies and nucleic acid binding fragments (e.g., scFvs) thereof. As described in the Examples, 22 antibodies incorporating different combinations of these humanized VL and VH sequences were made, all of which bound nucleic acids. Further, when human leukemia cells were exposed to complexes formed between these antibodies and a RIG-I agonist polynucleotide, all of the complexes were able to generate a Type I IFN response, suggesting that all of the tested antibodies were able to deliver functional polynucleotides into the cells and affect a RIG-I mediated response.

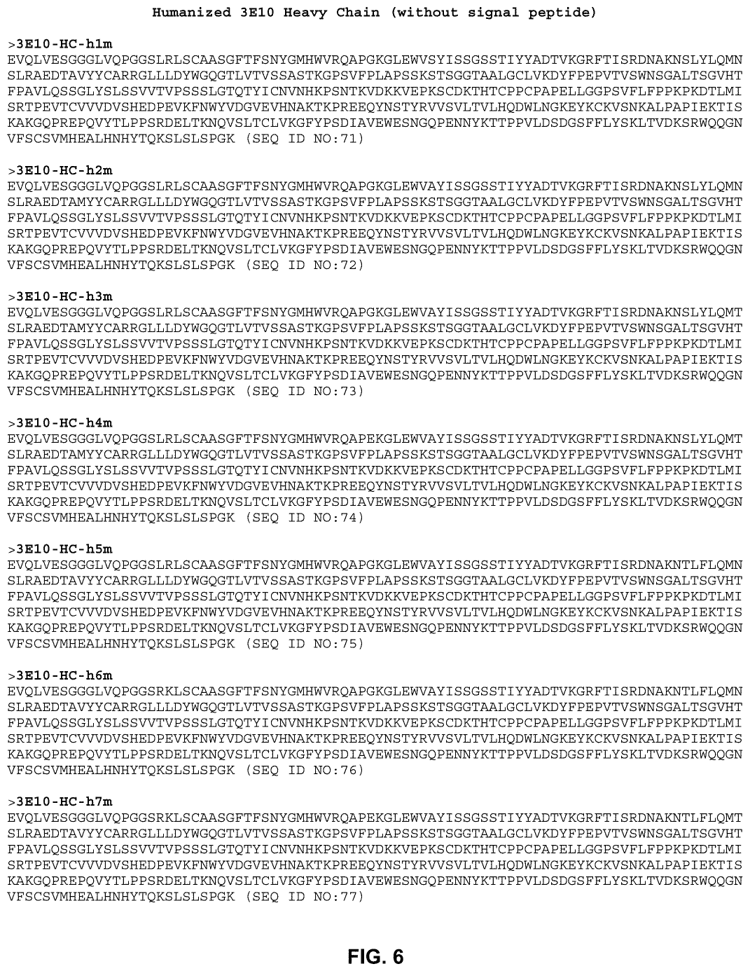

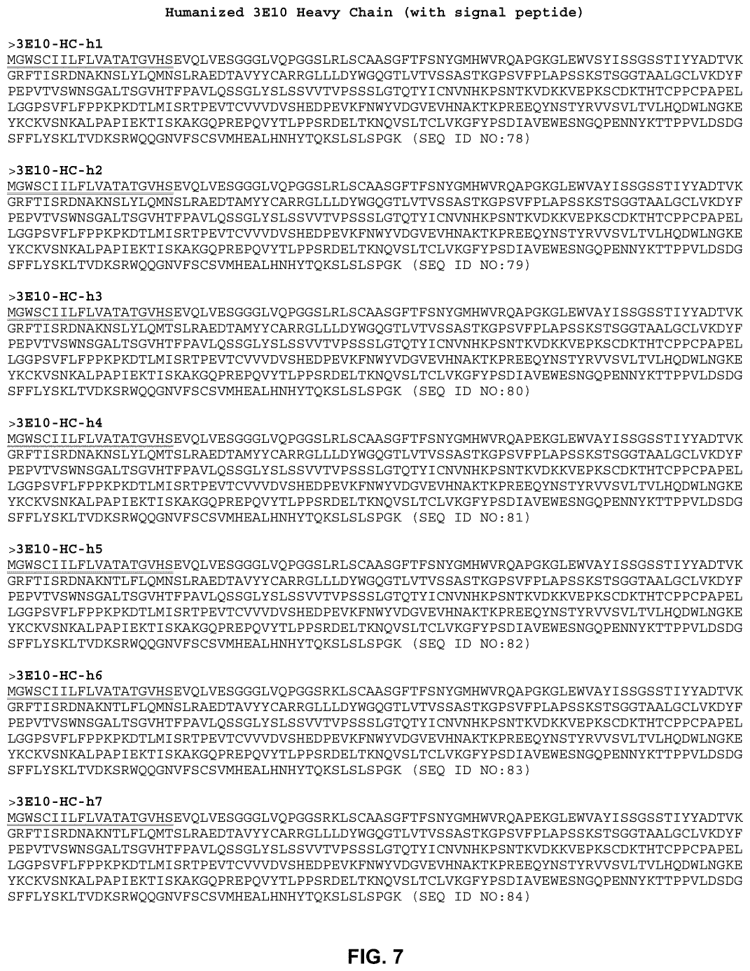

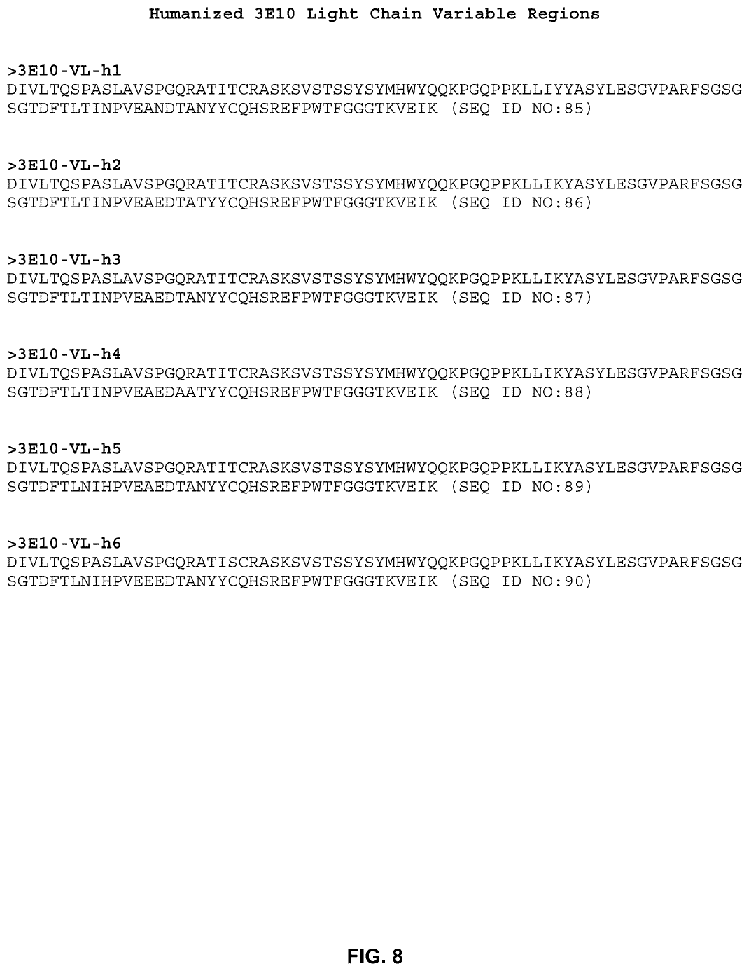

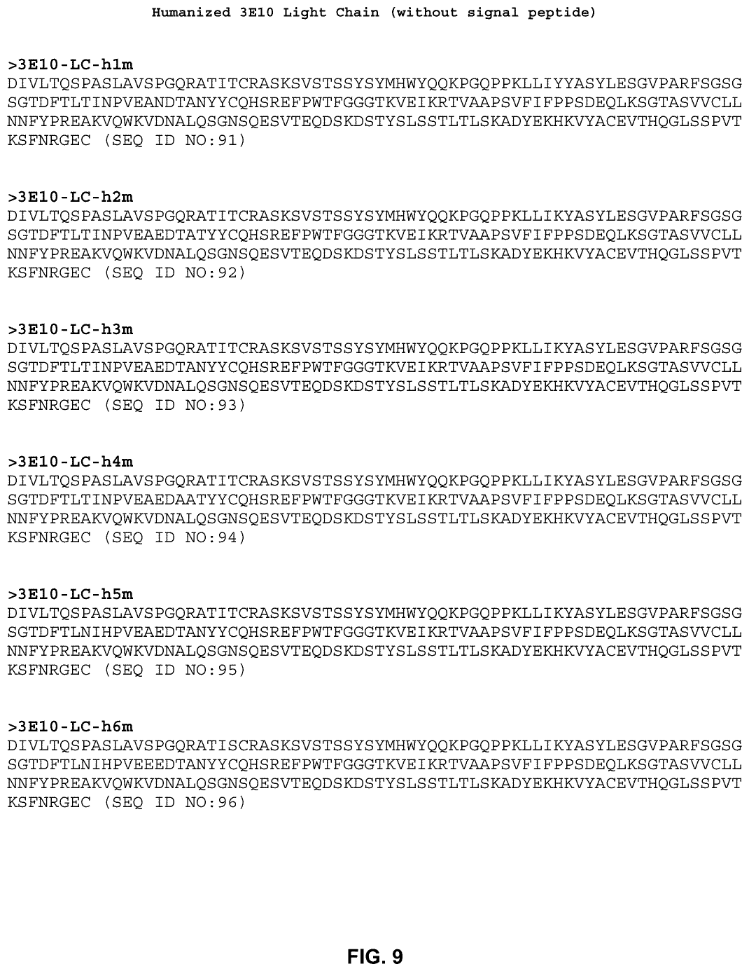

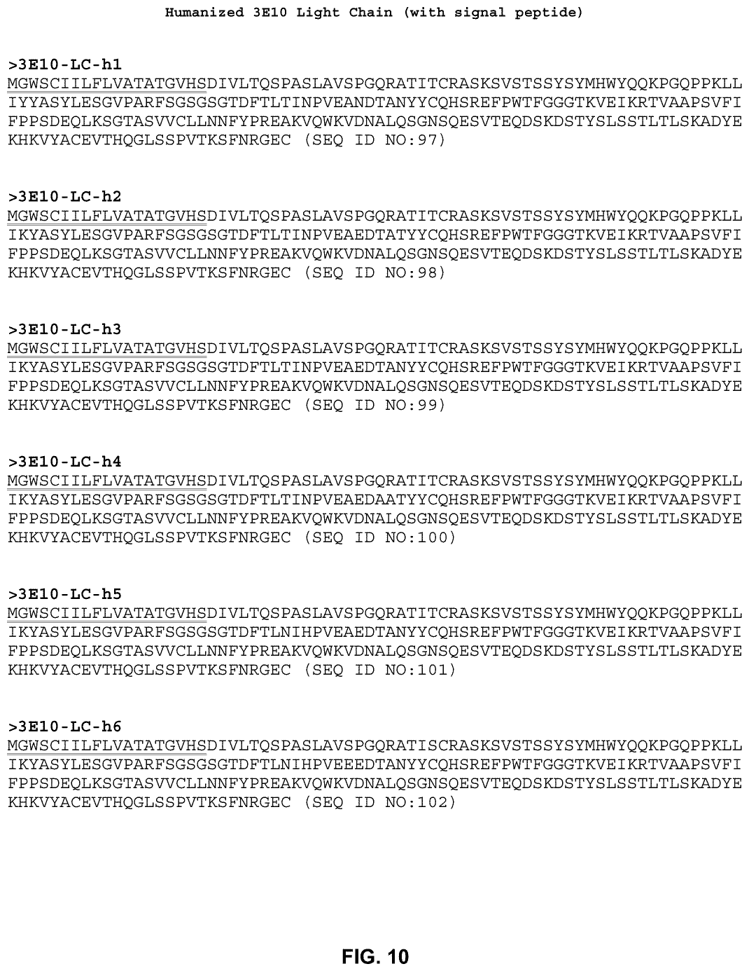

Accordingly, in some embodiments the disclosure provides humanized antibodies and antigen binding fragments thereof that incorporate any combination of the humanized VL and VH sequences shown in FIGS. 5-10, as well as VL and VH sequences having sequence identity thereto, e.g., having at least 90%, 91%, 92%, 93%, 94%, 95%, 96%, 97%, 98%, or 99% identity to a VH or VL sequence shown in FIGS. 5-10.

In some embodiments, a humanized 3E10 antibody or antigen binding fragment thereof, described herein includes a light chain variable domain (3E10-VL) comprising an amino acid sequence that is at least 97% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90) and a heavy chain variable domain (3E10-VH) comprising an amino acid sequence that is at least 95% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In some embodiments, a humanized 3E10 antibody or antigen binding fragment thereof, described herein includes a light chain variable domain (3E10-VL) comprising an amino acid sequence that is at least 97% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90) and a heavy chain variable domain (3E10-VH) comprising an amino acid sequence that is at least 97% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In some embodiments, a humanized 3E10 antibody or antigen binding fragment thereof, described herein includes a light chain variable domain (3E10-VL) comprising an amino acid sequence that is at least 98% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90) and a heavy chain variable domain (3E10-VH) comprising an amino acid sequence that is at least 98% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In some embodiments, a humanized 3E10 antibody or antigen binding fragment thereof, described herein includes a light chain variable domain (3E10-VL) comprising an amino acid sequence that is at least 99% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90) and a heavy chain variable domain (3E10-VH) comprising an amino acid sequence that is at least 99% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

In some embodiments, the sequence of the 3E10-VL is at least 97% identical to 3E10-VL-h1 (SEQ ID NO:85). In some embodiments, the sequence of the 3E10-VL is at least 98% identical to 3E10-VL-h1 (SEQ ID NO:85). In some embodiments, the sequence of the 3E10-VL is at least 99% identical to 3E10-VL-h1 (SEQ ID NO:85). In some embodiments, the sequence of the 3E10-VL is 3E10-VL-h1 (SEQ ID NO:85).

In some embodiments, the sequence of the 3E10-VL is at least 97% identical to 3E10-VL-h2 (SEQ ID NO:86). In some embodiments, the sequence of the 3E10-VL is at least 98% identical to 3E10-VL-h2 (SEQ ID NO:86). In some embodiments, the sequence of the 3E10-VL is at least 99% identical to 3E10-VL-h2 (SEQ ID NO:86). In some embodiments, the sequence of the 3E10-VL is 3E10-VL-h2 (SEQ ID NO:86).

In some embodiments, the sequence of the 3E10-VL is at least 97% identical to 3E10-VL-h3 (SEQ ID NO:87). In some embodiments, the sequence of the 3E10-VL is at least 98% identical to 3E10-VL-h3 (SEQ ID NO:87). In some embodiments, the sequence of the 3E10-VL is at least 99% identical to 3E10-VL-h3 (SEQ ID NO:87). In some embodiments, the sequence of the 3E10-VL is 3E10-VL-h3 (SEQ ID NO:87).

In some embodiments, the sequence of the 3E10-VL is at least 97% identical to 3E10-VL-h4 (SEQ ID NO:88). In some embodiments, the sequence of the 3E10-VL is at least 98% identical to 3E10-VL-h4 (SEQ ID NO:88). In some embodiments, the sequence of the 3E10-VL is at least 99% identical to 3E10-VL-h4 (SEQ ID NO:88). In some embodiments, the sequence of the 3E10-VL is 3E10-VL-h4 (SEQ ID NO:88).

In some embodiments, the sequence of the 3E10-VL is at least 97% identical to 3E10-VL-h5 (SEQ ID NO:89). In some embodiments, the sequence of the 3E10-VL is at least 98% identical to 3E10-VL-h5 (SEQ ID NO:89). In some embodiments, the sequence of the 3E10-VL is at least 99% identical to 3E10-VL-h5 (SEQ ID NO:89). In some embodiments, the sequence of the 3E10-VL is 3E10-VL-h5 (SEQ ID NO:89).

In some embodiments, the sequence of the 3E10-VL is at least 97% identical to 3E10-VL-h6 (SEQ ID NO:90). In some embodiments, the sequence of the 3E10-VL is at least 98% identical to 3E10-VL-h6 (SEQ ID NO:90). In some embodiments, the sequence of the 3E10-VL is at least 99% identical to 3E10-VL-h6 (SEQ ID NO:90). In some embodiments, the sequence of the 3E10-VL is 3E10-VL-h6 (SEQ ID NO:90).

In some embodiments, the sequence of the 3E10-VH is at least 95% identical to 3E10-VH-h1 (SEQ ID NO:64). In some embodiments, the sequence of the 3E10-VH is at least 96% identical to 3E10-VH-h1 (SEQ ID NO:64). In some embodiments, the sequence of the 3E10-VH is at least 97% identical to 3E10-VH-h1 (SEQ ID NO:64). In some embodiments, the sequence of the 3E10-VH is at least 98% identical to 3E10-VH-h1 (SEQ ID NO:64). In some embodiments, the sequence of the 3E10-VH is at least 99% identical to 3E10-VH-h1 (SEQ ID NO:64). In some embodiments, the sequence of the 3E10-VH is 3E10-VH-h1 (SEQ ID NO:64).

In some embodiments, the sequence of the 3E10-VH is at least 95% identical to 3E10-VH-h2 (SEQ ID NO:65). In some embodiments, the sequence of the 3E10-VH is at least 96% identical to 3E10-VH-h2 (SEQ ID NO:65). In some embodiments, the sequence of the 3E10-VH is at least 97% identical to 3E10-VH-h2 (SEQ ID NO:65). In some embodiments, the sequence of the 3E10-VH is at least 98% identical to 3E10-VH-h2 (SEQ ID NO:65). In some embodiments, the sequence of the 3E10-VH is at least 99% identical to 3E10-VH-h2 (SEQ ID NO:65). In some embodiments, the sequence of the 3E10-VH is 3E10-VH-h2 (SEQ ID NO:65).

In some embodiments, the sequence of the 3E10-VH is at least 95% identical to 3E10-VH-h3 (SEQ ID NO:66). In some embodiments, the sequence of the 3E10-VH is at least 96% identical to 3E10-VH-h3 (SEQ ID NO:66). In some embodiments, the sequence of the 3E10-VH is at least 97% identical to 3E10-VH-h3 (SEQ ID NO:66). In some embodiments, the sequence of the 3E10-VH is at least 98% identical to 3E10-VH-h3 (SEQ ID NO:66). In some embodiments, the sequence of the 3E10-VH is at least 99% identical to 3E10-VH-h3 (SEQ ID NO:66). In some embodiments, the sequence of the 3E10-VH is 3E10-VH-h3 (SEQ ID NO:66).

In some embodiments, the sequence of the 3E10-VH is at least 95% identical to 3E10-VH-h4 (SEQ ID NO:67). In some embodiments, the sequence of the 3E10-VH is at least 96% identical to 3E10-VH-h4 (SEQ ID NO:67). In some embodiments, the sequence of the 3E10-VH is at least 97% identical to 3E10-VH-h4 (SEQ ID NO:67). In some embodiments, the sequence of the 3E10-VH is at least 98% identical to 3E10-VH-h4 (SEQ ID NO:67). In some embodiments, the sequence of the 3E10-VH is at least 99% identical to 3E10-VH-h4 (SEQ ID NO:67). In some embodiments, the sequence of the 3E10-VH is 3E10-VH-h4 (SEQ ID NO:67).

In some embodiments, the sequence of the 3E10-VH is at least 95% identical to 3E10-VH-h5 (SEQ ID NO:68). In some embodiments, the sequence of the 3E10-VH is at least 96% identical to 3E10-VH-h5 (SEQ ID NO:68). In some embodiments, the sequence of the 3E10-VH is at least 97% identical to 3E10-VH-h5 (SEQ ID NO:68). In some embodiments, the sequence of the 3E10-VH is at least 98% identical to 3E10-VH-h5 (SEQ ID NO:68). In some embodiments, the sequence of the 3E10-VH is at least 99% identical to 3E10-VH-h5 (SEQ ID NO:68). In some embodiments, the sequence of the 3E10-VH is 3E10-VH-h5 (SEQ ID NO:68).

In some embodiments, the sequence of the 3E10-VH is at least 95% identical to 3E10-VH-h6 (SEQ ID NO:69). In some embodiments, the sequence of the 3E10-VH is at least 96% identical to 3E10-VH-h6 (SEQ ID NO:69). In some embodiments, the sequence of the 3E10-VH is at least 97% identical to 3E10-VH-h6 (SEQ ID NO:69). In some embodiments, the sequence of the 3E10-VH is at least 98% identical to 3E10-VH-h6 (SEQ ID NO:69). In some embodiments, the sequence of the 3E10-VH is at least 99% identical to 3E10-VH-h6 (SEQ ID NO:69). In some embodiments, the sequence of the 3E10-VH is 3E10-VH-h6 (SEQ ID NO:69).

In some embodiments, the sequence of the 3E10-VH is at least 95% identical to 3E10-VH-h7 (SEQ ID NO:70). In some embodiments, the sequence of the 3E10-VH is at least 96% identical to 3E10-VH-h7 (SEQ ID NO:70). In some embodiments, the sequence of the 3E10-VH is at least 97% identical to 3E10-VH-h7 (SEQ ID NO:70). In some embodiments, the sequence of the 3E10-VH is at least 98% identical to 3E10-VH-h7 (SEQ ID NO:70). In some embodiments, the sequence of the 3E10-VH is at least 99% identical to 3E10-VH-h7 (SEQ ID NO:70). In some embodiments, the sequence of the 3E10-VH is 3E10-VH-h7 (SEQ ID NO:70).

In some embodiments, a humanized 3E10 antibody or antigen binding fragment thereof, described herein includes a light chain (3E10-LC) comprising an amino acid sequence that is at least 95% identical to an amino acid sequence selected from the group consisting of 3E10-LC-h1m (SEQ ID NO:91) 3E10-LC-h1m (SEQ ID NO:91), 3E10-LC-h2m (SEQ ID NO:92), 3E10-LC-h3m (SEQ ID NO:93), 3E10-LC-h4m (SEQ ID NO:94), 3E10-LC-h5m (SEQ ID NO:95), and 3E10-LC-h6m (SEQ ID NO:96) and a heavy chain (3E10-HC) comprising an amino acid sequence that is at least 95% identical to an amino acid sequence selected from the group consisting of 3E10-HC-h1m (SEQ ID NO:71), 3E10-HC-h2m (SEQ ID NO:72), 3E10-HC-h3m (SEQ ID NO:73), 3E10-HC-h4m (SEQ ID NO:74), 3E10-HC-h5m (SEQ ID NO:75), 3E10-HC-h6m (SEQ ID NO:76), and 3E10-HC-h7m (SEQ ID NO:77).

In some embodiments, the sequence of the 3E10-LC is at least 95% identical to 3E10-LC-h1m (SEQ ID NO:91). In some embodiments, the sequence of the 3E10-LC is at least 96% identical to 3E10-LC-h1m (SEQ ID NO:91). In some embodiments, the sequence of the 3E10-LC is at least 97% identical to 3E10-LC-h1m (SEQ ID NO:91). In some embodiments, the sequence of the 3E10-LC is at least 98% identical to 3E10-LC-h1m (SEQ ID NO:91). In some embodiments, the sequence of the 3E10-LC is at least 99% identical to 3E10-LC-h1m (SEQ ID NO:91). In some embodiments, the sequence of the 3E10-LC is 3E10-LC-h1m (SEQ ID NO:91).

In some embodiments, the sequence of the 3E10-LC is at least 95% identical to 3E10-LC-h2m (SEQ ID NO:92). In some embodiments, the sequence of the 3E10-LC is at least 96% identical to 3E10-LC-h2m (SEQ ID NO:92). In some embodiments, the sequence of the 3E10-LC is at least 97% identical to 3E10-LC-h2m (SEQ ID NO:92). In some embodiments, the sequence of the 3E10-LC is at least 98% identical to 3E10-LC-h2m (SEQ ID NO:92). In some embodiments, the sequence of the 3E10-LC is at least 99% identical to 3E10-LC-h2m (SEQ ID NO:92). In some embodiments, the sequence of the 3E10-LC is 3E10-LC-h2m (SEQ ID NO:92).

In some embodiments, the sequence of the 3E10-LC is at least 95% identical to 3E10-LC-h3m (SEQ ID NO:93). In some embodiments, the sequence of the 3E10-LC is at least 96% identical to 3E10-LC-h3m (SEQ ID NO:93). In some embodiments, the sequence of the 3E10-LC (SEQ ID NO:7) is at least 97% identical to 3E10-LC-h3m (SEQ ID NO:93). In some embodiments, the sequence of the 3E10-LC is at least 98% identical to 3E10-LC-h3m (SEQ ID NO:93). In some embodiments, the sequence of the 3E10-LC is at least 99% identical to 3E10-LC-h3m (SEQ ID NO:93). In some embodiments, the sequence of the 3E10-LC is 3E10-LC-h3m (SEQ ID NO:93).

In some embodiments, the sequence of the 3E10-LC is at least 95% identical to 3E10-LC-h4m (SEQ ID NO:94). In some embodiments, the sequence of the 3E10-LC is at least 96% identical to 3E10-LC-h4m (SEQ ID NO:94). In some embodiments, the sequence of the 3E10-LC is at least 97% identical to 3E10-LC-h4m (SEQ ID NO:94). In some embodiments, the sequence of the 3E10-LC (SEQ ID NO:7) is at least 98% identical to 3E10-LC-h4m (SEQ ID NO:94). In some embodiments, the sequence of the 3E10-LC is at least 99% identical to 3E10-LC-h4m (SEQ ID NO:94). In some embodiments, the sequence of the 3E10-LC is 3E10-LC-h4m (SEQ ID NO:94).

In some embodiments, the sequence of the 3E10-LC is at least 95% identical to 3E10-LC-h5m (SEQ ID NO:95). In some embodiments, the sequence of the 3E10-LC is at least 96% identical to 3E10-LC-h5m (SEQ ID NO:95). In some embodiments, the sequence of the 3E10-LC is at least 97% identical to 3E10-LC-h5m (SEQ ID NO:95). In some embodiments, the sequence of the 3E10-LC is at least 98% identical to 3E10-LC-h5m (SEQ ID NO:95). In some embodiments, the sequence of the 3E10-LC is at least 99% identical to 3E10-LC-h5m (SEQ ID NO:95). In some embodiments, the sequence of the 3E10-LC is 3E10-LC-h5m (SEQ ID NO:95).

In some embodiments, the sequence of the 3E10-LC is at least 95% identical to 3E10-LC-h6m (SEQ ID NO:96). In some embodiments, the sequence of the 3E10-LC is at least 96% identical to 3E10-LC-h6m (SEQ ID NO:96). In some embodiments, the sequence of the 3E10-LC is at least 97% identical to 3E10-LC-h6m (SEQ ID NO:96). In some embodiments, the sequence of the 3E10-LC is at least 98% identical to 3E10-LC-h6m (SEQ ID NO:96). In some embodiments, the sequence of the 3E10-LC is at least 99% identical to 3E10-LC-h6m (SEQ ID NO:96). In some embodiments, the sequence of the 3E10-LC is 3E10-LC-h6m (SEQ ID NO:96).

In some embodiments, a humanized 3E10 antibody or antigen binding fragment thereof described herein, includes a light chain (3E10-LC) comprising an amino acid sequence that is at least 95% identical to an amino acid sequence selected from the group consisting of 3E10-LC-h1m (SEQ ID NO:97), 3E10-LC-h2 (SEQ ID NO:98), 3E10-LC-h3 (SEQ ID NO:99), 3E10-LC-h4 (SEQ ID NO:100), 3E10-LC-h5 (SEQ ID NO:101), and 3E10-LC-h6 (SEQ ID NO:102) and a heavy chain (3E10-HC) comprising an amino acid sequence that is at least 95% identical to an amino acid sequence selected from the group consisting of 3E10-HC-h1 (SEQ ID NO:78), 3E10-HC-h2 (SEQ ID NO:79), 3E10-HC-h3 (SEQ ID NO:80), 3E10-HC-h4 (SEQ ID NO:81), 3E10-HC-h5 (SEQ ID NO:82), 3E10-HC-h67 (SEQ ID NO:83), and 3E10-HC-h7 (SEQ ID NO:84).

In some embodiments, the sequence of the 3E10-LC is at least 95% identical to 3E10-LC-h1m (SEQ ID NO:97). In some embodiments, the sequence of the 3E10-LC is at least 96% identical to 3E10-LC-h1m (SEQ ID NO:97). In some embodiments, the sequence of the 3E10-LC is at least 97% identical to 3E10-LC-h1m (SEQ ID NO:97). In some embodiments, the sequence of the 3E10-LC is at least 98% identical to 3E10-LC-h1m (SEQ ID NO:97). In some embodiments, the sequence of the 3E10-LC is at least 99% identical to 3E10-LC-h1m (SEQ ID NO:97). In some embodiments, the sequence of the 3E10-LC is 3E10-LC-h1m (SEQ ID NO:97).

In some embodiments, the sequence of the 3E10-LC is at least 95% identical to 3E10-LC-h2 (SEQ ID NO:98). In some embodiments, the sequence of the 3E10-LC is at least 96% identical to 3E10-LC-h2 (SEQ ID NO:98). In some embodiments, the sequence of the 3E10-LC is at least 97% identical to 3E10-LC-h2 (SEQ ID NO:98). In some embodiments, the sequence of the 3E10-LC is at least 98% identical to 3E10-LC-h2 (SEQ ID NO:98). In some embodiments, the sequence of the 3E10-LC is at least 99% identical to 3E10-LC-h2 (SEQ ID NO:98). In some embodiments, the sequence of the 3E10-LC is 3E10-LC-h2 (SEQ ID NO:98).

In some embodiments, the sequence of the 3E10-LC is at least 95% identical to 3E10-LC-h3 (SEQ ID NO:99). In some embodiments, the sequence of the 3E10-LC is at least 96% identical to 3E10-LC-h3 (SEQ ID NO:99). In some embodiments, the sequence of the 3E10-LC is at least 97% identical to 3E10-LC-h3 (SEQ ID NO:99). In some embodiments, the sequence of the 3E10-LC is at least 98% identical to 3E10-LC-h3 (SEQ ID NO:99). In some embodiments, the sequence of the 3E10-LC is at least 99% identical to 3E10-LC-h3 (SEQ ID NO:99). In some embodiments, the sequence of the 3E10-LC is 3E10-LC-h3 (SEQ ID NO:99).

In some embodiments, the sequence of the 3E10-LC is at least 95% identical to 3E10-LC-h4 (SEQ ID NO:100). In some embodiments, the sequence of the 3E10-LC is at least 96% identical to 3E10-LC-h4 (SEQ ID NO:100). In some embodiments, the sequence of the 3E10-LC is at least 97% identical to 3E10-LC-h4 (SEQ ID NO:100). In some embodiments, the sequence of the 3E10-LC is at least 98% identical to 3E10-LC-h4 (SEQ ID NO:100). In some embodiments, the sequence of the 3E10-LC is at least 99% identical to 3E10-LC-h4 (SEQ ID NO:100). In some embodiments, the sequence of the 3E10-LC is 3E10-LC-h4 (SEQ ID NO:100).

In some embodiments, the sequence of the 3E10-LC is at least 95% identical to 3E10-LC-h5 (SEQ ID NO:101). In some embodiments, the sequence of the 3E10-LC is at least 96% identical to 3E10-LC-h5 (SEQ ID NO:101). In some embodiments, the sequence of the 3E10-LC is at least 97% identical to 3E10-LC-h5 (SEQ ID NO:101). In some embodiments, the sequence of the 3E10-LC is at least 98% identical to 3E10-LC-h5 (SEQ ID NO:101). In some embodiments, the sequence of the 3E10-LC is at least 99% identical to 3E10-LC-h5 (SEQ ID NO:101). In some embodiments, the sequence of the 3E10-LC is 3E10-LC-h5 (SEQ ID NO:101).

In some embodiments, the sequence of the 3E10-LC is at least 95% identical to 3E10-LC-h6 (SEQ ID NO:102). In some embodiments, the sequence of the 3E10-LC is at least 96% identical to 3E10-LC-h6 (SEQ ID NO:102). In some embodiments, the sequence of the 3E10-LC is at least 97% identical to 3E10-LC-h6 (SEQ ID NO:102). In some embodiments, the sequence of the 3E10-LC is at least 98% identical to 3E10-LC-h6 (SEQ ID NO:102). In some embodiments, the sequence of the 3E10-LC is at least 99% identical to 3E10-LC-h6 (SEQ ID NO:102). In some embodiments, the sequence of the 3E10-LC is 3E10-LC-h6 (SEQ ID NO:102).

In some embodiments, the sequence of the 3E10-HC is at least 95% identical to 3E10-HC-h1m (SEQ ID NO:71). In some embodiments, the sequence of the 3E10-HC is at least 96% identical to 3E10-HC-h1m (SEQ ID NO:71). In some embodiments, the sequence of the 3E10-HC is at least 97% identical to 3E10-HC-h1m (SEQ ID NO:71). In some embodiments, the sequence of the 3E10-HC is at least 98% identical to 3E10-HC-h1m (SEQ ID NO:71). In some embodiments, the sequence of the 3E10-HC is at least 99% identical to 3E10-HC-h1m (SEQ ID NO:71). In some embodiments, the sequence of the 3E10-HC is 3E10-HC-h1m (SEQ ID NO:71).

In some embodiments, the sequence of the 3E10-HC is at least 95% identical to 3E10-HC-h2m (SEQ ID NO:72). In some embodiments, the sequence of the 3E10-HC is at least 96% identical to 3E10-HC-h2m (SEQ ID NO:72). In some embodiments, the sequence of the 3E10-HC is at least 97% identical to 3E10-HC-h2m (SEQ ID NO:72). In some embodiments, the sequence of the 3E10-HC is at least 98% identical to 3E10-HC-h2m (SEQ ID NO:72). In some embodiments, the sequence of the 3E10-HC is at least 99% identical to 3E10-HC-h2m (SEQ ID NO:72). In some embodiments, the sequence of the 3E10-HC is 3E10-HC-h2m (SEQ ID NO:72).

In some embodiments, the sequence of the 3E10-HC is at least 95% identical to 3E10-HC-h3m (SEQ ID NO:73). In some embodiments, the sequence of the 3E10-HC is at least 96% identical to 3E10-HC-h3m (SEQ ID NO:73). In some embodiments, the sequence of the 3E10-HC is at least 97% identical to 3E10-HC-h3m (SEQ ID NO:73). In some embodiments, the sequence of the 3E10-HC is at least 98% identical to 3E10-HC-h3m (SEQ ID NO:73). In some embodiments, the sequence of the 3E10-HC is at least 99% identical to 3E10-HC-h3m (SEQ ID NO:73). In some embodiments, the sequence of the 3E10-HC is 3E10-HC-h3m (SEQ ID NO:73).

In some embodiments, the sequence of the 3E10-HC is at least 95% identical to 3E10-HC-h4m (SEQ ID NO:74). In some embodiments, the sequence of the 3E10-HC is at least 96% identical to 3E10-HC-h4m (SEQ ID NO:74). In some embodiments, the sequence of the 3E10-HC is at least 97% identical to 3E10-HC-h4m (SEQ ID NO:74). In some embodiments, the sequence of the 3E10-HC is at least 98% identical to 3E10-HC-h4m (SEQ ID NO:74). In some embodiments, the sequence of the 3E10-HC is at least 99% identical to 3E10-HC-h4m (SEQ ID NO:74). In some embodiments, the sequence of the 3E10-HC is 3E10-HC-h4m (SEQ ID NO:74).

In some embodiments, the sequence of the 3E10-HC is at least 95% identical to 3E10-HC-h5m (SEQ ID NO:75). In some embodiments, the sequence of the 3E10-HC is at least 96% identical to 3E10-HC-h5m (SEQ ID NO:75). In some embodiments, the sequence of the 3E10-HC is at least 97% identical to 3E10-HC-h5m (SEQ ID NO:75). In some embodiments, the sequence of the 3E10-HC is at least 98% identical to 3E10-HC-h5m (SEQ ID NO:75). In some embodiments, the sequence of the 3E10-HC is at least 99% identical to 3E10-HC-h5m (SEQ ID NO:75). In some embodiments, the sequence of the 3E10-HC is 3E10-HC-h5m (SEQ ID NO:75).

In some embodiments, the sequence of the 3E10-HC is at least 95% identical to 3E10-HC-h6m (SEQ ID NO:76). In some embodiments, the sequence of the 3E10-HC is at least 96% identical to 3E10-HC-h6m (SEQ ID NO:76). In some embodiments, the sequence of the 3E10-HC is at least 97% identical to 3E10-HC-h6m (SEQ ID NO:76). In some embodiments, the sequence of the 3E10-HC is at least 98% identical to 3E10-HC-h6m (SEQ ID NO:76). In some embodiments, the sequence of the 3E10-HC is at least 99% identical to 3E10-HC-h6m (SEQ ID NO:76). In some embodiments, the sequence of the 3E10-HC is 3E10-HC-h6m (SEQ ID NO:76).

In some embodiments, the sequence of the 3E10-HC is at least 95% identical to 3E10-HC-h7m (SEQ ID NO:77). In some embodiments, the sequence of the 3E10-HC is at least 96% identical to 3E10-HC-h7m (SEQ ID NO:77). In some embodiments, the sequence of the 3E10-HC is at least 97% identical to 3E10-HC-h7m (SEQ ID NO:77). In some embodiments, the sequence of the 3E10-HC is at least 98% identical to 3E10-HC-h7m (SEQ ID NO:77). In some embodiments, the sequence of the 3E10-HC is at least 99% identical to 3E10-HC-h7m (SEQ ID NO:77). In some embodiments, the sequence of the 3E10-HC is 3E10-HC-h7m (SEQ ID NO:77)

In some embodiments, the sequence of the 3E10-HC is at least 95% identical to 3E10-HC-h1 (SEQ ID NO:78). In some embodiments, the sequence of the 3E10-HC is at least 96% identical to 3E10-HC-h1 (SEQ ID NO:78). In some embodiments, the sequence of the 3E10-HC is at least 97% identical to 3E10-HC-h1 (SEQ ID NO:78). In some embodiments, the sequence of the 3E10-HC is at least 98% identical to 3E10-HC-h1 (SEQ ID NO:78). In some embodiments, the sequence of the 3E10-HC is at least 99% identical to 3E10-HC-h1 (SEQ ID NO:78). In some embodiments, the sequence of the 3E10-HC is 3E10-HC-h1 (SEQ ID NO:78).

In some embodiments, the sequence of the 3E10-HC is at least 95% identical to 3E10-HC-h2 (SEQ ID NO:79). In some embodiments, the sequence of the 3E10-HC is at least 96% identical to 3E10-HC-h2 (SEQ ID NO:79). In some embodiments, the sequence of the 3E10-HC is at least 97% identical to 3E10-HC-h2 (SEQ ID NO:79). In some embodiments, the sequence of the 3E10-HC is at least 98% identical to 3E10-HC-h2 (SEQ ID NO:79). In some embodiments, the sequence of the 3E10-HC is at least 99% identical to 3E10-HC-h2 (SEQ ID NO:79). In some embodiments, the sequence of the 3E10-HC is 3E10-HC-h2 (SEQ ID NO:79).

In some embodiments, the sequence of the 3E10-HC is at least 95% identical to 3E10-HC-h3 (SEQ ID NO:80). In some embodiments, the sequence of the 3E10-HC is at least 96% identical to 3E10-HC-h3 (SEQ ID NO:80). In some embodiments, the sequence of the 3E10-HC is at least 97% identical to 3E10-HC-h3 (SEQ ID NO:80). In some embodiments, the sequence of the 3E10-HC is at least 98% identical to 3E10-HC-h3 (SEQ ID NO:80). In some embodiments, the sequence of the 3E10-HC is at least 99% identical to 3E10-HC-h3 (SEQ ID NO:80). In some embodiments, the sequence of the 3E10-HC is 3E10-HC-h3 (SEQ ID NO:80).

In some embodiments, the sequence of the 3E10-HC is at least 95% identical to 3E10-HC-h4 (SEQ ID NO:81). In some embodiments, the sequence of the 3E10-HC is at least 96% identical to 3E10-HC-h4 (SEQ ID NO:81). In some embodiments, the sequence of the 3E10-HC is at least 97% identical to 3E10-HC-h4 (SEQ ID NO:81). In some embodiments, the sequence of the 3E10-HC is at least 98% identical to 3E10-HC-h4 (SEQ ID NO:81). In some embodiments, the sequence of the 3E10-HC is at least 99% identical to 3E10-HC-h4 (SEQ ID NO:81). In some embodiments, the sequence of the 3E10-HC is 3E10-HC-h4 (SEQ ID NO:81).

In some embodiments, the sequence of the 3E10-HC is at least 95% identical to 3E10-HC-h5 (SEQ ID NO:82). In some embodiments, the sequence of the 3E10-HC is at least 96% identical to 3E10-HC-h5 (SEQ ID NO:82). In some embodiments, the sequence of the 3E10-HC is at least 97% identical to 3E10-HC-h5 (SEQ ID NO:82). In some embodiments, the sequence of the 3E10-HC is at least 98% identical to 3E10-HC-h5 (SEQ ID NO:82). In some embodiments, the sequence of the 3E10-HC is at least 99% identical to 3E10-HC-h5 (SEQ ID NO:82). In some embodiments, the sequence of the 3E10-HC is 3E10-HC-h5 (SEQ ID NO:82).

In some embodiments, the sequence of the 3E10-HC is at least 95% identical to 3E10-HC-h67 (SEQ ID NO:83). In some embodiments, the sequence of the 3E10-HC is at least 96% identical to 3E10-HC-h67 (SEQ ID NO:83). In some embodiments, the sequence of the 3E10-HC is at least 97% identical to 3E10-HC-h67 (SEQ ID NO:83). In some embodiments, the sequence of the 3E10-HC is at least 98% identical to 3E10-HC-h67 (SEQ ID NO:83). In some embodiments, the sequence of the 3E10-HC is at least 99% identical to 3E10-HC-h67 (SEQ ID NO:83). In some embodiments, the sequence of the 3E10-HC is 3E10-HC-h67 (SEQ ID NO:83).

In some embodiments, the sequence of the 3E10-HC is at least 95% identical to 3E10-HC-h7 (SEQ ID NO:84). In some embodiments, the sequence of the 3E10-HC is at least 96% identical to 3E10-HC-h7 (SEQ ID NO:84). In some embodiments, the sequence of the 3E10-HC is at least 97% identical to 3E10-HC-h7 (SEQ ID NO:84). In some embodiments, the sequence of the 3E10-HC is at least 98% identical to 3E10-HC-h7 (SEQ ID NO:84). In some embodiments, the sequence of the 3E10-HC is at least 99% identical to 3E10-HC-h7 (SEQ ID NO:84). In some embodiments, the sequence of the 3E10-HC is 3E10-HC-h7 (SEQ ID NO:84).

In some embodiments, a humanized 3E10 antibody or antigen binding fragment thereof described herein comprises a combination of a heavy chain variable domain (VH) and a light chain variable domain (VL) comprising amino acid sequences having at least 97% sequence identity to a pair of VL and VH selected from 3E10-VH-h1 (SEQ ID NO:64) and 3E10-VL-h1 (SEQ ID NO:85), 3E10-VH-h1 (SEQ ID NO:64) and 3E10-VL-h2 (SEQ ID NO:86), 3E10-VH-h1 (SEQ ID NO:64) and 3E10-VL-h3 (SEQ ID NO:87), 3E10-VH-h1 (SEQ ID NO:64) and 3E10-VL-h4 (SEQ ID NO:88), 3E10-VH-h2 (SEQ ID NO:65) and 3E10-VL-h1 (SEQ ID NO:85), 3E10-VH-h2 (SEQ ID NO:65) and 3E10-VL-h2 (SEQ ID NO:86), 3E10-VH-h2 (SEQ ID NO:65) and 3E10-VL-h3 (SEQ ID NO:87), 3E10-VH-h2 (SEQ ID NO:65) and 3E10-VL-h4 (SEQ ID NO:88), 3E10-VH-h3 (SEQ ID NO:66) and 3E10-VL-h1 (SEQ ID NO:85), 3E10-VH-h3 (SEQ ID NO:66) and 3E10-VL-h2 (SEQ ID NO:86), 3E10-VH-h3 (SEQ ID NO:66) and 3E10-VL-h3 (SEQ ID NO:87), 3E10-VH-h3 (SEQ ID NO:66) and 3E10-VL-h4 (SEQ ID NO:88), 3E10-VH-h4 (SEQ ID NO:67) and 3E10-VL-h1 (SEQ ID NO:85), 3E10-VH-h4 (SEQ ID NO:67) and 3E10-VL-h2 (SEQ ID NO:86), 3E10-VH-h4 (SEQ ID NO:67) and 3E10-VL-h3 (SEQ ID NO:87), 3E10-VH-h4 (SEQ ID NO:67) and 3E10-VL-h4 (SEQ ID NO:88), 3E10-VH-h5 (SEQ ID NO:68) and 3E10-VL-h5 (SEQ ID NO:89), 3E10-VH-h5 (SEQ ID NO:68) and 3E10-VL-h6 (SEQ ID NO:90), 3E10-VH-h6 (SEQ ID NO:69) and 3E10-VL-h5 (SEQ ID NO:89), 3E10-VH-h6 (SEQ ID NO:69) and 3E10-VL-h6 (SEQ ID NO:90), 3E10-VH-h7 (SEQ ID NO:70) and 3E10-VL-h5 (SEQ ID NO:89), and 3E10-VH-h7 (SEQ ID NO:70) and 3E10-VL-h6 (SEQ ID NO:90).

In some embodiments, a humanized 3E10 antibody or antigen binding fragment thereof described herein comprises a combination of a heavy chain variable domain (VH) and a light chain variable domain (VL) comprising amino acid sequences having at least 98% sequence identity to a pair of VL and VH selected from 3E10-VH-h1 (SEQ ID NO:64) and 3E10-VL-h1 (SEQ ID NO:85), 3E10-VH-h1 (SEQ ID NO:64) and 3E10-VL-h2 (SEQ ID NO:86), 3E10-VH-h1 (SEQ ID NO:64) and 3E10-VL-h3 (SEQ ID NO:87), 3E10-VH-h1 (SEQ ID NO:64) and 3E10-VL-h4 (SEQ ID NO:88), 3E10-VH-h2 (SEQ ID NO:65) and 3E10-VL-h1 (SEQ ID NO:85), 3E10-VH-h2 (SEQ ID NO:65) and 3E10-VL-h2 (SEQ ID NO:86), 3E10-VH-h2 (SEQ ID NO:65) and 3E10-VL-h3 (SEQ ID NO:87), 3E10-VH-h2 (SEQ ID NO:65) and 3E10-VL-h4 (SEQ ID NO:88), 3E10-VH-h3 (SEQ ID NO:66) and 3E10-VL-h1 (SEQ ID NO:85), 3E10-VH-h3 (SEQ ID NO:66) and 3E10-VL-h2 (SEQ ID NO:86), 3E10-VH-h3 (SEQ ID NO:66) and 3E10-VL-h3 (SEQ ID NO:87), 3E10-VH-h3 (SEQ ID NO:66) and 3E10-VL-h4 (SEQ ID NO:88), 3E10-VH-h4 (SEQ ID NO:67) and 3E10-VL-h1 (SEQ ID NO:85), 3E10-VH-h4 (SEQ ID NO:67) and 3E10-VL-h2 (SEQ ID NO:86), 3E10-VH-h4 (SEQ ID NO:67) and 3E10-VL-h3 (SEQ ID NO:87), 3E10-VH-h4 (SEQ ID NO:67) and 3E10-VL-h4 (SEQ ID NO:88), 3E10-VH-h5 (SEQ ID NO:68) and 3E10-VL-h5 (SEQ ID NO:89), 3E10-VH-h5 (SEQ ID NO:68) and 3E10-VL-h6 (SEQ ID NO:90), 3E10-VH-h6 (SEQ ID NO:69) and 3E10-VL-h5 (SEQ ID NO:89), 3E10-VH-h6 (SEQ ID NO:69) and 3E10-VL-h6 (SEQ ID NO:90), 3E10-VH-h7 (SEQ ID NO:70) and 3E10-VL-h5 (SEQ ID NO:89), and 3E10-VH-h7 (SEQ ID NO:70) and 3E10-VL-h6 (SEQ ID NO:90).

In some embodiments, a humanized 3E10 antibody or antigen binding fragment thereof described herein comprises a combination of a heavy chain variable domain (VH) and a light chain variable domain (VL) comprising amino acid sequences having at least 99% sequence identity to a pair of VL and VH selected from 3E10-VH-h1 (SEQ ID NO:64) and 3E10-VL-h1 (SEQ ID NO:85), 3E10-VH-h1 (SEQ ID NO:64) and 3E10-VL-h2 (SEQ ID NO:86), 3E10-VH-h1 (SEQ ID NO:64) and 3E10-VL-h3 (SEQ ID NO:87), 3E10-VH-h1 (SEQ ID NO:64) and 3E10-VL-h4 (SEQ ID NO:88), 3E10-VH-h2 (SEQ ID NO:65) and 3E10-VL-h1 (SEQ ID NO:85), 3E10-VH-h2 (SEQ ID NO:65) and 3E10-VL-h2 (SEQ ID NO:86), 3E10-VH-h2 (SEQ ID NO:65) and 3E10-VL-h3 (SEQ ID NO:87), 3E10-VH-h2 (SEQ ID NO:65) and 3E10-VL-h4 (SEQ ID NO:88), 3E10-VH-h3 (SEQ ID NO:66) and 3E10-VL-h1 (SEQ ID NO:85), 3E10-VH-h3 (SEQ ID NO:66) and 3E10-VL-h2 (SEQ ID NO:86), 3E10-VH-h3 (SEQ ID NO:66) and 3E10-VL-h3 (SEQ ID NO:87), 3E10-VH-h3 (SEQ ID NO:66) and 3E10-VL-h4 (SEQ ID NO:88), 3E10-VH-h4 (SEQ ID NO:67) and 3E10-VL-h1 (SEQ ID NO:85), 3E10-VH-h4 (SEQ ID NO:67) and 3E10-VL-h2 (SEQ ID NO:86), 3E10-VH-h4 (SEQ ID NO:67) and 3E10-VL-h3 (SEQ ID NO:87), 3E10-VH-h4 (SEQ ID NO:67) and 3E10-VL-h4 (SEQ ID NO:88), 3E10-VH-h5 (SEQ ID NO:68) and 3E10-VL-h5 (SEQ ID NO:89), 3E10-VH-h5 (SEQ ID NO:68) and 3E10-VL-h6 (SEQ ID NO:90), 3E10-VH-h6 (SEQ ID NO:69) and 3E10-VL-h5 (SEQ ID NO:89), 3E10-VH-h6 (SEQ ID NO:69) and 3E10-VL-h6 (SEQ ID NO:90), 3E10-VH-h7 (SEQ ID NO:70) and 3E10-VL-h5 (SEQ ID NO:89), and 3E10-VH-h7 (SEQ ID NO:70) and 3E10-VL-h6 (SEQ ID NO:90).

In some embodiments, a humanized 3E10 antibody or antigen binding fragment thereof described herein comprises a combination of a heavy chain variable domain (VH) and a light chain variable domain (VL) comprising amino acid sequences selected from 3E10-VH-h1 (SEQ ID NO:64) and 3E10-VL-h1 (SEQ ID NO:85), 3E10-VH-h1 (SEQ ID NO:64) and 3E10-VL-h2 (SEQ ID NO:86), 3E10-VH-h1 (SEQ ID NO:64) and 3E10-VL-h3 (SEQ ID NO:87), 3E10-VH-h1 (SEQ ID NO:64) and 3E10-VL-h4 (SEQ ID NO:88), 3E10-VH-h2 (SEQ ID NO:65) and 3E10-VL-h1 (SEQ ID NO:85), 3E10-VH-h2 (SEQ ID NO:65) and 3E10-VL-h2 (SEQ ID NO:86), 3E10-VH-h2 (SEQ ID NO:65) and 3E10-VL-h3 (SEQ ID NO:87), 3E10-VH-h2 (SEQ ID NO:65) and 3E10-VL-h4 (SEQ ID NO:88), 3E10-VH-h3 (SEQ ID NO:66) and 3E10-VL-h1 (SEQ ID NO:85), 3E10-VH-h3 (SEQ ID NO:66) and 3E10-VL-h2 (SEQ ID NO:86), 3E10-VH-h3 (SEQ ID NO:66) and 3E10-VL-h3 (SEQ ID NO:87), 3E10-VH-h3 (SEQ ID NO:66) and 3E10-VL-h4 (SEQ ID NO:88), 3E10-VH-h4 (SEQ ID NO:67) and 3E10-VL-h1 (SEQ ID NO:85), 3E10-VH-h4 (SEQ ID NO:67) and 3E10-VL-h2 (SEQ ID NO:86), 3E10-VH-h4 (SEQ ID NO:67) and 3E10-VL-h3 (SEQ ID NO:87), 3E10-VH-h4 (SEQ ID NO:67) and 3E10-VL-h4 (SEQ ID NO:88), 3E10-VH-h5 (SEQ ID NO:68) and 3E10-VL-h5 (SEQ ID NO:89), 3E10-VH-h5 (SEQ ID NO:68) and 3E10-VL-h6 (SEQ ID NO:90), 3E10-VH-h6 (SEQ ID NO:69) and 3E10-VL-h5 (SEQ ID NO:89), 3E10-VH-h6 (SEQ ID NO:69) and 3E10-VL-h6 (SEQ ID NO:90), 3E10-VH-h7 (SEQ ID NO:70) and 3E10-VL-h5 (SEQ ID NO:89), and 3E10-VH-h7 (SEQ ID NO:70) and 3E10-VL-h6 (SEQ ID NO:90).

In some embodiments, a humanized 3E10 antibody or antigen binding fragment thereof described herein comprises a heavy chain variable domain (VH) comprising an amino acid sequence having at least 97% sequence identity to 3E10-VH-h6 (SEQ ID NO:69) and a light chain variable domain (VL) comprising an amino acid sequence having at least 97% sequence identity to 3E10-VL-h6 (SEQ ID NO:90). In some embodiments, a humanized 3E10 antibody or antigen binding fragment thereof described herein comprises a heavy chain variable domain (VH) comprising an amino acid sequence having at least 98% sequence identity to 3E10-VH-h6 (SEQ ID NO:69) and a light chain variable domain (VL) comprising an amino acid sequence having at least 98% sequence identity to 3E10-VL-h6 (SEQ ID NO:90). In some embodiments, a humanized 3E10 antibody or antigen binding fragment thereof described herein comprises a heavy chain variable domain (VH) comprising an amino acid sequence having at least 99% sequence identity to 3E10-VH-h6 (SEQ ID NO:69) and a light chain variable domain (VL) comprising an amino acid sequence having at least 99% sequence identity to 3E10-VL-h6 (SEQ ID NO:90). In some embodiments, a humanized 3E10 antibody or antigen binding fragment thereof described herein comprises a heavy chain variable domain (VH) comprising an amino acid sequence of 3E10-VH-h6 (SEQ ID NO:69) and a light chain variable domain (VL) comprising an amino acid sequence of 3E10-VL-h6 (SEQ ID NO:90).

In some embodiments, a humanized 3E10 antibody or antigen binding fragment thereof described herein has CDR sequences corresponding to those in the parent 3E10 antibody, shown in FIG. 1 (SEQ ID NO:1-12), optionally including a D31N amino acid substitution in the VH CDR1, as shown in FIG. 2. Accordingly, in some embodiments, a humanized 3E10 antibody or antigen binding fragment thereof includes a light chain variable domain (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO: 11), a heavy chain variable domain (VH) CDR1 comprising the amino acid sequence of 3E10-VH-CDR1a (SEQ ID NO:16), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In some embodiments, a humanized 3E10 antibody or antigen binding fragment thereof described herein includes CDR sequences from a variant humanized 3E10 antibody that includes a D31N amino acid substitution in the VH CDR1, as shown in FIG. 2.

In some embodiments, a humanized 3E10 antibody or antigen binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than ten amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than nine amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than eight amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than seven amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than six amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than five amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than four amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than three amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than two amino acid substitutions, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

In some embodiments, a humanized 3E10 antibody or antigen binding fragment thereof described herein includes a set of complementarity determining regions (CDRs) collectively having no more than one amino acid substitution, relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO: 11), 3E10-VH-CDR1_D31N (SEQ ID NO:15), 3E10-VH-CDR2 (SEQ ID NO:4), and 3E10-VH-CDR3 (SEQ ID NO:5).

Accordingly, in some embodiments, the a humanized 3E10 antibody or antigen binding fragment thereof includes a light chain variable domain (VL) complementarity determining region (CDR) 1 comprising the amino acid sequence of 3E10-VL-CDR1 (SEQ ID NO:9), a VL CDR2 comprising the amino acid sequence of 3E10-VL-CDR2 (SEQ ID NO:10), a VL CDR3 comprising the amino acid sequence of 3E10-VL-CDR3 (SEQ ID NO:11), a heavy chain variable domain (VH) CDR1 comprising the amino acid sequence of 3E10-VH-CDR1_D31N (SEQ ID NO: 15), a VH CDR2 comprising the amino acid sequence of 3E10-VH-CDR2 (SEQ ID NO:4), and a VH CDR3 comprising the amino acid sequence of 3E10-VH-CDR3 (SEQ ID NO:5).

In some embodiments, a humanized 3E10 antibody or antigen binding fragment thereof described herein includes a set of complementarity determining regions (CDRs) collectively having no more than 10, 9, 8, 7, 6, 5, 4, 3, 2, or 1 amino acid substitutions, relative to the CDR sequences of 3E10-D31N variant (shown in FIG. 2), selected from, but not limited to, a G to S substitution at position 5 of VH CDR2, a T to S substitution at position 14 of VH CDR2, an S to T substitution at position 5 of VL CDR1, an M to L substitution at position 14 of VL CDR1, an H to A substitution at position 15 of VL CDR1, and an E to Q substitution at position 6 of VL CDR2.

Accordingly, in some embodiments, a humanized 3E10 antibody or antigen binding fragment thereof includes VH CDR2 comprising the amino acid sequence of 3E10-VH-CDR2.1 (SEQ ID NO:26) or 3E10-VH-CDR2.2 (SEQ ID NO:27). In some embodiments, the humanized 3E10 antibody or antigen binding fragment thereof further includes VL CDRs 1-3, and VH CDRs 1 and 3 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 3 according to the 3E10-D31N variant (as shown in FIG. 2A (SEQ ID NO:13-25)).

Similarly, in some embodiments, a humanized 3E10 antibody or antigen binding fragment thereof includes VL CDR1 comprising the amino acid sequence of 3E10-VL-CDR1.1 (SEQ ID NO:28) or 3E10-VL-CDR1.1 (SEQ ID NO:29). In some embodiments, the humanized 3E10 antibody or antigen binding fragment thereof further includes VL CDRs 2 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 2 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof includes VL CDR2 comprising the amino acid sequence of 3E10-VL-CDR2.1 (SEQ ID NO:30). In some embodiments, the humanized 3E10 antibody or antigen binding fragment thereof further includes VL CDRs 1 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 3, and VH CDRs 1-3 according to the 3E10-D31N variant (as shown in FIG. 2A (SEQ ID NO:13-25)).

While some of the amino acid substitutions described above are fairly conservative substitutions—e.g., an S to T substitution at position 5 of VL CDR1—other substitutions are to amino acids that have vastly different properties—e.g., an M to L substitution at position 14 of VL CDR1, an H to A substitution at position 15 of VL CDR1, and an E to Q substitution at position 6 of VL CDR2. This suggests, without being bound by theory, that at least these positions within the 3E10 CDR framework are tolerant to other amino acid substitutions.

Accordingly, in some embodiments, a humanized 3E10 antibody or antigen binding fragment thereof includes VH CDR2 comprising the amino acid sequence of 3E10-VH-CDR2.3 (SEQ ID NO:31). In some embodiments, the humanized 3E10 antibody or antigen binding fragment thereof further includes VL CDRs 1-3, and VH CDRs 1 and 3 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 3 according to the 3E10-D31N variant (as shown in FIG. 2A (SEQ ID NO:13-25)).

Similarly, in some embodiments, a humanized 3E10 antibody or antigen binding fragment thereof includes VL CDR1 comprising the amino acid sequence of 3E10-VL-CDR1.3 (SEQ ID NO:32). In some embodiments, the humanized 3E10 antibody or antigen binding fragment thereof further includes VL CDRs 2 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 2 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof, includes VL CDR2 comprising the amino acid sequence of 3E10-VL-CDR2.2 (SEQ ID NO:33). In some embodiments, the humanized 3E10 antibody or antigen binding fragment thereof further includes VL CDRs 1 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 3, and VH CDRs 1-3 according to the 3E10-D31N variant (as shown in FIG. 2A (SEQ ID NO:13-25)).

Accordingly, in some embodiments, a humanized 3E10 antibody or antigen binding fragment thereof includes VH CDR1 comprising the amino acid sequence of 3E10-VH-CDR1.c1 (SEQ ID NO:34), 3E10-VH-CDR1.c2 (SEQ ID NO:35), 3E10-VH-CDR1.c3 (SEQ ID NO:36), 3E10-VH-CDR1.c4 (SEQ ID NO:37), or 3E10-VH-CDR1.c5 (SEQ ID NO:38). In some embodiments, the humanized 3E10 antibody or antigen binding fragment thereof further includes VL CDRs 1-3, and VH CDRs 2 and 3 according to the parent 3E10 antibody (as shown in FIG. 1 (SEQ ID NO:1-12)).

Similarly, in some embodiments, a humanized 3E10 antibody or antigen binding fragment thereof includes VH CDR2 comprising the amino acid sequence of 3E10-VH-CDR2.c1 (SEQ ID NO:39), 3E10-VH-CDR2.c2 (SEQ ID NO:40), or 3E10-VH-CDR2.c3 (SEQ ID NO:41). In some embodiments, the humanized 3E10 antibody or antigen binding fragment thereof further includes VL CDRs 1-3, and VH CDRs 1 and 3 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 3 according to the 3E10-D31N variant (as shown in FIG. 2A (SEQ ID NO:13-25)).

Similarly, in some embodiments, a humanized 3E10 antibody or antigen binding fragment thereof includes VH CDR3 comprising the amino acid sequence of 3E10-VH-CDR3.c1 (SEQ ID NO:42), 3E10-VH-CDR3.c2 (SEQ ID NO:43), or 3E10-VH-CDR3.c3 (SEQ ID NO:44). In some embodiments, the humanized 3E10 antibody or antigen binding fragment thereof further includes VL CDRs 1-3, and VH CDRs 1 and 2 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 2 according to the 3E10-D31N variant (as shown in FIG. 2A (SEQ ID NO:13-25)).

Similarly, in some embodiments, a humanized 3E10 antibody or antigen binding fragment thereof includes VL CDR1 comprising the amino acid sequence of 3E10-VL-CDR1.c1 (SEQ ID NO:45), 3E10-VL-CDR1.c2 (SEQ ID NO:46), 3E10-VL-CDR1.c3 (SEQ ID NO:47), 3E10-VL-CDR1.c4 (SEQ ID NO:48), 3E10-VL-CDR1.c5 (SEQ ID NO:49), or 3E10-VL-CDR1.c6 (SEQ ID NO:50). In some embodiments, the humanized 3E10 antibody or antigen binding fragment thereof further includes VL CDRs 2 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 2 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof includes VL CDR2 comprising the amino acid sequence of 3E10-VL-CDR2.c1 (SEQ ID NO:51). In some embodiments, the humanized 3E10 antibody or antigen binding fragment thereof further includes VL CDRs 1 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof includes VL CDR3 comprising the amino acid sequence of 3E10-VL-CDR3.c1 (SEQ ID NO:52), 3E10-VL-CDR3.c2 (SEQ ID NO:53), 3E10-VL-CDR3.c3 (SEQ ID NO:54), 3E10-VL-CDR3.c4 (SEQ ID NO:55), 3E10-VL-CDR3.c5 (SEQ ID NO:56), or 3E10-VL-CDR3.c6 (SEQ ID NO:57). In some embodiments, the humanized 3E10 antibody or antigen binding fragment thereof further includes VL CDRs 1 and 2, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 2, and VH CDRs 1-3 according to the 3E10-D31N variant (as shown in FIG. 2A (SEQ ID NO:13-25)).

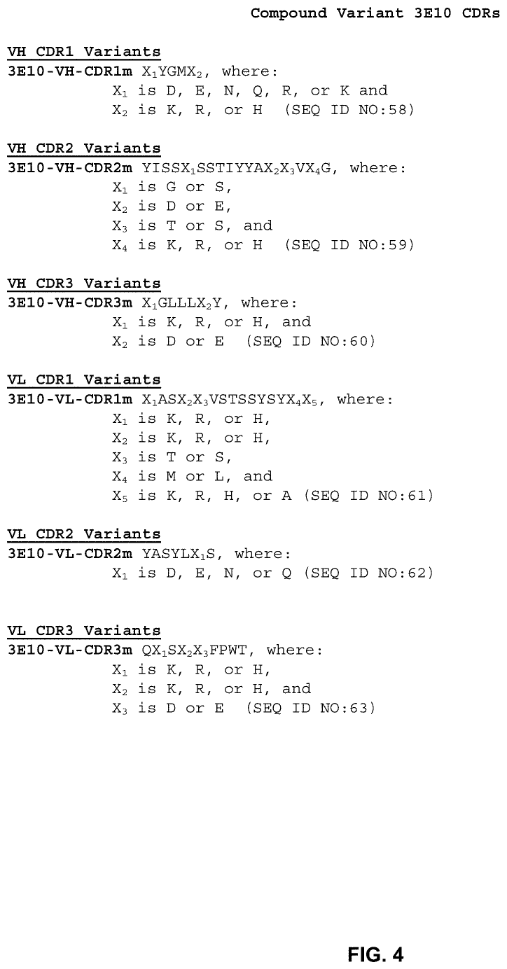

It is also contemplated that a humanized 3E10 antibody or antigen binding fragment thereof, as described herein, includes no more than 7, 6, 5, 4, 3, 2, or 1 CDR amino acid substitutions of the CDR amino acid substitutions described above. Further examples of 3E10 variant CDR sequences described herein are shown in FIG. 4.

Accordingly, in some embodiments, a humanized 3E10 antibody or antigen binding fragment thereof includes VH CDR1 comprising the amino acid sequence of 3E10-VH-CDR1m (SEQ ID NO:58). In some embodiments, the humanized 3E10 antibody or antigen binding fragment thereof further includes VL CDRs 1-3, and VH CDRs 2 and 3 according to the parent 3E10 antibody (as shown in FIG. 1 (SEQ ID NO:1-12)).

Similarly, in some embodiments, a humanized 3E10 antibody or antigen binding fragment thereof includes VH CDR2 comprising the amino acid sequence of 3E10-VH-CDR2m (SEQ ID NO:59). In some embodiments, the humanized 3E10 antibody or antigen binding fragment thereof further includes VL CDRs 1-3, and VH CDRs 1 and 3 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 3 according to the 3E10-D31N variant (as shown in FIG. 2A (SEQ ID NO:13-25)).

Similarly, in some embodiments, a humanized 3E10 antibody or antigen binding fragment thereof includes VH CDR3 comprising the amino acid sequence of 3E10-VH-CDR3m (SEQ ID NO:60). In some embodiments, the humanized 3E10 antibody or antigen binding fragment thereof further includes VL CDRs 1-3, and VH CDRs 1 and 2 according to the parent 3E10 antibody (as shown in FIG. 1 (SEQ ID NO:1-12)). In some embodiments, the humanized 3E10 antibody or antigen binding fragment thereof further includes VL CDRs 1-3, and VH CDRs 1 and 2 according to the 3E10-D31N variant (as shown in FIG. 2A (SEQ ID NO:13-25)).

Similarly, in some embodiments, a humanized 3E10 antibody or antigen binding fragment thereof includes VL CDR1 comprising the amino acid sequence of 3E10-VL-CDR1m (SEQ ID NO:61). In some embodiments, the humanized 3E10 antibody or antigen binding fragment thereof further includes VL CDRs 2 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 2 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof includes VL CDR2 comprising the amino acid sequence of 3E10-VL-CDR2m (SEQ ID NO:62). In some embodiments, the humanized 3E10 antibody or antigen binding fragment thereof further includes VL CDRs 1 and 3, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 3, and VH CDRs 1-3 according to the 3E10-D31N variant (as shown in FIG. 2A (SEQ ID NO:13-25)).

Similarly, in some embodiments, a humanized 3E10 antibody or antigen binding fragment thereof includes VL CDR3 comprising the amino acid sequence of 3E10-VL-CDR3m (SEQ ID NO:63). In some embodiments, the humanized 3E10 antibody or antigen binding fragment thereof further includes VL CDRs 1 and 2, and VH CDRs 1-3 according to the parent 3E10 antibody (as shown in FIG. 1 (SEQ ID NO:1-12)). In some embodiments, the humanized 3E10 antibody or antigen binding fragment thereof further includes VL CDRs 1 and 2, and VH CDRs 1-3 according to the 3E10-D31N variant (as shown in FIG. 2A (SEQ ID NO:13-25)).

In some embodiments, a humanized 3E10 antibody or antigen binding fragment thereof described herein includes a light chain variable domain (3E10-VL) comprising an amino acid sequence that is at least 90% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90), where the light chain variable domain (3E10-VL) comprises one or more amino acid residues selected from proline (Pro) at position 15, threonine (Thr) at position 22, tyrosine (Tyr) at position 49, Thr at position 74, asparagine (Asn) at position 76, alanine (Ala) at position 80, Asn at position 81, Thr at position 83, Asn at position 85, and valine (Val) at position 104, of the 3E10-VL according to Kabat numbering, and a set of 3E10-VL (SEQ ID NO:8) CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), and where the antibody includes a set of 3E10-VL CDRs collectively having no more than 6 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof includes a set of 3E10-VL CDRs comprising no more than 5 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 4 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 3 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 2 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs comprising no more than 1 amino acid substitution relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11). In some embodiments, the humanized 3E10 antibody or antigen binding fragment thereof includes a set of 3E10-VL CDRs having the amino acid sequences of 3E10-VL-CDR1 (SEQ ID NO:9), 3E10-VL-CDR2 (SEQ ID NO:10), 3E10-VL-CDR3 (SEQ ID NO:11).

In some embodiments, the humanized 3E10 antibody or antigen binding fragment thereof has a lysine (Lys) residue at position 49 of the 3E10-VL according to Kabat numbering. In some embodiments, the humanized 3E10 antibody or antigen binding fragment thereof has a glutamic acid (Glu) residue at position 81 of the 3E10-VL according to Kabat numbering. In some embodiments, the humanized 3E10 antibody or antigen binding fragment thereof has a proline (Pro) residue at position 15 of the 3E10-VL according to Kabat numbering. In some embodiments, the humanized 3E10 antibody or antigen binding fragment thereof has a valine (Val) residue at position 104 of the 3E10-VL according to Kabat numbering.

In some embodiments, a humanized 3E10 antibody or antigen binding fragment thereof described herein includes a heavy chain variable domain (3E10-VH) comprising an amino acid sequence that is at least 90% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70), where the heavy chain variable domain (3E10-VH) comprises one or more amino acid residues selected from glutamine (Gln) at position 13, leucine (Leu) at position 18, arginine (Arg) at position 19, glycine (Gly) at position 42, serine (Ser) at position 49, Ser at position 77, tyrosine (Tyr) at position 79, Asn at position 82, Ala at position 84, Val at position 89, leucine (Leu) at position 108, Val at position 109, and Ser at position 113, of the 3E10-VH according to Kabat numbering, and where the antibody includes a set of 3E10-VH CDRs collectively having no more than 6 amino acid substitutions relative to the set of CDRs having the amino acid sequences of 3E10-VH-CDR1 D31N (SEQ ID NO:15), 3E10-VH-CDR2 (SEQ ID NO:4), and 3E10-VH-CDR3 (SEQ ID NO:5).

In some embodiments, the humanized 3E10 antibody or antigen binding fragment thereof includes a set of 3E10-VH CDRs comprising no more than 5 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 4 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 3 amino acid substitutions relative to the set of CDRs having the amino acid sequences of 3E10-VH-CDR1 D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes set of 3E10-VH CDRs comprising no more than 2 amino acid substitutions relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs comprising no more than 1 amino acid substitution relative to the set of CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5). In some embodiments, the humanized 3E10 antibody or antigen binding fragment thereof includes a set of 3E10-VH CDRs having the amino acid sequences of 3E10-VH-CDR1_D31N (SEQ ID NO:15), 3E10-VH-CDR2 (SEQ ID NO:4), and 3E10-VH-CDR3 (SEQ ID NO:5).

In some embodiments, the humanized 3E10 antibody or antigen binding fragment thereof has an arginine (Arg) residue at position 18 of the 3E10-VH according to Kabat numbering. In some embodiments, the humanized 3E10 antibody or antigen binding fragment thereof has a (Lys) residue at position 19 of the 3E10-VH (SEQ ID NO:2) according to Kabat numbering. In some embodiments, the humanized 3E10 antibody or antigen binding fragment thereof has an alanine (Ala) residue at position 49 of the 3E10-VH according to Kabat numbering. In some embodiments, the humanized 3E10 antibody or antigen binding fragment thereof with a glutamine (Gln) residue at position 13 of the 3E10-VH (SEQ ID NO:2) according to Kabat numbering. In some embodiments, the humanized 3E10 antibody or antigen binding fragment thereof has a leucine (Leu) residue at position 108 of the 3E10-VH according to the Kabat numbering. In some embodiments, the humanized 3E10 antibody or antigen binding fragment thereof with a Val residue at position 109 of the 3E10-VH according to Kabat numbering. In some embodiments, the humanized 3E10 antibody or antigen binding fragment thereof has a serine (Ser) residue at position 113 of the 3E10-VH according to Kabat numbering.

In some embodiments, a humanized 3E10 antibody or antigen binding fragment thereof of the present disclosure has a nucleic acid binding affinity for a polynucleotide, e.g., a DNA, RNA, PNA, morpholino, etc., that is similar to the affinity of a reference 3E10 antibody or antigen binding fragment thereof, for example, the 3E10 monoclonal antibody produced by ATCC No. PTA 2439 hybridoma or a D31N variant thereof, has for the same polynucleotide. Accordingly, in some embodiments, the humanized 3E10 antibody or antigen binding fragment thereof, has an affinity for a polynucleotide that is from 0.05-fold to 50-fold as strong as the affinity the reference 3E10 antibody has for the same polynucleotide.

In some embodiments, a humanized 3E10 antibody or antigen binding fragment thereof, of the present disclosure has a nucleic acid binding affinity for a polynucleotide, e.g., a DNA, RNA, PNA, morpholino, etc., that is less than the affinity of a reference 3E10 antibody or antigen binding fragment thereof, for example, the 3E10 monoclonal antibody produced by ATCC No. PTA 2439 hybridoma or a D31N variant thereof, has for the same polynucleotide. Accordingly, in some embodiments, the humanized 3E10 antibody or antigen binding fragment thereof, has an affinity for a polynucleotide that is from 0.00001-fold to less than 1-fold as strong as the affinity the reference 3E10 antibody has for the same polynucleotide. In some embodiments, the humanized 3E10 antibody or antigen binding fragment thereof, has an affinity for a polynucleotide that is from 0.00001-fold to 0.001-fold as strong as the affinity the reference 3E10 antibody has for the same polynucleotide. In some embodiments, the humanized 3E10 antibody or antigen binding fragment thereof, has an affinity for a polynucleotide that is from 0.0001-fold to 0.01-fold as strong as the affinity the reference 3E10 antibody has for the same polynucleotide. In some embodiments, the humanized 3E10 antibody or antigen binding fragment thereof, has an affinity for a polynucleotide that is from 0.001-fold to 0.1-fold as strong as the affinity the reference 3E10 antibody has for the same polynucleotide. In some embodiments, the humanized 3E10 antibody or antigen binding fragment thereof, has an affinity for a polynucleotide that is from 0.01-fold to less than 1-fold as strong as the affinity the reference 3E10 antibody has for the same polynucleotide.

In some embodiments, a humanized 3E10 antibody or antigen binding fragment thereof, of the present disclosure has a nucleic acid binding affinity for a polynucleotide, e.g., a DNA, RNA, PNA, morpholino, etc., that is greater than the affinity of a reference 3E10 antibody or antigen binding fragment thereof, for example, the 3E10 monoclonal antibody produced by ATCC No. PTA 2439 hybridoma or a D31N variant thereof, has for the same polynucleotide. Accordingly, in some embodiments, the humanized 3E10 antibody or antigen binding fragment thereof, has an affinity for a polynucleotide that is from greater than 1-fold to 10,000-fold as strong as the affinity the reference 3E10 antibody has for the same polynucleotide. In some embodiments, the humanized 3E10 antibody or antigen binding fragment thereof, has an affinity for a polynucleotide that is from 100-fold to 10,000-fold as strong as the affinity the reference 3E10 antibody has for the same polynucleotide. In some embodiments, the humanized 3E10 antibody or antigen binding fragment thereof, has an affinity for a polynucleotide that is from 10-fold to 1000-fold as strong as the affinity the reference 3E10 antibody has for the same polynucleotide. In some embodiments, the humanized 3E10 antibody or antigen binding fragment thereof, has an affinity for a polynucleotide that is from greater than 1-fold to 100-fold as strong as the affinity the reference 3E10 antibody has for the same polynucleotide.

Binding affinity may be determined by association (Ka) and dissociation (Kd) rate. Equilibrium affinity constant, KD, is the ratio of Ka/Kd. A humanized 3E10 antibody or antigen binding fragment thereof having the same binding affinity as another 3E10 antibody, means that the dissociation constant (Kd) for each antibody is within about 1 to 10-fold (1-10 fold greater affinity or 1-10 fold less affinity, or any numerical value or range or value within such ranges). Example affinities for a target antigen (DNA (e.g., single-stranded and/or double-stranded DNA)) have a dissociation constant (Kd) less than 5×10⁻² M, less than 10⁻² M, less than 5×10⁻³ M, less than 10⁻³ M, less than 5×10⁻⁴ M, less than 10⁻⁴ M, less than 5×10⁻⁵ M, less than 10⁻⁵ M, less than 5×10⁻⁸ M, less than 10⁻⁸ M, less than 5×10⁻⁷ M, less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹ M, or less than 10⁻¹⁵ M. In some embodiments, the binding affinity (Kd) for a target is less than 10⁻⁷ M, less than 5×10⁻⁸ M, less than 10⁻⁸ M, less than 5×10⁻⁹ M, less than 10⁻⁹ M, less than 5×10⁻¹⁰ M, less than 10⁻¹⁰ M, less than 5×10⁻¹¹ M, less than 10⁻¹¹ M, less than 5×10⁻¹² M, or less than 10⁻¹² M.

In some embodiments, binding affinity is evaluated by determining a solution concentration of the humanized 3E10 antibody or antigen binding fragment thereof needed to achieve half maximum binding (EC50) of a polynucleotide coated on a solid surface. In some embodiments, the nucleic acid is a poly-dT oligonucleotide. In some embodiments, binding of the oligonucleotide, e.g., a poly-dT oligonucleotide, is detected using an ELISA assay with a secondary anti-human immunoglobulin antibody.

In some embodiments, a humanized 3E10 antibody or antigen binding fragment thereof described herein has a weak binding affinity for a poly-dT oligonucleotide. In some embodiments, the weak binding affinity is an EC50 value that is at least 100 times greater than the EC50 value of a reference 3E10 antibody, e.g., a chimeric 3E10 antibody with a D31N amino acid substitution. In some embodiments, the weak binding affinity is an EC50 value that is from 100 times greater to 10,000 times greater than the EC50 value of a reference 3E10 antibody, e.g., a chimeric 3E10 antibody with a D31N amino acid substitution. In some embodiments, the weak binding affinity is an EC50 value that is from 100 times greater to 5000 times greater than the EC50 value of a reference 3E10 antibody, e.g., a chimeric 3E10 antibody with a D31N amino acid substitution. In some embodiments, the weak binding affinity is an EC50 value that is from 100 times greater to 1000 times greater than the EC50 value of a reference 3E10 antibody, e.g., a chimeric 3E10 antibody with a D31N amino acid substitution. In some embodiments, the weak binding affinity is an EC50 value that is from 100 times greater to 500 times greater than the EC50 value of a reference 3E10 antibody, e.g., a chimeric 3E10 antibody with a D31N amino acid substitution.

In some embodiments, a humanized 3E10 antibody or antigen binding fragment thereof described herein has an intermediate binding affinity for a poly-dT oligonucleotide. In some embodiments, the intermediate binding affinity is an EC50 value that is from 25 times to 100 times greater than the EC50 value of a reference 3E10 antibody, e.g., a chimeric 3E10 antibody with a D31N amino acid substitution.

In some embodiments, a humanized 3E10 antibody or antigen binding fragment thereof described herein has an intermediate binding affinity for a poly-dT oligonucleotide. In some embodiments, the intermediate binding affinity is an EC50 value that is no more than 25 times greater than the EC50 value of a reference 3E10 antibody, e.g., a chimeric 3E10 antibody with a D31N amino acid substitution. In some embodiments, the intermediate binding affinity is an EC50 value that is no more than 20 times greater than the EC50 value of a reference 3E10 antibody, e.g., a chimeric 3E10 antibody with a D31N amino acid substitution. In some embodiments, the intermediate binding affinity is an EC50 value that is no more than 15 times greater than the EC50 value of a reference 3E10 antibody, e.g., a chimeric 3E10 antibody with a D31N amino acid substitution. In some embodiments, the intermediate binding affinity is an EC50 value that is no more than 10 times greater than the EC50 value of a reference 3E10 antibody, e.g., a chimeric 3E10 antibody with a D31N amino acid substitution. In some embodiments, the intermediate binding affinity is an EC50 value that is no more than 5 times greater than the EC50 value of a reference 3E10 antibody, e.g., a chimeric 3E10 antibody with a D31N amino acid substitution.

In some embodiments, a humanized 3E10 antibody or antigen binding fragment thereof described herein has a weak binding affinity for a 3p-hpRNA RIG-I agonist having the nucleotide sequence 5′-pppGGAGCAAAAGCAGGGUGACAAAGACAUAAUGGAUCCAAACACUGUGUCAAGCUUUCAGGUA GAUUGCUUUCUUUGGCAUGUCCGCAAAC-3′ (SEQ ID NO:103).

In some embodiments, a humanized 3E10 antibody or antigen binding fragment thereof described herein has an intermediate binding affinity for the 3p-hpRNA RIG-I agonist having the nucleotide sequence 5′-pppGGAGCAAAAGCAGGGUGACAAAGACAUAAUGGAUCCAAACACUGUGUCAAGCUUUCAGGUA GAUUGCUUUCUUUGGCAUGUCCGCAAAC-3′ (SEQ ID NO:103).

In some embodiments, a humanized 3E10 antibody or antigen binding fragment thereof described herein has a strong binding affinity for the 3p-hpRNA RIG-I agonist having the nucleotide sequence 5′-pppGGAGCAAAAGCAGGGUGACAAAGACAUAAUGGAUCCAAACACUGUGUCAAGCUUUCAGGUA GAUUGCUUUCUUUGGCAUGUCCGCAAAC-3′ (SEQ ID NO:103).

In some embodiments, a humanized 3E10 antibody or antigen binding fragment thereof described herein comprises a fragment crystallizable (Fc) region. In some embodiments, the Fc region is a human IgG1 Fc, a human IgG2a Fc, a human IgG2b Fc, a human IgG3 Fc, and a human IgG4 Fc.

In some embodiments, a humanized 3E10 antibody or antigen binding fragment thereof described herein has a human IgG1 Fc domain. In some embodiments, the human IgG1 domain comprises an amino acid sequence having high sequence identity, e.g., at least 95% identity, at least 96% identity, at least 97% identity, at least 98% identity, at least 99% identity, at least 99.5% identity, or 100% identity to

(SEQ ID NO: 115)
ASTKGPSVFPLAPSSKSTSGGTAALGCLVKDYFPEPVTVSWNSGALT
SGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSV
LTVLHQDWLNGKEYKCKVSNKALPAPIEKTISKAKGQPREPQVYTLP
PSRDELTKNQVSLTCLVKGFYPSDIAVEWESNGQPENNYKTTPPVLD
SDGSFFLYSKLTVDKSRWQQGNVFSCSVMHEALHNHYTQKSLSLSPG
K.

In some embodiments, the IgG1 Fc domain sequence comprises one or more natural amino acid variants found in the human population, e.g., it is an IgG1 Fc allotype. Non-limiting examples of IgG1 Fc allotype amino acid variants include G1m (z,a), G1m (f), and G1m (f,a). The G1m (f) allele is only found in Caucasians, whereas the G1m (f,a) allele is common in Asian populations but other variants, G1m (z,a,x) and G1m (z,a,v), have also been described (See, Vidarsson et al. Front. Immunol., October 2014, Vol. 5, Article 520, the disclosures of which is incorporated herein by reference in its entirety.

In some embodiments, a humanized 3E10 antibody or antigen binding fragment thereof has an IgG1 domain containing one or more engineered amino acid substitutions, e.g., to reduce Fc effector function, improve half-life in vivo, and/or otherwise alter the properties of the antibody in vivo. Several approved antibodies have demonstrated potent in vitro CDC activity such as the anti-CD20 mAbs rituximab and ofatumumab, and there are numerous ways that investigators have utilized Fc engineering to enhance complement-based effector function. Idusogie et al. demonstrated that K326W/E333S enhanced C1q binding and CDC activity relative to an IgG1, leading the authors to suggest that these two residues play a structural role in interactions between C1q and IgG. Moore et al. demonstrated that the Fc mutations S267E/H268F/S324T enhanced C1q binding 47-fold and CDC activity 6.9-fold over IgG1 (See, Teeling J. L. et al. J. Immunol. 2006 177:362-371, Idusogie E. E. et al. J. Immunol. 2001 166:2571-2575, Moore G. L. et al. MAbs. 2010 2:181-189, and Wang et al. Protein & Cell, Volume 9, Issue 1, 2018, 63-73, the disclosures of which is incorporated herein by reference in its entirety.

Accordingly, in some embodiments, the IgG1 Fc domain comprises an amino acid substitution at a position selected from L234A, according to the EU index as in the Kabat numbering scheme. In some embodiments, the IgG1 Fc domain comprises an amino acid substitution selected from L235A. In some embodiments, the IgG1 Fc domain comprises a combination of amino acid substitutions selected from L234A/L235A. In some embodiments, the IgG1 Fc domain comprises a combination of amino acid substitutions selected from N297D. In some embodiments, the IgG1 Fc domain comprises a combination of amino acid substitutions selected from L234A/L235A/N297D.

In some embodiments, a humanized 3E10 antibody or antigen binding fragment thereof described herein has a human IgG1 Fc domain comprising L234A/L235A amino acid substitutions. In some embodiments, the IgG1 Fc domain comprises an amino acid sequence comprising residues 234A/235A and having high sequence identity, e.g., at least 95% identity, at least 96% identity, at least 97% identity, at least 98% identity, at least 99% identity, at least 99.5% identity, or 100% identity to

(SEQ ID NO: 116)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In embodiments, a humanized 3E10 antibody or antigen binding fragment thereof described herein has a human IgG1 Fc domain comprising an N297D amino acid substitution. In some embodiments, the IgG1 Fc domain comprises an amino acid sequence comprising residue 297D and having high sequence identity, e.g., at least 95% identity, at least 96% identity, at least 97% identity, at least 98% identity, at least 99% identity, at least 99.5% identity, or 100% identity to

(SEQ ID NO: 117)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPELLGGPSVFLFPPKPKDTLMISRTPEVTCVVV
DVSHEDPEVKFNWYVDGVEVHNAKTKPREEQYDSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, a humanized 3E10 antibody or antigen binding fragment thereof described herein has a human IgG1 Fc domain comprising L234A/L235A/N297D amino acid substitutions. amino acid substitutions. In some embodiments, the IgG1 Fc domain comprises an amino acid sequence comprising residues 234A/235A/297D and having high sequence identity, e.g., at least 95% identity, at least 96% identity, at least 97% identity, at least 98% identity, at least 99% identity, at least 99.5% identity, or 100% identity to

(SEQ ID NO: 118)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSCDKTHTCPPCPAPEAAGGPSVFLFPPKPKDTLMISRTPEVTCVVV
DVSHEDPEVKFNWYVDGVEVHNAKTKPREEQYDSTYRVVSVLTVLHQDW
LNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLT
VDKSRWQQGNVFSCSVMHEALHNHYTQKSLSLSPGK.

In some embodiments, the IgG1 constant heavy region 1 comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 154)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV.

In some embodiments, the IgG1 hinge region comprises an amino acid sequence comprising high sequence identity, e.g., at least 95% identity, at least 96% identity, at least 97% identity, at least 98% identity, at least 99% identity, at least 99.5% identity, or 100% identity to EPKSCDKTHTCP (SEQ ID NO:155).

In some embodiments, the IgG1 L2345A/L235A constant heavy region 2 comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 156)
PCPAPEAAGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAK.

In some embodiments, the IgG1 constant heavy region 3 comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 157)
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPGK.

In some embodiments, the IgG1 N297D constant heavy region 2 comprises an amino acid sequence comprising high sequence identity, e.g., at least 95% identity, at least 96% identity, at least 97% identity, at least 98% identity, at least 99% identity, at least 99.5% identity, or 100% identity to

(SEQID NO: 158)
PCPAPELLGGPSVFLFPPKPKDTLMISRTPEVTCVVVDVSHEDPEVKFN
WYVDGVEVHNAKTKPREEQYDSTYRVVSVLTVLHQDWLNGKEYKCKVSN
KALPAPIEKTISKAK.

In some embodiments, the IgG1 L2345A/L235A/N297D constant heavy region 2 comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 159)
PCPAPEAAGGPSVFLFPPKPKDTLMISRTPEVTCVVVDVSHEDPEVKFN
WYVDGVEVHNAKTKPREEQYDSTYRVVSVLTVLHQDWLNGKEYKCKVSN
KALPAPIEKTISKAK (IgG1 L2345A/L235A/N297D

In some embodiments, the unmodified constant heavy region 2 comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 160)
PCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSN
KALPAPIEKTISKAK.

In some embodiments, the light chain full length sequence comprises an amino acid sequence comprising high sequence identity, e.g., at least 95% identity, at least 96% identity, at least 97% identity, at least 98% identity, at least 99% identity, at least 99.5% identity, or 100% identity to

(SEQ ID NO: 161)
DIQMTQSPSSLSASLGDRATITCRASKTVSTSSYSYMHWYQQKPGQPPK
LLIKYASYLESGVPSRFSGSGSGTDFTLTISSLQPEDAATYYCQHSREF
PWTFGGGTKLEIKRTVAAPSVFIFPPSDEQLKSGTASVVCLLNNFYPRE
AKVQWKVDNALQSGNSQESVTEQDSKDSTYSLSSTLTLSKADYEKHKVY
ACEVTHQGLSSPVTKSFNRGEC.

In some embodiments, a humanized 3E10 antibody or antigen binding fragment thereof described herein has a human IgG4 Fc domain. In some embodiments, the human IgG4 domain comprises an amino acid sequence having high sequence identity, e.g., at least 95% identity, at least 96% identity, at least 97% identity, at least 98% identity, at least 99% identity, at least 99.5% identity, or 100% identity to

(SEQ ID NO: 104)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In some embodiments, the IgG4 Fc domain sequence comprises one or more natural amino acid variants found in the human population, e.g., it is an IgG4 Fc allotype. Non-limiting examples of IgG4 Fc allotype amino acid variants include nG4m (a) and nG4m (b) (See, Vidarsson et al. Front. Immunol., October 2014, Vol. 5, Article 520, the disclosures of which is incorporated herein by reference in its entirety/

In some embodiments, a humanized 3E10 antibody or antigen binding fragment thereof has an IgG4 domain containing one or more engineered amino acid substitutions, e.g., to reduce Fc effector function, improve half-life in vivo, and/or otherwise alter the properties of the antibody in vivo. In fact, the majority of IgG4-based therapeutic antibodies approved for marketing or in late-stage clinical trials, contain at least one such amino acid substitution. For review of the amino acid substitutions in such antibodies see, for example, Dumet C. et al., MABS, 11 (8):1341-50 (2019), the disclosure of which is incorporated herein by reference in its entirety. Other IgG4 Fc mutations have been suggested for reducing immune effector functions while retaining other Fc characteristics. For example, Tam S. H. et al., Antibodies, 6(12):1-34 (2017), the disclosure of which is incorporated by reference herein in its entirety, reports on the characterization of two IgG4 Fc variant designs, huIgG4 σ1 and huIgG4 σ2. The huIgG4 σ1 construct includes S228P, F234A, L235A, G237A, and P238S amino acid substitutions while the huIgG4 σ2 construct includes a G236>del, in addition to the S228P, F234A, L235A, G237A, and P238S amino acid substitutions. Other IgG4 Fc mutations are described in Liu R. et al., Antibodies, 9(64):1-34 (2020), the disclosure of which is hereby incorporated by reference herein, in its entirety. These mutations include M252Y, S254T, T256E, H433K, and N434F.

Yet other IgG4 Fc amino acid substitutions that may be integrated into an IgG4 Fc domain of a humanized 3E10 antibody or antigen binding fragment thereof are suggested in the art including, without limitation, WO 1989/007142, U.S. Pat. No. 5,885,573, WO 1994/029351, U.S. Pat. No. 6,407,214, US 2006/0024298, U.S. Pat. No. 7,863,419, US 2007/0041972, U.S. Pat. Nos. 8,961,967, 9,187,552, 8,969,526, 9,359,437, WO 2017/079369, U.S. Pat. Nos. 7,371,826, 7,083,784, WO 2004/035752, U.S. Pat. Nos. 9,200,079, 11,046,784, 8,802,820, US 2020/0255502, US 2010/0098730, U.S. RE45992, US 2010/0204454, U.S. Pat. No. 8,637,641, US 2014/0302028, GB 201302878, US 2015/0065690, US 2014/0294812, US 2020/0071423, U.S. Pat. No. 11,319,383, US 2018/0037634, KR 101792191, US 2019/0010243, U.S. Pat. No. 8,911,726, US 2010/0267934, U.S. Pat. No. 9,688,762, US 2012/0100140, U.S. Pat. Nos. 9,085,625, 10,562,966, US 2017/029521, U.S. Pat. No. 11,254,753, and WO 2018/119380, the disclosures of which are hereby incorporated by reference herein, in their entireties, for all purposes.

Accordingly, in some embodiments, the IgG4 Fc domain comprises an amino acid substitution at a position selected from 196, 228, 234, 234, 235, 235, 236, 237, 238, 252, 254, 256, 265, 296, 233, 310, 331, 356, 409, 428, 433, 434, 435, 445, 446, and K447, according to the EU index as in the Kabat numbering scheme. In some embodiments, the IgG4 Fc domain comprises an amino acid substitution selected from K196Q, S228P, F234A, F234V, L235A, L235E, G236>del, G237A, P238S, M252Y, S254T, T256E, D265A, F296Y, E233P, T307Q, H310Q, P331S, E356K, R409K, M428L, H433K, N434A, N434F, N434S, H435R, L445P, G446>del, and K447>del. In some embodiments, the IgG4 Fc domain comprises a combination of amino acid substitutions selected from S228P/L234A/L235A, L234F/L235E/P331S, M252Y/S254T/T256E, M252Y/S254T/T256E/H433K/N434F, S228P/F234A/L235A/H310Q, S228P/F234A/L235A/M252Y/S254T/T256E, S228P/F234A/L235A/T307Q/N434A, S228P/F234A/L235A/G237A/P238S, and S228P/F234A/L235A/G236>del/G237A/P238S.

In some embodiments, a humanized 3E10 antibody or antigen binding fragment thereof described herein has a human IgG4 Fc domain comprising S228P/F234A/L235A amino acid substitutions. In some embodiments, the IgG4 Fc domain comprises an amino acid sequence comprising residues 228P/234A/235A and having high sequence identity, e.g., at least 95% identity, at least 96% identity, at least 97% identity, at least 98% identity, at least 99% identity, at least 99.5% identity, or 100% identity to

(SEQ ID NO: 105)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In some embodiments, a humanized 3E10 antibody or antigen binding fragment thereof described herein has a human IgG4 Fc domain comprising S228P/F234A/L235A/T307Q/N434A amino acid substitutions. In some embodiments, the IgG4 Fc domain comprises an amino acid sequence comprising residues 228P/234A/235A/307Q/434A having high sequence identity, e.g., at least 95% identity, at least 96% identity, at least 97% identity, at least 98% identity, at least 99% identity, at least 99.5% identity, or 100% identity to

(SEQ ID NO: 106)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLQVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHAHYTQKSLSLSLGK.

In some embodiments, a humanized 3E10 antibody or antigen binding fragment thereof described herein has a human IgG4 Fc domain comprising S228P/F234A/L235A/M252Y/S254T/T256E amino acid substitutions. In some embodiments, the IgG4 Fc domain comprises an amino acid sequence comprising residues 228P/234A/235A/252Y/254T/256E having high sequence identity, e.g., at least 95% identity, at least 96% identity, at least 97% identity, at least 98% identity, at least 99% identity, at least 99.5% identity, or 100% identity to

(SEQ ID NO: 107)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLYITREPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In some embodiments, a humanized 3E10 antibody or antigen binding fragment thereof described herein has a human IgG4 Fc domain comprising S228P/F234A/L235A/H310Q amino acid substitutions. In some embodiments, the IgG4 Fc domain comprises an amino acid sequence comprising residues 228P/234A/235A/310Q having high sequence identity, e.g., at least 95% identity, at least 96% identity, at least 97% identity, at least 98% identity, at least 99% identity, at least 99.5% identity, or 100% identity to

(SEQ ID NO: 108)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEAAGGPSVFLFPPKPKDTLMISRTPEVTCVVVDVS
QEDPEVQFNWYVDGVEVHNAKTKPREEQFNSTYRVVSVLTVLQQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

In some embodiments, the present disclosure provides humanized 3E10 antibodies and antigen binding fragments thereof comprising a heavy chain constant domain (CH)1.

In some embodiments, the humanized 3E10 antibody or antigen binding fragment thereof comprises an Fc region selected from a human γ1 CH1, a human γ2 CH1, a human γ3 CH1, and a human γ4 CH1.

In some embodiments, the present disclosure provides a humanized 3E10 antibody or antigen binding fragment thereof comprising a light chain constant domain (CL).

In another aspect, the humanized 3E10 antibody or antigen binding fragment thereof comprises an Fc region selected from the group consisting of a human λ CL and a human κ CL.

In various embodiments, the humanized 3E10 antibody or antigen binding fragment thereof is covalently linked to a therapeutic moiety.

In some embodiments, the therapeutic moiety is a therapeutic polypeptide, a cytotoxic moiety, a chemotherapeutic moiety, or a moiety that is detectable.

### Cell Penetration and Nuclear Localization

The disclosed compositions and methods typically utilize antibodies that maintain the ability to penetrate cells, and optionally nuclei.

The mechanisms of cellular internalization by autoantibodies are diverse. Some are taken into cells through electrostatic interactions or FcR-mediated endocytosis, while others utilize mechanisms based on association with cell surface myosin or calreticulin, followed by endocytosis (Ying-Chyi et al., Eur. J. Immunol. 38, 3178-3190 (2008), Yanase et al., J Clin Invest 100, 25-31 (1997)). 3E10 penetrates cells in an Fc-independent mechanism (as evidenced by the ability of 3E10 fragments lacking an Fc to penetrate cells) but involves presence of the nucleoside transporter ENT2 (Weisbart et al., Scientific Reports volume 5, Article number: 12022 (2015), Zack et al., J Immunol 157, 2082-2088 (1996), Hansen et al., J Biol Chem 282, 20790-20793 (2007)). Thus, in some embodiments, the antibodies utilized in the disclosed compositions and methods are ones that penetrate cells in an Fc-independent mechanism but involves presence of the nucleoside transporter ENT2.

Mutations in 3E10 that interfere with its ability to bind nucleic acids may render the antibody incapable of nuclear penetration. Thus, typically the disclosed variants and humanized forms of the antibody maintain the ability to bind nucleic acids. In addition, 3E10 scFv has previously been shown capable of penetrating into living cells and nuclei in an ENT2-dependent manner, with efficiency of uptake impaired in ENT2-deficient cells (Hansen, et al., J. Biol. Chem. 282, 20790-20793 (2007)). Thus, in some embodiments, the disclosed humanized forms and variants of the antibody maintain the ability to penetrate into cell nuclei in an ENT-dependent, preferably ENT2-dependent manner.

As discussed in US 2021/0054102 and US 2021/0137960 some humanized 3E10 variant were found to penetrate cell nuclei more efficiently than the original murine 3E10 (D31N) di-scFv, while others were found to have lost the ability to penetrate nuclei. In particular, variants 10 and 13 penetrated nuclei very well compared to the murine antibody.

Potential bipartite nuclear localization signals (NLS) in humanized 3E10 VL have been identified and may include part or all of the following sequences:

(SEQ ID NO: 109)
RASKSVSTSSYSYMHWYQQKPGQPPKLLIKY;
(SEQ ID NO: 110)
RASKTVSTSSYSYMHWYQQKPGQPPKLLIKY;
or
(SEQ ID NO: 111)
RVTITCRASKSVSTSSYSYMHWYQQKPGKAPKL.

An example consensus NLS can be, or include, (X) RASKTVSTSSYSYMHWYQQKPGQPPKLL (X) KY (where (X)=any residue, but preferentially is a basic residue (R or K) (SEQ ID NO:112) or a variant thereof with at least 60, 65, 70, 75, 80, 85, 90, 95, 96, 97, 98, 99 percent sequence identity to SEQ ID NO:112.

Thus, in some embodiments, particularly where nuclear importation is important, the disclosed antibodies may include the sequence of any one of SEQ ID NOs:109-112, or fragments and variants thereof (e.g., at least 70, 75, 80, 85, 90, 95, 96, 97, 98, 99, or 100% amino acid sequence identity with any one of SEQ ID NOs:109-112) that can translocate into the nucleus of a cell.

Presence of an NLS indicates that a humanized 3E10 antibody or antigen binding fragment thereof may cross the nuclear envelope via the nuclear import pathway. In some embodiments, the NLS improves importation by interacting with one or more members of the import pathway. Thus, in some embodiments, the NLS can bind to importin-β, an importin-β/importin-α heterodimer, or a combination thereof.

### Nucleic Acid Binding

In some embodiments, the disclosed compositions and methods utilize humanized 3E10 antibodies and antigen binding fragments thereof that maintain the ability to bind nucleic acids such as DNA, RNA, or a combination thereof.

The Examples below illustrate molecular modeling of wild type 3E10 sequences and additional 3E10 variants. Molecular modeling of 3E10 (Pymol) revealed a putative Nucleic Acid Binding pocket (NAB1) (see, e.g., FIGS. 11A and 11B), and illustrated with underlining the sequences below.

WT HEAVY CHAIN scFv SEQUENCE
(SEQ ID NO: 113)
E VQLVESGGGL VKPGGSRKLS CAASGFTFSD YGMHWVRQAP EKGLEWVAYI SSGSSTIYYA
DTVKGRFTIS RDNAKNTLFL QMTSLRSEDT AMYYCARRGL LLDYWGQGTT LTVS
LIGHT CHAIN scFv SEQUENCE
(SEQ ID NO: 114)
D IVLTQSPASL AVSLGQRATI SCRASKSVST SSYSYMHWYQ QKPGQPPKLL IKYASYLESG
VPARFSGSGS GTDFTLNIHP VEEEDAATYY CQHSREFPWT FGGGTKLEIK RADAAPGGGG
SGGGGSGGGGS

In some embodiments, the disclosed humanized 3E10 antibodies include some or all of the underlined NAB1 sequences. In some embodiments, the antibodies include a variant sequence that has an altered ability to bind nucleic acids. In some embodiments, the mutations (e.g., substitutions, insertions, and/or deletions) in the NAB1 improve binding of the antibody to nucleic acids such as DNA, RNA, or a combination thereof. In some embodiments, the mutations are conservative substitutions. In some embodiments, the mutations increase the cationic charge of the NAB1 pocket.

As discussed and exemplified herein, mutation of aspartic acid at residue 31 of CDR1 to asparagine increased the cationic charge of this residue and enhanced nucleic acid binding and delivery in vivo (3E10-D31N).

Additional example variants include mutation of aspartic acid at residue 31 of CDR1 to arginine (3E10-D31R), which modeling indicates expands cationic charge, or lysine (3E10-D31K) which modeling indicates changes charge orientation. Thus, in some embodiments, the 3E10 binding protein includes a D31R or D31K substitution.

Additional example variants include mutation of arginine (R) 96 to asparagine (N), and/or serine (S) 30 to aspartic acid (D) alone or in combination with D31N, D31R, or D31K.

All of the sequences disclosed herein having the residue corresponding to 3E10 D31 or N31, are expressly disclosed with a D31R or D31K or N31R or N31K substitution.

Molecular modeling of 3E10 (Pymol) revealed a putative Nucleic Acid Binding pocket (NAB1) (FIGS. 11A-11B). Mutation of aspartic acid at residue 31 of CDR1 to asparagine increased the cationic charge of this residue and enhanced nucleic acid binding and delivery in vivo (3E10-D31N).

Mutation of aspartic acid at residue 31 of CDR1 to arginine (3E10-D31R), further expanded the cationic charge while mutation to lysine (3E10-D31K) changed charge orientation (FIG. 11A).

NAB1 amino acids predicted from molecular modeling have been underlined in the heavy and light chain sequences above. FIG. 11B is an illustration showing molecular modeling of 3E10-scFv (Pymol) with NAB1 amino acid residues illustrated with punctate dots.

All of the sequences disclosed herein having the residue corresponding with R96 are expressly disclosed with R96N substitution.

All of the sequence disclosed herein having the residue corresponding to S30 are expressly disclosed with S30D.

Any of the substitutions can be included in any combination. The sequence having two or three substitution at any combination of residues 31, 30, and 96 are expressly provided. In particular embodiments, the sequence has 31N, 31K, or 31R alone or in combination with 30D, and without the R96N substitution. Thus, in some embodiments, the residue corresponding to 96 is not N, and in more specific embodiments remains R.

### Fragments, Variants, and Fusion Proteins

The anti-nucleic acid antibody can be composed of an antibody fragment or fusion protein including an amino acid sequence of a variable heavy chain and/or variable light chain that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the variable heavy chain and/or light chain of a humanized 3E10 form thereof, e.g., any of SEQ ID NOS:64-102.

The anti-nucleic acid antibody can be composed of an antibody fragment or fusion protein that includes one or more CDR(s) that is at least 45%, at least 50%, at least 55%, at least 60%, at least 65%, at least 70%, at least 75%, at least 80%, at least 85%, at least 90%, at least 95%, at least 99%, or 100% identical to the amino acid sequence of the CDR(s) of a humanized 3E10, or a variant thereof, e.g., the CDR(s) of any of SEQ ID NOS:3-5, 9-11, 15-18, 22-24, and 26-63. The determination of percent identity of two amino acid sequences can be determined by BLAST protein comparison. In some embodiments, the antibody includes one, two, three, four, five, or all six of the 3E10 CDRs described herein.

Preferably, the antibody includes one of each of a heavy chain CDR1, CDR2, and CDR3 in combination with one of each of a light chain CDR1, CDR2, and CDR3.

Predicted complementarity determining regions (CDRs) of the light chain variable sequence for 3E10 are provided above. See also GenBank: AAA65681.1—immunoglobulin light chain, partial [Mus musculus] and GenBank: L34051.1—Mouse Ig rearranged kappa-chain mRNA V-region. Predicted complementarity determining regions (CDRs) of the heavy chain variable sequence for 3E10 are provide above. See also, for example, Zack, et al., Immunology and Cell Biology, 72:513-520 (1994), GenBank Accession number AAA65679.1. Zach, et al., J. Immunol. 154 (4), 1987-1994 (1995) and GenBank: L16982.1—Mouse Ig rearranged H-chain gene, partial cds.

Also included are fragments of antibodies which have nucleic acid delivery activity. The fragments, whether attached to other sequences or not, include insertions, deletions, substitutions, or other selected modifications of particular regions or specific amino acids residues, provided the activity of the fragment is not significantly altered or impaired compared to the nonmodified antibody or antibody fragment.

Techniques can also be adapted for the production of single-chain antibodies specific for nucleic acids of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

The anti-nucleic acid antibodies can be modified to improve their nucleic acid delivery potential. For example, in some embodiments, the cell-penetrating anti-nucleic acid antibody is conjugated to another antibody specific for a therapeutic target in the cytoplasm and/or nucleus of a target cell. For example, the cell-penetrating anti-nucleic acid antibody can be a fusion protein containing 3E10 Fv and a single chain variable fragment of a monoclonal antibody that specifically binds the therapeutic target. In other embodiments, the cell-penetrating anti-nucleic acid antibody is a bispecific antibody having a first heavy chain and a first light chain from 3E10 and a second heavy chain and a second light chain from a monoclonal antibody that specifically binds a therapeutic target.

Bispecific antibodies and other binding proteins having a first heavy chain and a first light chain from 3E10 and a second heavy chain and a second light chain from a monoclonal antibody that specifically binds a target are discussed in Weisbart, et al., Mol. Cancer Ther., 11 (10):2169-73 (2012), and Weisbart, et al., Int. J. Oncology, 25:1113-8 (2004), and U.S. Patent Application No. 2013/0266570, which are specifically incorporated by reference herein in their entireties. In some embodiments, the target is specific for a target cell-type, tissue, organ etc. Thus, the second heavy chain and second light chain can serve as a targeting moiety that targets the complex to the target cell-type, tissue, organ. In some embodiments, the second heavy chain and second light chain target, hematopoietic stem cells, CD34⁺ cells, T cells or any another cell type, e.g., by targeting a receptor or ligand expressed on the cell type. In some embodiments, the second heavy chain and second light chain target the thymus, spleen, or cancer cells.

In some embodiments, particularly those for targeting T cells in vivo, for example, for in vivo production of antigen-specific T cells, CAR T cells, immune cell, or T cell markers such as CD3, CD7, or CD8 can be targeted. For example, anti-CD8 antibodies and anti-CD3 Fab fragments have both been used to target T cells in vivo (Pfeiffer, et al., EMBO Mol Med., 10(11) (2018). pii: e9158. doi: 10.15252/emmm.201809158., Smith, et al., Nat Nanotechnol., 12(8):813-820 (2017). doi: 10.1038/nnano.2017.57). Thus, in some embodiments, the 3E10 antibody or antigen binding fragment or fusion protein is a bispecific antibody part of which can bind specifically to CD3, CD7, CD8, or another immune cell (e.g., T cell) marker, or a marker for a specific tissue such as the thymus, spleen, or liver.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies. In some embodiments, the anti-nucleic acid antibody may contain two or more linked single chain variable fragments of 3E10 (e.g., 3E10 di-scFv, 3E10 tri-scFv), or conservative variants thereof. In some embodiments, the anti-nucleic acid antibody is a diabody or triabody (e.g., 3E10 diabody, 3E10 triabody). Sequences for single and two or more linked single chain variable fragments of 3E10 are provided in US 2019/0247515 and US 2017/0291961.

The function of the antibody may be enhanced by coupling the antibody or a fragment thereof with a therapeutic agent. Such coupling of the antibody or fragment with the therapeutic agent can be achieved by making an immunoconjugate or by making a fusion protein, or by linking the antibody or fragment to a nucleic acid such as DNA or RNA (e.g., siRNA), comprising the antibody or antibody fragment and the therapeutic agent.

In some embodiments, the cell-penetrating antibody is modified to alter its half-life. In some embodiments, it is desirable to increase the half-life of the antibody so that it is present in the circulation or at the site of treatment for longer periods of time. For example, it may be desirable to maintain titers of the antibody in the circulation or in the location to be treated for extended periods of time. In other embodiments, the half-life of the anti-nucleic acid antibody is decreased to reduce potential side effects. Antibody fragments, such as 3E10Fv may have a shorter half-life than full size antibodies. Other methods of altering half-life are known and can be used in the described methods. For example, antibodies can be engineered with Fc variants that extend half-life, e.g., using Xtend™ antibody half-life prolongation technology (Xencor, Monrovia, CA).

### Linkers

The term “linker” as used herein includes, without limitation, peptide linkers. The peptide linker can be any size provided it does not interfere with the binding of the epitope by the variable regions. In some embodiments, the linker includes one or more glycine and/or serine amino acid residues. Monovalent single-chain antibody variable fragments (scFvs) in which the C-terminus of one variable domain are typically tethered to the N-terminus of the other variable domain via a 15 to 25 amino acid peptide or linker. The linker is chosen to permit the heavy chain and light chain to bind together in their proper conformational orientation. Linkers in diabodies, triabodies, etc., typically include a shorter linker than that of a monovalent scFv as discussed above. Di-, tri-, and other multivalent scFvs typically include three or more linkers. The linkers can be the same, or different, in length and/or amino acid composition. Therefore, the number of linkers, composition of the linker(s), and length of the linker(s) can be determined based on the desired valency of the scFv as is known in the art. The linker(s) can allow for or drive formation of a di-, tri-, and other multivalent scFv.

For example, a linker can include 4-8 amino acids. In a particular embodiment, a linker includes the amino acid sequence GQSSRSS (SEQ ID NO:119). In another embodiment, a linker includes 15-20 amino acids, for example, 18 amino acids. In a particular embodiment, the linker includes the amino acid sequence GQSSRSSSGGGSSGGGGS (SEQ ID NO:120). Other flexible linkers include, but are not limited to, the amino acid sequences Gly-Ser, Gly-Ser-Gly-Ser (SEQ ID NO:121), Ala-Ser, Gly-Gly-Gly-Ser (SEQ ID NO:122), (Gly₄-Ser)₂ (SEQ ID NO:123) and (Gly₄-Ser)₄ (SEQ ID NO:124), and (Gly-Gly-Gly-Gly-Ser)₃ (SEQ ID NO:125).

Other example linkers include, for example, RADAAPGGGGSGGGGSGGGGS (SEQ ID NO:126) and ASTKGPSVFPLAPLESSGS (SEQ ID NO:127).

### Nucleic Acid Cargo

As used in some embodiments of the methods and composition provided herein, the humanized 3E10 antibody or antigen binding fragment thereof is complexed with a nucleic acid cargo (a polynucleotide). In some embodiments, the polynucleotide is non-covalently bound to the humanized 3E10 antibody or antigen binding fragment thereof. In some embodiments, the polynucleotide is covalently conjugated to the humanized 3E10 antibody or antigen binding fragment thereof.

The nucleic acid cargo can be single stranded or double stranded. The nucleic acid cargo can be or include DNA, RNA, nucleic acid analogs, or a combination thereof. As discussed in more detail below, nucleic acid analogs can be modified at the base moiety, sugar moiety, or phosphate backbone. Such modification can improve, for example, stability, hybridization, or solubility of the nucleic acid.

The nucleic acid cargo is typically functional in the sense that it is or encodes an agent that is biologically active once delivered into cells. Example cargo is discussed in more detail below, but includes, for example, mRNA or DNA encoding polypeptides of interest including, for example expression constructs and vectors, inhibitory nucleic acids such as siRNA, or nucleic acid encoding the inhibitory nucleic acid including, for example expression constructs and vectors.

The disclosed compositions can include a plurality of a single nucleic acid cargo molecule. In some embodiments, the compositions include a plurality of a multiplicity (e.g., 2, 3, 4, 5, 6, 7, 8, 9 10, or more) of different nucleic acid molecules.

In some embodiments, the cargo molecules are about 0.001, about 0.01, about 1, 10's, 100's, 1,000's, 10,000's, and/or 100,000's of kilobases in length.

In some embodiments, e.g., the cargo is between 0.001 kb and 100 kb, or between 0.001 kb and 50 kb, or between 0.001 kb and 25 kb, or between 0.001 kb and 12.5 kb, or between 0.001 kb and 10 kb, or between 0.001 kb and 8 kb, or 0.001 kb and 5 kb, or between 0.001 kb and 2.5 kb, or between 0.001 kb and 1 kb, or between 0.01 kb and 100 kb, or between 0.01 kb and 50 kb, or between 0.01 kb and 25 kb, or between 0.01 kb and 12.5 kb, or between 0.01 kb and 10 kb, or between 0.01 kb and 8 kb, or 0.01 kb and 5 kb, or between 0.01 kb and 2.5 kb, or between 0.01 kb and 1 kb, or between 0.1 kb and 100 kb, or between 0.1 kb and 50 kb, or between 0.1 kb and 25 kb, or between 0.1 kb and 12.5 kb, or between 0.1 kb and 10 kb, or between 0.1 kb and 8 kb, or 0.1 kb and 5 kb, or between 0.1 kb and 2.5 kb, or between 0.1 kb and 1 kb, or between 1 kb and 100 kb, or between 1 kb and 50 kb, or between 1 kb and 25 kb, or between 1 kb and 12.5 kb, or between 1 kb and 10 kb, or between 1 kb and 8 kb, or 1 kb and 5 kb, or between 1 kb and 2.5 kb, each inclusive.

In some embodiments, e.g., the cargo is between about 0.001 kb and about 100 kb, or between about 0.001 kb and about 50 kb, or between about 0.001 kb and about 25 kb, or between about 0.001 kb and about 12.5 kb, or between about 0.001 kb and about 10 kb, or between about 0.001 kb and about 8 kb, or about 0.001 kb and about 5 kb, or between about 0.001 kb and about 2.5 kb, or between about 0.001 kb and about 1 kb, or between about 0.01 kb and about 100 kb, or between about 0.01 kb and about 50 kb, or between about 0.01 kb and about 25 kb, or between about 0.01 kb and about 12.5 kb, or between about 0.01 kb and about 10 kb, or between about 0.01 kb and about 8 kb, or about 0.01 kb and about 5 kb, or between about 0.01 kb and about 2.5 kb, or between about 0.01 kb and about 1 kb, or between about 0.1 kb and about 100 kb, or between about 0.1 kb and about 50 kb, or between about 0.1 kb and about 25 kb, or between about 0.1 kb and about 12.5 kb, or between about 0.1 kb and about 10 kb, or between about 0.1 kb and about 8 kb, or between about 0.1 kb and about 5 kb, or between about 0.1 kb and about 2.5 kb, or between about 0.1 kb and about 1 kb, or between about 1 kb and about 100 kb, or between about 1 kb and about 50 kb, or between about 1 kb and about 25 kb, or between about 1 kb and about 12.5 kb, or between about 1 kb and about 10 kb, or between about 1 kb and about 8 kb, or between about 1 kb and about 5 kb, or between about 1 kb and about 2.5 kb, each inclusive.

In some embodiments, e.g., the cargo is between 0.2 kb and 10 kb, or between 0.2 kb and 5 kb, or between 0.2 kb and 2.5 kb, or between 0.2 kb and 1 kb, or between 0.2 kb and 0.5 kb, or between 0.2 kb and 0.25 kb, or between 0.5 kb and 10 kb, or between 0.5 kb and 5 kb, or between 1 kb and 5 kb, or between 1 kb and 3 kb, or between 2 kb and 10 kb, or between 3 kb and 5 kb.

In some embodiments, e.g., the cargo is between about 0.2 kb and about 10 kb, or between about 0.2 kb and about 5 kb, or between about 0.2 kb and about 2.5 kb, or between about 0.2 kb and about 1 kb, or between about 0.2 kb and about 0.5 kb, or between about 0.2 kb and about 0.25 kb, or between about 0.5 kb and about 10 kb, or between about 0.5 kb and about 5 kb, or between about 1 kb and about 5 kb, or between about 1 kb and about 3 kb, or between about 2 kb and about 10 kb, or between about 3 kb and about 5 kb.

It will be appreciated that for specific application the nucleic acid cargo may be one or more discrete lengths that, for example, falls within one of the foregoing ranges (inclusive), the specific values for each are expressly disclosed. For example, the size can be as small as a single nucleotide or nucleobase. In an example application the cargo is a cyclic dinucleotide like cGAMP, which is a STING agonist. In other embodiments, the cargo is a short oligomer. For example, oligomers as short as 8-mers can be used for anti-sense or splice switching. Slightly longer ones (e.g., 18 to 20 mers) can be used for gene editing.

### Immunostimulatory Oligonucleotides

Macromolecular stimulators of the innate immune system, particularly polynucleotide agonists of pattern recognition receptors (PRRs), hold great promise for the treatment of cancer. Pattern recognition receptors (PRRs) recognize pathogen-associated as well as endogenous damage-associated molecular patterns. Once ligand binding occurs, signaling cascades develop within the cells to activate effector molecules, resulting in the recruitment and activation of anti-tumor immune cells and release of inflammatory cytokines. As such, PRR agonists have been used with success as immunotherapies for the treatment of a wide range of cancers. For a review of PRRs and the use of PRR agonists in cancer immunotherapies see, for example, Bai L., et al., “Promising targets based on pattern recognition receptors for cancer immunotherapy,” Pharmacological Research, 159 (2020) 105017, the contents of which are incorporated by reference herein in their entirety.

In some embodiments, a humanized 3E10 antibody or antigen binding fragment thereof is complexed with a polynucleotide immunostimulant, e.g., a polynucleotide capable of stimulating a pattern recognition receptor (PRR). In some embodiments, the polynucleotide immunostimulant is non-covalently bound to the humanized 3E10 antibody or antigen binding fragment thereof. In some embodiments, the polynucleotide immunostimulant is covalently conjugated to the humanized 3E10 antibody or antigen binding fragment thereof.

### Pattern Recognition Receptor (PRR) Agonists

In one aspect, the present disclosure relates to compositions and methods for treating cancer in subject by administering to the subject a therapeutically effective amount of a composition including a complex formed between (i) a humanized 3E10 antibody or antigen binding fragment thereof, and (ii) a polynucleotide ligand capable of stimulating a pattern recognition receptor (PRR), as described herein. As recognized in the art, stimulation of the innate immune system, e.g., through activation of pattern recognition receptors, represents a promising therapeutic avenue, particularly for treating cancer. Generally, PRRs stimulate the innate immune system following recognition of pathogen-associated patterns (PAMPs) and/or damage-associated patterns (DAMPs). Conventionally, PRRs are grouped into five categories: Toll-like receptors (TLRs), C-type lectin receptors (CLRs), RIG-I-like receptors (RLRs), Nucleotide-binding Oligomerization Domain (NOD)-like receptors (NLRs), and cytosolic DNA sensors (CDS). The method and compositions described herein act through any of these classes of PRRs the recognize, and are activated by, a polynucleotide antigen.

RIG-I like receptors (RLRs) are a family of RNA helicases that function as cytoplasmic sensors of pathogen-associated molecular patterns (PAMPs) within viral RNA. Accordingly, in another aspect, compositions and methods are provided for treating a cancer by administering to the subject a therapeutically effective amount of a composition including a complex formed between (i) a humanized 3E10 antibody or antigen binding fragment thereof (ii) a polynucleotide ligand capable of stimulating a RIG-I-like receptor (RLR). Identified RLRs include RIG-I (retinoic acid-inducible gene I), MDA5 (melanoma differentiation associated factor 5), and LGP2 (laboratory of genetics and physiology 2). Accordingly, in some embodiments, the disclosure provides a composition formed between (i) a humanized 3E10 antibody or antigen binding fragment thereof (ii) a polynucleotide ligand capable of stimulating a RIG-I. Methods for treating cancer by administering such compositions to a subject in need thereof are also provided. In some embodiments, the complex comprises a covalent conjugate between the humanized 3E10 antibody or antigen binding fragment thereof and the polynucleotide ligand.

Example RIG-I ligands include, but are not limited to, 5′ppp-dsRNA, a specific agonist of RIG-I; 3p-hpRNA, a specific agonist of RIG-I; Poly(I:C)/LyoVec complexes that are recognized by RIG-I and/or MDA-5 depending of the size of poly(I:C); Poly(dA:dT)/LyoVec complexes that are indirectly recognized by RIG-I. In some embodiments, the 3p-hpRNA is a 5′ triphosphate hairpin RNA that was generated by in vitro transcription of a sequence from the influenza A (H1N1). In some embodiments, the 3p-hpRNA is an RNA oligonucleotide that contains an uncapped 5′ triphosphate extremity and a double-strand fragment. In some embodiments, the 3p-hpRNA is about 50 bp, about 55 bp, about 60 bp, about 65 bp, about 70 bp, about 75 bp, about 80 bp, about 85 bp, about 90 bp, about 100 bp, or more. In some embodiments, the 3p-hpRNA is 89 bp long.

In some embodiments, a polynucleotide capable of stimulating RIG-I is non-covalently bound to the humanized 3E10 antibody or antigen binding fragment thereof. In some embodiments, a polynucleotide capable of stimulating RIG-I is covalently conjugated to the humanized 3E10 antibody or antigen binding fragment thereof.

Accordingly, in some embodiments, compositions and methods are provided for treating a cancer by administering to the subject a therapeutically effective amount of a composition including a complex formed between (i) a humanized 3E10 antibody or antigen binding fragment thereof, and (ii) an RNA molecule that is at least partially double-stranded and is capable of stimulating RIG-I. In some embodiments, the at least partially double-stranded RNA molecule comprises two separate RNA strands that anneal to form a double-stranded portion of the molecule. In other embodiments, the at least partially double-stranded RNA molecule is a single RNA strand with self-complementarity, such that under physiological conditions it anneals to itself to form a double-stranded portion of the molecule, e.g., thereby forming one or more hairpin structures.

Similarly, in some embodiments, compositions and methods are provided for treating a cancer by administering to the subject a therapeutically effective amount of a composition including a complex formed between (i) a humanized 3E10 antibody or antigen binding fragment thereof, and (ii) a polynucleotide that is at least partially double-stranded, contains at least one 5′ triphosphate moiety, and is capable of stimulating RIG-I. In some embodiments, the at least partially double-stranded RNA molecule comprises two separate RNA strands that anneal to form a double-stranded portion of the molecule. In other embodiments, the at least partially double-stranded RNA molecule is a single RNA strand with self-complementarity, such that under physiological conditions it anneals to itself to form a double-stranded portion of the molecule, e.g., thereby forming one or more hairpin structures. Examples of polynucleotide RIG-I agonists are provided in the literature. Generally, any one of these polynucleotide RIG-I agonists finds use in the methods and compositions described herein.

In some embodiments, the RIG-I agonist is a 5′ triphosphate hairpin RNA that was generated by in vitro transcription of a sequence from the influenza A (H1N1) virus, a single-stranded negative-sense RNA virus (3p-hpRNA) having the sequence 5′-pppGGAGCAAAAGCAGGGUGACAAAGACAUAAUGGAUCCAAACACUGUGUCAAGC UUUCAGGUAGAUUGCUUUCUUUGGCAUGUCCGCAAAC-3′ (SEQ ID NO:103), or a highly conserved nucleotide sequence thereto. See, for example, Rehwinkel J. et al., Cell, 140:397-408 (2010) and Liu G. et al., J Virol. 89(11):6067-79 (2015), the contents of which are incorporated herein by reference, in their entireties, for all purposes. Accordingly, in some embodiments, compositions and methods are provided for treating a cancer by administering to the subject a therapeutically effective amount of a composition including a complex formed between (i) a humanized 3E10 antibody or antigen binding fragment thereof, and (ii) a polynucleotide having the sequence of 3p-hpRNA (SEQ ID NO:103) that is capable of stimulating RIG-I.

In some embodiments, the RIG-I agonist has a sequence that is at least 80% identical to the sequence of 3p-hpRNA (SEQ ID NO:103). In some embodiments, the RIG-I agonist has a sequence that is at least 85% identical to the sequence of 3p-hpRNA (SEQ ID NO:103). In some embodiments, the RIG-I agonist has a sequence that is at least 90% identical to the sequence of 3p-hpRNA (SEQ ID NO:103). In some embodiments, the RIG-I agonist has a sequence that is at least 95% identical to the sequence of 3p-hpRNA (SEQ ID NO:103). In some embodiments, the RIG-I agonist has a sequence that is at least 96% identical to the sequence of 3p-hpRNA (SEQ ID NO:103). In some embodiments, the RIG-I agonist has a sequence that is at least 97% identical to the sequence of 3p-hpRNA (SEQ ID NO:103). In some embodiments, the RIG-I agonist has a sequence that is at least 98% identical to the sequence of 3p-hpRNA (SEQ ID NO:103). In some embodiments, the RIG-I agonist has a sequence that is at least 99% identical to the sequence of 3p-hpRNA (SEQ ID NO:103). Accordingly, in some embodiments, compositions and methods are provided for treating a cancer by administering to the subject a therapeutically effective amount of a composition including a complex formed between (i) a humanized 3E10 antibody or antigen binding fragment thereof, and (ii) a polynucleotide having a sequence that is at least 80%, at least 85%, at least 90%, at least 95%, at least 96%, at least 97%, at least 98%, or at least 99% identical to the nucleotide sequence of 3p-hpRNA (SEQ ID NO:103) that is capable of stimulating RIG-I.

Other RIG-I agonists that find use in the methods and compositions disclosed herein are known in the art. For example, useful RIG-I agonists that can be complexed with the humanized 3E10 antibodies and antigen binding fragments thereof disclosed herein are described in WO 2023/278897, US20100178272, U.S. Pat. No. 9,738,680, US 2011/0184045, U.S. Pat. No. 10,059,943, US 2018/0195063, U.S. Pat. Nos. 11,382,966, 11,542,505, WO 2020/260547, US 2021/0260093, U.S. Pat. No. 9,226,959, US 2014/0286998, U.S. Pat. Nos. 9,861,574, 9,775,894, US 2021/0046168, US 2019/0076463, U.S. Pat. Nos. 11,499,157, 10,907,161, and US 2022/0333113, the disclosures of which are hereby incorporated herein the reference, in their entireties.

In some embodiments, compositions and methods are provided for treating a cancer by administering to the subject a therapeutically effective amount of a composition including a complex formed between (i) a humanized 3E10 antibody or antigen binding fragment thereof, and (ii) a polynucleotide ligand capable of stimulating a Toll-like receptor (TLR). At least 13 Toll-like receptors have been identified, including TLR1, TLR2, TLR3, TLR4, TLR5, TLR6, TLR7, TLR8, TLR9, TLR10, TLR11, TLR12, and TLR13. Each of these Toll-like receptors has affinity for a different antigen. In accordance with various embodiments of the present disclosure, the methods and compositions described herein include use a polynucleotide agonist of a TLR. For example, each of TLR3, TLR7, TLR8, and TLR9 have affinity for, and are activated by, various polynucleotides. Accordingly, in some embodiments, the agonists used in the methods and compositions described herein are capable of stimulating TLR3, TLR7, TLR8, or TLR9. In some embodiments, the complex is a non-covalent complex. In some embodiments, the complex comprises a covalent conjugate between the humanized 3E10 antibody or antigen binding fragment thereof and the polynucleotide ligand.

For example, unmethylated CpG sites can be detected by TLR9 on plasmacytoid dendritic cells and B cells in humans (Zaida, et al., Infection and Immunity, 76(5):2123-2129, (2008)). Therefore, the sequence of oligonucleotide can include one or more unmethylated cytosine-guanine (CG or CpG, used interchangeably) dinucleotide motifs. The ‘p’ refers to the phosphodiester backbone of DNA, however, in some embodiments, oligonucleotides including CG can have a modified backbone, for example a phosphorothioate (PS) backbone.

In some embodiments, an oligonucleotide can contain more than one CG dinucleotide, arranged either contiguously or separated by intervening nucleotide(s). The CpG motif(s) can be in the interior of the oligonucleotide sequence. Numerous nucleotide sequences stimulate TLR9 with variations in the number and location of CG dinucleotide(s), as well as the precise base sequences flanking the CG dimers.

Typically, CG ODNs are classified based on their sequence, secondary structures, and effect on human peripheral blood mononuclear cells (PBMCs). The five classes are Class A (Type D), Class B (Type K), Class C, Class P, and Class S (Vollmer, J & Krieg, A M, Advanced Drug Delivery Reviews 61 (3): 195-204 (2009), incorporated herein by reference). CG ODNs can stimulate the production of Type I interferons (e.g., IFNα) and induce the maturation of dendritic cells (DCs). Some classes of ODNs are also strong activators of natural killer (NK) cells through indirect cytokine signaling. Some classes are strong stimulators of human B cell and monocyte maturation (Weiner, G L, PNAS USA 94(20): 10833-7 (1997); Dalpke, A H, Immunology 106(1): 102-12 (2002); Hartmann, G, J. of Immun. 164(3):1617-2 (2000), each of which is incorporated herein by reference).

In some embodiments, the polynucleotide immunostimulant is cyclic-GMP-AMP synthase (cGAS) or STimulator of Interferon Qenes (STING).

### Polynucleotides Encoding Effector Polypeptides

In some embodiments, a humanized 3E10 antibody or antigen binding fragment thereof disclosed herein is complexed, covalently or non-covalently, with a therapeutic polynucleotide that encodes a protein or peptide, e.g., an effector polypeptide, for cancer therapy. In some embodiments, the therapeutic polynucleotide is non-covalently bound to the humanized 3E10 antibody or antigen binding fragment thereof. In some embodiments, the therapeutic polynucleotide is covalently conjugated to the humanized 3E10 antibody or antigen binding fragment thereof.

In some embodiments, the polynucleotide is conjugated to or further encodes one or more of RNAi agents, siRNAs, shRNAs, miRNAs, miRNA binding sites, antisense RNAs, ribozymes, catalytic DNA, tRNA, RNAs that induce triple helix formation, aptamers or vectors, and the like.

In other embodiments, the polynucleotide(s) may be designed to encode one or more effector polypeptide(s) or fragments thereof to a cancerous tissue. Such effector polypeptide(s) may include, but is not limited to, whole polypeptides, a plurality of polypeptides, or fragments of polypeptides, which independently may be encoded by one or more regions or parts or the whole of an effector polynucleotide.

An effector polypeptide refers to any polypeptide which is selected to be encoded within, or whose function is affected by, the polynucleotides of the present disclosure. The effector polypeptide can modulate the activity of the immune system and effect the treatment of a cancer, either directly or indirectly, for example by slowing down progression of the cancer, inducing cellular death of cancer cells, inducing senescence of cancer cells, and the like. For example, in some embodiments, an effector polypeptide stimulates immune cells to upregulate the production of cytokines, causing targeting of cancerous cells resulting in cell death. In other embodiments, an effector polypeptide expresses, or stimulates tumor cells to upregulate expression, of tumor antigens which are markers for immune cells to identify tumor cells. For a review of effector polypeptides see, for example, Esensten et al., “CD28 costimulation: from mechanism to therapy,” Immunity Review, 44, (2016) 973; Immunity, 2016, 44, 973-988; Smolle et al., Noncoding RNAs and immune checkpoints, FEBS Journal, 2017, 284, 1952-1966; Chen et al., Anti-PD-1—PD-L1 therapy of human cancer past, present, and future, Journal of Clinical Investigation, 2015, Volume 125, 9, 3384-3391; Rowshanravan et al., CTLA-4 a moving target in immunotherapy, Blood, 2018, Volume 131, 1, 58-67; and Dougall et al., TIGIT and CD96 New checkpoint receptor targets for cancer immunotherapy, Immunological Reviews, 2017, 276, 112-120, the content of each of which is incorporated herein by references, in its entirety, for all purposes.

Accordingly, in one aspect, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide that encodes a protein or peptide, e.g., an effector polypeptide, for cancer therapy, as described herein.

### Tumor Antigens

In some embodiments, the present disclosure provides compositions, as well as methods for treating a cancer in a subject by administering to the subject a therapeutically effective amount of the compositions, including a complex formed between (i) a humanized 3E10 antibody or antigen binding fragment thereof, and (ii) a therapeutic polynucleotide encoding a tumor associated antigen, as described herein. In some embodiments, the therapeutic polynucleotide is non-covalently bound to the humanized 3E10 antibody or antigen binding fragment thereof. In some embodiments, the therapeutic polynucleotide is covalently conjugated to the humanized 3E10 antibody or antigen binding fragment thereof.

Tumor antigens are peptides that are presented almost exclusively on the cell surface of cancerous cells, thereby distinguishing cancerous cells from non-cancerous cells that do not display the tumor antigen. When cancerous cells die, these tumor antigens are released into the tumor microenvironment and can be recognized by the immune system as foreign peptides, altered self-peptides, or self-peptides. Upon release of a sufficient amount of a tumor antigen, the immune system can generate antitumor immunity by generating an immune response to the tumor antigen. Specifically, the immune system targets and destroys cancerous cells displaying the tumor antigen that was used to generate the immune response.

Several classes of antigens have been exploited experimentally to generate antitumor immunity. Specifically, tumor antigens are exogenously administered, as either a peptide or a nucleic acid encoding the antigen, to a patient with a cancer displaying the tumor antigen on its cell surface. In turn, the immune system is presented with sufficient amounts of the antigen to generate an immune response against the tumor antigen, resulting in antitumor immunity. Examples of classes of tumor antigens include oncoviral protein antigens, neoantigens, and antigens derived from a cancer-germline gene. Often, the tumor antigen, or polynucleotide encoding the tumor antigen, is co-administered with an adjuvant that activated dendritic cells, or with dendritic cells themselves, to promote generation of the antitumor immunity. For review, see, for example, Haen et al., “Towards new horizons Characterization, classification and implications of the tumor antigenic repertoire,” Nature Reviews-Clinical Oncology, 2020, Volume 17, 595-610; Saxena M. et al., Nat. Rev. Cancer 21, 360-378 (2021), all the contents of which are incorporated herein by reference.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding an oncoviral protein antigen, as described herein.

At least six oncoviruses, cancer-causing viruses, have been known so far, which include hepatitis B virus, hepatitis C virus, Epstein-Barr virus (EBV or HHV-4), human papillomavirus, human T lymphotropic virus type 1, Kaposi's sarcoma-associated herpesvirus (KSHV or HHV-8), but the pathogenic mechanism is far from being completely understood.

An oncoviral protein antigen is an antigen presented on the cell surface of a cancer that is derived from an oncogenic virus associated with the cancer. For instance, a vast majority of cervical cancers are associated, if not caused by, HPV infection. Accordingly, an antigen derived from HPV that is presented on the cell surface of a cervical cancer cell represents an oncoviral protein antigen. Non-limiting examples of oncoviral protein antigens and examples of cancers associated with these antigens are presented in Table 1.

TABLE 1
Examples of oncoviral proteins and associated cancer types.

| Virus | Associated cancer types | Protein Antigens |
|---|---|---|
| Human | HPV types 16 and 18 are | E6/E7-associated protein |
| papillomaviruses | associated with cancers of cervix, | |
| (HPV) | anus, penis, vulva, vagina, | |
| | and HPV-positive oropharyngeal | |
| | cancers. | |
| | Human papillomavirus types 8, 18, | |
| | and 5 associated with squamous | |
| | cell carcinoma | |
| Epstein-Barr | Burkitt's lymphoma, Hodgkin's | EBV nuclear antigens (EBNAs) 1, 2, |
| virus (EBV) | lymphoma, post-transplant | 3a, 3b, 3c and LP, BARF1, latent |
| | lymphoproliferative | membrane proteins (LMPs) 1, 2a, and |
| | disease, nasopharyngeal | 2b, BamHI A rightward transcripts |
| | carcinoma and a subtype | (BARTs), EBV-encoded RNAs |
| | of stomach cancer. | (EBERs). |
| Hepatitis B virus | Hepatocellular carcinoma | Hepatitis B viral protein (HBcAg) |
| (HBV), hepatitis | | |
| C virus (HCV) | | |
| Human T- | Adult T-cell leukemia | Tax |
| lymphotropic | | |
| virus 1 (HTLV-1) | | |

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding an oncoviral protein antigen derived from a viral protein listed in Table 1, as described herein. In some embodiments, the therapeutically effective amount of the composition is co-administered with an adjuvant. In some embodiments, a therapeutically effective amount of the composition is co-administered with dendritic cells.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding a neoantigen, as described herein. Neoantigens are peptides, presented on the surface of a cancer cell, having an amino acid sequence that is novel to a cancerous tissue. That is, the neoantigen has an amino acid sequence that is not present in the germline (wildtype) human genome. Neoantigens are created by mutation of the genome during or after development of the cancer and, in this fashion, are specific to an individual patient.

Deep-sequencing technologies can be used to identify mutations present within the exome of an individual tumor to predict neoantigens. This is done by identifying neoantigens that can be recognized by T cells. For example, in some embodiments, tumor material is analyzed for nonsynonymous somatic mutations. RNA sequencing data are used to focus on mutations in expressed genes. Peptide stretches containing any of the identified nonsynonymous mutations are generated in silico and are either left unfiltered, filtered using a predictive algorithm, or used to identify MHC-associated neoantigens in mass spectrometry data generated from the patient's cancerous tissue. Modeling of the effect of mutations on the resulting peptide-MHC complex may be used as an additional filter, to identify particularly promising neoantigens. Resulting epitope sets can also be used to identify physiologically occurring neoantigen-specific T cell responses by MHC multimer-based screens. See, e.g., Science, 3 Apr. 2015: Vol. 348, Issue 6230, pp. 69-74. However, other techniques, including exomic analysis and proteomic analysis can also be used to identify novel genomic or novel peptide sequences corresponding to a neoantigen, respectively. Non-limiting examples of neoantigens identified from individual cancers, using transcriptomic, exomic, and proteomic analyses are described, for example, in Haen et al., Towards new horizons Characterization, classification and implications of the tumor antigenic repertoire, Nature Reviews-Clinical Oncology, 2020, Volume 17, 595-610.

Thus, in some embodiments, the present disclosure provides a method for treating a cancer in a subject by first identifying a neoantigen of the cancer in the subject, and second administering to the subject a therapeutically effective amount of a composition including a complex formed between (i) a humanized 3E10 antibody or antigen binding fragment thereof, and (ii) a therapeutic polynucleotide encoding the identified neoantigen, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding an antigen derived from a cancer-germline gene, as described herein. Cancer germline antigens are a class of immunogenic tumor antigens encoded by genes expressed in gametogenic cells of the testis and/or ovary and in human cancer. Examples of cancer germline antigens include, but are not limited to, antigens derived from synovial sarcoma X-2 (SSX-2), New York-esophageal squamous cell carcinoma-1 (NY-ESO-1), melanoma associated antigen 1 (MAGA1), and melanoma associated antigen 3 (MAGA3), each which are over-expressed in different human cancers such as in melanoma and lung cancer.

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding an antigen derived from an SSX-2, NY-ESO-1, MAGA1, or MAGA3 protein, as described herein. In some embodiments, the cancer is melanoma. In some embodiments, the cancer is a lung cancer.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding a tumor-associated antigen (TAA), as described herein. In some embodiments, the therapeutically effective amount of the composition is co-administered with an adjuvant. In some embodiments, the therapeutically effective amount of the composition is co-administered with dendritic cells.

Tumor-associated antigens are peptides derived from wild-type protein sequences or glycoprotein synthesized by thetumorcell. TAA proteins can reside in any subcellular compartment of the tumor cell; for example, they may be membrane-bound, cytoplasmic, nuclear-localized, or even secreted by the tumor cells. TAAs are primarily generated by genetic amplification or post-translational modifications, that cause the underlying protein to be differentially expressed within cancer cells, relative to non-cancerous cells, and allow for a preferential recognition of tumor cells by specific T cells or immunoglobulins. Non-limiting examples of tumor associated antigens that have been identified are presented in Tables 2-3.

TABLE 2
Examples of tumor associated antigens (TAAs)
and associated cancers.

| Tumor Associated Antigen | Cancers |
|---|---|
| Alphafetoprotein (AFP) | Germ cell tumors |
| | Hepatocellular carcinoma |
| Carcinoembryonic antigen (CEA) | Bowel cancers |
| CA-125 | Ovarian cancer |
| MUC-1 | Breast cancer |
| Epithelial tumor antigen (ETA) | Breast cancer |
| Tyrosinase | Malignant melanoma |

TABLE 3
Examples of tumor associated antigens (TAAs) and associated references, all of which are
incorporated herein by reference for the purpose of identifying the referenced tumor markers.

| Tumor Antigen | Example Reference |
|---|---|
| 5-alpha reductase | Delos et al. (1998) Int. J. Cancer, 75: 6 840-846 |
| a-fetoprotein | Esteban et al. (1996) Tumor Biol., 17(5): 299-305 |
| AM-1 | Harada et al. (1996) Tohoku J Exp Med., 180(3): 273-288 |
| APC | Dihlmannet al. (1997) Oncol. Res., 9(3) 119-127 |
| APRIL | Sordat et al. (1998) Exp Med., 188(6): 1185-1190 |
| BAGE | Boel et al. (1995) Immunity, 2: 167-175. |
| β-catenin | Hugh et al. (1999) Int Cancer, 82(4): 504-11 |
| Bc12 | Koty et al. (1999) Lung Cancer, 23(2): 115-127 |
| bcr-abl (b3a2) | Verfaillie et al. (1996) Blood, 87(11): 4770-4779 |
| CA-125 | Bastet al. (1998) Int. Biol. Markers, 13(4): 179-187 |
| CASP-8/FLICE | Mandruzzato et al. (1997) J Exp. Med., 186(5): 785-793. |
| Cathepsins | Thomssen et al. (1995) Clin. Cancer Res., 1 (7): 741-746 |
| CD19 | Scheuermann et al. (1995) Leuk. Lymphoma, 18(5-6): 385-397 |
| CD20 | Knox et al. (1996) Clin. Cancer Res., 2(3): 457-470 |
| CD21, CD23 | Shubinsky et al. (1997) Leuk. Lymphoma, 25(5-6): 521-530 |
| CD22, CD38 | French et al. (1995) Br. J. Cancer, 71 (5): 986-994 |
| CD33 | Nakase et al. (1996) Am. J. Clin. Pathol., 105(6): 761-768 |
| CD35 | Yamakawa et al. Cancer, 73(11): 2808-2817 |
| CD44 | Naot et al. (1997) Adv. Cancer Res., 71: 241-319 |
| CD45 | Buzzi et al. (1992) Cancer Res., 52(14): 4027-4035 |
| CD46 | Yamakawa et al. (1994) Cancer, 73(11): 2808-2817 |
| CD5 | Stein et al. (1991) Clin. Exp. Immunol., 85(3): 418-423 |
| CD52 | Ginaldi et al. (1998) Leuk. Res., 22(2): 185-191 |
| CD55 | Spendlove et al. (1999) Cancer Res., 59: 2282-2286. |
| CD59 (791Tgp72) | Jarvis et al. (1997) Int. J. Cancer, 1049-1055. |
| CDC27 | Wang et al. (1999) Science, 284(5418): 1351-1354 |
| CDK4 | Wolfel et al. (1995) Science, 269(5228): 1281-1284 |
| CEA | Kass et al. (1999) Cancer Res., 59(3): 676-683 |
| c-myc | Watson et al. (1991) Cancer Res., 51 (15): 3996-4000 |
| Cox-2 | Tsujii et al. (1998) Cell, 93: 705-716 |
| DCC | Gotley et al. (1996) Oncogene, 13(4): 787-795 |
| DcR3 | Pitti et al. (1998) Nature, 396: 699-703 |
| E6/E7 | Steller et al. (1996) Cancer Res., 56(21): 5087-5091 |
| EGFR | Yang et al. (1999) Cancer Res., 59(6): 1236-1243. |
| EMBP | Shiina et al. (1996) Prostate, 29(3): 169-176. |
| Ena78 | Arenberg et al. (1998) 102: 465-472. |
| FGF8b and FGF8a | Dorkin et al. (1999) Oncogene, 18(17): 2755-2761 |
| FLK-1/KDR | Annie and Fong (1999) Cancer Res., 59: 99-106 |
| Folic Acid Receptor | Dixon et al. (1992) J. Biol. Chem., 267(33): 24140-72414 |
| G250 | Divgi et al. (1998) Clin. Cancer Res., 4(11): 2729-2739 |
| GAGE−Family | De Backer et al. (1999) Cancer Res., 59(13): 3157-3165 |
| gastrin 17 | Watson et al. (1995) Int. J. Cancer, 61 (2): 233-240 |
| Gastrin-releasing | Wang et al. (1996) Int. J. Cancer, 68(4): 528-534 |
| hormone (bombesin) | |
| GD2/GD3/GM2 | Wiesner and Sweeley (1995) Int. J. Cancer, 60(3): 294-299 |
| GnRH | Bahk et al. (1998) Urol. Res., 26(4): 259-264 |
| GnTV | Hengstler et al. (1998) Recent Results Cancer Res. 154: 47-85 |
| gp100/Pmell7 | Wagner et al. (1997) Cancer Immunol. Immunother., 44(4): 239-247 |
| gp-100-in4 | Kirkin et al. (1998) APMIS, 106(7): 665-679 |
| gpis | Maeurer et al. (1996) Melanoma Res., 6(1): 11-24 |
| gp75/TRP-1 | Lewis et al. (1995) Semin. Cancer Biol., 6(6): 321-327 |
| hCG | Hoermann et al. (1992) Cancer Res., 52(6): 1520-1524 |
| Heparanase | Vlodaysky et al. (1999) Nat. Med., 5(7): 793-802 |
| Her2/neu Her3 | Lewis et al. (1995) Semin Cancer Biol., 6(6): 321-327 |
| HMTV | Kahl et al. (1991) Br J Cancer, 63(4): 534-540 |
| Hsp70 | Jaattela et al. (1998) EMBO J., 17(21): 6124-6134 |
| hTERT (telomerase) | Vonderheide et al. (1999) Immunity. 10: 673-679. 1999. |
| IGFR1 | Ellis et al. (1998) Breast Cancer Res. Treat., 52: 175-184 |
| IL-13R | Murata et al. (1997) Biochem. Biophys. Res. Commun., 238(1): 90-94 |
| iNOS | Klotz et al. (1998) Cancer, 82(10): 1897-1903 |
| Ki 67 | Gerdes et al. (1983) Int. J. Cancer, 31: 13-20 |
| KIAA0205 | Gueguen et al. (1998) J. Immunol., 160(12): 6188-6194 |
| K-ras, H-ras, N-ras | Abrams et al. (1996) Semin. Oncol., 23(1): 118-134 |
| KSA (CO17-1A) | Zhang et al. (1998) Clin. Cancer Res., 4(2): 295-302 |
| LDLR-FUT | Caruso et al. (1998) Oncol. Rep., 5(4): 927-930 |
| MAGE Family | Marchand et al. (1999) Int. J. Cancer, 80(2): 219-230 |
| (MAGE1, MAGE3, | |
| etc.) | |
| Mammaglobin | Watson et al. (1999) Cancer Res., 59: 13 3028-3031 |
| MAP 17 | Kocher et al. (1996) Am. J. Pathol., 149(2): 493-500 |
| Melan-A/MART-1 | Lewis and Houghton (1995) Semin. Cancer Biol., 6(6): 321-327 |
| mesothelin | Chang et al. (1996) Proc. Natl. Acad. Sci. 93(1): 136-140 |
| MIC A/B | Groh et al. (1998) Science, 279: 1737-1740 |
| MT-MMPs, such as | Sato and Seiki (1996) J. Biochem. (Tokyo), 119(2): 209-215 |
| MMP2, MMP3, | |
| MMP7, MMP9 | |
| Moxl | Candia et al. (1992) Development, 116(4): 1123-1136 |
| Mucin, such as MUC-1, | Lewis and Houghton (1995) Semin. Cancer Biol., 6(6): 321-327 |
| MUC-2, MUC-3, | |
| and MUC-4 | |
| MUM-1 | Kirkin et al. (1998) APMIS, \06(T)\ 665-679 |
| NY-ESO-1 | Jager et al. (1998) J. Exp. Med. 187: 265-270 |
| Osteonectin | Graham et al. (1997) Eur. J. Cancer. 33(10): 1654-1660 |
| p15 | Yoshida et al. (1995) Cancer Res. 55(13): 2756-2760 |
| P170/MDR1 | Trock et al. (1997) J. Natl. Cancer Inst., 89(13): 917-931 |
| p53 | Roth et al. (1996) Proc. Natl. Acad. Sci., 93(10): 4781-4786. |
| p97/melanotransferrin | Furukawa et al. (1989) J. Exp. Med., 169(2): 585-590 |
| PAI-1 | Grondahl-Hansen et al. (1993) Cancer Res. 53(11): 2513-2521 |
| PDGF | Vassbotn et al. (1993) Mol. Cell Biol., 13(7): 4066-4076 |
| Plasminogen (uPA) | Naitoh et al. (1995) Jpn. J. Cancer Res., 86(1): 48-56 |
| PRAME | Kirkin et al. (1998) APIS, 106(7): 665-679 |
| Probasin | Matuo et al. (1985) Biochem, Biophys. Res. Commun.,130(1): 293-300 |
| Progenipoietin | Sanda et al. (1999) Urology, 53(2): 260-266. |
| PSA | Kawakami et al. (1997) Cancer Res., 57(12): 2321-2324 |
| PSM | Gaugler et al. (1996) Immunogenetics, 44(5): 323-330 |
| RAGE−1 | Dosaka-Akita et al. (1997) Cancer, 79(7): 1329-1337 |
| Rb | Sonoda et al. (1996) Cancer, 77(8) 1501-1509. |
| RCAS1 | Kikuchi et al. (1999) Int. J. Cancer, 81 (3): 459-466 |
| SART-1 | Gure et al. (1997) Int. J. Cancer, 72(6): 965-971 |
| SSX gene | Bromberg et al. (1999) Cell, 98(3): 295-303 |
| family | Sandmaier et al. (1999) J. Immunother., 22(1): 54-66 |
| STAT3 (mucin assoc.) | Kuroki et al. (1990) Cancer Res., 50(16): 4872-4879 |
| TAG-72 | Imanishi et al. (1989) Br. J. Cancer, 59(5): 761-765 |
| TGF-ct | Picon et al. (1998) Cancer Epidemiol Biomarkers Prey, 7(6): 497-504 |
| TGF-β | Bao et al. (1996) Nature Medicine. 2(12), 1322-1328 |
| Thymosin β 15 | Moradi et al. (1993) Cancer, 72(8): 2433-2440 |
| IFN-ct | Maulard et al. (1994) Cancer, 73(2): 394-398 |
| TPA | Nishida et al. (1984) Cancer Res 44(8): 3324-9 |
| TPI | Parkhurst et al. (1998) Cancer Res. 58(21) 4895-4901 |
| TRP-2 | Kirkin et al. (1998) APMIS, 106(7): 665-679 |
| Tyrosinase | Hyodo et al. (1998) Eur. J. Cancer, 34(13): 2041-2045 |
| VEGF | Sanchez et al. (1999) Science, 283(5409): 1914-1919 |
| ZAG | Sanda et al. (1999) Urology, 53(2): 260-266 |
| p16INK4 | Kawakami et al. (1997) Cancer Res., 57(12): 2321-2324 |
| Glutathione S- | Gaugler et al. (1996) Immunogenetics, 44(5): 323-330 |
| transferase | |

Illustrative TAAs include, for example, membrane bound complement regulatory glycoproteins: CD46, CD55 and CD59, which have been found to be expressed on most tumor cells in vivo and in vitro. Human mucins, e.g., MUC1, which are known tumor markers, as are gp100, tyrosinase, and MAGE, which are found in melanoma, see Tables 2-3. Wild-type Wilms' tumor gene WT1 is expressed at high levels not only in most of acute myelocytic, acute lymphocytic, and chronic myelocytic leukemia, but also in various types of solid tumors including lung cancer.

Acute lymphocytic leukemia has been characterized by the TAAs HLA-Dr, CD1, CD2, CD5, CD7, CD 19, and CD20. Acute myelogenous leukemia has been characterized by the TAAs HLA-Dr, CD7, CD13, CD14, CD15, CD33, and CD34. Breast cancer has been characterized by the markers EGFR, HER2, MUC1, Tag-72. Various carcinomas have been characterized by the markers MUC1, TAG-72, and CEA. Chronic lymphocytic leukemia has been characterized by the markers CD3, CD19, CD20, CD21, CD25, and HLA-DR. Hairy cell leukemia has been characterized by the markers CD19, CD20, CD21, CD25. Hodgkin's disease has been characterized by the Leu-Ml marker. Various melanomas have been characterized by the HMB 45 marker. Non-Hodgkin's lymphomas have been characterized by the CD20, CD19, and 1a marker. And various prostate cancers have been characterized by the PSMA and SE10 markers.

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding a tumor-associated antigen derived from a protein listed in Tables 2-3, as described herein. In some embodiments, the therapeutically effective amount of the composition is co-administered with an adjuvant. In some embodiments, the therapeutically effective amount of the composition is co-administered with dendritic cells.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a therapeutic polynucleotide encoding a respective tumor-associated antigen derived from a protein listed in Table 2, as described herein, where the cancer is a cancer associated with the respective tumor-associated antigen within Table 2. In some embodiments, the therapeutically effective amount of the composition is co-administered with an adjuvant. In some embodiments, the therapeutically effective amount of the composition is co-administered with dendritic cells.

In some embodiments, the tumor antigen is an oncoviral protein antigen, a neoantigen, or an antigen derived from a cancer-germline gene.

In some embodiments, the oncoviral protein antigen is a folate receptor, HER2, papillomavirus oncoprotein E6 and papillomavirus oncoprotein E7 carcinoembryonic antigen (CEA), mucin 1, EGFR, squamous cell carcinoma antigen recognized by T cells 3 (SART3), beta-human chorionic gonadotropin (beta-hCG), Wilms' Tumor antigen 1 (WT1), Surviving, MAGE3, p53, ring finger protein 43 and translocase of the outer mitochondrial membrane 34 (TOMM34), prostate-specific antigen (PSA)-TRICOM, or KRAS.

In some embodiments, the tumor associated antigen (TAA) is 5-alpha reductase, a-fetoprotein, AM-1, APC, APRIL, BAGE, β-catenin, Bcl2, bcr-abl (b3a2), CA-125, CASP-8/FLICE, Cathepsins, CD19, CD20, CD21, CD23, CD22, CD38, CD33, CD35, CD44, CD45, CD46, CD5, CD52, CD55, CD59 (791Tgp72), CDC27, CDK4, CEA, c-myc, Cox-2, DCC, DcR3, E6/E7, EGFR, EMBP, Ena78, FGF8b and FGF8a, FLK-1/KDR, Folic Acid Receptor, G250, GAGE-Family, gastrin 17, Gastrin-releasing hormone (bombesin), GD2/GD3/GM2, GnRH, GnTV, gp100/Pmell7, gp-100-in4, gpis, gp75/TRP-1, hCG, Heparanase, Her2/neu Her3, HMTV, Hsp70, hTERT (telomerase), IGFR1, IL-13R, iNOS, Ki 67, KIAA0205, K-ras, H-ras, N-ras, KSA (CO17-1A), LDLR-FUT, MAGE Family (MAGE1, MAGE3, etc.), Mammaglobin, MAP 17, Melan-A/MART-1, mesothelin, MIC A/B, MT-MMPs, such as MMP2, MMP3, MMP7, MMP9, Moxl, Mucin, such as MUC-1, MUC-2, MUC-3, and MUC-4, MUM-1, NY-ESO-1, Osteonectin, p15, P170/MDR1, p53, p97/melanotransferrin, PAI-1, PDGF, Plasminogen (uPA), PRAME, Probasin, Progenipoietin, PSA, PSM, RAGE-1, Rb, RCAS1, SART-1, SSX gene family, STAT3 (mucin assoc.), TAG-72, TGF-ct, TGF-β, Thymosin β 15, IFN-ct, TPA, TPI, TRP-2, Tyrosinase, VEGF, ZAG, pl6INK4, or Glutathione S-transferase

In some embodiments, the neoantigen is BRCA1, BRCA2 BRAF, KRAS, EGFR, IDH1, PIK3CA, ROS1, HLA, JAK1, JAK2, PARK2, ATM, p53, TP53, erbb2 interacting protein (ERBB2IP), Beta-2-Microglobulin (β2m), cyclin-dependent kinase inhibitor 2A (CDKN2A), alternate reading frame (ARF), or cyclin-dependent kinase 4 (CDK4).

In some embodiments, the cancer germline gene is MAGEA1, MAGEA2, MAGEA3, MAGEA4, MAGEA5, MAGEA6, MAGEA8, MAGEA9, MAGEA10, MAGEA11, MAGEA12, BAGE, BAGE2, BAGE3, BAGE4, BAGE5, MAGEB1, MAGEB2, MAGEB5, MAGEB6, MAGEB3, MAGEB4, GAGE1, GAGE2A, GAGE3, GAGE4, GAGE5, GAGE6, GAGE7, GAGE8, SSX1, SSX2, SSX2b, SSX3, SSX4, CTAG1B, LAGE-1b, CTAG2, MAGEC1, MAGEC3, SYCP1, BRDT, MAGEC2, SPANXA1, SPANXB1, SPANXC, SPANXD, SPANXN1, SPANXN2, SPANXN3, SPANXN4, SPANXN5, XAGE1D, XAGE1C, XAGE1B, XAGE1, XAGE2, XAGE3, XAGE-3b, XAGE-4/RP11-167P23.2, XAGE5, DDX43, SAGE1, ADAM2, PAGE5, CT16.2, PAGE1, PAGE2, PAGE2B, PAGE3, PAGE4, LIPI, VENTXP1, IL13RA2, TSP50, CTAGE1, CTAGE-2, CTAGE5, SPA17, ACRBP, CSAG1, CSAG2, DSCR8, MMA1b, DDX53, CTCFL, LUZP4, CASC5, TFDP3, JARID1B, LDHC, MORC1, DKKL1, SPO11, CRISP2, FMR1NB, FTHL17, NXF2, TAF7L, TDRD1, TDRD6, TDRD4, TEX15, FATE1, TPTE, CT45A1, CT45A2, CT45A3, CT45A4, CT45A5, CT45A6, HORMAD1, HORMAD2, CT47A1, CT47A2, CT47A3, CT47A4, CT47A5, CT47A6, CT47A7, CT47A8, CT47A9, CT47A10, CT47A11, CT47B1, SLCO6A1, TAG, LEMD1, HSPB9, CCDC110, ZNF165, SPACA3, CXorf48, THEG, ACTL8, NLRP4, COX6B2, LOC348120, CCDC33, LOC196993, PASD1, LOC647107, TULP2, CT66/AA884595, PRSS54, RBM46, CT69/BC040308, CT70/BI818097, SPINLW1, TSSK6, ADAM29, CCDC36, LOC440934, SYCE1, CPXCR1, TSPY3, TSGA10, HIWI, MIWI, PIWI, PIWIL2, ARMC3, AKAP3, Cxorf61, PBK, C21orf99, OIP5, CEP290, CABYR, SPAG9, MPHOSPH1, ROPN1, PLAC1, CALR3, PRM1, PRM2, CAGE1, TTK, LY6K, IMP-3, AKAP4, DPPA2, KIAA0100, DCAF12, SEMG1, POTED, POTEE, POTEA, POTEG, POTEB, POTEC, POTEH, GOLGAGL2 FA, CDCA1, PEPP2, OTOA, CCDC62, GPATCH2, CEP55, FAM46D, TEX14, CTNNA2, FAM133A, LOC130576, ANKRD45, ELOVL4, IGSF11, TMEFF1, TMEFF2, ARX, SPEF2, GPAT2, TMEM108, NOL4, PTPN20A, SPAG4, MAEL, RQCD1, PRAME, TEX101, SPATA19, ODF1, ODF2, ODF3, ODF4, ATAD2, ZNF645, MCAK, SPAG1, SPAG6, SPAG8, SPAG17, FBXO39, RGS22, cyclin A1, C15orf60, CCDC83, TEKT5, NR6A1, TMPRSS12, TPPP2, PRSS55, DMRT1, EDAG, NDR, DNAJB8, CSAG3B, CTAG1A, GAGE12B, GAGE12C, GAGE12D, GAGE12E, GAGE12F, GAGE12G, GAGE12H, GAGE12L, GAGE12J, GAGE13, LOC728137, MAGEA2B, MAGEA9B/LOC728269, NXF2B, SPANXA2, SPANXB2, SPANXE, SSX4B, SSX5, SSX6, SSX7, SSX9, TSPY1D, TSPY1E, TSPY1F, TSPY1G, TSPY1H, TSPY1I, TSPY2, or XAGE1E.

CD28 is a member of a subfamily of costimulatory molecules characterized by an extracellular variable immunoglobulin-like domain. Human CD28 is composed of four exons encoding a protein of 220 amino acids that is expressed on the cell surface as a glycosylated, disulfide-linked homodimer of 44 kDa. Members of the CD28 family share a number of common features such as, for example, paired V-set immunoglobulin superfamily (IgSF) domains attached to single transmembrane domains and cytoplasmic domains that contain critical signaling motifs. (Esensten et al., Immunity Review (2016)). CD28 has been reported to regulate T-cell activation via interaction with the signaling motifs. For example, tyrosine phosphorylation of CD28 plays a role in the early signaling events that characterize CD28 costimulation and consequent regulation of T-cell activation. Thus, in some embodiments, the compositions for treating a cancer provided herein include a complex formed between (i) a humanized 3E10 antibody or antigen binding fragment thereof, and (ii) a polynucleotide encoding a signaling motif of a costimulatory molecule having paired V-set immunoglobulin superfamily (IgSF) domains attached to single transmembrane domains and cytoplasmic domains.

### Proinflammatory Cytokines

Proinflammatory cytokines limit tumor cell growth by a direct anti-proliferative or pro-apoptotic activity, or indirectly by stimulating the cytotoxic activity of immune cells against tumor cells. The pro-inflammatory cytokines are secreted from Th1 cells, CD4+ cells, macrophages, and dendritic cells. They are characterized by production of several Interleukins (IL), IL-1, IL-2, IL-12, IL-17, IL-18, IFN-7, and TNF-α. The key pro-inflammatory cytokines are IL-1, IL-6, and TNF-α. These cytokines signal via type I cytokine receptors (CCR1) that are structurally divergent from other cytokine receptor types. They are crucial for coordinating cell mediated immune response and play a critical role in modulating the immune system. Pro-inflammatory cytokines generally regulate growth, cell activation, differentiation, and homing of the immune cells to the sites of infection with the aim to control and eradicate the intracellular pathogens, including viruses.

IL-1 is subdivided in IL-1α and IL-1β. IL-1β is potent pro-inflammatory cytokine, induced mainly by lymphocytes, macrophages, and monocytes in response to microbial molecules. Upon viral infection, the pattern recognition receptors (PPR) and toll-like receptors (TLRs) are expressed which in turn lead to enhanced expression of IL-1β. IL-1β stimulate CD4+ cells and differentiate them towards Th17 cells. In addition to the stimulatory effect of the IL-1 family, there are also members (IL-1RA and IL-1R2) that can inhibit or suppress the IL-1 cytokine expression. IL-1RA is secreted from neutrophils, macrophages, monocytes, and hepatocytes aiming to decrease the inflammation. However, the expression of IL-RA needs to be expressed up to 1,000-fold in order to efficiently inhibit or suppress the expression of IL-1β.

IL-2 was approved for the treatment of advanced renal cell carcinoma (RCC) and metastatic melanoma, and IFN-α was approved for the treatment of hairy cell leukemia, follicular non-Hodgkin lymphoma, melanoma and AIDS-related Kaposi's sarcoma (Berraondo et al., Cytokines in clinical cancer immunotherapy. British Journal of Cancer, 2019, 120, 6-15; Fyfe et al., Results of treatment of 255 patients with metastatic renal cell carcinoma who received high-dose recombinant interleukin-2 therapy. J. Clin. Oncol., 1995, 13, 688-696; Atkins et al., High-dose recombinant interleukin 2 therapy for patients with metastatic melanoma: analysis of 270 patients treated between 1985 and 1993. J. Clin. Oncol., 1999, 17, 2105-2116; Golomb et al., α-2 interferon therapy of hairy-cell leukemia: a multi-center study of 64 patients. J. Clin. Oncol., 1986, 4, 900-905; Solal-Celigny et al., Recombinant interferon alfa-2b combined with a regimen containing doxorubicin in patients with advanced follicular lymphoma. Groupe d'Etude des Lymphomes de l'Adulte. New Engl. J. Med., 1993, 329, 1608-1614; Kirkwood et al., Interferon alfa-2b adjuvant therapy of high-risk resected cutaneous melanoma: the Eastern Cooperative Oncology Group Trial EST 1684. J. Clin. Oncol., 1996, 14, 7-17; Groopman et al., Recombinant alpha-2 interferon therapy for Kaposi's sarcoma associated with the acquired immunodeficiency syndrome. Ann. Intern. Med., 1984, 100, 671-676).

IL-6 is a pleiotropic cytokine that not only affects the immune system, but also acts in other biological systems and many physiological events, such as regulating cell growth, as well as gene activation, proliferation, survival, and differentiation. IL-6 is produced by a variety of cell types including monocytes, fibroblast, and endothelial cells. Upon stimulation, IL-6 is secreted by many additional cell types including macrophages, T cells, B cells, mast cells, glial cells, eosinophils, keratinocytes, and granulocytes. IL-6 stimulates several types of leukocytes and the production of acute phase proteins in the liver. It is particularly important in inducing B-cells to differentiate into antibody-forming cells (plasma cells). Binding of IL-6 to its receptor initiates cellular events including activation of JAK (Janus Kinase) kinases and activation of Ras-mediated signaling.

Like other Th1 pro-inflammatory cytokines, TNF-α has an important role comprising the inflammatory response both locally and in the circulation. TNF-α triggers the expression of vascular endothelial cells as well as enhances the leukocyte adhesion molecules that stimulate immune cell infiltration. It has a crucial role in early response against viral infection by enhancing the infiltration of lymphocyte to the site of infection

Accordingly, in some embodiments, the compositions and methods for treating cancer described herein includes a humanized 3E10 antibody or antigen binding fragment thereof and a polynucleotide that encodes a proinflammatory cytokine.

In some embodiments, the present disclosure provides compositions, and methods for treating cancer by administering such compositions to a subject in need thereof, including a complex formed between a (i) a humanized 3E10 antibody or antigen binding fragment thereof, and (ii) a therapeutic polynucleotide encoding a proinflammatory cytokine, as described herein. In some embodiments, the therapeutic polynucleotide is non-covalently bound to the humanized 3E10 antibody or antigen binding fragment thereof. In some embodiments, the therapeutic polynucleotide is covalently conjugated to the humanized 3E10 antibody or antigen binding fragment thereof.

In some embodiments, the cytokine is IL-1, IL-6, IL-8, IL-12, IFN-γ, IL-18, IL-15, IL-2, TNF-α, IL-10, TGF-b, CSF-1, CCL2, CCL3, CCL5, or VEGF.

### Gene Regulating Polynucleotides

In some embodiments, a humanized 3E10 antibody or antigen binding fragment thereof, as described herein, is used to deliver a gene-regulating polynucleotide that reduces or silences expression a gene product that promotes cancer growth and/or progression, e.g., by targeting the gene or a transcript thereof. Non-limiting examples of gene-regulating polynucleotides include siRNA, miRNA, saRNA, antagomirs, antisense oligonucleotides, and decoy oligonucleotides. In some embodiments, the gene-regulating polynucleotide is a non-replicating modified or unmodified mRNA. In some embodiments, the gene-regulating polynucleotide is a self-amplifying mRNA. In some embodiments, the gene-regulating polynucleotide is a plasmid encoding a protein or peptide. In some embodiments, the gene-regulating polynucleotide is an expression-regulating polynucleotide. For a review of the various types of gene-regulating polynucleotides that have been researched for therapeutic capability see, for example, Roberts T C, Langer R, Wood M J A, “Advances in oligonucleotide drug delivery,” Nat. Rev. Drug Discov., 19(10):673-94 (2020), the content of which is incorporated herein by reference.

Accordingly, in one aspect, the present disclosure relates to compositions, as well as methods for treating a cancer in a subject by administering to the subject a therapeutically effective amount of a composition, including a complex formed between (i) a humanized 3E10 antibody or antigen binding fragment thereof (ii) a gene-regulating polynucleotide, as described herein. In some embodiments, the therapeutic polynucleotide is non-covalently bound to the humanized 3E10 antibody or antigen binding fragment thereof. In some embodiments, the therapeutic polynucleotide is covalently conjugated to the humanized 3E10 antibody or antigen binding fragment thereof. In some embodiments, the gene-regulating polynucleotide is non-covalently bound to the humanized 3E10 antibody or antigen binding fragment thereof. In some embodiments, the gene-regulating polynucleotide is covalently conjugated to the humanized 3E10 antibody or antigen binding fragment thereof.

siRNA

The present disclosure relates to compositions and methods for treating a cancer and includes a complex formed between (i) a humanized 3E10 antibody or antigen binding fragment thereof, and (ii) an siRNA, as described herein. Small interfering RNA (siRNA), also known as short interfering RNA or silencing RNA, is a class of double-stranded RNA non-coding RNA molecules, typically 20-27 base pairs in length, and operating within the RNA interference (RNAi) pathway. Gene-regulating nucleic acid drugs such as siRNA can regulate post-transcriptional gene expression, and silence targeted genes, further regulating intracellular signaling pathway involved in cancer progression (Zhou et al., Delivery of nucleic acid therapeutics for cancer immunotherapy, Medicine in Drug Discovery, Mar. 24, 2020; Dahlman et al., In vivo endothelial siRNA delivery using polymeric nanoparticles with low molecular weight, Nature Nanotechnol. 2014; 9(8):648-655). In some embodiments, the siRNA is non-covalently bound to the humanized 3E10 antibody or antigen binding fragment thereof. In some embodiments, the siRNA is covalently conjugated to the humanized 3E10 antibody or antigen binding fragment thereof.

siRNAs can, thus, be used for modulating the expression of immune checkpoint molecules, such as those described herein, by regulating the post-translational gene expression and/or silencing corresponding genes. Similarly, siRNAs can be used for indirectly regulating the activity of immune checkpoint molecules by modulating the expression of agonists or inhibitors of immune checkpoint molecules. Accordingly, in some embodiments, the composition for treatment of cancer described herein includes a complex formed between (i) a humanized 3E10 antibody or antigen binding fragment thereof, and (ii) an siRNA targeting an mRNA transcript from a gene encoding an immune checkpoint molecule, as described herein.

PD-1 and certain homologs thereof such as, for example, PD-L1, suppress T-cell responses, especially in the tumor microenvironment. Thus, inhibitors of PD-1 and/or PD-L1 may improve efficacy of T-cells in attacking and killing tumor cells. Suppression of PD-1 and/or PD-L1 activity can be accomplished, for example, by inhibiting the production of PD-1 and/or PD-L1 within the cells. Accordingly, in some embodiments, the compositions for a cancer provided herein include a complex formed between (i) a humanized 3E10 antibody or antigen binding fragment thereof, and (ii) an siRNA targeting an mRNA transcript for PD-1 or PD-L1.

Suppression of Cytotoxic T-lymphocyte-associated antigen 4 (CTLA-4) activity is known to result in rapid infiltration of T cells. Thus, inhibitors of CTLA-4 may result in promoting T-cell responses. Accordingly, in some embodiments, the compositions for treating a cancer provided herein include a complex formed between (i) a humanized 3E10 antibody or antigen binding fragment thereof, and (ii) siRNA targeting an mRNA transcript for CTLA-4.

siRNAs can similarly be used for silencing genes regulating tumor growth or angiogenesis. For example, siRNAs have been used to target vascular endothelial growth factor (VEGF) and kinesin spindle protein (KSP) (for solid tumors, e.g., liver metastasis from colon cancer). Other genetic targets that can be silenced using siRNAs include, but are not limited to, genes encoding protein kinase N3 (PKN3) (e.g., for metastatic pancreatic cancer), M2 subunit of ribonucleotide reductase (RRM2) (e.g., for solid tumors), Myc oncoprotein (e.g., for hepatocellular carcinoma), ephrin type-A receptor 2 (EphA2) (e.g., for advanced cancers), and KRAS G12D mutation (e.g., for advanced pancreatic cancers). See, e.g., International Journal of Nanomedicine 2019:143111-3128. Accordingly, in some embodiments, the compositions for treating a cancer provided herein include a complex formed between (i) a humanized 3E10 antibody or antigen binding fragment thereof, and (ii) siRNA targeting an mRNA transcript for VEGF, KSP, PKN3, RRM2, EphA2, ERBB2/HER2, SOCS1, PLK1, or KRAS.

Other examples of siRNA that find use in the methods and compositions described herein include, but are not limited to, siRNA targeting an mRNA transcript from a gene encoding CD25 (EL-2 receptor) to downregulate EL-2 signaling in CD8+ T-cells.

Other non-limiting examples of siRNA and associated cancer types being studied are provided in Table 4 below (See, e.g., Int. J. Mol. Sci. 22 (2021) 3295):

TABLE 4
Example siRNA and associated cancer types.

| _Drug/Therapeutic | Target | Cancer |
|---|---|---|
| KRAS G12D siRNA | KRASG12D | Pancreatic cancer |
| EphA2-targeting DOPC- | EPHA2 | Solid tumors |
| encapsulated siRNA | | |
| APN401 | CBLB | Brain cancer, melanoma, pancreatic |
| | | cancer, renal cell cancer |
| Proteosome siRNA and tumor | LMP2, LMP7, | Melanoma |
| antigen RNA-transfected | MECL1 | |
| dendritic cells | | |
| TKM-080301 | PLK1 | Cancer with hepatic metastases, liver |
| | | cancer, hepatocellular cancer, |
| | | adrenocortical cancer |
| Atu027 | PNK3 | Solid tumors, pancreatic cancer |
| DCR-MYC | MYC | Solid tumors, hepatocellular cancer |
| CALAA-01 | M2 subunit of | Solid tumors |
| | ribonucleotide | |
| | reductase 2 | |
| siG12D LODER | KRASG12D | Pancreatic cancer |
| ARO-HIF2 | HIF2A | Clear cell renal cell carcinoma |
| SV40 vectors carrying siRNA | Unknown | Chronic myeloid leukemia |

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an siRNA listed in Table 4, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a respective siRNA listed in Table 4, as described herein, where the cancer is a cancer associated with the respective siRNA in Table 4.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an siRNA targeting a transcript from a gene listed in Table 4, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an siRNA targeting a transcript from a respective gene listed in Table 4, as described herein, where the cancer is a cancer associated with the respective gene in Table 4.

miRNA

In some embodiments, the compositions and methods for treating a cancer described herein includes a complex formed between (i) a humanized 3E10 antibody or antigen binding fragment thereof, and (ii) an miRNA, as described herein. MicroRNAs (miRNAs) are a class of non-coding RNAs that play important roles in regulating gene expression. miRNA is endogenous small non-coding RNA of about 18-24 nt in length that can regulate target gene expression by a mechanism similar to siRNA (Zhou et al., Delivery of nucleic acid therapeutics for cancer immunotherapy, Medicine in Drug Discovery, Mar. 24, 2020; Xiao et al., MicroRNA control in the immune system; basic principles, Cell, 2009; 136(1):26-36). One main challenge of miRNA delivery is to deliver them into tumor tissue with deep tissue penetration efficiently. Moreover, the complexation of tumor microenvironment also prevents miRNA from efficient intracellular delivery into target tumor cells (Rupaimoole et al., MiRNA deregulation in cancer cells and the tumor microenvironment. Cancer Discov. 2016; 6(3):235-46). In some embodiments, the miRNA is non-covalently bound to the humanized 3E10 antibody or antigen binding fragment thereof. In some embodiments, the miRNA is covalently conjugated to the humanized 3E10 antibody or antigen binding fragment thereof.

Advantageously, however, expression of miRNAs is specific to distinct tumors, and miRNAs are involved in early regulation of immune responses. One approach to treating cancer is to modulate the expression of immune checkpoint molecules, such as those described herein, by modulating levels of miRNAs. Examples of miRNAs regulating immune checkpoint-related processes include, but are not limited to, miR-15a, -15b, -16, -195, -424, -497, and -503, which regulate the expression of PD-L1 and CD80. Another example of miRNA with tumor-suppressive function is miR-28, which inhibits the expression of TIM3, BTLA, and PD-1 in T-cells by binding to their respective 3′ UTRs. Yet another example of miRNA is miR-138 which inhibits the expression of PD-1 and CTLA-4 on the surface of both effector and regulatory T-cells. miR-34 family which includes miR-34a, -34b and -34c, inhibits expression of PD-L1.

Expression of miR-138-5p is known to impede proliferation of CRC cell, block their transition from G1 to S phase of the cell cycle and directly inhibit PD-L1 expression.

Other miRNAs such as, for example, miR-20b, -21, and -130b, which are overexpressed in certain types of cancer cells may be effective in indirectly mitigating T-cell activation in tumor microenvironment by expression of PTEN.

Accordingly, the compositions and methods for treating a cancer described herein includes a complex formed between (i) a humanized 3E10 antibody or antigen binding fragment thereof (ii) an miRNA which directly or indirectly modulates the expression of immune checkpoint molecules, as described herein.

In some embodiments, the miRNA is miR-15a, miR-15b, miR-16, miR-20b, miR-21, miR-28, miR-34a, miR-34b, miR-34c, miR-125b, miR-130b, miR-138, miR-138-5p, miR-155, miR-195, miR-197, miR-200, miR-210, miR-221, miR-222, miR-424, miR-497, miR-503, or miR-513.

In some embodiments, the compositions and methods for treating a cancer described herein includes a complex formed between (i) a humanized 3E10 antibody or antigen binding fragment thereof, and (ii) an miRNA mimicking molecules which directly or indirectly modulates the expression of immune checkpoint molecules, as described herein. The miRNAs may be double-stranded synthetic RNAs that mimic endogenous miRNAs because of the same sequence.

In some embodiments, the compositions and methods for treating a cancer described herein includes a complex formed between (i) a humanized 3E10 antibody or antigen binding fragment thereof, and (ii) an miRNA expression vector encoding an miRNA which directly or indirectly modulates the expression of immune checkpoint molecules, as described herein.

In some embodiments, the compositions and methods for treating a cancer described herein includes a complex formed between (i) a humanized 3E10 antibody or antigen binding fragment thereof, and (ii) an LNA-modified antisense oligodeoxyribonucleotide (ASO) targeting an miRNA which directly or indirectly modulates the expression of immune checkpoint molecules, as described herein. An LNA is a bicyclic RNA analog with the ribose locked in C3′-endo conformation by the introduction of a 2′-O, 4′-C methylene bridge.

In some embodiments, the compositions and methods for treating a cancer described herein includes a complex formed between (i) a humanized 3E10 antibody or antigen binding fragment thereof, and (ii) an antagomir targeting an miRNA which directly or indirectly modulates the expression of immune checkpoint molecules, as described herein. An antagomir may be a single-stranded 23-nucleotide RNA molecule complementary to the targeted miRNA that has been modified with a partial phosphorothioate backbone in addition to 2′-O-methoxyethyl. This is known to increase the stability of miRNA by protecting it from degradation.

In some embodiments, the compositions and methods for treating a cancer described herein includes a complex formed between (i) a humanized 3E10 antibody or antigen binding fragment thereof, and (ii) an antisense oligodeoxyribonucleotide (ASO) targeting an miRNA which directly or indirectly modulates the expression of immune checkpoint molecules, as described herein.

In some embodiments, composition and methods for treating a cancer described herein includes a complex formed between (i) a humanized 3E10 antibody or antigen binding fragment thereof, and (ii) an miRNA sponge which directly or indirectly modulates the expression of immune checkpoint molecules, as described herein. A miRNA sponge may be an RNA containing multiple tandem binding sites for the miRNA of interest transcribed from expression vectors.

Similarly, miRNA modulating the expression of proteins that are associated with tumor growth or angiogenesis may also be delivered by complexing with a humanized 3E10 antibody or antigen binding fragment thereof, as described herein. Non-limiting examples of miRNA and cancer they are associated with are given in Table 5.

TABLE 5
Example miRNAs being studied for treatment of cancers (see, e.g., Journal of
the International Federation of Clinical Chemistry and Laboratory Medicine
(2019) Vol. 30, No. 2, pp. 114-127)

| miRNA | Cancer |
|---|---|
| miR-122 | HCV |
| miR-155 | Lymphoma and leukemia |
| miR-16 | Mesothelioma |
| miRNA | Cancer |
| miR-34 | Renal cell carcinoma, acral melanoma, hepatocellular |
| | carcinoma |
| MRX34 | Liver cancer, lung cancer, lymphoma, melanoma, |
| | multiple myeloma, renal cell cancer |
| INT-1B3 | Solid tumor |
| TargomiRs | Malignant pleural mesothelioma, non-small cell lung cancer |
| Cobomarsen | Cutaneous T-cell lymphoma, colorectal cancer |
| (MRG-106) | |

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an miRNA listed in Table 5, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a respective miRNA listed in Table 5, as described herein, where the cancer is a cancer associated with the respective miRNA in Table 5.

saRNA

In some embodiments, the compositions and methods for treating a cancer described herein includes a complex formed between (i) a humanized 3E10 antibody or antigen binding fragment thereof, and (ii) an saRNA, as described herein. Small-activating RNA (saRNA) is a class of noncoding dsRNA about 21 nt in length with 2 nt overhangs at both end (Zhou et al., Medicine in Drug Discovery, Mar. 24, 2020; Kwok et al., Ther. Deliv. 2019; 10(3):151-64). Although it shares similar structure with siRNA, it has the opposite mechanism of gene regulation. An saRNA in the cytoplasm is specifically loaded to an AGO2 protein and this RNA-AGO2 complex is transported to the nucleus to induce targeted gene promoters for gene activation (Li et al., Proc. Natl. Acad. Sci. USA. 2006; 103(46): 17337-42). It has been reported that saRNA-AGO2 complex in the nucleus recruits essential protein for transcription initiation such as RNA helicase A, RNA polymerase-associated protein CTR9 homolog (CTR9) and RNA polymerase II-associated factor 1 homolog (PAF1) (Portnoy et al., Cell Res. 2016; 26(3): 320-35). Due to its ability of gene upregulation, saRNA shows the potential for applications such as cancer immunotherapy. Accordingly, in some embodiments, the composition for treatment of cancer described herein includes a complex formed between (i) a humanized 3E10 antibody or antigen binding fragment thereof, and (ii) an saRNA inducing activation of a gene encoding an immune checkpoint molecule, as described herein. In some embodiments, the saRNA is non-covalently bound to the humanized 3E10 antibody or antigen binding fragment thereof. In some embodiments, the saRNA is covalently conjugated to the humanized 3E10 antibody or antigen binding fragment thereof.

In one example, an saRNA can upregulate the transcription factor CCATT/enhancer binding protein alpha (CEBPA) which leads to an increase in functional C/EBP protein and albumin and inhibits growth of liver cancer in a rat model. Other non-limiting examples of saRNAs being studied for treating cancer are listed in Table 6.

TABLE 6
saRNAs being studied for treatment of cancers

| saRNA | Cancer |
|---|---|
| C/EBPα-saRNA | Hepatocellular carcinoma, pancreatic |
| | ductal adenocarcinoma |
| dsP21-322 | Bladder tumor |

### Antagomirs

In some embodiments, the compositions and methods for treating a cancer described herein includes a complex formed between (i) a humanized 3E10 antibody or antigen binding fragment thereof, and (ii) an antagomir, as described herein. An antagomir is a small synthetic RNA that is complementary to the specific miRNA target with either mispairing at the cleavage site of Ago2 or some sort of base modification to inhibit Ago2 cleavage. Antagomirs are sequestered specific endogenous microRNA in competition with cellular target mRNAs, inducing miRNA repression and preventing mRNA target degradation via RISC. Thus, antagomirs can be used in treatments where miRNA loss of function is advantageous. In some embodiments, the antagomir is non-covalently bound to the humanized 3E10 antibody or antigen binding fragment thereof. In some embodiments, the antagomir is covalently conjugated to the humanized 3E10 antibody or antigen binding fragment thereof.

An example of an antagomir is anti-miR21. Studies have shown that silencing of miR21 through use of anti-miR21 affected viability, apoptosis and the cell cycle in colon cancer cells (Song et al., “The anti-miR21 antagomir, a therapeutic tool for colorectal cancer, has a potential synergistic effect by perturbing an angiogenesis-associated miR30,” Front. Genet, January 2014).

Another example of an antagomir is antagomir-221. Studies have shown that antagomir-221 was able to reduce cellular proliferation by suppressing the function of miR-221 which plays an important role in HCC as it inhibits tumor-suppressive target proteins such as P27KIP1, P57KIP2, and phosphatase and tensin homolog (PTEN). Likewise, several studies have shown that antagomir-21 reversed epitheliam-mesenchymal transition (EMT) through inactivation of AKT serine/threonine kinase 1 (AKT) and ERK1/2 pathways by targeting PTEN. This action of antagomir-21 can potentially be used to target the causal mechanism of the malignant propensity of breast cancers. See, e.g., Atri, et al., AGO-Driven Non-Coding RNAs (2019).

AntagomiR that targets miR-155, is in phase 1 (NCT02580552) and phase 2 clinical trials (NCT03713320). miR-155 regulates differentiation and proliferation of blood and lymphoid cells and is a suitable target for treating certain kinds of lymphoma and leukemia. See, e.g., “RNA-Based Therapeutics: From Antisense Oligonucleotides to miRNAs,” Cells 9 (2020), 137.

Accordingly, in some embodiments, the composition for treatment of cancer described herein includes a complex formed between (i) a humanized 3E10 antibody or antigen binding fragment thereof, and (ii) an antagomir targeting microRNA modulating translation of a tumor-associated mRNA, as described herein. Non-limiting examples of antagomirs include antagomir-221, antagomir-21 and antagomir-155.

### Antisense Oligonucleotides (ASOs)

In some embodiments, the composition for treatment of cancer described herein includes a complex formed between (i) a humanized 3E10 antibody or antigen binding fragment thereof, and (ii) an antisense oligonucleotide, as described herein. Antisense oligonucleotides (ASOs) are short, synthetic, chemically modified chains of nucleotides that have the potential to target any gene product of interest. Typically, an ASO is a single-stranded sequence complementary to the sequence of the target gene's transcribed messenger RNA (mRNA) within a cell (Rinaldi et al., “Antisense oligonucleotides: the next frontier for treatment of neurological disorders,” Nat. Rev. Neurol. 2018; 14(1):9-21; Bennett, Therapeutic Antisense Oligonucleotides Are Coming of Age. Ann. Rev. Med. 2019; 70:307-321). An ASO targets the corresponding mRNA to degrade the targeted complex by mechanisms such as endogenous cellular RNase H. In some embodiments, the antisense oligonucleotide is non-covalently bound to the humanized 3E10 antibody or antigen binding fragment thereof. In some embodiments, the antisense oligonucleotide is covalently conjugated to the humanized 3E10 antibody or antigen binding fragment thereof.

One example of an ASO being used for cancer therapy is an ASO targeting CD39 mRNA so as to improve CD8+ T cell proliferation, thereby improving antitumor immune responses. Zhou, et al. Medicine in Drug Discovery, 6:(2020) 100023.

Other non-limiting Examples of ASO and cancer they are associated with are given in Table 7.

TABLE 7
Example ASOs being studied for treatment of cancers (See, e.g., Int. J. Mol. Sci 22 (2021) 3295)

| Drug/Therapeutic | Target | Cancer |
|---|---|---|
| AEG35156 | AEG35156 | Hepatocellular Cancer, Pancreatic Cancer, |
| | | Breast Cancer, Non-Small Cell Lung Cancer, |
| | | Leukemia, Lymphoma |
| Apatorsen (OGX-427) | HSP27 | Urologic Cancer, Bladder Cancer, Prostate |
| | | Cancer, Urothelial Cancer, Non-Small Cell |
| | | Lung Cancer |
| ARRx (AZD5312) | AR | Prostate cancer |
| AZD4785 | KRAS | Non-Small Cell Lung Cancer |
| AZD8701 | FOXP3 | Advanced cancer |
| AZD9150 | STAT3 | Bladder cancer, lymphoma, malignancies |
| BP1001 | GRB2 | Ph1 Positive Leukemia, Acute Myeloid |
| | | Leukemia, Chronic Myelogenous Leukemia |
| Cenersen (EL625) | TP53 | Acute Myelogenous Leukemia, lymphoma |
| CpG 7909 (PF03512676) | TLR9 | Melanoma, Breast Cancer, Renal Cancer, |
| | | Lymphoma, Non-Small Cell Lung Cancer, |
| | | Esophageal Cancer, Prostate Cancer |
| CpG ODN (GNKG168) | TLR9 | Leukemia |
| CpG Oligonucleotide | TLR9 | Breast cancer |
| CpG-ODN | TLR9 | Glioblastoma |
| Custirsen (OGX-011) | ApoJ | Prostate cancer, breast cancer, non-small cell |
| | | lung cancer |
| Danvatirsen (AZD9150, ISIS | STAT3 | Advanced cancers |
| STAT3Rx) | | |
| EGFR Antisense DNA | EGFR | Head and Neck Squamous Cell Cancer, Gastric |
| | | Cancer, Ovarian Cancer, Prostate Cancer |
| EZN-2968 | HIF1A | Hepatocellular cancer, lymphoma |
| (RO7070179, SPC2968) | | |
| G4460 | CMYB | Leukemia, hematologic malignancies |
| IGF-1R/AS ODN | IGF1 | Glioma |
| IGV-001 containing | IGF1R | Glioblastoma |
| autologous GBM cells treated | | |
| with antisense | | |
| oligonucleotide (IMV-001) | | |
| IMO-2055 (EMD 1201081) | TLR9 | Renal cell cancer, colorectal cancer, non-small |
| | | cell lunch cancer, head and neck cancer |
| ION251 | IRF4 | Myeloma |
| ION537 | YAP1 | Advanced solid tumors |
| ISIS 183750(ISIS-EIF4ERx, | EIF4E | Castrate-resistant prostate cancer, non-small |
| LY2275796) | | cell lung cancer, colorectal cancer |
| ISIS 2503 | HRAS | Colorectal cancer, pancreatic cancer |
| ISIS 5132 | CRAF | Ovarian cancer |
| L-Bcl-2 antisense | BCL2 | Advanced lymphoid malignancies |
| oligonucleotide | | |
| LErafAON | CRAF | Cancers |
| Lucanix | TGFB2 | Non-small cell lung cancer |
| LY2181308 | BIRC5 | Non-small cell lung cancer |
| LY900003 (ISIS 3521, | PKCA | Melanoma, lung cancer, non-small cell lung |
| Affinitak) | | cancer, breast cancer |
| MTL-CEBPA | CEBPA | Hepatocellular cancer |
| Oblimersen (G3139) | BCL2 | Cancers |
| OGX-427 | HSP27 | Cancers |
| PNT2258 | BCL2 | Prostate cancer, lymphoma, melanoma |
| SPC2996 | BCL2 | Chronic lymphocytic leukemia |
| Drug/Therapeutic | Target | Cancer |
| TGF 2 Antisense-GMCSF | TGFB2 | Advanced cancers |
| Gene Modified Autologous | | |
| Tumor Cell (TAG) Vaccine | | |
| SD-101 | TLR9 | Cancers |
| Trabedersen (AP 12009, | TGFB2 | Glioblastoma, anaplastic astrocytoma, |
| OT-101) | | pancreatic cancer, melanoma, colorectal cancer |
| VEGF-Antisense | VEGF | Mesothelioma |
| Oligonucleotide | | |

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an antisense oligonucleotide listed in Table 7, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) a respective an antisense oligonucleotide listed in Table 7, as described herein, where the cancer is a cancer associated with the respective an antisense oligonucleotide in Table 7.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an antisense oligonucleotide targeting a transcript from a gene listed in Table 7, as described herein.

In some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an antisense oligonucleotide targeting a transcript from a respective gene listed in Table 7, as described herein, where the cancer is a cancer associated with the respective gene in Table 7.

### Decoy Oligonucleotides

In some embodiments, the present disclosure relates to compositions, as well as methods for treating a cancer in a subject by administering to the subject a therapeutically effective amount of a composition, including a complex formed between (i) a humanized 3E10 antibody or antigen binding fragment thereof, and (ii) a decoy oligonucleotide or a polynucleotide that encodes a decoy oligonucleotide. Transfection of cis-element double-stranded oligonucleotides, referred to as decoy oligodeoxynucleotides, has been reported to be a powerful tool that provides a new class of antigene strategies for gene therapy (Crinelli et al., Design and characterization of decoy oligonucleotides containing locked nucleic acids. Nucleic Acid Res. 2002; 30(11): 2435-2443). One such example is STAT3 decoy oligonucleotide, which is a double-stranded 15-mer oligonucleotide, corresponding closely to the signal transducer and activator of transcription 3 (STAT3) response element within the c-fos promoter, with potential antineoplastic activity. STAT3 decoy oligonucleotide binds specifically to activated STAT3 and blocks binding of STAT3 to DNA sequences on a variety of STAT3-responsive promoters, which results in the inhibition of STAT3-mediated transcription and, potentially, the inhibition of tumor cell proliferation. STAT3 is constitutively activated in a variety of cancers including squamous cell carcinoma of the head and neck, contributing to the loss of cell growth control and neoplastic transformation. In some embodiments, the decoy oligonucleotide or polynucleotide that encodes the decoy oligonucleotide is non-covalently bound to the humanized 3E10 antibody or antigen binding fragment thereof. In some embodiments, the decoy oligonucleotide or polynucleotide that encodes the decoy oligonucleotide is covalently conjugated to the humanized 3E10 antibody or antigen binding fragment thereof.

Examples of polynucleotides that encode complexes that can perform genome editing are described in the following sections.

### Zinc Finger Nucleases

In some embodiments, a humanized 3E10 antibody or antigen binding fragment thereof described herein is complexed to a polynucleotide that encodes a zinc-finger nuclease. Zinc-finger nucleases are genome editing nucleases. They are artificial restriction enzymes generated by fusing a zinc finger DNA-binding domain to a DNA-cleavage domain. The binding specificity of the designed zinc-finger domain directs the zinc-finger nuclease to a specific genomic site.

In some embodiments, the present disclosure relates to compositions, as well as methods for gene editing in a subject in need thereof by administering to the subject a therapeutically effective amount of a composition, including a complex formed between (i) a humanized 3E10 antibody or antigen binding fragment thereof, and (ii) a polynucleotide that encodes a zinc-finger nuclease. In some embodiments, the polynucleotide that encodes a zinc-finger nuclease is non-covalently bound to the humanized 3E10 antibody or antigen binding fragment thereof. In some embodiments, the polynucleotide that encodes a zinc-finger nuclease is covalently conjugated to the humanized 3E10 antibody or antigen binding fragment thereof.

With its success in multiple organisms, including the fruit fly, zebrafish, and rats, ZFN technology has also been successfully used in human cell culture such as K562 cells, T cells, and hPSCs. It was first demonstrated ZFN-driven targeted gene addition to the endogenous CCR5 locus in hESCs to generate green fluorescent protein (GFP) reporter cell lines that stably express GFP for at least 2 months in both the undifferentiated and differentiated state. The eGFP gene introduced to OCT4 (also known as POU5F1) locus was reported to faithfully reflect its transcriptional status, resulting in an endogenous pluripotency reporter cell line. ZFN-driven gene addition has also been successfully used to introduce fluorescence reporter genes and drug-resistance genes into ubiquitous loci like AAVS1 for constitutive or inducible expression or differentiated lineage-specific loci, such as PITX3, to monitor pluripotency and track cellular differentiation. In all of these studies, specific and stable gene addition was achieved with high efficiency without losing pluripotency and, more importantly, the integrated gene retained high expression level as the stem cells differentiated.

Despite its success in target genome editing in hPSCs, ZFN technology has several limitations. First of all, two ZFNs and the homologous DNA have to be co-delivered into the hPSCs, requiring efficient delivery methods, typically employing viral vectors. However, the viral vectors can randomly integrate the viral sequence into the target genome, disrupting critical genes in hPSCs. Transfection and electroporation methods or even direct protein delivery methods have been used to circumvent the viral vectors to introduce ZFNs and DNA into cells of interest. Another obstacle to successfully applying ZFN technology is that the design of ZFNs is always difficult and time-consuming due to the imperfect modular nature of the tandem zinc fingers in which the assembled ZFNs do not necessarily have high affinity for the targeted sequence that is the composite of the 3-bp binding sequence of each individual zinc finger. In addition, the imperfect modular structure of zinc-finger assembly and nonspecific site binding of the FokI cleavage domain also increases the risk of off-target activity and cellular toxicity. To address this problem, structure and selection-based approaches, including oligomerized pool engineering and directed evolution, have been applied to generate improved ZFNs with optimized DNA-binding specificity and reduced cellular toxicity.

### Transcription Activator-Like Effector Nucleases (TALEN)

In some embodiments, a humanized 3E10 antibody or antigen binding fragment thereof described herein is complexed to a polynucleotide that encodes a Transcription Activator-like Effector Nucleases (TALEN). TALENs are artificial endonucleases (e.g., restriction enzymes) and are produced by the fusion of a transcription activator-like effector (TALE) DNA binding domain with a DNA cleavage domain. TALENs can be engineered to bind any DNA sequence of interest. In some embodiments, the present disclosure relates to compositions, as well as methods for gene editing in a subject by administering to the subject a therapeutically effective amount of a composition, including a complex formed between (i) a humanized 3E10 antibody or antigen binding fragment thereof, and (ii) a polynucleotide that encodes a TALEN. In some embodiments, the polynucleotide that encodes a TALEN is non-covalently bound to the humanized 3E10 antibody or antigen binding fragment thereof. In some embodiments, the polynucleotide that encodes a TALEN is covalently conjugated to the humanized 3E10 antibody or antigen binding fragment thereof.

In one embodiment, an engineered TALEN comprises from the N-terminus to the C-terminus, a first spacer, a TALE DNA binding domain, a second spacer, and a FokI nuclease catalytic domain fused to the C-terminus. The DNA cleavage domain cuts DNA strands and, so the fusion with a TALE DNA binding domain can be specific for a DNA sequence of interest to edit genomes by inducing double strand breaks. TALENs can function alone, in pairs, or in a plurality of pairs. For example, the TALE DNA binding domain can bind to targets positioned opposite of one another, across a spacer wherein the FokI domains come together to create the break in the DNA. In an aspect, TALE DNA binding domains can be designed for use in the disclosed TALENs. A single TALEN (also referred to herein as a monomeric TALEN or a TALEN monomer) comprises a TALE DNA binding domain and a FokI nuclease catalytic domain fused to the C-terminus. A TALEN can be engineered to be used in a TALEN pair (or also referred to herein as a pair of TALENs or TALEN pairs) designed to bind to a target nucleotide sequence configured from the N-terminus to the C-terminus on opposing strands of DNA. TALENs in a TALEN pair can have the same sequence or can be different in sequence.

### CRISPR/Cas System

In some embodiments, a humanized 3E10 antibody or antigen binding fragment thereof described herein is complexed to a polynucleotide that encodes Cas endonuclease. In some embodiments, the present disclosure relates to compositions, as well as methods for gene editing in a subject by administering to the subject a therapeutically effective amount of a composition, including a complex formed between (i) a humanized 3E10 antibody or antigen binding fragment thereof, and (ii) a polynucleotide that encodes a Cas endonuclease. In some embodiments, the polynucleotide that encodes a Cas endonuclease is non-covalently bound to the humanized 3E10 antibody or antigen binding fragment thereof. In some embodiments, the polynucleotide that encodes a Cas endonuclease is covalently conjugated to the humanized 3E10 antibody or antigen binding fragment thereof.

In some embodiments, a humanized 3E10 antibody or antigen binding fragment thereof described herein is complexed to a CRISPR/Cas guide polynucleotide, e.g., a single guide RNA (sgRNA). In some embodiments, the present disclosure relates to compositions, as well as methods for gene editing in a subject by administering to the subject a therapeutically effective amount of a composition, including a complex formed between (i) a humanized 3E10 antibody or antigen binding fragment thereof, and (ii) a CRISPR/Cas guide polynucleotide. In some embodiments, the CRISPR/Cas guide polynucleotide is non-covalently bound to the humanized 3E10 antibody or antigen binding fragment thereof. In some embodiments, the CRISPR/Cas guide polynucleotide is covalently conjugated to the humanized 3E10 antibody or antigen binding fragment thereof.

CRISPR (Clustered Regularly Interspaced Short Palindromic Repeats) and CRISPR-associated (Cas) endonucleases were originally discovered as adaptive immunity systems evolved by bacteria and archaea to protect against viral and plasmid invasion. Naturally occurring CRISPR/Cas systems in bacteria are composed of one or more Cas genes and one or more CRISPR arrays consisting of short palindromic repeats of base sequences separated by genome-targeting sequences acquired from previously encountered viruses and plasmids (called spacers). (Wiedenheft, B., et. al. Nature. 2012; 482:331; Bhaya, D., et. al., Annu. Rev. Genet. 2011; 45:231; and Terms, M. P. et. al., Curr. Opin. Microbiol. 2011; 14:321). Bacteria and archaea possessing one or more CRISPR loci respond to viral or plasmid challenge by integrating short fragments of foreign sequence (protospacers) into the host chromosome at the proximal end of the CRISPR array. Transcription of CRISPR loci generates a library of CRISPR-derived RNAs (crRNAs) containing sequences complementary to previously encountered invading nucleic acids (Haurwitz, R. E., et. al., Science. 2012:329; 1355; Gesner, E. M., et. al. Nat. Struct. Mol. Biol. 2001:18; 688; Jinek, M., et. al., Science. 2012:337; 816-21). Target recognition by crRNAs occurs through complementary base pairing with target DNA, which directs cleavage of foreign sequences by means of Cas proteins. (Jinek et. al. 2012 “A Programmable dual-RNA-guided DNA endonuclease in adaptive bacterial immunity.” Science. 2012:337; 816-821).

There are at least six main CRISPR system types (Type I, II, III, IV, V, and VI) and at least 16 distinct subtypes. See, for example, Makarova, K. S., et al., Nat. Rev. Microbiol. 2015. Nat. Rev. Microbiol. 13, 722-736, and Liu, Z., Dong, H., Cui, Y. et al., Application of different types of CRISPR/Cas-based systems in bacteria. Microb Cell Fact 19, 172 (2020). CRISPR systems are also classified based on their effector proteins. Class 1 systems possess multi-subunit crRNA-effector complexes, whereas in class 2 systems all functions of the effector complex are carried out by a single protein (e.g., Cas9 or Cpf1). In some embodiments, the present disclosure teaches using type II and/or type V single-subunit effector systems. Thus, in some embodiments, the present disclosure teaches using class 2 CRISPR systems.

### CRISPR/Cas9

In some embodiments, the present disclosure provides methods of gene editing using a Type II CRISPR system. In some embodiments, the Type II CRISPR system uses a Cas9 enzyme. Type II systems rely on a i) single endonuclease protein, ii) a transactivating crRNA (tracrRNA), and iii) a crRNA where a ^{˜}20-nucleotide (nt) portion of the 5′ end of crRNA is complementary to a target nucleic acid. The region of a CRISPR crRNA strand that is complementary to its target DNA protospacer is hereby referred to as “guide sequence.”

In some embodiments, the tracrRNA and crRNA components of a Type II system can be replaced by a single-guide RNA (sgRNA). The sgRNA can include, for example, a nucleotide sequence that comprises an at least 12-20 nucleotide sequence complementary to the target DNA sequence (guide sequence) and can include a common scaffold RNA sequence at its 3′ end. As used herein, “a common scaffold RNA” refers to any RNA sequence that mimics the tracrRNA sequence or any RNA sequences that function as a tracrRNA.

Cas9 endonucleases produce blunt end DNA breaks and are recruited to target DNA by a combination of a crRNA and a tracrRNA oligos, which tether the endonuclease via complementary hybridization of the RNA CRISPR complex.

In some embodiments, DNA recognition by the crRNA/endonuclease complex requires additional complementary base-pairing with a protospacer adjacent motif (PAM) (e.g., 5′-NGG-3′) located in a 3′ portion of the target DNA, downstream from the target protospacer. (Jinek, M., et. al., Science. 2012:337; 816-821). In some embodiments, the PAM motif recognized by a Cas9 varies for different Cas9 proteins.

### Aptamers

In some embodiments, a humanized 3E10 antibody or antigen binding fragment thereof described herein is complexed with an aptamer or a polynucleotide encoding an aptamer. Nucleic acid aptamers are single-stranded (ss) oligonucleotide molecules (DNA or RNA), that fold into distinct secondary or tertiary structures, giving them high affinity and specific binding abilities toward their corresponding targets (Zhu et al., “Nucleic Acid Aptamer-Mediated Drug Delivery for Targeted Cancer Therapy”, ChemMedChem 2015, 10, 39-45). Aptamers are selected from a random library of 1013-1016 ssDNA or ssRNA molecules through an in vitro technology known as SELEX (systematic evolution of ligands by exponential enrichment) (Ellington et al., Nature 1990, 346, 818-822; Tuerk et al., Science 1990, 249, 505-510).

After an aptamer sequence is identified by SELEX, modified nucleotides may be incorporated into the sequence, e.g., to promote stability and/or resistance to nuclease degradation and/or to increase the efficiency of the aptamer. For instance, aptamer APTA-12 includes a gemcitabine residue, which is a 2′, 2′-difluoro analogue of 2′deoxycytidine. See, for example, Park J Y et al., Mol. Ther. Nucleic Acids 2018, 12, 543-553.

Generally, aptamers can be used in cancer therapy to either directly inhibit the activity of a target molecule (where the aptamer is acting as the functional therapeutic molecule), or to target a therapeutic molecule, e.g., a chemotherapeutic or other anti-cancer agent, to a cancerous tissue. In some embodiments, the aptamers used in the methods and compositions described herein directly inhibit the activity of a target molecule, rather than target a cancerous tissue. This is because the humanized 3E10 antibody or antigen binding fragment thereof complexed with the aptamer already targets various cancerous tissues, as described herein. Commonly, therapeutic aptamers used for cancer therapy act as antagonists of oncoproteins or their ligands by binding to one of them, thereby blocking protein-protein or receptor-ligand interactions that promote cancer development and/or progression. For review of the use of aptamers for treatment of cancer see, for example. Han et al., Application and development of aptamer in cancer—from clinical diagnosis to cancer therapy, Journal of Cancer, 2020, 11, 6902-6915; Zhu et al., “Nucleic Acid Aptamer-Mediated Drug Delivery for Targeted Cancer Therapy”, ChemMedChem 2015, 10, 39-45; and Subjakova et al., ‘Polymer Nanoparticles and Nanomotors Modified by DNA RNA Aptamers and Antibodies in Targeted Therapy of Cancer”, Polymers, 2021, 13, 341; Morita Y et al., Cancers (Basel), 2018; 10(3):80, the disclosures of which are incorporated herein by reference.

In some embodiments, the present disclosure relates to compositions, as well as methods for treating a subject in need thereof (e.g., for cancer) by administering to the subject a therapeutically effective amount of a composition, including a complex formed between (i) a humanized 3E10 antibody or antigen binding fragment thereof, and (ii) an aptamer, as described herein. In some embodiments, the aptamer is non-covalently bound to the humanized 3E10 antibody or antigen binding fragment thereof. In some embodiments, the aptamer is covalently conjugated to the humanized 3E10 antibody or antigen binding fragment thereof.

In some embodiments, the aptamer is a PSMA aptamer, a HER2 aptamer, a MUC1 aptamer, a CD117 aptamer, a PTK7 aptamer, CTLA-4 aptamer, TS11a aptamer, PD-1 aptamer, a PD-i aptamer, a Macugen aptamer, AS1411, Sgc8, TD05, ARC1779, a-Thrombin (TBA), Macugen, E10030, AS1411, ARC1779, NU172, NOX-A12, NOX-E36, NOX-H94, ARC1905, REG1, ARC 19499, AS1411, AS1411, EpCAM, A10-3-J1, Sgc8c, TSA14, 5TR1, Endo28, EGFR, A10, Sgc8c, AS1411, NOX-A2, KH1C2, K19, TD05, AS1411, HB5, HeA2_3, H2, S6, SYL3C, APTA-12, M17, S-1, SL2, CAA01, CA50 A02, CA72-4 A01, APT-43, TA6, CA125.1, Apt928, R13, HF3-58, or HA5-68. In some embodiments, the aptamer targets an immune checkpoint regulatory protein. In some embodiments, the immune checkpoint regulatory protein is B7-H3, B7-H4, BTLA, CD160, CTLA4, KIP, LAG3, PD-1, PD-L1, PD-L2, TIM3, or TIGIT.

Non-limiting examples of aptamers that have been studied for the treatment of cancer are presented in Table 8 below.

TABLE 8
Example aptamers studied for the treatment of cancer.

| | Molecular | | |
|---|---|---|---|
| Aptamer | Target | Cancer Type | Aptamer Sequence |
| Sgc8c | Protein tyrosine | Leukemia; | ATC TAA CTG CTG CGC CGC |
| | kinase 7 (PTK-7) | acute lymphoblastic | CGG GAA AAT ACT GTA CGG |
| | | leukemia | TTA GA (SEQ ID NO: 128) |
| AS1411 | Nucleolin; | Leukemia; | GGT GGT GGT GGT TGT GGT |
| | MCF7 cells | acute lymphoblastic | GGT GGT GG (SEQ ID NO: 129) |
| | | leukemia; | |
| | | breast cancer | |
| NOX-A12 | CXCL12, | Leukemia; | GCG UGG UGU GAU CUA GAU |
| | MS-5 cells | Chronic lymphocytic | GUA UUG GCU GAU CCU AGU |
| | | leukemia | CAG GUA CGC (SEQ ID NO: 130) |
| KH1C12 | HL60 cells | Leukemia; | ATC CAG AGT GAC GCA GCA |
| | | Acute myeloid | TGC CCT AGT TAC TAC TAC |
| | | leukemia | TCT TTT TAG CAA ACG CCC |
| | | | TCG CTT TGG ACA CGG TGG |
| | | | CTT AGT (SEQ ID NO: 131) |
| K19 | Sigles-5, | Leukemia; | AAG GGG TTG GGT GGG TTT |
| | NB4 cells | Acute myeloid | ATA CAA ATT AAT TAA TAT |
| | | leukemia | TGT ATG GTA TAT TT (SEQ ID |
| | | | NO: 132) |
| TD05 | IgM, | Leukemia; | ACC GGG AGG ATA GTT CGG |
| | Ramos cells | Burkitt's lymphoma | TGG CTG TTC AGG GTC TCC |
| | | | TCC CGG TG (SEQ ID NO: 133) |
| HB5 | HER2 | Breast cancer | AAC CGC CCA AAT CCC TAA |
| | | | GAG TCT GCA CTT GTC ATT |
| | | | TTG TAT ATG TAT TTG GTT |
| | | | TTT GGC TCT CAC AGA CAC |
| | | | ACT ACA CAC GCA CAT G |
| | | | (SEQ ID NO: 134) |
| HeA2_3 | HER2 | Breast cancer | TCT AAA AGG ATT CTT CCC |
| | | | AAG GGG ATC CAA TTC AAA |
| | | | CAG C (SEQ ID NO: 135) |
| H2 | Her2 | Breast cancer | GGG CCG TCG AAC ACG AGC |
| | | | ATG GTG CGT GGA CCT AGG |
| | | | ATG ACC TGA GTA CTG TCC |
| | | | (SEQ ID NO: 136) |
| S6 | SK-BR-3 cells | Breast cancer | TGG ATG GGG AGA TCC GTT |
| | | | GAG TAA GCG GGC GTG TCT |
| | | | CTC TGC CGC CTT GCT ATG |
| | | | GGG (SEQ ID NO: 137) |
| SYL3C | EpCAM | Breast cancer | CAC TAC AGA GGT TGC GTC |
| | | | TGT CCC ACG TTG TCA TGG |
| | | | GGG GTT GGC CTG (SEQ ID |
| | | | NO: 138) |
| APTA-12 | Nucleolin | Pancreatic cancer | GGT GGT GGT GGT TZ*T GGT |
| | | | GGT GGT GG (SEQ ID NO: 139) |
| | | | Z* = gemcitabine |
| M17 | MMP14 | Pancreatic cancer | AGG GCC CGA CGT GAC GGC |
| | proteinase, | | ACG TCG GAT ATC TCA TGC |
| | MIA PaCa-2 and | | GTG T (SEQ ID NO: 140) |
| | PANC-1 cell | | |
| | lines | | |
| S-1 | DHX9, RNA | Colorectal cancer | GCC CAG CAT GCA TTA CTG |
| | helicase | | ATC GTG GTG TTT GCT TAG |
| | | | CCC A (SEQ ID NO: 141) |
| SL2B | VEGF | Colorectal cancer | TTT TTT TTT ACA TTC CTA |
| | | | AGT CTG AAA CAT TAC AGC |
| | | | TTG CTA CAC GAG AAG AGC |
| | | | CGC CAT AGT A (SEQ ID |
| | | | NO: 142) |
| CAA01 | CEA | Colorectal cancer | GGG UCG UGU CGG AUC CAG |
| | | | GCA CGA CGC AUA GCC UUG |
| | | | GGA GCG AGG AAA GCU UCU |
| | | | AAG GUA ACG AU (SEQ ID |
| | | | NO: 143) |
| CA50 A02 | CA50 | Colorectal cancer | GGG UCG UGU CGG AUC CAG |
| | | | CUC GAA AGU GGG CUG GCG |
| | | | AUG UGU CCC GAA GCU UCU |
| | | | AAG GUA ACG AU (SEQ ID |
| | | | NO: 144) |
| CA72-4 A01 | CA72-4 | Colorectal cancer | GGG UCG UGU CGG AUC CUG |
| | | | CGA AGG GGG GCA GAG GUU |
| | | | UGA CGC GAG AAA GCU UCU |
| | | | AAG GUA ACG AU (SEQ ID |
| | | | NO: 145) |
| APT-43 | Lung cancer | Lung cancer | CTA TAG CAA TGG TAC GGT |
| | marker | | ACT TCC TCT CAG GTG GGT |
| | | | GTA TGT GGG CTC CCT TTA |
| | | | CTG ATT GGG TCA AAA GTG |
| | | | CAC GCT ACT TTG CTA A (SEQ |
| | | | ID NO: 146) |
| N/A | A549 cell line | Lung cancer | GGT TGC ATG CCG TGG GGA |
| | | | GGG GGG TGG GTT TTA TAG |
| | | | CGT ACT CAG (SEQ ID NO: 147) |
| TA6 | CD44, | Lung cancer | TTG GGA CGG TGT TAA ACGA |
| | SKOV3, | | AAG GGG ACG AC (SEQ ID |
| | IGROV, and | | NO: 148) |
| | A2780 cell lines | | |
| CA125.1 | CA125 | Lung cancer | AAA AUG CAU GGA GCG AAG |
| | | | GUG UGG GGG AUA CCA ACC |
| | | | GCG CCG UG (SEQ ID NO: 149) |
| Apt928 | CD70 | Lung cancer | GCT GTG TGA CTC CTG CAA |
| | | | GCG GGA AGA GGG CAG GGG |
| | | | AGG GAG GGT GAC GCG GAA |
| | | | GAG GCA AGC AGC TGT ATC |
| | | | TTG TCT CC (SEQ ID NO: 150) |
| R13 | A2780, | Lung cancer | CTC TAG TTA TTG AGT TTT |
| | SKOV3 cells | | CTT TTA TGG GTG GGT GGG |
| | | | GGG TTT TT (SEQ ID NO: 151) |
| HF3-58 | A2780T cells | Lung cancer | TTG GAG CAG CGT GGA GGA |
| | | | TAT GCT TTC CGA CCG TGT |
| | | | TCG TTT GTT ATA ACG CTG |
| | | | CTC C (SEQ ID NO: 152) |
| HA5-68 | A2780T cells | Lung cancer | TTA AGG AGC AGC GTG GAG |
| | | | GAT ATC GGT GTT TAT GGT |
| | | | GTC TGT CTT CCT CCA GTT |
| | | | TCC TTC TGC GCC TT (SEQ ID |
| | | | NO: 153) |
| ARC126 | PDGF-B | | Akiyama, Kachi et al., 2006 |
| (RNA) | | | |
| AX102 | PDGF-B | | Sennino, Falcon et al., 2007 |
| (RNA) | | | |
| SL (2)-B | VEGF-165 | | Kaur, Li et al., 2013 |
| (DNA) | | | |
| RNV66 | VEGF-165 | | Gantenbein, Sarikaya et al., 2015 |
| (DNA) | | | |
| FCL-II | Nucleolin | | Fan, Sun et al., 2017 |
| (DNA, | | | |
| modified | | | |
| form | | | |
| AS1411) | | | |
| NOX-A12 | CXCL12 | | Vater, Sahlmann et al., 2013; Liu, |
| (RNA) | | | Alomran et al., 2014; |
| | | | Hoellenriegel, Zboralski et al., |
| | | | 2014; Zboralski, Hoehlig et al., |
| | | | 2017 |
| E0727 | EGFR | | Li, Nguyen et al., 2011 [8], |
| (RNA) | | | Esposito, Passaro et al., 2011 [7], |
| CL428 | EGFR | | Wan, |
| (RNA) | | | Tamuly et al., 2013; Buerger, |
| KDI130 | EGFR | | Nagel-Wolfrum et al., 2003; Wang, |
| (RNA) | | | Song et al., 2014 |
| TuTu2231 | EGFR | | |
| (RNA) | | | |
| Trimeric | HER2 | | Kim and Jeong 2011; Mahlknecht, |
| apt (DNA) | | | Maron et al., 2013 |
| PNDA-3 | Periostin | | Lee, Kim et al., 2013 |
| (DNA) | | | |
| TTA140,41 | TN-C | | Hicke, Stephens et al., 2006; |
| (DNA) | | | Daniels, Chen et al., 2003; Hicke, |
| | | | Marion et al., 2001 |
| GBI-1042 | TN-C | | Hicke, Stephens et al., 2006; |
| (DNA) | | | Daniels, Chen et al., 2003; Hicke, |
| | | | Marion et al., 2001 |
| NAS - 24 | Vimentin | | Zamay, Kolovskaya et al., 2014 |
| (DNA) | | | |
| YJ-1 (RNA) | CEA | | Lee, Han et al., 2012 |
| AGE-apt | AGE | | Ojima, Matsui et al., 2014 |
| (DNA) | | | |
| A-P50 | NF-KB | | Mi, Zhang et al., 2008 |
| (RNA) | | | |
| GL21.T | Axl | | Cerchia, Esposito et al., 2012 |
| (RNA) | | | |
| OPN-R3 | OPN | | Mi, Guo et al., 2009; Talbot, Mi et |
| (RNA) | | | al., 2011 |
| AGC03 | HGC-27 | | Zhang, Zhang et al., 2014; Cao, |
| (DNA) | | | Yuan et al., 2014 |
| cy-apt | HGC-27 | | Zhang, Zhang et al., 2014; Cao, |
| (DNA) | | | Yuan et al., 2014 |
| BC15 (DNA) | hnRNP A1 | | Li, Wang et al., 2012 |
| A9g (RNA) | PSMA | | Dassie, Hernandez et al., 2014 |
| ESTA | E-selectin | | Mann, Somasunderam et al., 2010; |
| (DNA) | | | Kang, Hasan et al., 2015; Kang, |
| | | | Blache et al., 2016; Morita, Kamal |
| | | | et al., 2016 |
| M12-23 | 4-1 BB | | McNamara, Kolonias et al., 2008 |
| (RNA) | | | |
| OX40-apt | OX40 | | Dollins, Nair et al., 2008; Pratico, |
| (RNA) | | | Sullenger et al., 2013 |
| CD28-apt | CD28 | | Pastor, Soldevilla et al., 2013 |
| (RNA) | | | |
| De160 | CTLA-4 | | Santulli-Marotto, Nair et al., 2003 |
| (RNA) | | | |
| PSMA-4- | PSMA/4-1BB | | Pastor, Kolonias et al., 2011 |
| 1BB-apt | | | |
| (RNA) | | | |
| CD16a/c- | CD16a/c-Met | | Eder, VandeWoude et al., 2009 |
| Met-apt | | | |
| (RNA) | | | |
| VEGF-4- | VEGF/4-1BB | | Schrand, Berezhnoy et al., 2014 |
| 1BB apt | | | |
| (DNA) | | | |
| MP7 (DNA) | PD-1 | | Prodeus, Abdul-Wahid et al., 2015 |
| aptPD-L1 | PD-L1 | | Lai, Huang et al., 2016 |
| (DNA) | | | |
| R5A1 (RNA) | IL 10R | | Berezhnoy, Stewart et al., 2012 |
| CL-42 | IL4R | | Roth, De La Fuente et al., 2012 |
| (RNA) | | | |
| CD44- | CD44/EpCAM | | Zheng, Zhao et al., 2017 |
| EpCAM | | | |
| aptamer | | | |
| (RNA) | | | |
| TIM3 Apt | TIM3 | | Hervas-Stubbs, Soldevilla et al., |
| (RNA) | | | 2016 |
| CD40apt | CD40 | | Soldevilla, Villanueva et al., 2015 |
| (RNA) | | | |
| AptCTLA-4 | CTLA-4 | | Huang, Lai et al., 2017 |
| (DNA) | | | |
| AON-D211- | C5a | | Ajona, Ortiz-Espinosa et al., 2017 |
| Aptamer | | | |
| (RNA/DNA) | | | |

Accordingly, in some embodiments, the present disclosure relates to compositions and methods for treating a cancer in a subject by administering to the subject a therapeutically effective amount of a composition including a complex formed between (i) a humanized 3E10 antibody or antigen binding fragment thereof, and (ii) an aptamer selected from those aptamers listed in Table 8, as described herein. In some embodiments, the aptamer is non-covalently bound to the humanized 3E10 antibody or antigen binding fragment thereof. In some embodiments, the aptamer is covalently conjugated to the humanized 3E10 antibody or antigen binding fragment thereof.

In some embodiments, the present disclosure provides compositions, as well as methods for treating a cancer in a subject by administering to the subject a therapeutically effective amount of a composition, including a complex formed between (i) a humanized 3E10 antibody or antigen binding fragment thereof, and (ii) an aptamer that specifically binds to a respective molecular target selected from those molecular targets listed in Table 8, as described herein, where the cancer is a cancer associated with the molecular target within Table 8. In some embodiments, the aptamer is non-covalently bound to the humanized 3E10 antibody or antigen binding fragment thereof. In some embodiments, the aptamer is covalently conjugated to the humanized 3E10 antibody or antigen binding fragment thereof.

### Ribozymes

In some embodiments, humanized 3E10 antibodies and antigen binding fragments thereof described herein are complexed with a ribozyme or a polynucleotide that encodes a ribozyme. Ribozymes are catalytically active RNA molecules. Ribozymes occur naturally in various sizes and shapes. They catalyze cleavage and ligation of specific phosphodiester bonds. Peptide bond formation during protein synthesis on the ribosome is catalyzed by ribosomal RNA. The biological functions of ribozymes are diverse and they play central roles during transfer RNA maturation, intron splicing, replication of RNA viruses or viroids, the regulation of messenger RNA stability, and protein synthesis (Westhof et al. in Encyclopedia of Virology. (Third Edition), 2008). In some embodiments, the ribozyme targets human telomerase reverse transcriptase (hTERT) RNA.

Accordingly, in some embodiments, the present disclosure provides compositions, as well as methods for treating a disorder (such as cancer) by administering to a subject in need thereof a therapeutically effective amount of a composition, including a complex formed between (i) a humanized 3E10 antibody or antigen binding fragment thereof, and (i) a ribozyme or a polynucleotide that encodes a ribozyme. In some embodiments, the ribozyme or polynucleotide that encodes the ribozyme is non-covalently bound to the humanized 3E10 antibody or antigen binding fragment thereof. In some embodiments, the ribozyme or polynucleotide that encodes the ribozyme is covalently conjugated to the humanized 3E10 antibody or antigen binding fragment thereof.

### Methods of Treatment

In some embodiments, the present disclosure provides compositions, as well as methods for treating a disorder by administering to a subject in need thereof a therapeutically effective amount of a composition, including a complex formed between (i) a humanized 3E10 antibody or antigen binding fragment thereof, and (i) a therapeutic agent. In some embodiments, the therapeutic agent is covalently conjugated to the humanized 3E10 antibody or antigen binding fragment thereof.

In some embodiments, the therapeutic agent is non-covalently associated to the humanized 3E10 antibody or antigen binding fragment thereof. In some embodiments, the humanized 3E10 antibody or antigen binding fragment thereof is associated, e.g., conjugated, with a delivery vehicle for the therapeutic agent. In some embodiments, the delivery vehicle is a liposome, a lipid nanoparticle, a nanoparticle, a microparticle, a beaded system, a micelle, a biomimetic exosome, or a dendrimer. For a review of drug delivery systems see, for example, Tiwari G. et al., International Journal of Pharmaceutical Investigation, 2(1):2-11 (2012), the content of which is incorporated herein by reference in its entirety. For a review of strategies for functionalizing nanoparticulate drug delivery systems see, for example, Seidu T A et al., Pharmaceutics, 14(5):1113 (2022), the content of which is incorporated herein by reference in its entirety.

In some embodiments, the therapeutic agent is a DNA damage inducing agent, a DNA repair inhibitor, an immune modulatory molecule, an alkylating agent, a microtubule inhibitor, an immune checkpoint inhibitor, an angiogenesis inhibitor, adoptive cell therapy, or a topoisomerase inhibitor. In some embodiment, the therapeutic agent is an anti-tumor drug. In some embodiments, the therapeutic agent is a maytansinoid, a benzodiazepine, an auristatin, a tecan, a taxoid, CC-1065, (4S)-4,11-Diethyl-4,9-dihydroxy-1,4-dihydro-3H,14H-pyrano[3′,4′:6,7]indolizino[1,2-b]quinoline-3,14-dione (SN38), exatecan, monomethyl auristatin E (MMAE), monomethyl auristatin F (MMAF), a pyrrolobenzodiazepine (PBD), PROteolysis TArgeting Chimera (PROTAC), deruxtecan (Dxd), a calicheamicin, a duocarmycin, a stimulator of interferon genes (STING) agonist, PNU-159682, NMS249, IMGN Camp 1, duocarmycin hydroxybenzamide azaindole (DUBA), or a prodrug thereof. In some embodiments, the therapeutic agent is a maytansinoid. In some embodiments, the therapeutic agent is N(2′)-deacetyl-N(2′)-(3-mercapto-1-oxopropyl)-maytansine (DM1). In some embodiments, the therapeutic agent is N2′-deacetyl-N2′-(4-mercapto-4-methyl-1-oxopentyl) maytansine (DM4). In some embodiments, the therapeutic agent is (4S)-4,11-Diethyl-4,9-dihydroxy-1,4-dihydro-3H,14H-pyrano[3′,4′:6,7]indolizino[1,2-b]quinoline-3,14-dione (SN38). In some embodiments, the therapeutic agent is PNU-159682. In some embodiments, the therapeutic agent is PNU-159682. In some embodiments, the therapeutic agent is NMS249.

In some embodiments, the therapeutic agent is a therapeutic polynucleotide, e.g., as described herein. In some embodiments, the therapeutic polynucleotide is non-covalently bound to the humanized 3E10 antibody or antigen binding fragment thereof. In some embodiments, the therapeutic polynucleotide is covalently attached to the humanized 3E10 antibody or antigen binding fragment thereof. In some embodiments, the therapeutic polynucleotide is non-replicating unmodified mRNA. In some embodiments, the therapeutic polynucleotide is non-replicating modified mRNA. In some embodiments, the therapeutic polynucleotide is a self-amplifying mRNA. In some embodiments, the therapeutic polynucleotide is a plasmid encoding the protein or peptide. In some embodiments, wherein the therapeutic polynucleotide is a gene-regulating polynucleotide

In some embodiments, the disorder is cancer. In some embodiments, the cancer is a carcinoma, a sarcoma, a blastoma, a papilloma, or an adenoma. In some embodiments, the cancer is a metastatic cancer. In some embodiments, the cancer is bladder cancer, blood cancer, brain cancer, breast cancer, bone cancer, cervical cancer, colorectal cancer, endocrine cancer, esophageal cancer, gastric cancer, head and neck cancer, hepatobiliary cancer, leukemia, lung cancer, lymphoma, melanoma, myeloma, ovarian cancer, pancreatic cancer, prostate cancer, renal cancer, thyroid cancer, or uterine cancer.

In some embodiments, methods are provided for treating a subject suffering from a carcinoma, blastoma, and sarcoma, and certain leukemia or lymphoid malignancies, benign and malignant tumors, and malignancies e.g., sarcomas, carcinomas, and melanomas; hematologic cancers of the blood or bone marrow; hematological (or hematogenous) cancers, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia; solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma, or brain metastases.

In some embodiments, the disclosure provides methods for treating a cancer of the central nervous system by parenterally administering to the periphery of the subject a therapeutically effective amount of a composition including a complex formed between (i) a humanized 3E10 antibody or antigen binding fragment thereof and (ii) a therapeutic agent, as described herein.

In some embodiments, the cancer is a skin cancer. In some embodiments, the skin cancer is basal cell carcinoma, squamous cell carcinoma, or melanoma. In one embodiment, the cancer is melanoma.

In one aspect, the present disclosure provides methods for a treating skeletal muscle disease in a subject comprising administering to the subject a therapeutically effective amount of a composition comprising a non-covalent complex of (i) a humanized 3E10 antibody or antigen binding fragment thereof, and (i) and an mRNA encoding a protein mutated in a genetic skeletal muscle disease. In some embodiments, the mRNA is non-covalently bound to the humanized 3E10 antibody or antigen binding fragment thereof. In some embodiments, the mRNA is covalently conjugated to the humanized 3E10 antibody or antigen binding fragment thereof. In some embodiments, the mRNA encodes dystrophin (DMD) or a fragment thereof, e.g., a mini-dystrophin or micro-dystrophin construct.

### Stoichiometric Ratios of Therapeutic Polynucleotides

In some embodiments, e.g., where a therapeutic polynucleotide is non-covalently bound to a humanized 3E10 antibody or antigen binding fragment thereof, the compositions described herein have a molar ratio of humanized 3E10 antibody or antigen binding fragment thereof to therapeutic polynucleotide of at least 2:1. The use of molar ratios of humanized 3E10 antibody or antigen binding fragment thereof to therapeutic polynucleotide in the compositions described herein protects the therapeutic polynucleotide from degradation. For instance, the parental 3E10 and 3E10 (D31N) variant antibodies protect mRNA from RNAseA-mediated RNA degradation at molar ratios of 2:1 and 20:1, the protection afforded by the 20:1 molar ratio exceeds the protection afforded at 2:1.

In some embodiments, the compositions described here have a molar ratio of humanized 3E10 antibody or antigen binding fragment thereof to therapeutic polynucleotide that is at least 2:1. In some embodiments, the compositions described here have a molar ratio of humanized 3E10 antibody or antigen binding fragment thereof to therapeutic polynucleotide that is at least 2.5:1. In some embodiments, the compositions described here have a molar ratio of humanized 3E10 antibody or antigen binding fragment thereof to therapeutic polynucleotide that is at least 5:1. In some embodiments, the compositions described here have a molar ratio of humanized 3E10 antibody or antigen binding fragment thereof to therapeutic polynucleotide that is at least 7.5:1. In some embodiments, the compositions described here have a molar ratio of humanized 3E10 antibody or antigen binding fragment thereof to therapeutic polynucleotide that is at least 10:1. In some embodiments, the compositions described here have a molar ratio of humanized 3E10 antibody or antigen binding fragment thereof to therapeutic polynucleotide that is at least 15:1. In some embodiments, the compositions described here have a molar ratio of humanized 3E10 antibody or antigen binding fragment thereof to therapeutic polynucleotide that is at least 20:1. In some embodiments, the compositions described here have a molar ratio of humanized 3E10 antibody or antigen binding fragment thereof to therapeutic polynucleotide that is at least 25:1. In some embodiments, the compositions described here have a molar ratio of humanized 3E10 antibody or antigen binding fragment thereof to therapeutic polynucleotide that is at least 30:1. In some embodiments, the compositions described here have a molar ratio of humanized 3E10 antibody or antigen binding fragment thereof to therapeutic polynucleotide that is at least 40:1.

In some embodiments, the compositions described here have a molar ratio of humanized 3E10 antibody or antigen binding fragment thereof to therapeutic polynucleotide that is at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 21:1, at least 22:1, at least 23:1, at least 24:1, at least 25:1, at least 26:1, at least 27:1, at least 28:1, at least 29:1, at least 30:1, at least 31:1, at least 32:1, at least 33:1, at least 34:1, at least 35:1, at least 36:1, at least 37:1, at least 38:1, at least 39:1, at least 40:1, at least 41:1, at least 42:1, at least 43:1, at least 44:1, at least 45:1, or greater.

In some embodiments, the compositions described here have a molar ratio of humanized 3E10 antibody or antigen binding fragment thereof to therapeutic polynucleotide that is 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, or greater.

In some embodiments, the compositions described here have a molar ratio of humanized 3E10 antibody or antigen binding fragment thereof to therapeutic polynucleotide that is no more than 50:1. In some embodiments, the compositions described here have a molar ratio of humanized 3E10 antibody or antigen binding fragment thereof to therapeutic polynucleotide that is no more than 40:1. In some embodiments, the compositions described here have a molar ratio of humanized 3E10 antibody or antigen binding fragment thereof to therapeutic polynucleotide that is no more than 30:1. In some embodiments, the compositions described here have a molar ratio of humanized 3E10 antibody or antigen binding fragment thereof to therapeutic polynucleotide that is no more than 25:1. In some embodiments, the compositions described here have a molar ratio of humanized 3E10 antibody or antigen binding fragment thereof to therapeutic polynucleotide that is no more than 20:1. In some embodiments, the compositions described here have a molar ratio of humanized 3E10 antibody or antigen binding fragment thereof to therapeutic polynucleotide that is no more than 15:1. In some embodiments, the compositions described here have a molar ratio of humanized 3E10 antibody or antigen binding fragment thereof to therapeutic polynucleotide that is no more than 10:1.

In some embodiments, the compositions described here have a molar ratio of humanized 3E10 antibody or antigen binding fragment thereof to therapeutic polynucleotide that is no more than 50:1, no more than 49:1, no more than 48:1, no more than 47:1, no more than 46:1, no more than 45:1, no more than 44:1, no more than 43:1, no more than 42:1, no more than 41:1, no more than 40:1, no more than 39:1, no more than 38:1, no more than 37:1, no more than 36:1, no more than 35:1, no more than 34:1, no more than 33:1, no more than 32:1, no more than 31:1, no more than 30:1, no more than 29:1, no more than 28:1, no more than 27:1, no more than 26:1, no more than 25:1, no more than 24:1, no more than 23:1, no more than 22:1, no more than 21:1, no more than 20:1, no more than 19:1, no more than 18:1, no more than 17:1, no more than 16:1, no more than 15:1, no more than 14:1, no more than 13:1, no more than 12:1, no more than 11:1, no more than 10:1, no more than 9:1, no more than 8:1, no more than 7:1, no more than 6:1, no more than 5:1, or less.

In some embodiments, the compositions described here have a molar ratio of humanized 3E10 antibody or antigen binding fragment thereof to therapeutic polynucleotide that is of from 2:1 to 50:1. In some embodiments, the compositions described here have a molar ratio of humanized 3E10 antibody or antigen binding fragment thereof to therapeutic polynucleotide that is of from 2:1 to 40:1. In some embodiments, the compositions described here have a molar ratio of humanized 3E10 antibody or antigen binding fragment thereof to therapeutic polynucleotide that is of from 2:1 to 30:1. In some embodiments, the compositions described here have a molar ratio of humanized 3E10 antibody or antigen binding fragment thereof to therapeutic polynucleotide that is of from 2:1 to 25:1. In some embodiments, the compositions described here have a molar ratio of humanized 3E10 antibody or antigen binding fragment thereof to therapeutic polynucleotide that is of from 2:1 to 20:1. In some embodiments, the compositions described here have a molar ratio of humanized 3E10 antibody or antigen binding fragment thereof to therapeutic polynucleotide that is of from 2:1 to 15:1. In some embodiments, the compositions described here have a molar ratio of humanized 3E10 antibody or antigen binding fragment thereof to therapeutic polynucleotide that is of from 2:1 to 10:1. In some embodiments, the compositions described here have a molar ratio of humanized 3E10 antibody or antigen binding fragment thereof to therapeutic polynucleotide that is of from 2:1 to 7.5:1. In some embodiments, the compositions described here have a molar ratio of humanized 3E10 antibody or antigen binding fragment thereof to therapeutic polynucleotide that is of from 2:1 to 5:1. In some embodiments, the compositions described here have a molar ratio of humanized 3E10 antibody or antigen binding fragment thereof to therapeutic polynucleotide that is of from 2:1 to 5:1. In some embodiments, the compositions described here have a molar ratio of humanized 3E10 antibody or antigen binding fragment thereof to therapeutic polynucleotide that is of from 2:1 to 3:1.

In some embodiments, the compositions described here have a molar ratio of humanized 3E10 antibody or antigen binding fragment thereof to therapeutic polynucleotide that is of from 5:1 to 50:1. In some embodiments, the compositions described here have a molar ratio of humanized 3E10 antibody or antigen binding fragment thereof to therapeutic polynucleotide that is of from 5:1 to 40:1. In some embodiments, the compositions described here have a molar ratio of humanized 3E10 antibody or antigen binding fragment thereof to therapeutic polynucleotide that is of from 5:1 to 30:1. In some embodiments, the compositions described here have a molar ratio of humanized 3E10 antibody or antigen binding fragment thereof to therapeutic polynucleotide that is of from 5:1 to 25:1. In some embodiments, the compositions described here have a molar ratio of humanized 3E10 antibody or antigen binding fragment thereof to therapeutic polynucleotide that is of from 5:1 to 20:1. In some embodiments, the compositions described here have a molar ratio of humanized 3E10 antibody or antigen binding fragment thereof to therapeutic polynucleotide that is of from 5:1 to 15:1. In some embodiments, the compositions described here have a molar ratio of humanized 3E10 antibody or antigen binding fragment thereof to therapeutic polynucleotide that is of from 5:1 to 10:1. In some embodiments, the compositions described here have a molar ratio of humanized 3E10 antibody or antigen binding fragment thereof to therapeutic polynucleotide that is of from 5:1 to 7.5:1.

In some embodiments, the compositions described here have a molar ratio of humanized 3E10 antibody or antigen binding fragment thereof to therapeutic polynucleotide that is of from 10:1 to 50:1. In some embodiments, the compositions described here have a molar ratio of humanized 3E10 antibody or antigen binding fragment thereof to therapeutic polynucleotide that is of from 10:1 to 40:1. In some embodiments, the compositions described here have a molar ratio of humanized 3E10 antibody or antigen binding fragment thereof to therapeutic polynucleotide that is of from 10:1 to 30:1. In some embodiments, the compositions described here have a molar ratio of humanized 3E10 antibody or antigen binding fragment thereof to therapeutic polynucleotide that is of from 10:1 to 25:1. In some embodiments, the compositions described here have a molar ratio of humanized 3E10 antibody or antigen binding fragment thereof to therapeutic polynucleotide that is of from 10:1 to 20:1. In some embodiments, the compositions described here have a molar ratio of humanized 3E10 antibody or antigen binding fragment thereof to therapeutic polynucleotide that is of from 10:1 to 15:1.

In some embodiments, the compositions described here have a molar ratio of humanized 3E10 antibody or antigen binding fragment thereof to therapeutic polynucleotide that is of from 15:1 to 50:1. In some embodiments, the compositions described here have a molar ratio of humanized 3E10 antibody or antigen binding fragment thereof to therapeutic polynucleotide that is of from 15:1 to 40:1. In some embodiments, the compositions described here have a molar ratio of humanized 3E10 antibody or antigen binding fragment thereof to therapeutic polynucleotide that is of from 15:1 to 30:1. In some embodiments, the compositions described here have a molar ratio of humanized 3E10 antibody or antigen binding fragment thereof to therapeutic polynucleotide that is of from 15:1 to 25:1. In some embodiments, the compositions described here have a molar ratio of humanized 3E10 antibody or antigen binding fragment thereof to therapeutic polynucleotide that is of from 15:1 to 20:1.

In some embodiments, the compositions described here have a molar ratio of humanized 3E10 antibody or antigen binding fragment thereof to therapeutic polynucleotide that is of from 20:1 to 50:1. In some embodiments, the compositions described here have a molar ratio of humanized 3E10 antibody or antigen binding fragment thereof to therapeutic polynucleotide that is of from 20:1 to 40:1. In some embodiments, the compositions described here have a molar ratio of humanized 3E10 antibody or antigen binding fragment thereof to therapeutic polynucleotide that is of from 20:1 to 30:1. In some embodiments, the compositions described here have a molar ratio of humanized 3E10 antibody or antigen binding fragment thereof to therapeutic polynucleotide that is of from 20:1 to 25:1.

In some embodiments, the compositions described here have a molar ratio of humanized 3E10 antibody or antigen binding fragment thereof to therapeutic polynucleotide that is of from 25:1 to 50:1. In some embodiments, the compositions described here have a molar ratio of humanized 3E10 antibody or antigen binding fragment thereof to therapeutic polynucleotide that is of from 25:1 to 40:1. In some embodiments, the compositions described here have a molar ratio of humanized 3E10 antibody or antigen binding fragment thereof to therapeutic polynucleotide that is of from 25:1 to 30:1. In some embodiments, the compositions described here have a molar ratio of humanized 3E10 antibody or antigen binding fragment thereof to therapeutic polynucleotide that is of from 30:1 to 50:1. In some embodiments, the compositions described here have a molar ratio of humanized 3E10 antibody or antigen binding fragment thereof to therapeutic polynucleotide that is of from 30:1 to 40:1. In yet other embodiments, other ranges falling with the range of 2:1 to 50:1 are contemplated.

In some embodiments, the compositions described here have a molar ratio of humanized 3E10 antibody or antigen binding fragment thereof to therapeutic polynucleotide that is of from 2:1 to 50:1, from 2:1 to 40:1, from 2:1 to 30:1, from 2:1 to 25:1, from 2:1 to 20:1, from 2:1 to 15:1, from 2:1 to 10:1, from 2:1 to 7.5:1, from 2:1 to 5:1, from 5:1 to 50:1, from 5:1 to 40:1, from 5:1 to 30:1, from 5:1 to 25:1, from 5:1 to 20:1, from 5:1 to 15:1, from 5:1 to 10:1, from 5:1 to 7.5:1, from 10:1 to 50:1, from 10:1 to 40:1, from 10:1 to 30:1, from 10:1 to 25:1, from 10:1 to 20:1, from 10:1 to 15:1, from 15:1 to 50:1, from 15:1 to 40:1, from 15:1 to 30:1, from 15:1 to 25:1, from 15:1 to 20:1, from 20:1 to 50:1, from 20:1 to 40:1, from 20:1 to 30:1, from 20:1 to 25:1, from 25:1 to 50:1, from 25:1 to 40:1, from 25:1 to 30:1, from 30: to 50:1, from 30:1 to 40:1, or from 40:1 to 50:1. In yet other embodiments, other ranges falling with the range of from 2:1 to 50:1 are contemplated.

In some embodiments, the compositions described here have a molar ratio of humanized 3E10 antibody or antigen binding fragment thereof to therapeutic polynucleotide that is of from 1:1 to 50:1. In some embodiments, the compositions described here have a molar ratio of humanized 3E10 antibody or antigen binding fragment thereof to therapeutic polynucleotide that is of from 1:1 to 30:1. In some embodiments, the compositions described here have a molar ratio of humanized 3E10 antibody or antigen binding fragment thereof to therapeutic polynucleotide that is of from 1:1 to 20:1. In some embodiments, the compositions described here have a molar ratio of humanized 3E10 antibody or antigen binding fragment thereof to therapeutic polynucleotide that is of from 1:1 to 10:1. In some embodiments, the compositions described here have a molar ratio of humanized 3E10 antibody or antigen binding fragment thereof to therapeutic polynucleotide that is of from 1:1 to 5:1.

In some embodiments, the molar ratio is related to the size of the nucleic acid (i.e., the therapeutic polynucleotide). For instance, longer polynucleotides are complexed at higher molar ratios and shorter polynucleotides are complexed at lower molar ratios.

In some embodiments, the size of the therapeutic polynucleotide is about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 205 bp, 210 bp, 215 bp, 220 bp, 225 bp, 230 bp, 235 bp, 240 bp, 245 bp, 250 bp, 255 bp, 260 bp, 265 bp, 270 bp, 275 bp, 280 bp, 285 bp, 290 bp, 295 bp, 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, or more and any range in between.

In some embodiments, the molar ratios disclosed herein are related to the size of the therapeutic polynucleotide as disclosed herein. For instance, longer polynucleotides are complexed at higher molar ratios and shorter polynucleotides are complexed at lower molar ratios. In some embodiments, any molar ratio disclosed herein can be combined with any size of the therapeutic polynucleotide. Nonlimiting examples, include the composition that has a molar ratio of 3E10 antibody to therapeutic polynucleotide that is 1:1, 2:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, 200:1, 205:1, 210:1, 215:1, 220:1, 225:1, 230:1, 235:1, 240:1, 245:1, 250:1, or greater and any ranges in between, wherein the size therapeutic polynucleotide is about 10 bp, 15 bp, 20 bp, 25 bp, 30 bp, 35 bp, 40 bp, 45 bp, 50 bp, 55 bp, 60 bp, 65 bp, 70 bp, 75 bp, 80 bp, 85 bp, 90 bp, 95 bp, 100 bp, 105 bp, 110 bp, 115 bp, 120 bp, 125 bp, 130 bp, 135 bp, 140 bp, 145 bp, 150 bp, 155 bp, 160 bp, 165 bp, 170 bp, 175 bp, 180 bp, 185 bp, 190 bp, 195 bp, 200 bp, 205 bp, 210 bp, 215 bp, 220 bp, 225 bp, 230 bp, 235 bp, 240 bp, 245 bp, 250 bp, 255 bp, 260 bp, 265 bp, 270 bp, 275 bp, 280 bp, 285 bp, 290 bp, 295 bp, 300 bp, 305 bp, 310 bp, 315 bp, 320 bp, 325 bp, 330 bp, 335 bp, 340 bp, 345 bp, 350 bp, 355 bp, 360 bp, 365 bp, 370 bp, 375 bp, 380 bp, 385 bp, 390 bp, 395 bp, 400 bp, 405 bp, 410 bp, 415 bp, 420 bp, 425 bp, 430 bp, 435 bp, 440 bp, 445 bp, 450 bp, 455 bp, 460 bp, 465 bp, 470 bp, 475 bp, 480 bp, 485 bp, 490 bp, 495 bp, 500 bp, 505 bp, 510 bp, 515 bp, 520 bp, 525 bp, 530 bp, 535 bp, 540 bp, 545 bp, 550 bp, or more and any ranges in between.

All methods described herein can be performed in any suitable order unless otherwise indicated or otherwise clearly contradicted by context. The use of any and all examples, or example language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### EXAMPLES

### Example 1: Binding Kinetics and Affinity Measurements of Humanized 3E10 Antibodies

Briefly, seven humanized versions of the 3E10 variable heavy domain (VH-h1 to VH-h7) and six humanized versions of the 3E10 variable light domain (VL-h1-VL-h6) were constructed. FIGS. 5-10 provide the amino acid sequences for the humanized VH domains, the mature portion of the full HC incorporating the humanized VH, the full-length heavy chain incorporating the humanized VH, the humanized VL domains, the mature portion of the full LC incorporating the humanized VL, and the full-length heavy chain incorporating the humanized VL, respectively.

Out of the 42 possible humanized 3E10 antibodies that could be created from these humanized VH and VL domains, twenty-two constructs were generated, as outlined in Table 1. In Table 1, the variant number refers to the specific combination of humanized VH and VL. For example, Variant 12 refers to a humanized 3E10 antibody that includes humanized VH-h1 and humanized VL-h2 and Variant 41 refers to a humanized 3E10 antibody that includes humanized VH-h4 and humanized VL-h1.

TABLE 1
Humanized 3E10 antibody constructs

| Humanized | | |
|---|---|---|
| 3E10 Antibody | | |
| (n = 22) | 3E10-VH | 3E10-VL |
| Variant 11 | 3E10-VH-h1 | 3E10-VL-h1 |
| | (SEQ ID | (SEQ ID |
| | NO: 64) | NO: 85) |
| Variant 12 | 3E10-VH-h1 | 3E10-VL-h2 |
| | (SEQ ID | (SEQ ID |
| | NO: 64) | NO: 86) |
| Variant 13 | 3E10-VH-h1 | 3E10-VL-h3 |
| | (SEQ ID | (SEQ ID |
| | NO: 64) | NO: 87) |
| Variant 14 | 3E10-VH-h1 | 3E10-VL-h4 |
| | (SEQ ID | (SEQ ID |
| | NO: 64) | NO: 88) |
| Variant 21 | 3E10-VH-h2 | 3E10-VL-h1 |
| | (SEQ ID | (SEQ ID |
| | NO: 65) | NO: 85) |
| Variant 22 | 3E10-VH-h2 | 3E10-VL-h2 |
| | (SEQ ID | (SEQ ID |
| | NO: 65) | NO: 86) |
| Variant 23 | 3E10-VH-h2 | 3E10-VL-h3 |
| | (SEQ ID | (SEQ ID |
| | NO: 65) | NO: 87) |
| Variant 24 | 3E10-VH-h2 | 3E10-VL-h4 |
| | (SEQ ID | (SEQ ID |
| | NO: 65) | NO: 88) |
| Variant 31 | 3E10-VH-h3 | 3E10-VL-h1 |
| | (SEQ ID | (SEQ ID |
| | NO: 66) | NO: 85) |
| Variant 32 | 3E10-VH-h3 | 3E10-VL-h2 |
| | (SEQ ID | (SEQ ID |
| | NO: 66) | NO: 86) |
| Variant 33 | 3E10-VH-h3 | 3E10-VL-h3 |
| | (SEQ ID | (SEQ ID |
| | NO: 66) | NO: 87) |
| Variant 34 | 3E10-VH-h3 | 3E10-VL-h4 |
| | (SEQ ID | (SEQ ID |
| | NO: 66) | NO: 88) |
| Variant 41 | 3E10-VH-h4 | 3E10-VL-h1 |
| | (SEQ ID | (SEQ ID |
| | NO: 67) | NO: 85) |
| Variant 42 | 3E10-VH-h4 | 3E10-VL-h2 |
| | (SEQ ID | (SEQ ID |
| | NO: 67) | NO: 86) |
| Variant 43 | 3E10-VH-h4 | 3E10-VL-h3 |
| | (SEQ ID | (SEQ ID |
| | NO: 67) | NO: 87) |
| Variant 44 | 3E10-VH-h4 | 3E10-VL-h4 |
| | (SEQ ID | (SEQ ID |
| | NO: 67) | NO: 88) |
| Variant 55 | 3E10-VH-h5 | 3E10-VL-h5 |
| | (SEQ ID | (SEQ ID |
| | NO: 68) | NO: 89) |
| Variant 56 | 3E10-VH-h5 | 3E10-VL-h6 |
| | (SEQ ID | (SEQ ID |
| | NO: 68) | NO: 90) |
| Variant 65 | 3E10-VH-h6 | 3E10-VL-h5 |
| | (SEQ ID | (SEQ ID |
| | NO: 69) | NO: 89) |
| Variant 66 | 3E10-VH-h6 | 3E10-VL-h6 |
| | (SEQ ID | (SEQ ID |
| | NO: 69) | NO: 90) |
| Variant 75 | 3E10-VH-h7 | 3E10-VL-h5 |
| | (SEQ ID | (SEQ ID |
| | NO: 70) | NO: 89) |
| Variant 76 | 3E10-VH-h7 | 3E10-VL-h6 |
| | (SEQ ID | (SEQ ID |
| | NO: 70) | NO: 90) |

The nucleic acid affinities of the 22 humanized 3E10 antibodies were investigated using a solid-phase ELISA poly-dT binding assay. Briefly, microtiter plates were coated by incubating 100 μl of μg/ml streptavidin in PBS per well overnight at 4′° C. After removing the coating solution, the wells were washed with 0.05% PBST twice. The wells were then blocked by incubating with 1% bovine serum albumin (BSA) in PBS for 1 hour at 37° C. After removing the blocking solution the wells were washed once with 0.05% PBST. The wells were then coated with poly-dT by incubating 100 μl of 0.1 μM biotin-labeled poly-dT in ELISA buffer for 40 minutes at room temperature. After removing the coating solution, the wells were washed three times with 0.05% PBST. A 3-fold dilution series of antibody concentrations of from 100 μg/ml down to 1.69 ng/ml was established for each of the 22 humanized antibodies described above. 100 μl of each dilution were incubated in separate wells for four hours at 4° C. After removing the dilution solutions, the wells were again washed three times with 0.05% PBST. Antibody bound to the poly-dT was detected by incubating 100 μl of a 1:10,000 dilution of a horseradish peroxidase (HRP)-conjugated secondary mouse anti-human IgG Fc antibody in each well at room temperature for 1.5 hours. After removing the secondary antibody solution, the wells were again washed three times with 0.05% PBST. The secondary antibody was detected by incubating 3,3′,5,5′-Tetramethylbenzidine (TMB) in each well for 10 minutes.

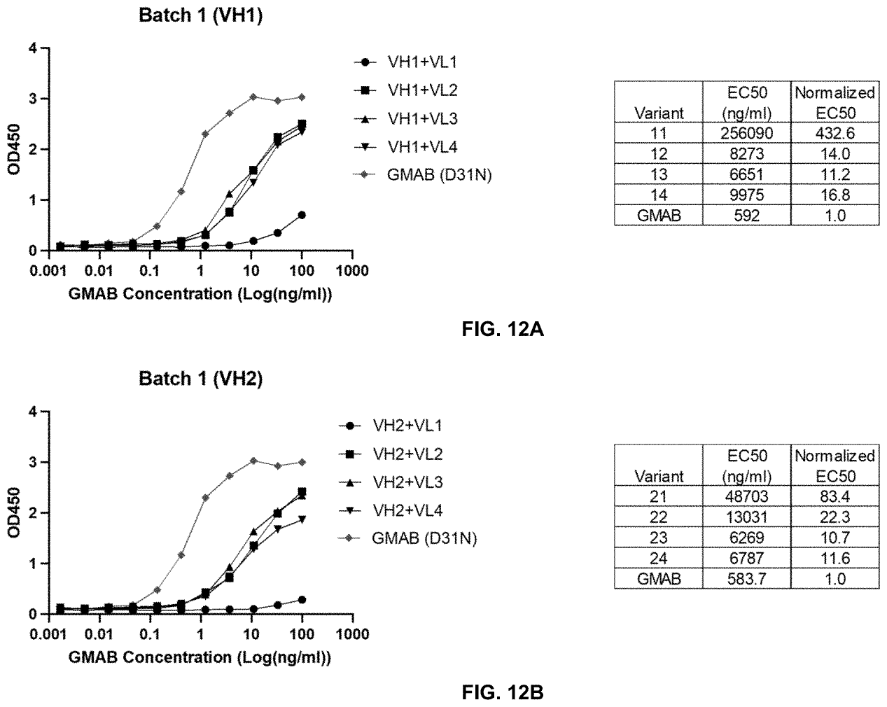

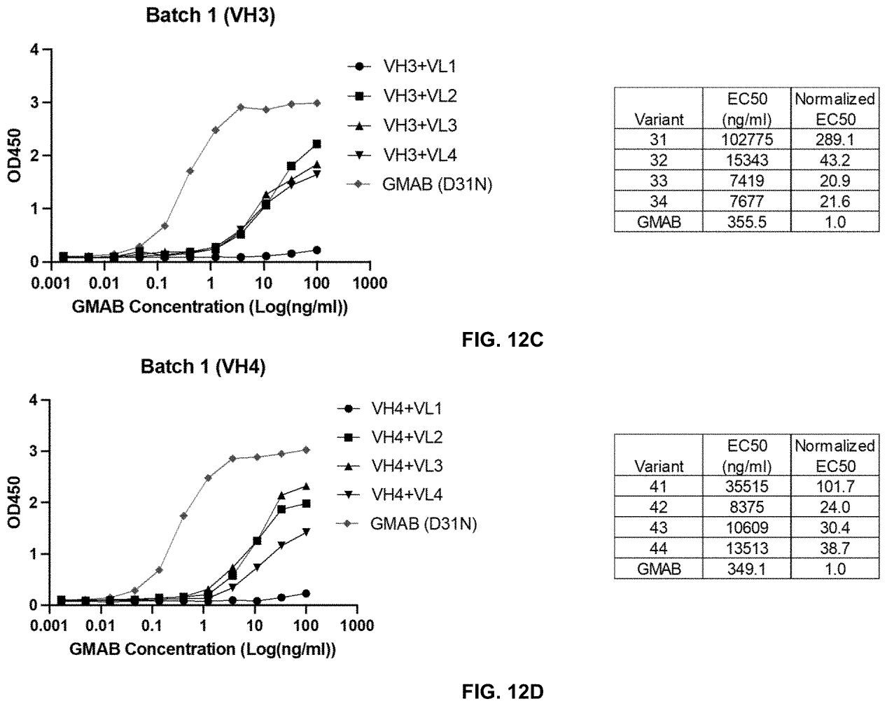

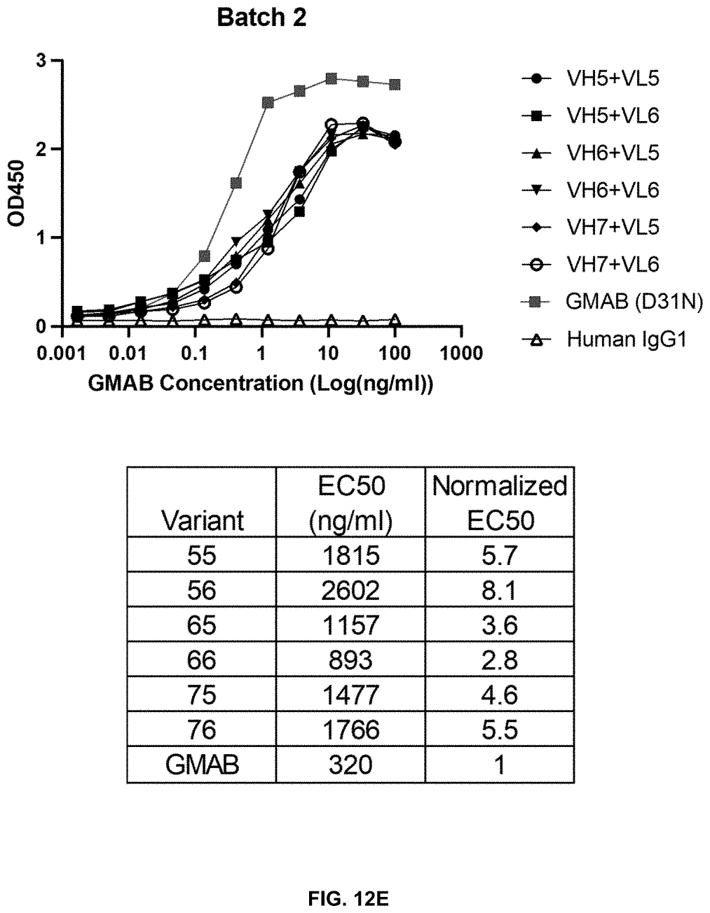

Binding curves for the various binding assays are illustrated in FIGS. 12A, 12B, 12C, 12D, and 12E. EC50 values, representing the concentration of antibody at which half maximum binding of the poly-dT was achieved, were calculated from the curves and then normalized against EC50 values determined for each experiment using a chimeric 3E10 D31N variant having the murine VH and VL sequences.

The results indicate that the lysine at position 72 in the 3E10 light chain significantly contributes to 3E10's nucleic acid binding activity. Mutation of this residue to tyrosine in the VL-h1 variant significantly reduces the affinity of the antibody for DNA binding. Similarly, arginine and lysine at positions 37 and 38, respectively, in the variable heavy chain also appear to contribute to 3E10's nucleic acid binding activity. Mutation of these residues to leucine and arginine, respectively, reduces the affinity of the antibody for DNA binding.

### Example 2: Humanized 3E10 Antibody-Mediated Delivery of RIG-I Ligand Induces Type-I IFN Response in THP-1 Monocytes (Low Affinity Candidates)

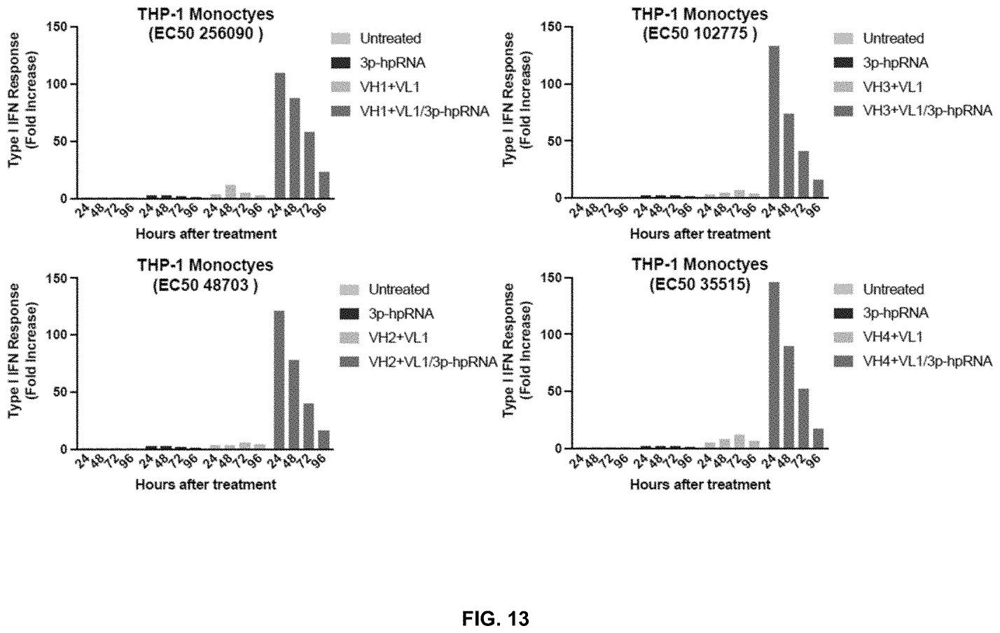

It was investigated whether humanized 3E10 antibody-mediated delivery of a RIG-I stimulating ligand into monocytic cells derived from an acute monocytic leukemia effectively induces a Type-I IFN response characteristic of immunotherapy. Briefly, THP-1 monocytes were seeded into wells and incubated in DMEM supplemented with 20% FBS and 1% P/S at 20,000 cells/well. Cells were then treated with PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in FIG. 13. Sample media was sampled at indicated timepoints and measured for luciferase activity (reporter for type-I IFN). IFN response was monitored for four consecutive days, 24 hours, 48 hours, 72 hours, and 96 hours, as shown in FIG. 13.

As shown in FIG. 13, exposure of the THP-1 monocytes with 3p-hpRNA RIG-I with 3E10 variants 11, 21, 31, or 41 resulted in an average peak increase (˜135-fold) in type-1 IFN response 24 hours after treatment, and steadily decreasing at 48 hours, 72 hours, and 96 hours when compared to the control samples (untreated, 3p-hpRNA alone, and 3E10 variants 11, 21, 31, or 41 alone).

### Example 3: Humanized 3E10 Antibody-Mediated Delivery of RIG-I Ligand Induces Type-I IFN Response in THP-1 Monocytes (Mid-Affinity Candidates)

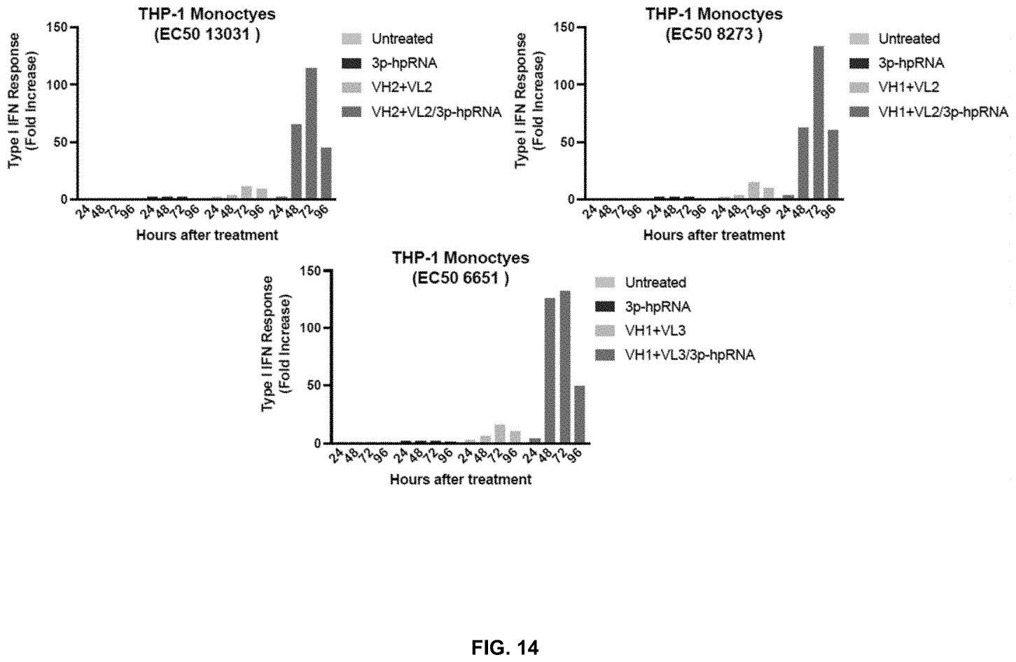

It was investigated whether humanized 3E10 antibody-mediated delivery of a RIG-I stimulating ligand into monocytic cells derived from an acute monocytic leukemia effectively induces a Type-I IFN response characteristic of immunotherapy. Briefly, THP-1 monocytes were seeded into wells and incubated in DMEM supplemented with 20% FBS and 1% P/S at 20,000 cells/well. Cells were then treated with PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in FIG. 14. Sample media was sampled at indicated timepoints and measured for luciferase activity (reporter for type-I IFN). IFN response was monitored for four consecutive days, 24 hours, 48 hours, 72 hours, and 96 hours, as shown in FIG. 14.

As shown in FIG. 14, exposure of the THP-1 monocytes with 3p-hpRNA RIG-I with 3E10 variant 22, 12, or 13 resulted in an average peak increase (˜132-fold) in type-1 IFN response 72 hours after treatment. At 96 hours, the IFN response begins to decrease. The data suggest a mechanism of controlled dissociation of payload, i.e., 3p-hpRNA from humanized 3E10 antibody over time when compared to the control samples (untreated, 3p-hpRNA alone, and 3E10 variants 22, 12, or 13 alone).

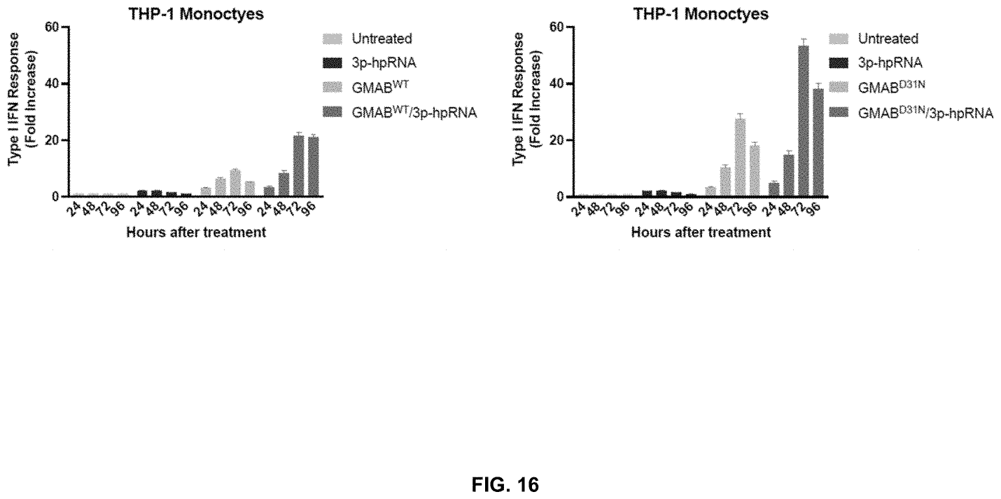

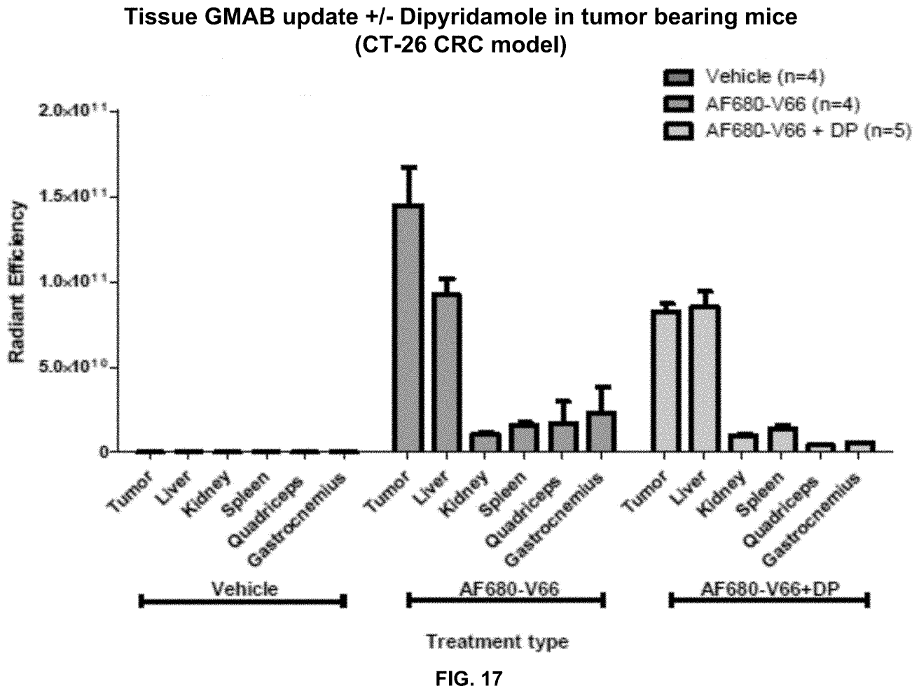

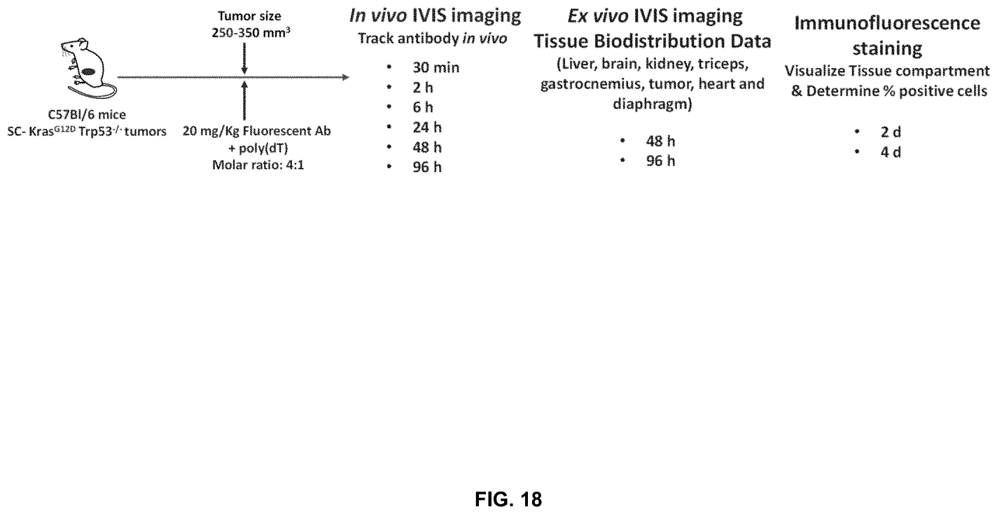

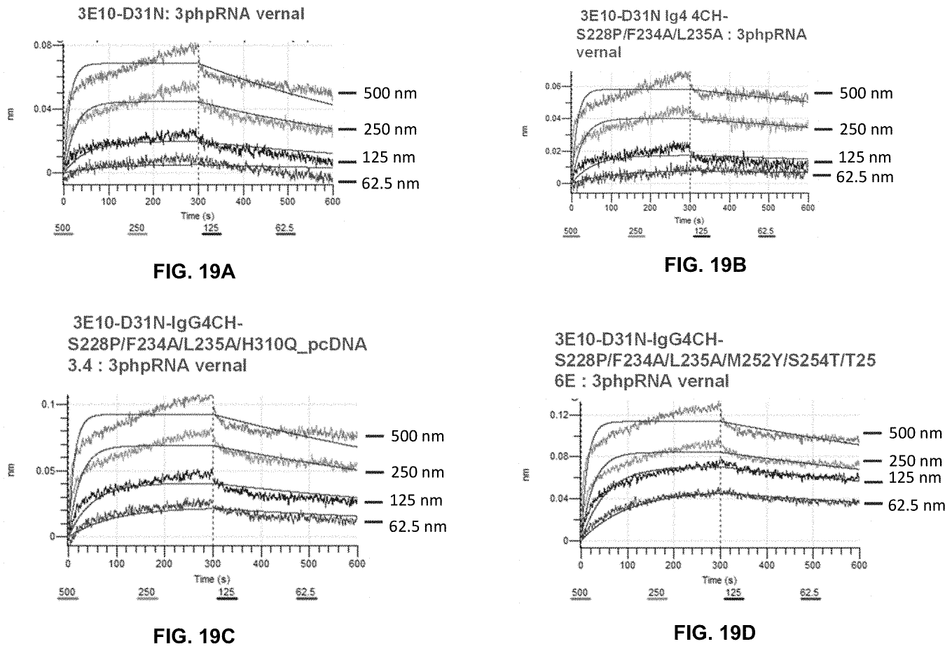

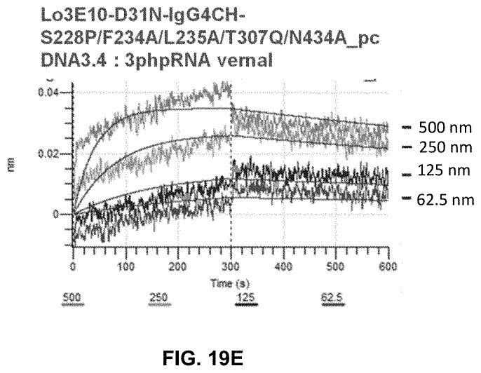

The experiments were repeated using non-humanized 3E10 WT and 3E10 D31N instead of humanized antibody. The results are shown in FIG. 16.

### Example 4: Humanized 3E10 Antibody-Mediated Delivery of RIG-I Ligand Induces Type-I IFN Response in THP-1 Monocytes (High Affinity Candidates)

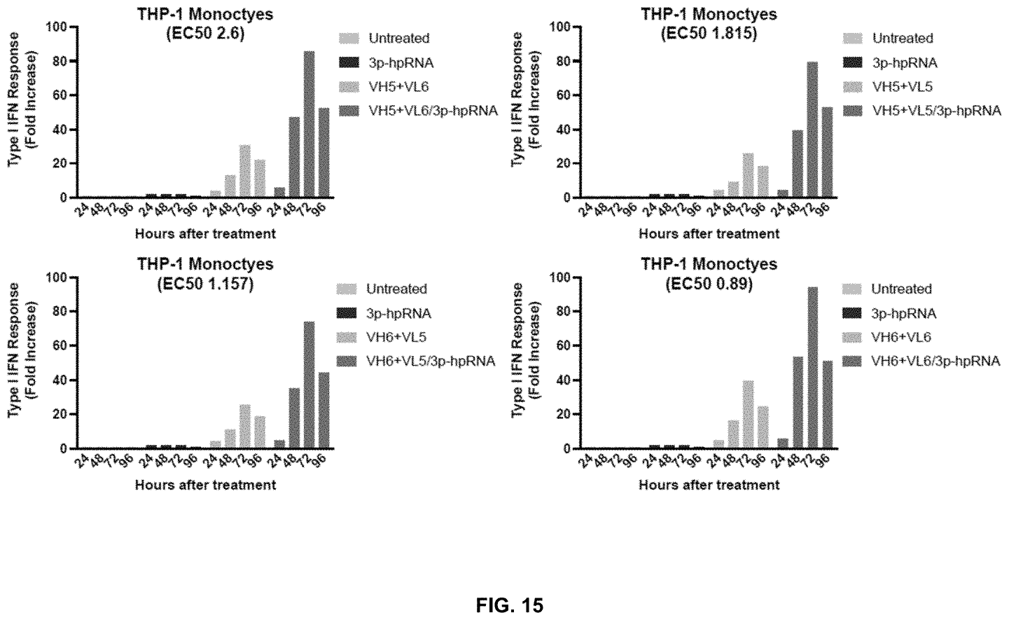

It was investigated whether humanized 3E10 antibody-mediated delivery of a RIG-I stimulating ligand into monocytic cells derived from an acute monocytic leukemia effectively induces a Type-I IFN response characteristic of immunotherapy. Briefly, THP-1 monocytes were seeded into wells and incubated in DMEM supplemented with 20% FBS and 1% P/S at 20,000 cells/well. Cells were then treated with PBS (control), the 3p-hpRNA RIG-I agonist alone (1 ug/well), increasing amounts of humanized 3E10 antibody alone, and humanized 3E10 antibody/3p-hpRNA (1 ug 3p-hpRNA/well), as indicated in FIG. 15. Sample media was sampled at indicated timepoints and measured for luciferase activity (reporter for type-I IFN). IFN response was monitored for four consecutive days, 24 hours, 48 hours, 72 hours, and 96 hours, as shown in FIG. 15.

As shown in FIG. 15, exposure of the THP-1 monocytes with 3p-hpRNA RIG-I with 3E10 variants 55, 56, 65, or 66 resulted in an average peak increase (˜93-fold) in type-1 IFN response increase in type-1 IFN response 72 hours after treatment. At 96 hours, the IFN response begins to decrease. The data suggest a mechanism of controlled dissociation of payload, i.e., 3p-hpRNA from humanized 3E10 antibody over time when compared to the control samples (untreated, 3p-hpRNA alone, and 3E10 variants 55, 56, 65, or 66 alone).

### Example 5: The Binding Kinetics and Affinity Measurements for a Humanized 3E10 Antibody Variant 66 Relative to 3E10-D31N

The binding kinetics of humanized 3E10 construct V66, which included 3E10-VL-h6 (SEQ ID NO:90) and 3E10-VH-h6 (SEQ ID NO:69), to the RIG-I agonist 3p-hpRNA (89 ribonucleotides with a triphosphate at the 5′ end) were analyzed by bio-layer interferometry, using an Octet R8 device (Sartorius). The original 3E10-D31N variant was determined to have a KD of 2.08E-9 M and the V66 variant transiently expressed in HEK293 cells was shown to have an affinity for 3php-RNA of 1.30E-9 M. The V66 variant transiently expressed in CHO cells was determined to have a KD value of 2.60E-9 M. The affinities were elucidated utilizing a 1:1 binding kinetics model. The R2 values (test of fit for actual measurement to predicted binding fit based on the 1:1 binding interaction) were within acceptable parameters (Table 2). These measurements were conducted in the Fc capture format in which the V66 was captured on an AHC2 (anti-human Fc) biosensor then a 4-point concentration titration of 3php-RNA was assayed to measure the binding affinity.

The results indicate that the affinity of the parental antibody (3E10-D31N variant) was preserved during the humanization process to create the V66 variant.

TABLE 2
Binding affinities for 3E10 antibodies

| Sample ID | KD (M) | ka (1/Ms) | kdis (1/s) | R\hat{ }2 |
|---|---|---|---|---|
| 3E10-D31N | 2.08E−09 | 2.29E+05 | 4.76E−04 | 0.9377 |
| V66 (HEK293) | 1.30E−09 | 2.42E+05 | 3.13E−04 | 0.9185 |
| V66 (CHO) | 2.60E−09 | 3.08E+05 | 7.99E−04 | 0.9420 |

### Example 6: Humanized 3E10 Construct (V66) is Tissue Selective and has ENT2-Dependent Bioavailability

Tissue uptake and bioavailability of the humanized 3E10 construct (V66) was investigated in a murine CT-26 model for colorectal cancer. To detect the humanized 3E10 construct (V66) in tissue, the antibody was fluorescently labeled with an Alexa Fluor 680 (AF680). Three experimental murine CT-26 model cohorts were tested and received systemic treatment of a vehicle, AF680-V66, or AF680-V66+dipyridamole (ENT2 inhibitor), respectively. Tissue biodistribution data was be collected by ex vivo IVIS imaging in the tumor, liver, kidney, spleen, quadriceps, and gastrocnemius.

The imaging data from the three cohorts indicated that V66 uptake in the liver, kidney, and spleen is not ENT2-dependent and clearance of V66 in these organs is non-specific, which consistent with antibody clearance of other biologics. Furthermore, it was shown that the tumor tissue has 8-10-fold higher bioavailability compared to muscle tissue V66 uptake.

### Example 7: Biodistribution of 3E10-D31N IgG4 Variants in Syngeneic Pancreatic Ductal Adenocarcinoma (PDAC) Mouse Model

The biodistribution 3E10-D31N IgG4 Variants will be analyzed in a Pancreatic ductal adenocarcinoma (PDAC) Mouse Model. C57Bl/6 mice with SC-KrasG12D Trp53−/− tumors will be injected with 20 mg/Kg a fluorescent 3E10 D3N IgG4 variant antibody (see Table 3)+poly(dT) at ratio: 4:1 when tumor size reaches 250-350 nm³. In vivo IVIS imaging will track 3E10 D31N Ig4 variant antibodies at 30 minutes, 2 hours, 6 hours, 24 hours, 48 hours, and 96 hours. Tissue biodistribution data will be collected by ex vivo IVIS imaging in the liver, brain, kidney, triceps, gastrocnemius, tumor, heart, and diaphragm. Immunofluorescence staining will be used to visualize tissue compartment and determine % positive cells.

TABLE 3
3E10 D31N Ig4 variants and effector functions

| 3E10 D31N Ig4 variants | Effector Functions |
|---|---|
| 3E10-D31N | Prevent Fab-arm exchange, |
| | Reduced binding to FcγRI, |
| | FcγRII, FcγRIII, and C1q |
| 3E10-D31N-IgG4CH-S228P- | Improved Half-life |
| F234A-L235A-T307Q-N434A | (Modest extension) |
| 3E10-D31N-IgG4CH-S228P-F234A- | Improved Half-life |
| L235A-M252Y-S254T-T256E | (Significant extension) |
| 3E10-D31N-IgG4CH-S228P- | Faster Clearance |
| F234A-L235A-H310Q | |

### Example 8: Affinity Analysis of K3E10-D31N IgG4 Variants

The binding kinetics of 3E10-D31N and four 3E10-D31 IgG4 variants to the RIG-I agonist 3p-hpRNA (89 ribonucleotides with a triphosphate at the 5′ end) were analyzed by bio-layer interferometry, using an Octet R8 device (Sartorius). The KD (M) measurement of 3E10-D31N was 1.052E-08 (Table 4).

TABLE 4
Binding Kinetics of 3E10-D31N and 3E10-D31N IgG4 Variants

| FIG. | Clone ID | KD (M) | Ka (1/Ms) | Kdis (1/s) | R\hat{ }2 |
|---|---|---|---|---|---|
| 19A | 3E10-D31N | 1.052E−08 | 1.50E05 | 1.581E−03 | 0.9612 |
| 19B | 3E10-D31N IgG4 4CH-S228P/F234A/ | 3.326E−09 | 1.43E05 | 4.767E−04 | 0.9665 |
| | L235A | | | | |
| 19C | 3E10-D31N-IgG4CH-S228P/F234A/ | 1.268E−08 | 4.79E04 | 6.070E−04 | 0.9069 |
| | L235A/T307Q/N434A_pcDNA3.4 | | | | |
| 19D | 3E10-D31N-IgG4CH-S228P/F234A/ | 5.00E−09 | 1.45E+05 | 7.28E−04 | 0.9633 |
| | L235A/M252Y/S254T/T256E | | | | |
| 19E | 3E10-D31N-IgG4CH-S228P/F234A/ | 6.58E−09 | 1.57E+05 | 1.03E−03 | 0.9584 |
| | L235A/H310Q_pcDNA3.4 | | | | |

The affinities were elucidated utilizing a 1:1 binding kinetics model. The R2 values (test of fit for actual measurement to predicted binding fit based on the 1:1 binding interaction) were within acceptable parameters (Table 3). These measurements were conducted in the Fc capture format in which the each of the 3E10-D31N IgG4 variants were captured on an AHC2 (anti-human Fc) biosensor then a 4-point concentration titrations (62.5 nm, 125 nm, 250 nm, and 500 nm of 3php-RNA were assayed to measure the binding affinity.

The results indicate that the affinity of a 3E10-D31N antibody with an IgG4 Fc variant is preserved.

### Example 9: Functional Delivery of GFP mRNA in KPC Syngeneic Tumor Model

The equivalence or superiority of the humanized 3E10 antibody construct (V66) was compared to chimeric 3E10 D31N by analyzing nucleic acid payload functional delivery models in vivo.

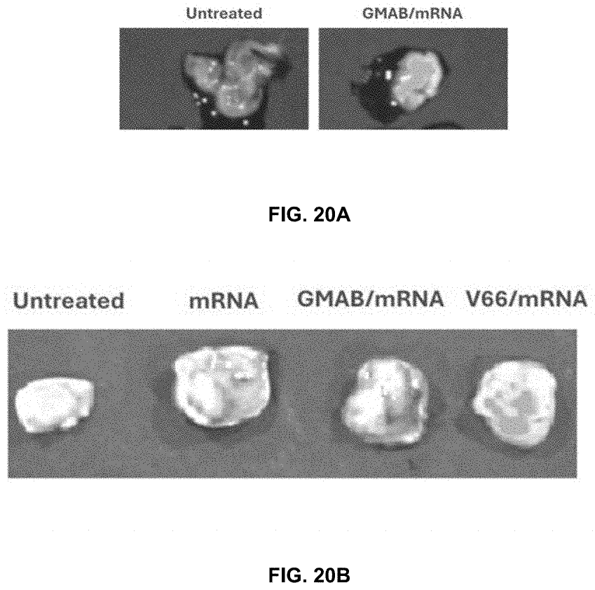

The first comparability study analyzed systemic targeted functional delivery of GFP mRNA payload to subcutaneous tumors in mice. Previous studies showed targeted functional delivery using 3E10 D31N in an MDA-MB-231 model (FIG. 20A). The current study used a subcutaneous KPC tumors. Each murine cohort was single dosed at a 10:1 weight:weight ratio (400:40 ug). Tumor/normal tissue was analyzed for GFP expression by IVIS after 24 hours.

As is shown in FIG. 20B, the humanized 3E10 antibody construct (V66), not the chimeric 3E10 D31N, delivered the GFP-mRNA to KPC tumors.

### Example 10: Tumor and Normal Tissue Expression for Targeted Functional Delivery of a GFP mRNA Payload

Tumor versus normal tissue (kidney, heart, liver, and skeletal muscle) expression was analyzed for a GFP mRNA payload using chimeric 3E10 D31N and the humanized 3E10 antibodies (V66), respectively. Dosing was 2 mg/kg for mRNA and 20 mg/kg for antibody.

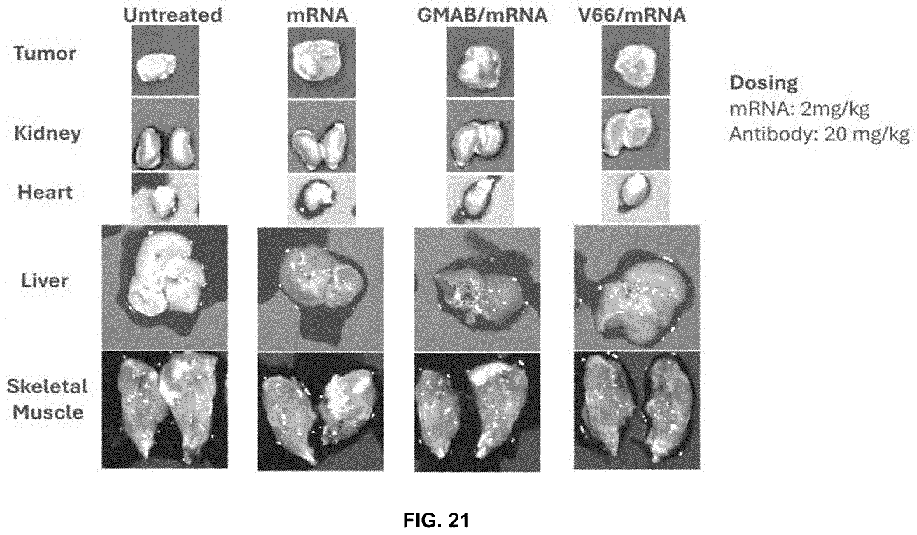

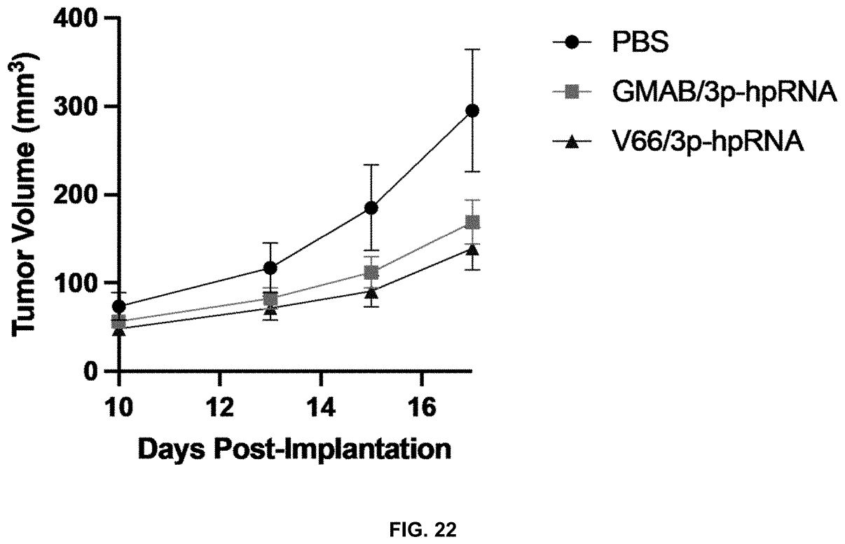

As shown in FIG. 21, detection of GFP was observed in the tumor and kidney of mice administered V66-GFP mRNA. It is postulated that functional GFP kidney expression may be due to targeted ENT2 delivery. Furthermore, absence of liver GFP expression may suggest protein catabolism, and not ENT2 targeted delivery.

### Example 11: Comparability of Chimeric 3E10 D31N and the Humanized 3E10 Antibodies (V66) in B16 Tumor Model

Tumor stasis was previously shown using a 4:1 molar ratio of chimeric 3E10 D31N:3p-hpRNA, using the antibody at a concentration of 20 mg/kg, and administering 4 doses on a 3 to 4-day interval. The current study used the same conditions for chimeric 3E10 D31N and the humanized 3E10 antibodies (V66), and a treatment control (PBS). Dose 1 was administered at day 10, dose 2 was administered on day 13, and dose 3 was administered on day 18. The study lasted for 30-35 days.

As is shown in FIG. 21, V66/3p-hpRNA demonstrated a comparable anti-tumor response to chimeric 3E10-D31N/3p-hpRNA after 2 doses in B16 model, reaching statistical significance on Day 17.

### Example 12: Pharmacokinetic (pK) Evaluation of 3E10-D31N Monoclonal Antibody (V66) in C57BL/6 Mice

Single dose and dose escalation harmacokinetic (pK) studies for the 3E10-D31N monoclonal antibody (V66) were performed in C57BL/6 mice. The antibody (V66) was administered to 3 groups of C57BL/6 mice (36 mice/group) at doses of 25 mg/kg (Group 1), 50 mg/kg (Group 2), and 100 mg/kg (Group 3), respectively. Antibody (V66) doses was administered to three mice per time point per group for a total of 12 timepoints recorded for each group. The tissues analyzed post-perfusion were liver, kidney, heart, brain, gastrocnemius, triceps, and skeletal muscle (deltoid). Serum and meso scale (MSD) immunoassays were used for detection of antibody (V66) in serum and selected tissues.

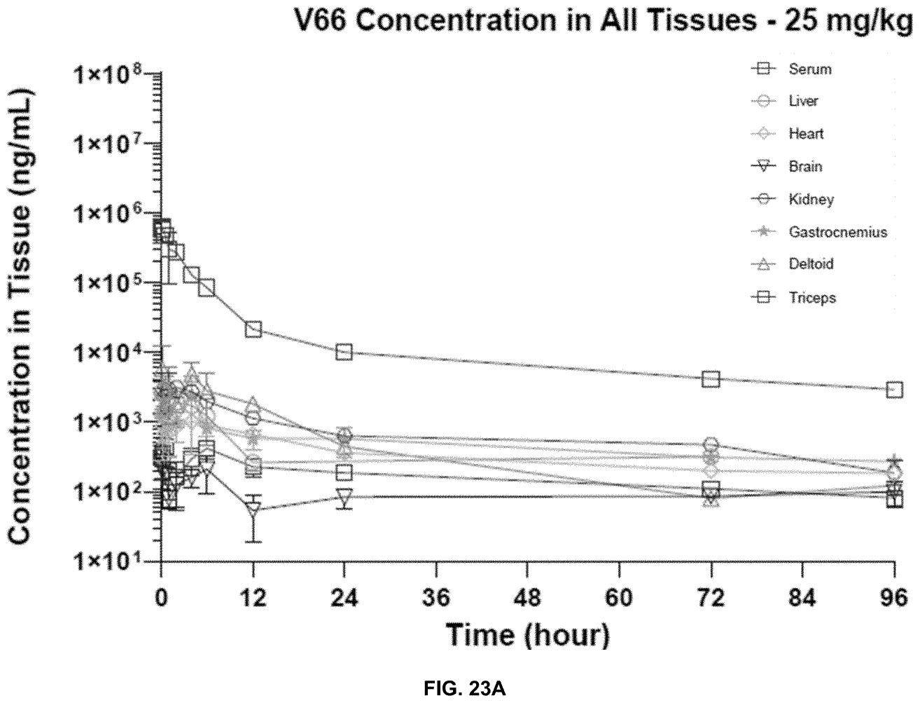

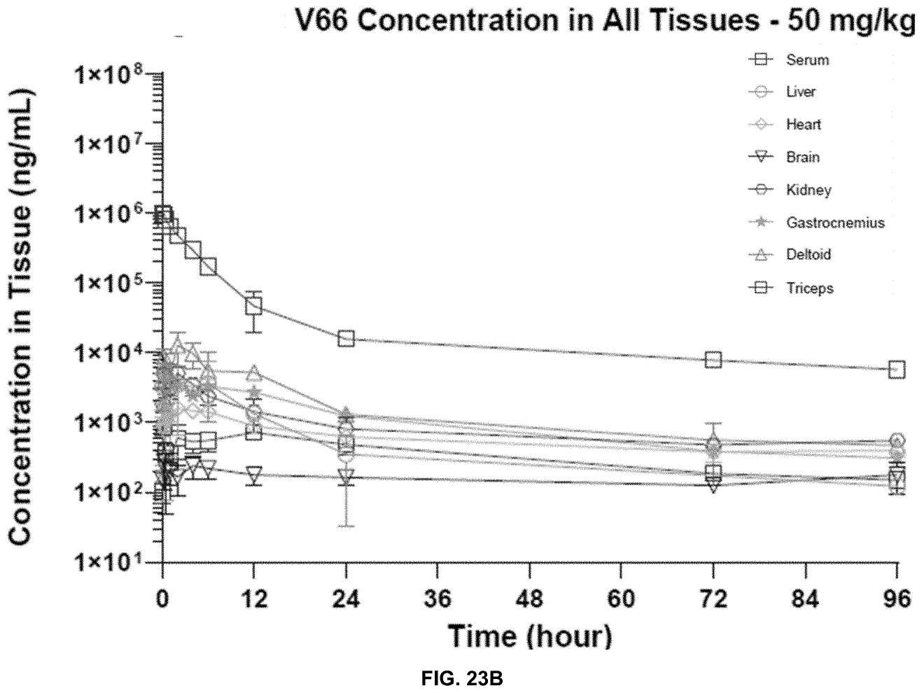

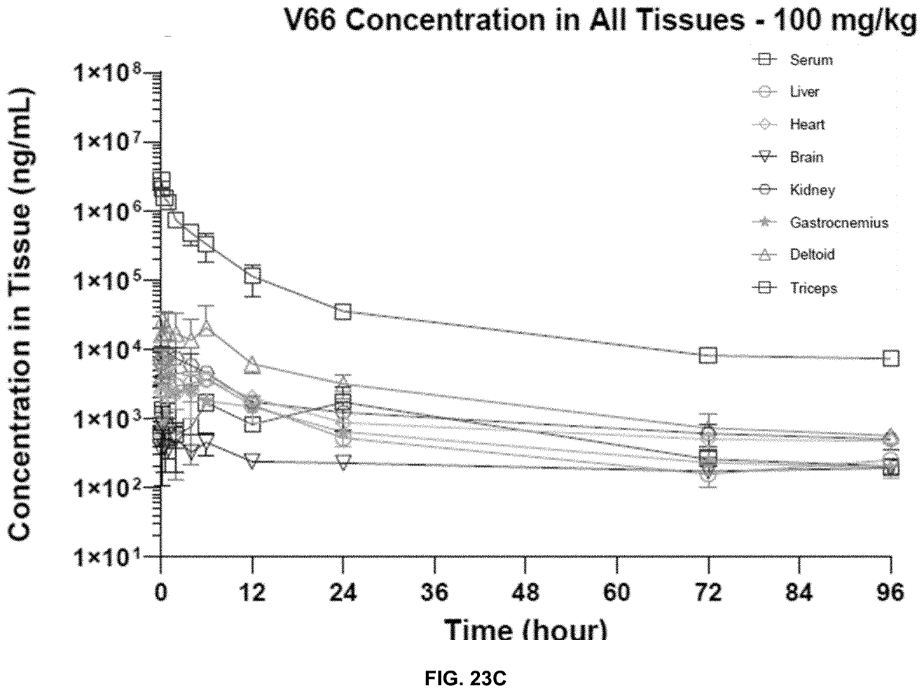

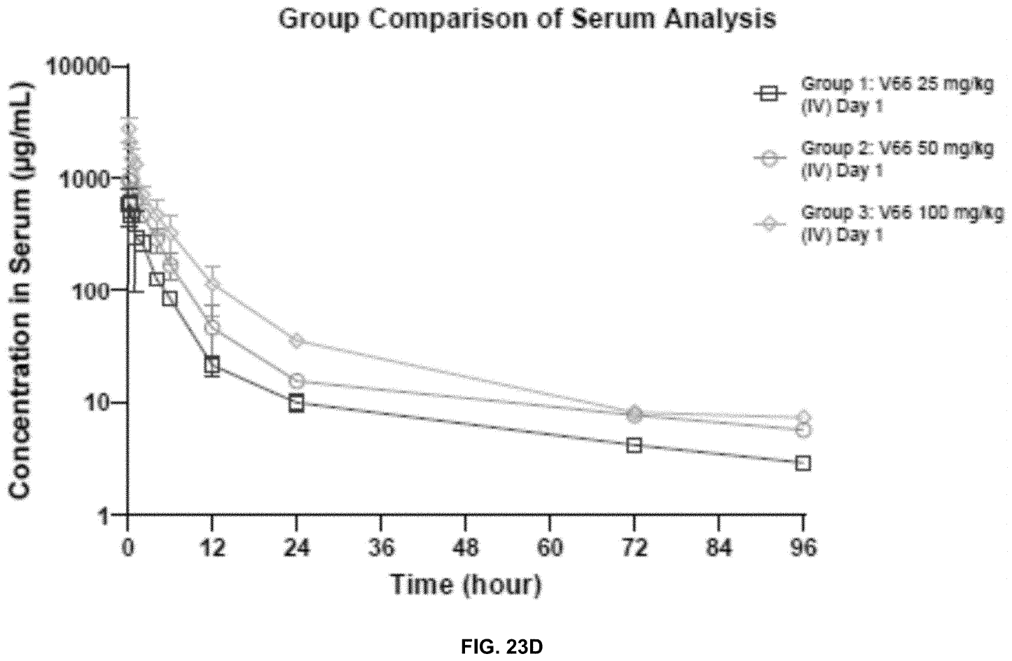

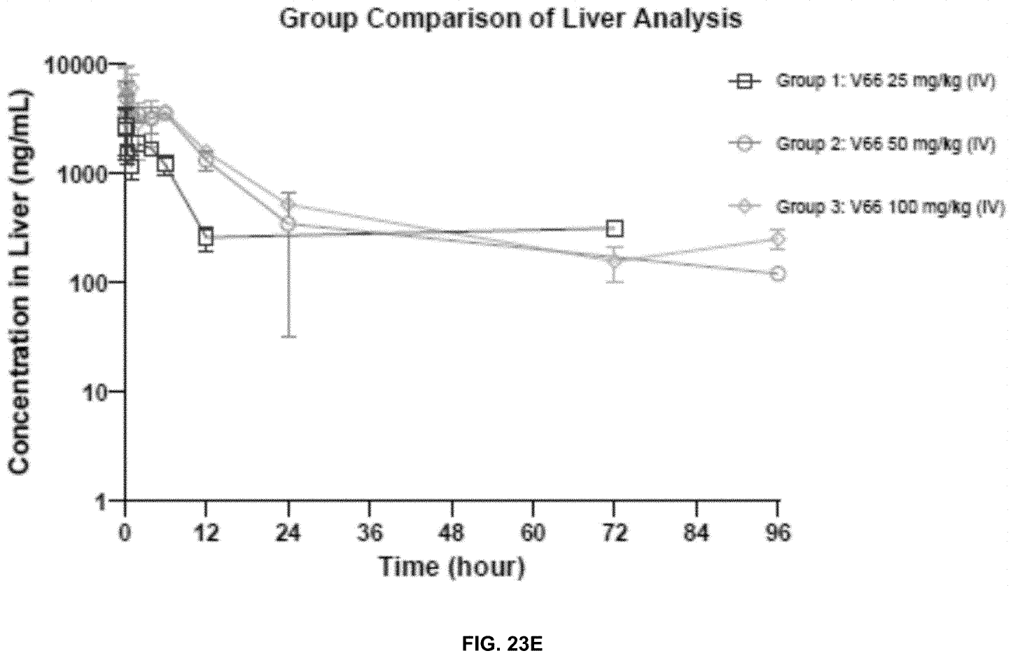

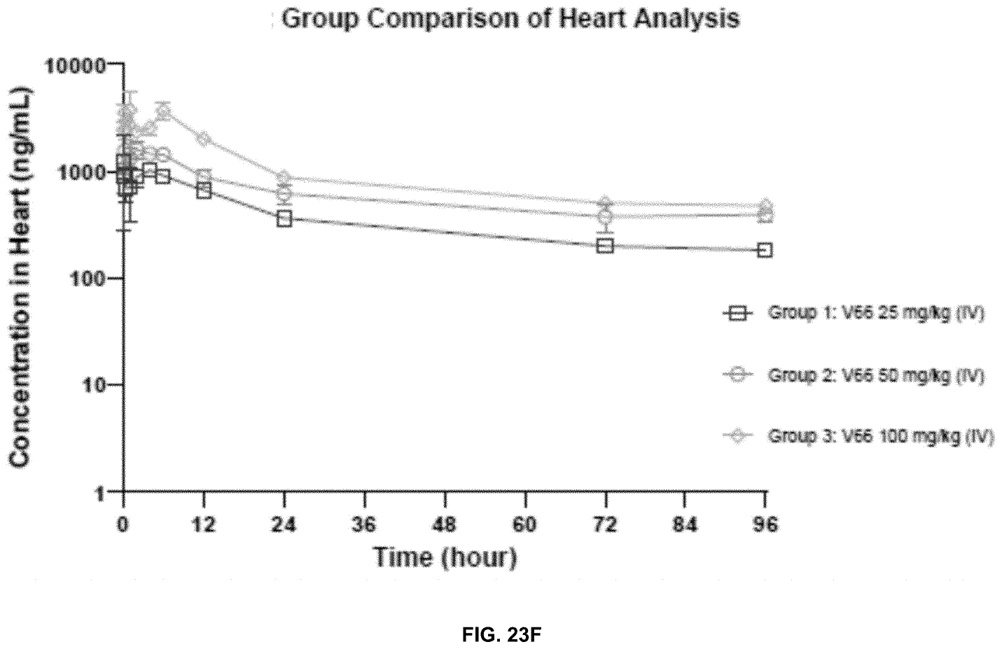

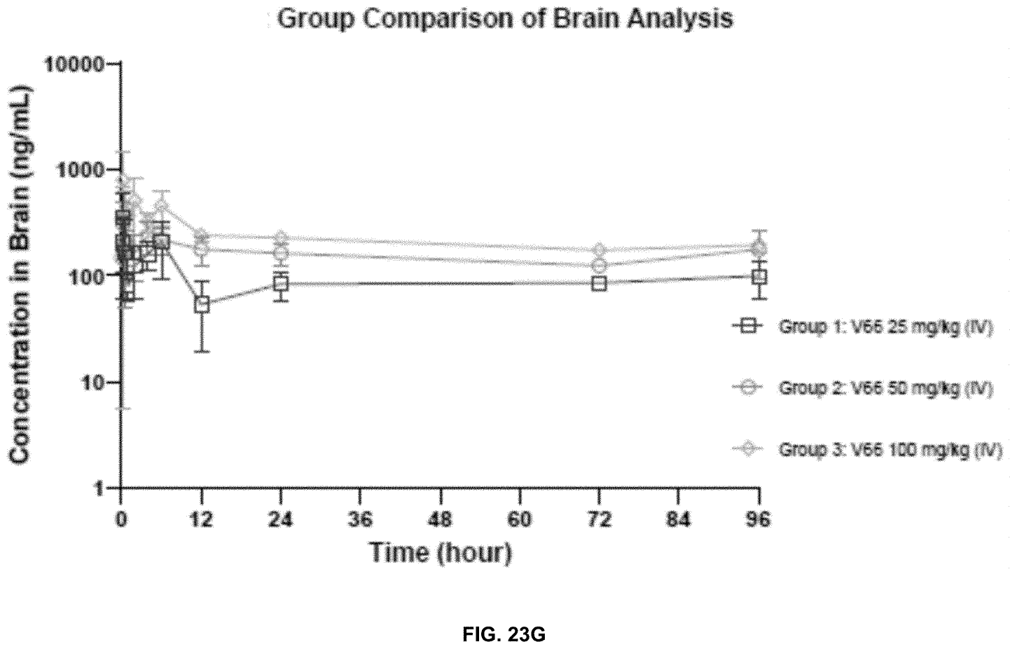

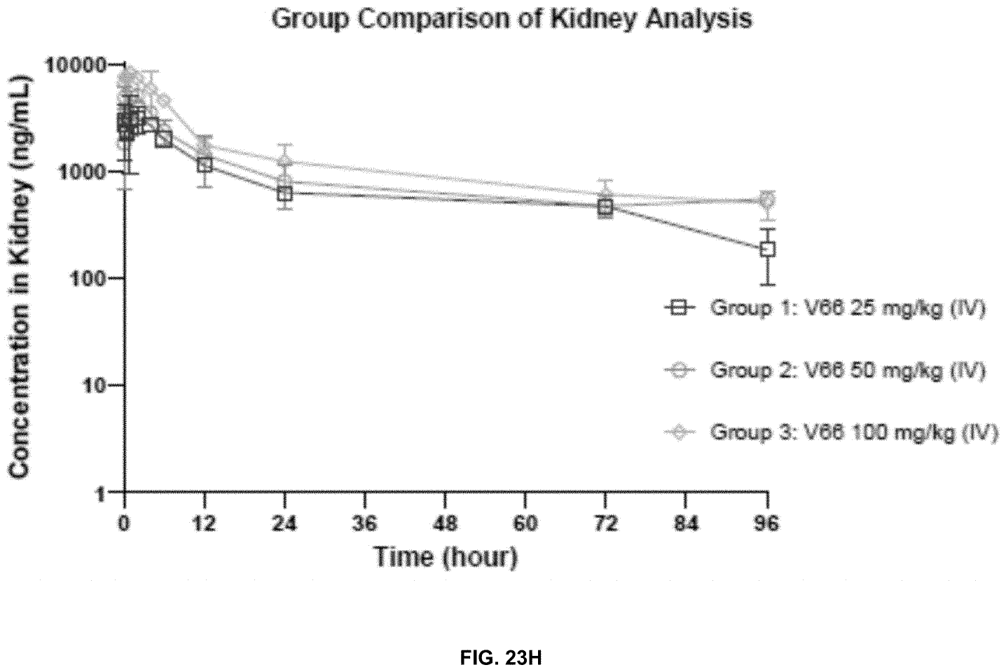

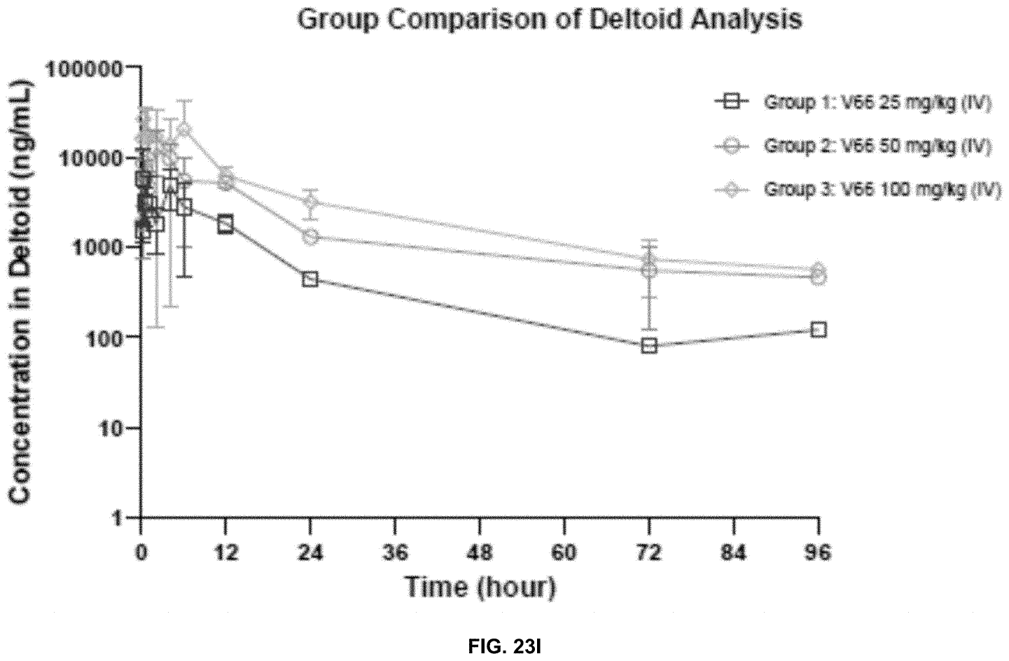

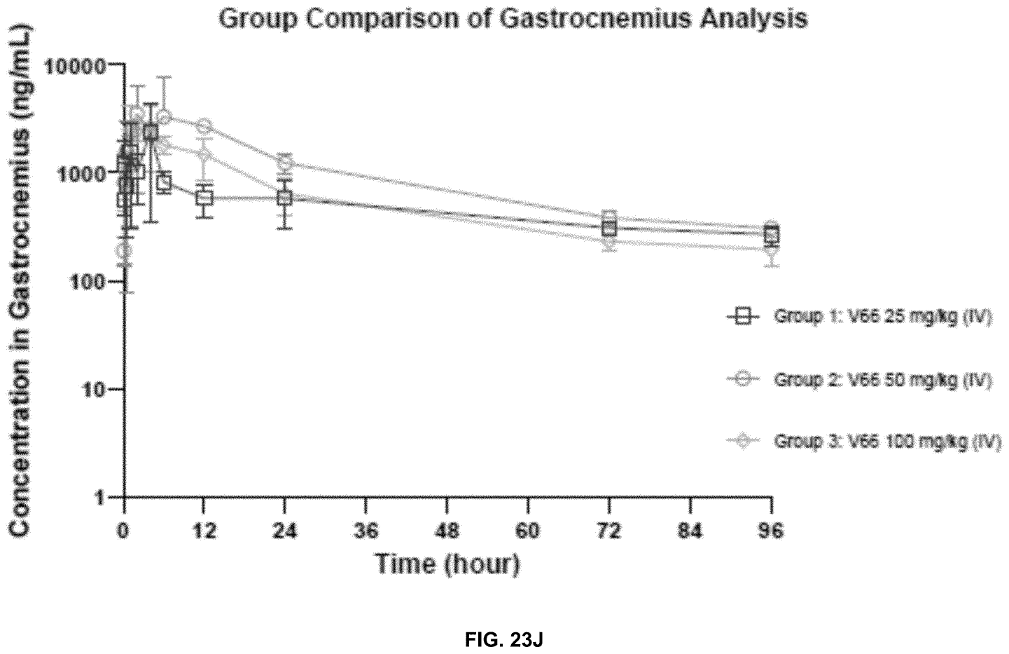

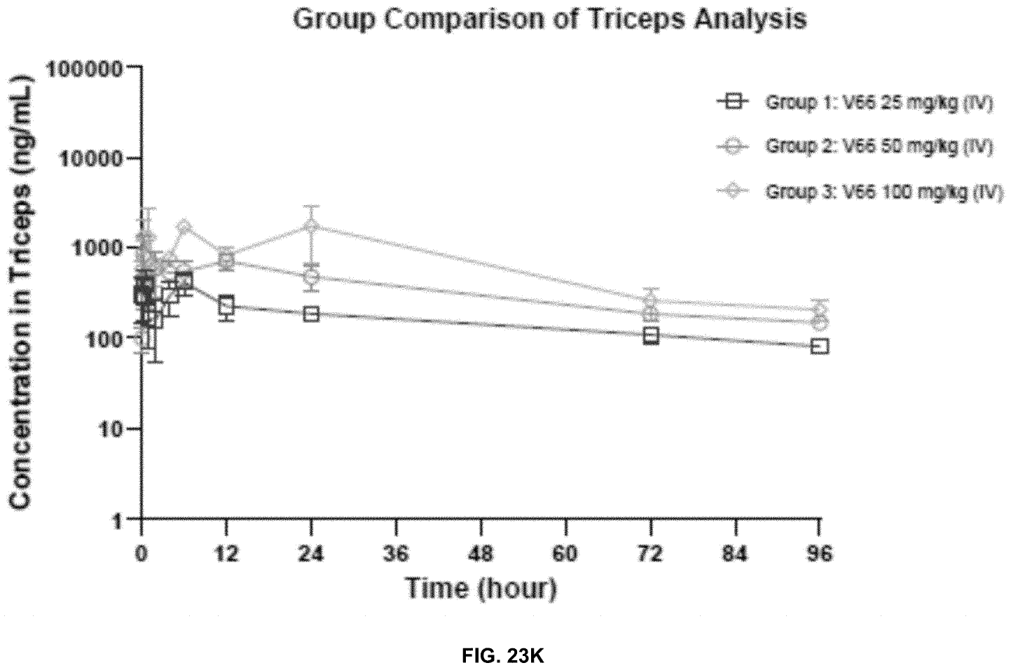

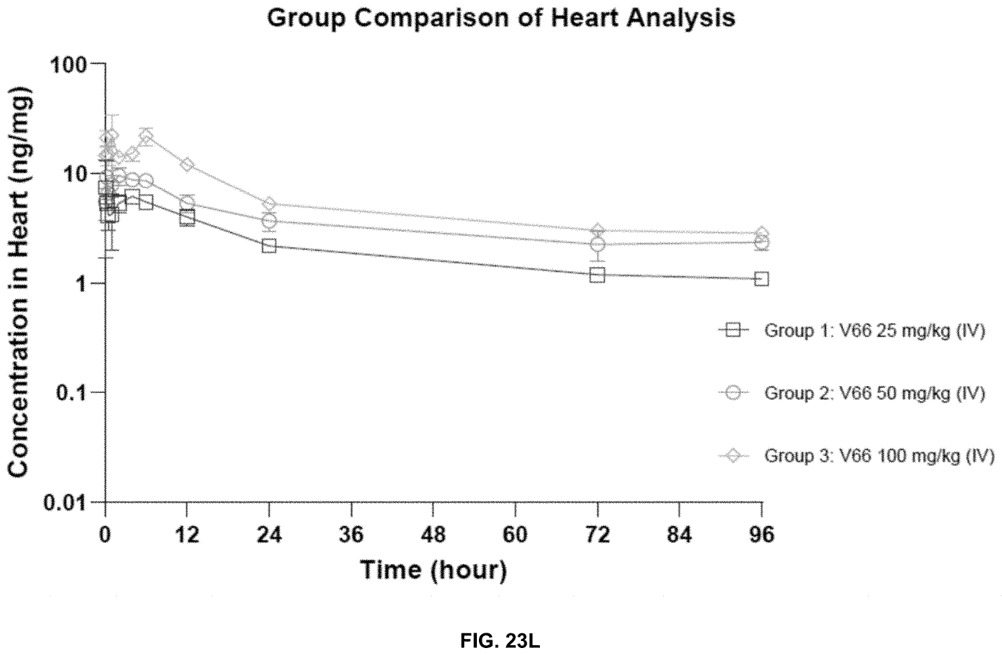

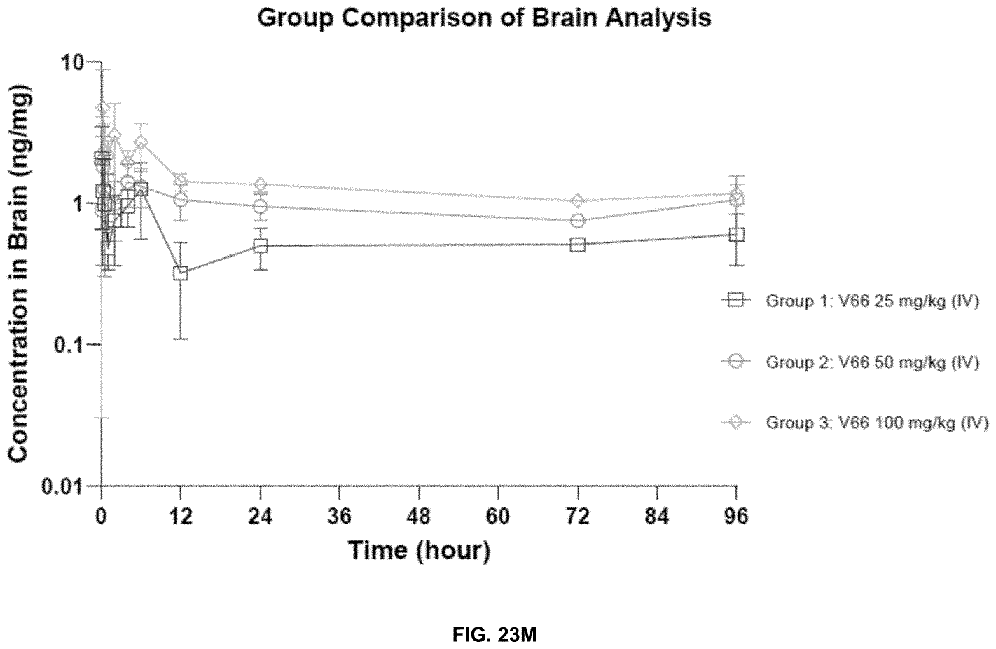

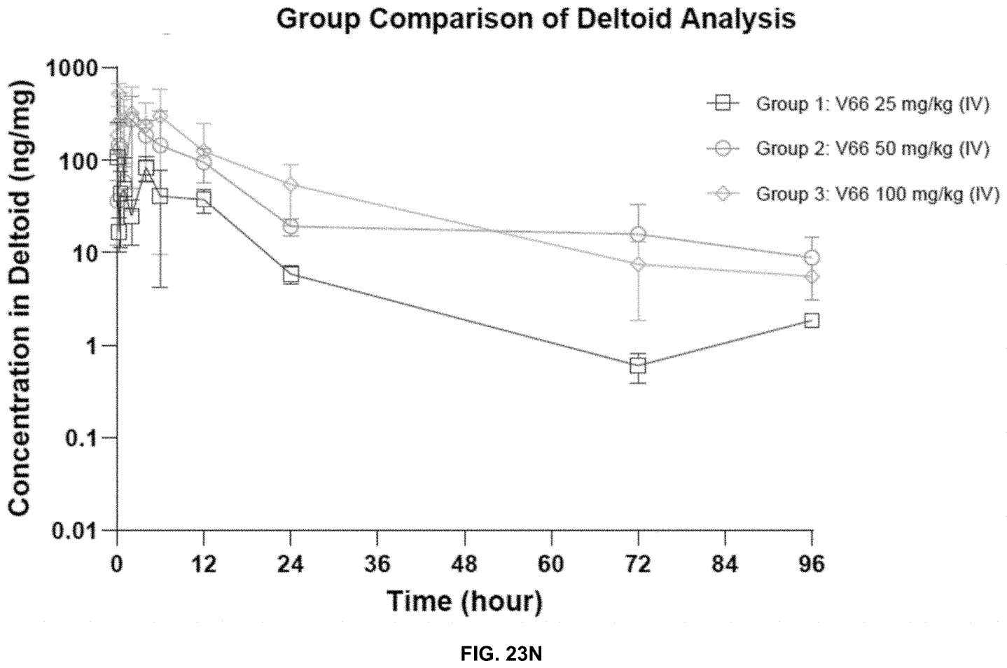

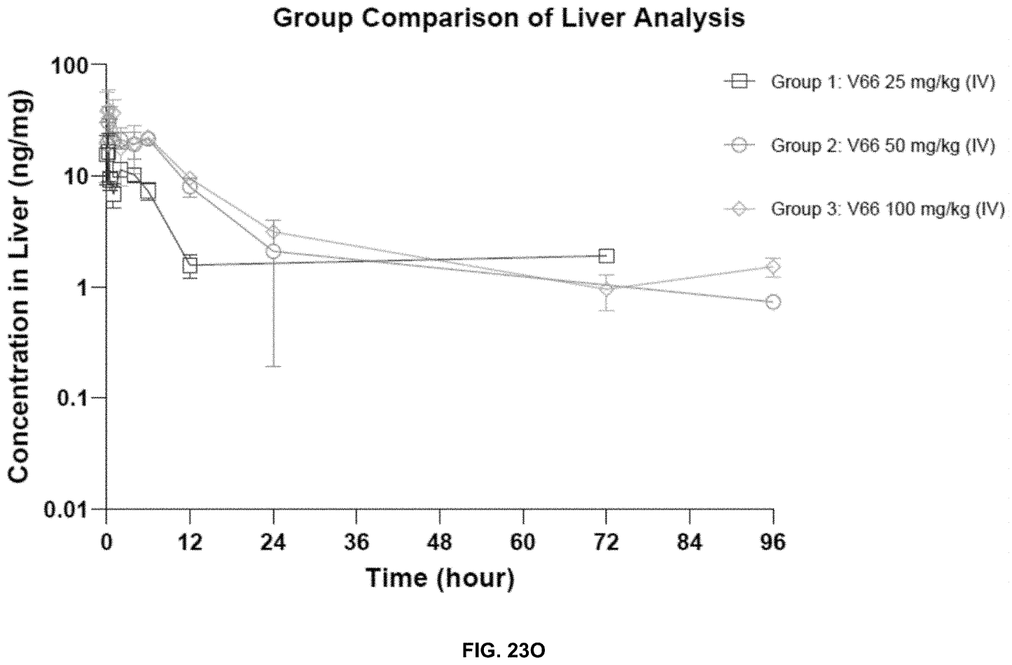

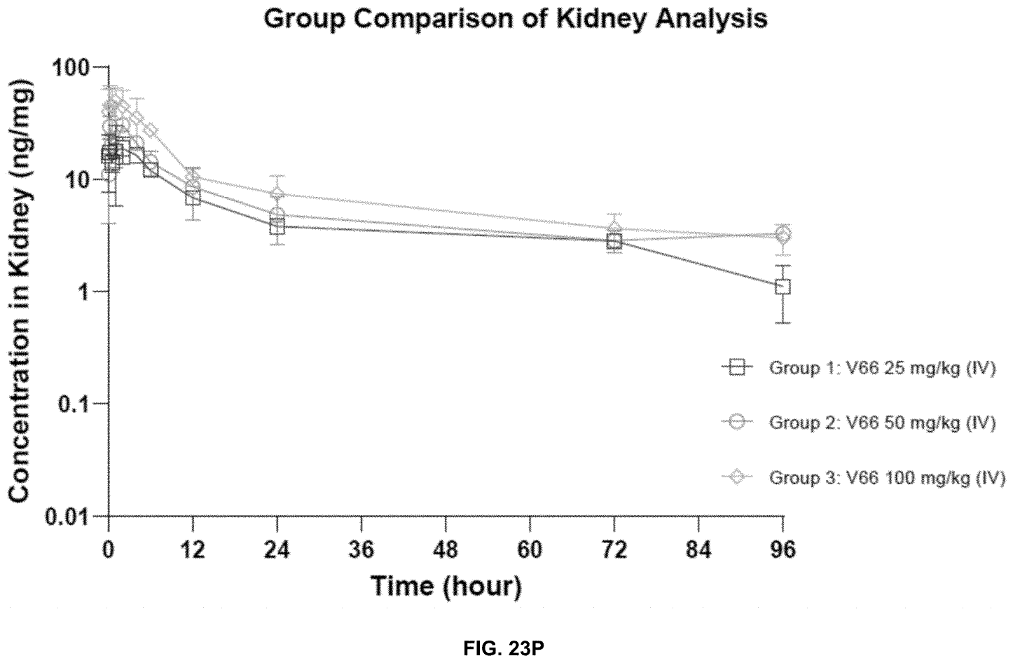

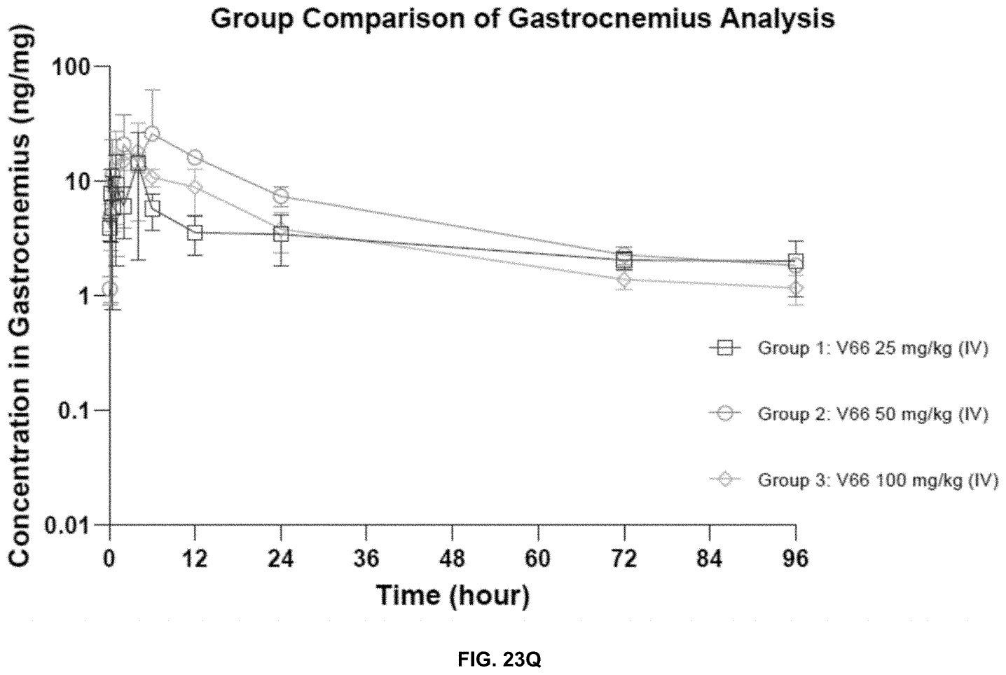

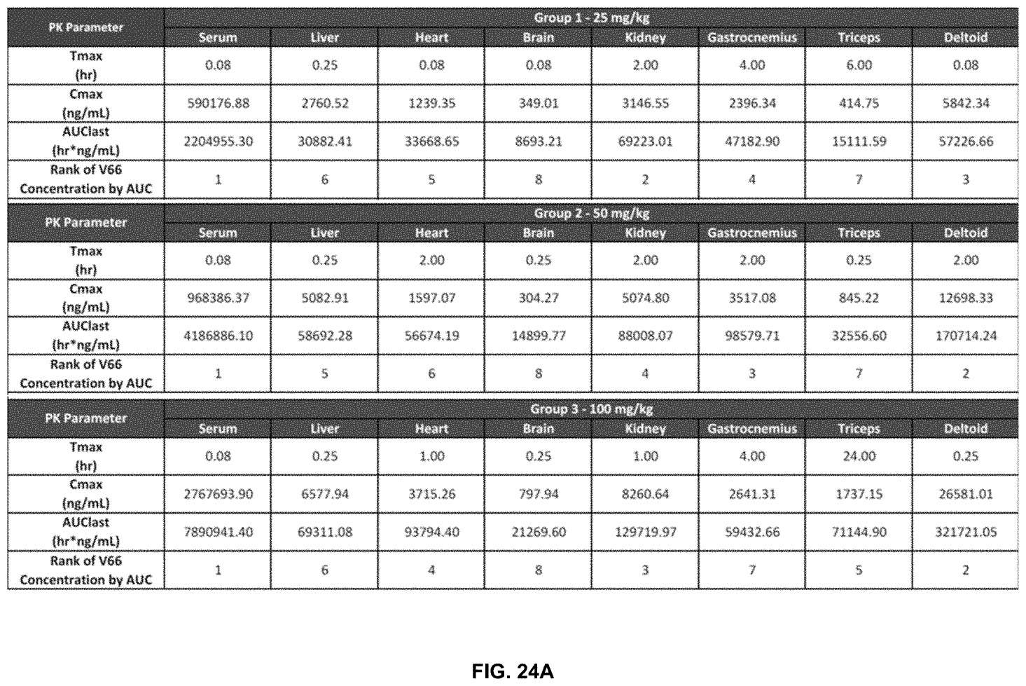

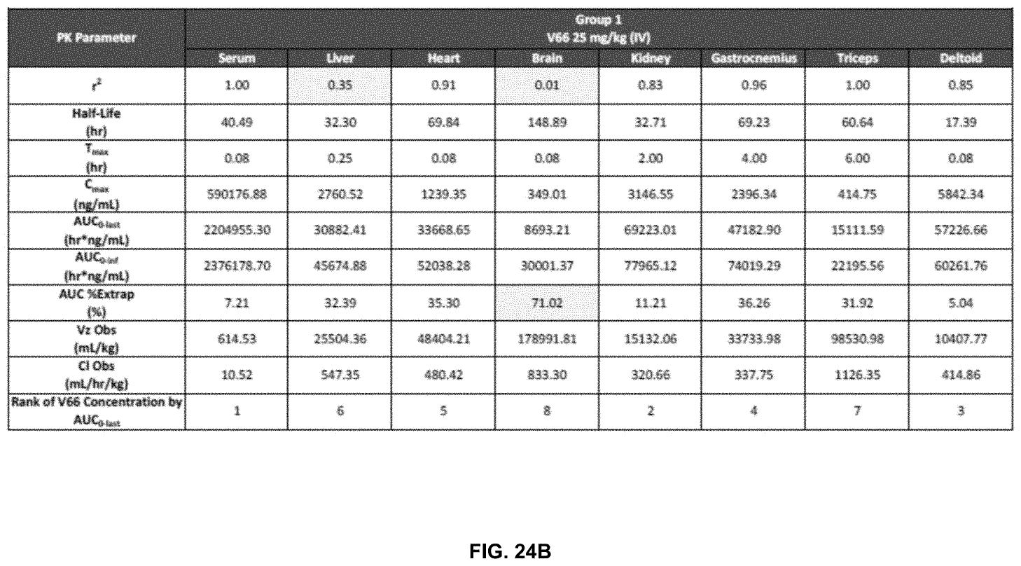

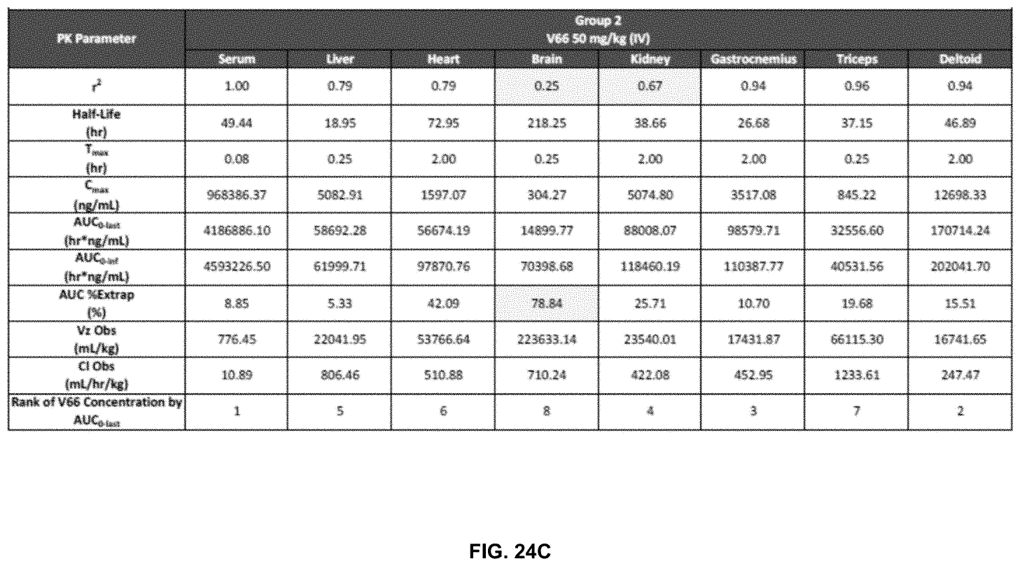

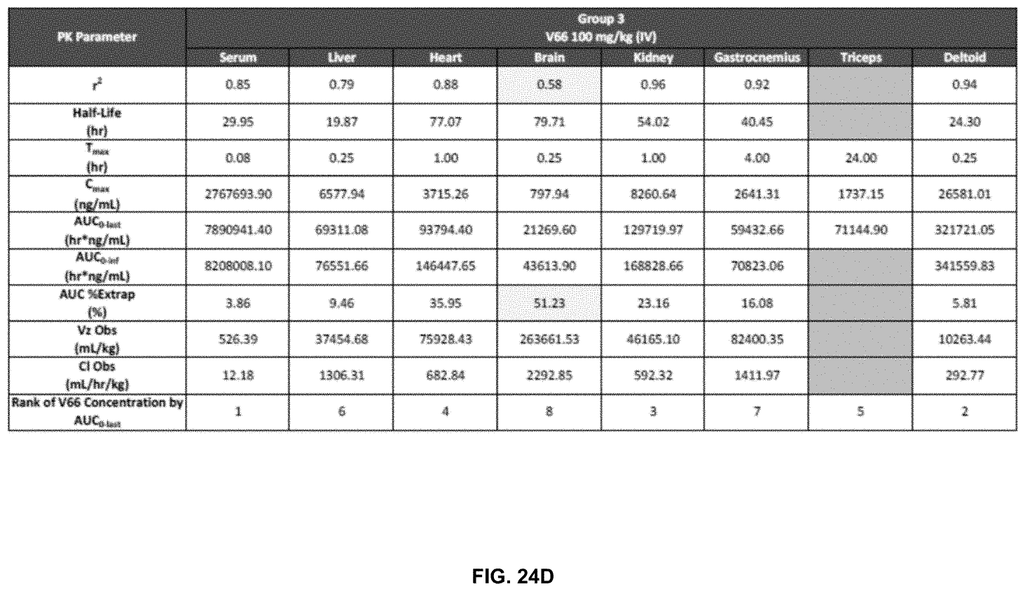

FIGS. 23 and 24 show pharmacokinetic analysis and summary statistics for each of the three groups, the circulating serum half-life was calculated to be 40-50 hours, approximately 1.7-2.1 days in the C57BL/6 mice. Furthermore, there was robust distribution Robust distribution to skeletal muscle tissue, particularly deltoid, but also gastrocnemius and triceps, confirming targeting of ENT2-positive skeletal muscle by 3E10-D31N monoclonal antibody (V66). 3E10-D31N monoclonal antibody (V66) accumulation was also observed in heart tissue. Notably, there was accumulation and retention of 3E10-D31N monoclonal antibody (V66) in brain, albeit lower than other tissues evaluated. Uptake and biodistribution of (V66) was also detected in the liver and kidney

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A humanized 3E10 antibody or antigen binding fragment thereof comprising a light chain variable domain (3E10-VL) and a heavy chain variable domain (3E10-VH), wherein:
the 3E10-VL comprises an amino acid sequence that is at least 97% identical to an amino acid sequence selected from the group consisting of 3E10-VL-h1 (SEQ ID NO:85), 3E10-VL-h2 (SEQ ID NO:86), 3E10-VL-h3 (SEQ ID NO:87), 3E10-VL-h4 (SEQ ID NO:88), 3E10-VL-h5 (SEQ ID NO:89), and 3E10-VL-h6 (SEQ ID NO:90), and
the 3E10-VH comprises an amino acid sequence that is at least 95% identical to an amino acid sequence selected from the group consisting of 3E10-VH-h1 (SEQ ID NO:64), 3E10-VH-h2 (SEQ ID NO:65), 3E10-VH-h3 (SEQ ID NO:66), 3E10-VH-h4 (SEQ ID NO:67), 3E10-VH-h5 (SEQ ID NO:68), 3E10-VH-h6 (SEQ ID NO:69), and 3E10-VH-h7 (SEQ ID NO:70).

2. The humanized 3E10 antibody or antigen binding fragment thereof of claim 1, wherein the humanized 3E10 antibody or antigen binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:91), 3E10-LC-h2m (SEQ ID NO:92), 3E10-LC-h3m (SEQ ID NO:93), 3E10-LC-h4m (SEQ ID NO:94), 3E10-LC-h5m (SEQ ID NO:95), and 3E10-LC-h6m (SEQ ID NO:96), and
the 3E10-HC comprises an amino acid sequence that is at least 95% identical to an amino acid sequence selected from the group consisting of 3E10-HC-h1m (SEQ ID NO:71), 3E10-HC-h2m (SEQ ID NO:72), 3E10-HC-h3m (SEQ ID NO:73), 3E10-HC-h4m (SEQ ID NO:74), 3E10-HC-h5m (SEQ ID NO:75), 3E10-HC-h6m (SEQ ID NO:76), and 3E10-HC-h7m (SEQ ID NO:77).

3. The humanized 3E10 antibody or antigen binding fragment thereof of claim 1, wherein the humanized 3E10 antibody or antigen binding fragment thereof comprises a light chain (3E10-LC) and a heavy chain (3E10-HC), wherein:
the 3E10-LC comprises an amino acid sequence that is at least 97% identical to an amino acid sequence selected from the group consisting of 3E10-LC-h1m (SEQ ID NO:97), 3E10-LC-h2 (SEQ ID NO:98), 3E10-LC-h3 (SEQ ID NO:99), 3E10-LC-h4 (SEQ ID NO:100), 3E10-LC-h5 (SEQ ID NO:101), and 3E10-LC-h6 (SEQ ID NO:102), and
the 3E10-HC comprises an amino acid sequence that is at least 95% identical to an amino acid sequence selected from the group consisting of 3E10-HC-h1 (SEQ ID NO:78), 3E10-HC-h2 (SEQ ID NO:79), 3E10-HC-h3 (SEQ ID NO:80), 3E10-HC-h4 (SEQ ID NO:81), 3E10-HC-h5 (SEQ ID NO:82), 3E10-HC-h6 (SEQ ID NO:83), and 3E10-HC-h7 (SEQ ID NO:84).

4. The humanized 3E10 antibody or antigen binding fragment thereof according to claim 1, wherein the 3E10-VL comprises an amino acid sequence that is at least 97%, 98%, 99%, or 100% identical to 3E10-VL-h1 (SEQ ID NO:85).

5. The humanized 3E10 antibody or antigen binding fragment thereof according to claim 1, wherein the 3E10-VL comprises an amino acid sequence that is at least 97%, 98%, 99%, or 100% identical to 3E10-VL-h2 (SEQ ID NO:86).

6. The humanized 3E10 antibody or antigen binding fragment thereof according to claim 1, wherein the 3E10-VL comprises an amino acid sequence that is at least 97%, 98%, 99%, or 100% identical to 3E10-VL-h3 (SEQ ID NO:87).

7. The humanized 3E10 antibody or antigen binding fragment thereof according to claim 1, wherein the 3E10-VL comprises an amino acid sequence that is at least 97%, 98%, 99%, or 100% identical to 3E10-VL-h4 (SEQ ID NO:88).

8. The humanized 3E10 antibody or antigen binding fragment thereof according to claim 1, wherein the 3E10-VL comprises an amino acid sequence that is at least 97%, 98%, 99%, or 100% identical to 3E10-VL-h5 (SEQ ID NO:89).

9. The humanized 3E10 antibody or antigen binding fragment thereof according to claim 1, wherein the 3E10-VL (SEQ-ID-N9) comprises an amino acid sequence that is at least 97%, 98%, 99%, or 100% identical to 3E10-VL-h6 (SEQ ID NO:90).

10. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h1 (SEQ ID NO:64).

11. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h2 (SEQ ID NO:65).

12. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h3 (SEQ ID NO:66).

13. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h4 (SEQ ID NO:67).

14. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h5 (SEQ ID NO:68).

15. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h6 (SEQ ID NO:69).

16. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h7 (SEQ ID NO:70).

17. The humanized 3E10 antibody or antigen binding fragment thereof according to claim 1, wherein the 3E10-VH comprises an amino acid sequence that is at least 95%, 96%, 97%, 98%, 99%, or 100% identical to 3E10-VH-h6 (SEQ ID NO:69) and the 3E10-VL comprises an amino acid sequence that is at least 97%, 98%, 99%, or 100% identical to 3E10-VL-h6 (SEQ ID NO:90).

18. The humanized 3E10 antibody or antigen binding fragment thereof of claim 17, wherein the 3E10-VH comprises the amino acid sequence of 3E10-VH-h6 (SEQ ID NO:69) and the 3E10-VL comprises the amino acid sequence of 3E10-VL-h6 (SEQ ID NO:90).

19. The humanized 3E10 antibody or antigen binding fragment thereof of claim 17, wherein the 3E10-HC comprises the amino acid sequence of 3E10-HC-h6m (SEQ ID NO:76) and the 3E10-LC comprises the amino acid sequence of 3E10-LC-h6m (SEQ ID NO:96).

20. The humanized 3E10 antibody or antigen binding fragment thereof of claim 17, wherein the 3E10-HC comprises the amino acid sequence of 3E10-HC-h67 (SEQ ID NO:83) and the 3E10-LC comprises the amino acid sequence of 3E10-LC-h6 (SEQ ID NO:102).

21. The humanized 3E10 antibody or antigen binding fragment thereof according to claim 1, comprising a set of complementarity determining regions (CDRs) collectively having no more than 7, 6, 5, 4, 3, 2, or 1 amino acid substitutions relative to the set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

22. The humanized 3E10 antibody or antigen binding fragment thereof according to claim 1, comprising a set of CDRs having the amino acid sequences of 3E10-VL-CDR1 (SEQ ID NO:9), 3E10-VL-CDR2 (SEQ ID NO:10), 3E10-VL-CDR3 (SEQ ID NO:11), 3E10-VH-CDR1_D31N (SEQ ID NO:15), 3E10-VH-CDR2 (SEQ ID NO:4), and 3E10-VH-CDR3 (SEQ ID NO:5).

23-77. (canceled)

78. A composition comprising a covalent or non-covalent complex of: (i) a humanized 3E10 antibody or antigen binding fragment thereof according to claim 1, and (ii) a polynucleotide.

79-135. (canceled)

136. A method for treating a cancer in a subject in need thereof, the method comprising administering to the subject a therapeutically effective amount of a humanized 3E10 antibody or antigen binding fragment thereof according to claim 1.

137-157. (canceled)

158. A polynucleotide encoding the humanized 3E10 antibody or antigen binding fragment thereof according to claim 1.

159. (canceled)


SEQUENCE LISTING (161 sequences)

SEQ ID NO: 1 - protein, 446 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 3 - protein, 5 aa, synthetic construct
DYGMH

SEQ ID NO: 4 - protein, 17 aa, synthetic construct
YISSGSSTIYYADTVKG

SEQ ID NO: 5 - protein, 7 aa, synthetic construct
RGLLLDY

SEQ ID NO: 6 - protein, 19 aa, synthetic construct
MGWSCIILFLVATATGVHS

SEQ ID NO: 7 - protein, 218 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 8 - protein, 111 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIK

SEQ ID NO: 9 - protein, 15 aa, synthetic construct
RASKSVSTSSYSYMH

SEQ ID NO: 10 - protein, 7 aa, synthetic construct
YASYLES

SEQ ID NO: 11 - protein, 9 aa, synthetic construct
QHSREFPWT

SEQ ID NO: 12 - protein, 19 aa, synthetic construct
MGWSCIILFLVATATGVHS

SEQ ID NO: 13 - protein, 446 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 116 aa, synthetic construct
EVQLVESGGGLVKPGGSRKLSCAASGFTFSNYGMHWVRQAPEKGLEWVAYISSGSSTIYYADTVKGRFTISRDNAKNTLFLQMTSLRSEDTAMYYCARRGLLLDYWGQGTTLTVSS

SEQ ID NO: 15 - protein, 5 aa, synthetic construct
NYGMH

SEQ ID NO: 16 - protein, 5 aa, synthetic construct
BYGMH

SEQ ID NO: 17 - protein, 17 aa, synthetic construct
YISSGSSTIYYADTVKG

SEQ ID NO: 18 - protein, 7 aa, synthetic construct
RGLLLDY

SEQ ID NO: 19 - protein, 19 aa, synthetic construct
MGWSCIILFLVATATGVHS

SEQ ID NO: 20 - protein, 218 aa, synthetic construct; identical to SEQ ID NO: 7

SEQ ID NO: 21 - protein, 111 aa, synthetic construct; identical to SEQ ID NO: 8

SEQ ID NO: 22 - protein, 15 aa, synthetic construct
RASKSVSTSSYSYMH

SEQ ID NO: 23 - protein, 7 aa, synthetic construct
YASYLES

SEQ ID NO: 24 - protein, 9 aa, synthetic construct
QHSREFPWT

SEQ ID NO: 25 - protein, 19 aa, synthetic construct
MGWSCIILFLVATATGVHS

SEQ ID NO: 26 - protein, 17 aa, synthetic construct
YISSGSSTIYYADSVKG

SEQ ID NO: 27 - protein, 17 aa, synthetic construct
YISSSSSTIYYADSVKG

SEQ ID NO: 28 - protein, 15 aa, synthetic construct
RASKSVSTSSYSYLA

SEQ ID NO: 29 - protein, 15 aa, synthetic construct
RASKTVSTSSYSYMH

SEQ ID NO: 30 - protein, 7 aa, synthetic construct
YASYLQS

SEQ ID NO: 31 - protein, 17 aa, synthetic construct
  VARIANT 5: note = X can be separately any amino acid
  VARIANT 14: note = X can be separately any amino acid
YISSXSSTIYYADXVKG

SEQ ID NO: 32 - protein, 15 aa, synthetic construct
  VARIANT 5: note = X can be separately any amino acid
  VARIANT 14..15: note = X can be separately any amino acid
RASKXVSTSSYSYXX

SEQ ID NO: 33 - protein, 7 aa, synthetic construct
  VARIANT 6: note = X can be any amino acid
YASYLXS

SEQ ID NO: 34 - protein, 5 aa, synthetic construct
QYGMH

SEQ ID NO: 35 - protein, 5 aa, synthetic construct
EYGMH

SEQ ID NO: 36 - skipped

SEQ ID NO: 37 - protein, 5 aa, synthetic construct
  VARIANT 5: note = X is K or R
QYGMX

SEQ ID NO: 38 - protein, 5 aa, synthetic construct
  VARIANT 5: note = X is K or R
EYGMX

SEQ ID NO: 39 - protein, 17 aa, synthetic construct
YISSGSSTIYYAETVKG

SEQ ID NO: 40 - protein, 17 aa, synthetic construct
  VARIANT 16: note = X is R or H
YISSGSSTIYYADTVXG

SEQ ID NO: 41 - protein, 17 aa, synthetic construct
  VARIANT 16: note = X is R or H
YISSGSSTIYYAETVXG

SEQ ID NO: 42 - protein, 7 aa, synthetic construct
  VARIANT 1: note = X is K or H
XGLLLDY

SEQ ID NO: 43 - protein, 7 aa, synthetic construct
RGLLLEY

SEQ ID NO: 44 - protein, 7 aa, synthetic construct
  VARIANT 1: note = X is K or H
XGLLLEY

SEQ ID NO: 45 - protein, 15 aa, synthetic construct
  VARIANT 1: note = X is K or H
XASKSVSTSSYSYMH

SEQ ID NO: 46 - protein, 15 aa, synthetic construct
  VARIANT 4: note = X is R or H
RASXSVSTSSYSYMH

SEQ ID NO: 47 - protein, 15 aa, synthetic construct
  VARIANT 15: note = X is K or R
RASKSVSTSSYSYMX

SEQ ID NO: 48 - protein, 15 aa, synthetic construct
  VARIANT 1: note = X is K or H
  VARIANT 4: note = X is R or H
XASXSVSTSSYSYMH

SEQ ID NO: 49 - protein, 15 aa, synthetic construct
  VARIANT 1: note = X is K or H
  VARIANT 15: note = X is K or R
XASKSVSTSSYSYMX

SEQ ID NO: 50 - protein, 15 aa, synthetic construct
  VARIANT 4: note = X is R or H
  VARIANT 15: note = X is K or R
RASXSVSTSSYSYMX

SEQ ID NO: 51 - protein, 7 aa, synthetic construct
YASYLDS

SEQ ID NO: 52 - protein, 9 aa, synthetic construct
  VARIANT 2: note = X is K or R
QXSREFPWT

SEQ ID NO: 53 - protein, 9 aa, synthetic construct
  VARIANT 4: note = X is K or H
QHSXEFPWT

SEQ ID NO: 54 - protein, 9 aa, synthetic construct
QHSRDFPWT

SEQ ID NO: 55 - protein, 9 aa, synthetic construct
  VARIANT 2: note = X is K or R
  VARIANT 4: note = X is K or H
QXSXEFPWT

SEQ ID NO: 56 - protein, 9 aa, synthetic construct
  VARIANT 2: note = X is K or R
QXSRDFPWT

SEQ ID NO: 57 - protein, 9 aa, synthetic construct
  VARIANT 4: note = X is K or H
QHSXDFPWT

SEQ ID NO: 58 - skipped

SEQ ID NO: 59 - protein, 17 aa, synthetic construct
  VARIANT 5: note = X is G or S
  VARIANT 13: note = X is D or E
  VARIANT 14: note = X is T or S
  VARIANT 16: note = X is K, R, or H
YISSXSSTIYYAXXVXG

SEQ ID NO: 60 - protein, 7 aa, synthetic construct
  VARIANT 1: note = X is K, R, or H
  VARIANT 6: note = X is D or E
XGLLLXY

SEQ ID NO: 61 - protein, 15 aa, synthetic construct
  VARIANT 1: note = X is K, R, or H
  VARIANT 4: note = X is K, R, or H
  VARIANT 5: note = X is T or S
  VARIANT 14: note = X is M or L
  VARIANT 15: note = X is K, R, H, or A
XASXXVSTSSYSYXX

SEQ ID NO: 62 - protein, 7 aa, synthetic construct
  VARIANT 6: note = X is D, E, N, or Q
YASYLXS

SEQ ID NO: 63 - protein, 9 aa, synthetic construct
  VARIANT 2: note = X is K, R, or H
  VARIANT 4: note = X is K, R, or H
  VARIANT 5: note = X is D or E
QXSXXFPWT

SEQ ID NO: 64 - protein, 116 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSS

SEQ ID NO: 65 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSS

SEQ ID NO: 66 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSS

SEQ ID NO: 67 - protein, 116 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSS

SEQ ID NO: 68 - protein, 116 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 69 - protein, 116 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSS

SEQ ID NO: 70 - protein, 116 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSS

SEQ ID NO: 71 - protein, 446 aa, synthetic construct
EVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 446 aa, synthetic construct
EVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 446 aa, synthetic construct
EVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 446 aa, synthetic construct
EVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 446 aa, synthetic construct
EVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 78 - protein, 465 aa, synthetic construct
MGWSCIILFLVATATGVHSEVQLVESGGGLVQPGGSLRLSCAASGFTFSNYGMHWVRQAPGKGLEWVSYISSGSSTIYYADTVKGRFTISRDNAKNSLYLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMNSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 81 - protein, 465 aa, synthetic construct
MGWSCIILFLVATATGVHSEVQLVESGGGLVQPGGSLRLSCAASGFTFSNYGMHWVRQAPEKGLEWVAYISSGSSTIYYADTVKGRFTISRDNAKNSLYLQMTSLRAEDTAMYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 82 - protein, 465 aa, synthetic construct
MGWSCIILFLVATATGVHSEVQLVESGGGLVQPGGSLRLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 83 - protein, 465 aa, synthetic construct
MGWSCIILFLVATATGVHSEVQLVESGGGLVQPGGSRKLSCAASGFTFSNYGMHWVRQAPGKGLEWVAYISSGSSTIYYADTVKGRFTISRDNAKNTLFLQMNSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 465 aa, synthetic construct
MGWSCIILFLVATATGVHSEVQLVESGGGLVQPGGSRKLSCAASGFTFSNYGMHWVRQAPEKGLEWVAYISSGSSTIYYADTVKGRFTISRDNAKNTLFLQMTSLRAEDTAVYYCARRGLLLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 111 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIK

SEQ ID NO: 86 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIK

SEQ ID NO: 87 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIK

SEQ ID NO: 88 - protein, 111 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIK

SEQ ID NO: 89 - protein, 111 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIK

SEQ ID NO: 90 - protein, 111 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIK

SEQ ID NO: 91 - protein, 218 aa, synthetic construct
DIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 218 aa, synthetic construct
DIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 218 aa, synthetic construct
DIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 218 aa, synthetic construct
DIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 237 aa, synthetic construct
MGWSCIILFLVATATGVHSDIVLTQSPASLAVSPGQRATITCRASKSVSTSSYSYMHWYQQKPGQPPKLLIYYASYLESGVPARFSGSGSGTDFTLTINPVEANDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLTINPVEAEDAATYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 237 aa, synthetic construct
MGWSCIILFLVATATGVHSDIVLTQSPASLAVSPGQRATITCRASKSVSTSSYSYMHWYQQKPGQPPKLLIKYASYLESGVPARFSGSGSGTDFTLNIHPVEAEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 237 aa, synthetic construct
MGWSCIILFLVATATGVHSDIVLTQSPASLAVSPGQRATISCRASKSVSTSSYSYMHWYQQKPGQPPKLLIKYASYLESGVPARFSGSGSGTDFTLNIHPVEEEDTANYYCQHSREFPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - RNA, 89 nt, synthetic construct
  source 1..89: mol_type = other RNA
ggagcaaaagcagggtgacaaagacataatggatccaaacactgtgtcaagctttcaggtagattgctttctttggcatgtccgcaaac

SEQ ID NO: 104 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 105 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 106 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLQVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHAHYTQKSLSLSLGK

SEQ ID NO: 107 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 108 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLQQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 31 aa, synthetic construct
RASKSVSTSSYSYMHWYQQKPGQPPKLLIKY

SEQ ID NO: 110 - protein, 31 aa, synthetic construct
RASKTVSTSSYSYMHWYQQKPGQPPKLLIKY

SEQ ID NO: 111 - protein, 33 aa, synthetic construct
RVTITCRASKSVSTSSYSYMHWYQQKPGKAPKL

SEQ ID NO: 112 - protein, 32 aa, synthetic construct
  VARIANT 1: note = X can be any residue, but preferentially a basic residue (R or K)
  VARIANT 30: note = X can be any residue, but preferentially a basic residue (R or K)
XRASKTVSTSSYSYMHWYQQKPGQPPKLLXKY

SEQ ID NO: 113 - protein, 115 aa, synthetic construct
EVQLVESGGGLVKPGGSRKLSCAASGFTFSDYGMHWVRQAPEKGLEWVAYISSGSSTIYYADTVKGRFTISRDNAKNTLFLQMTSLRSEDTAMYYCARRGLLLDYWGQGTTLTVS

SEQ ID NO: 114 - protein, 132 aa, synthetic construct
DIVLTQSPASLAVSLGQRATISCRASKSVSTSSYSYMHWYQQKPGQPPKLLIKYASYLESGVPARFSGSGSGTDFTLNIHPVEEEDAATYYCQHSREFPWTFGGGTKLEIKRADAAPGGGGSGGGGSGGGGS

SEQ ID NO: 115 - protein, 330 aa, synthetic construct [sequence omitted]

SEQ ID NO: 116 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 7 aa, synthetic construct
GQSSRSS

SEQ ID NO: 120 - protein, 18 aa, synthetic construct
GQSSRSSSGGGSSGGGGS

SEQ ID NO: 121 - protein, 4 aa, synthetic construct
GSGS

SEQ ID NO: 122 - protein, 4 aa, synthetic construct
GGGS

SEQ ID NO: 123 - protein, 10 aa, synthetic construct
GGGGSGGGGS

SEQ ID NO: 124 - protein, 20 aa, synthetic construct
GGGGSGGGGSGGGGSGGGGS

SEQ ID NO: 125 - protein, 15 aa, synthetic construct
GGGGSGGGGSGGGGS

SEQ ID NO: 126 - protein, 21 aa, synthetic construct
RADAAPGGGGSGGGGSGGGGS

SEQ ID NO: 127 - protein, 19 aa, synthetic construct
ASTKGPSVFPLAPLESSGS

SEQ ID NO: 128 - DNA, 41 bp, synthetic construct
  source 1..41: mol_type = other DNA
atctaactgctgcgccgccgggaaaatactgtacggttaga

SEQ ID NO: 129 - DNA, 26 bp, synthetic construct
  source 1..26: mol_type = other DNA
ggtggtggtggttgtggtggtggtgg

SEQ ID NO: 130 - RNA, 45 nt, synthetic construct
  source 1..45: mol_type = other RNA
gcgtggtgtgatctagatgtattggctgatcctagtcaggtacgc

SEQ ID NO: 131 - DNA, 78 bp, synthetic construct
  source 1..78: mol_type = other DNA
atccagagtgacgcagcatgccctagttactactactctttttagcaaacgccctcgctttggacacggtggcttagt

SEQ ID NO: 132 - DNA, 50 bp, synthetic construct
  source 1..50: mol_type = other DNA
aaggggttgggtgggtttatacaaattaattaatattgtatggtatattt

SEQ ID NO: 133 - DNA, 44 bp, synthetic construct
  source 1..44: mol_type = other DNA
accgggaggatagttcggtggctgttcagggtctcctcccggtg

SEQ ID NO: 134 - DNA, 88 bp, synthetic construct
  source 1..88: mol_type = other DNA
aaccgcccaaatccctaagagtctgcacttgtcattttgtatatgtatttggtttttggctctcacagacacactacacacgcacatg

SEQ ID NO: 135 - DNA, 40 bp, synthetic construct
  source 1..40: mol_type = other DNA
tctaaaaggattcttcccaaggggatccaattcaaacagc

SEQ ID NO: 136 - DNA, 54 bp, synthetic construct
  source 1..54: mol_type = other DNA
gggccgtcgaacacgagcatggtgcgtggacctaggatgacctgagtactgtcc

SEQ ID NO: 137 - DNA, 57 bp, synthetic construct
  source 1..57: mol_type = other DNA
tggatggggagatccgttgagtaagcgggcgtgtctctctgccgccttgctatgggg

SEQ ID NO: 138 - DNA, 48 bp, synthetic construct
  source 1..48: mol_type = other DNA
cactacagaggttgcgtctgtcccacgttgtcatggggggttggcctg

SEQ ID NO: 139 - DNA, 25 bp, synthetic construct
  source 1..25: mol_type = other DNA
ggtggtggtggtttggtggtggtgg

SEQ ID NO: 140 - DNA, 40 bp, synthetic construct
  source 1..40: mol_type = other DNA
agggcccgacgtgacggcacgtcggatatctcatgcgtgt

SEQ ID NO: 141 - DNA, 40 bp, synthetic construct
  source 1..40: mol_type = other DNA
gcccagcatgcattactgatcgtggtgtttgcttagccca

SEQ ID NO: 142 - DNA, 64 bp, synthetic construct
  source 1..64: mol_type = other DNA
tttttttttacattcctaagtctgaaacattacagcttgctacacgagaagagccgccatagta

SEQ ID NO: 143 - RNA, 65 nt, synthetic construct
  source 1..65: mol_type = other RNA
gggtcgtgtcggatccaggcacgacgcatagccttgggagcgaggaaagcttctaaggtaacgat

SEQ ID NO: 144 - RNA, 65 nt, synthetic construct
  source 1..65: mol_type = other RNA
gggtcgtgtcggatccagctcgaaagtgggctggcgatgtgtcccgaagcttctaaggtaacgat

SEQ ID NO: 145 - RNA, 65 nt, synthetic construct
  source 1..65: mol_type = other RNA
gggtcgtgtcggatcctgcgaaggggggcagaggtttgacgcgagaaagcttctaaggtaacgat

SEQ ID NO: 146 - DNA, 88 bp, synthetic construct
  source 1..88: mol_type = other DNA
ctatagcaatggtacggtacttcctctcaggtgggtgtatgtgggctccctttactgattgggtcaaaagtgcacgctactttgctaa

SEQ ID NO: 147 - DNA, 45 bp, synthetic construct
  source 1..45: mol_type = other DNA
ggttgcatgccgtggggaggggggtgggttttatagcgtactcag

SEQ ID NO: 148 - DNA, 30 bp, synthetic construct
  source 1..30: mol_type = other DNA
ttgggacggtgttaaacgaaaggggacgac

SEQ ID NO: 149 - RNA, 44 nt, synthetic construct
  source 1..44: mol_type = other RNA
aaaatgcatggagcgaaggtgtgggggataccaaccgcgccgtg

SEQ ID NO: 150 - DNA, 80 bp, synthetic construct
  source 1..80: mol_type = other DNA
gctgtgtgactcctgcaagcgggaagagggcaggggagggagggtgacgcggaagaggcaagcagctgtatcttgtctcc

SEQ ID NO: 151 - DNA, 44 bp, synthetic construct
  source 1..44: mol_type = other DNA
ctctagttattgagttttcttttatgggtgggtggggggttttt

SEQ ID NO: 152 - DNA, 58 bp, synthetic construct
  source 1..58: mol_type = other DNA
ttggagcagcgtggaggatatgctttccgaccgtgttcgtttgttataacgctgctcc

SEQ ID NO: 153 - DNA, 68 bp, synthetic construct
  source 1..68: mol_type = other DNA
ttaaggagcagcgtggaggatatcggtgtttatggtgtctgtcttcctccagtttccttctgcgcctt

SEQ ID NO: 154 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 155 - protein, 12 aa, synthetic construct
EPKSCDKTHTCP

SEQ ID NO: 156 - protein, 113 aa, synthetic construct
PCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 157 - protein, 107 aa, synthetic construct
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 158 - protein, 113 aa, synthetic construct
PCPAPELLGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 159 - protein, 113 aa, synthetic construct
PCPAPEAAGGPSVFLFPPKPKDTLMISRTPEVTCVVVDVSHEDPEVKFNWYVDGVEVHNAKTKPREEQYDSTYRVVSVLTVLHQDWLNGKEYKCKVSNKALPAPIEKTISKAK

SEQ ID NO: 160 - protein, 113 aa, synthetic construct
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 161 - protein, 218 aa, synthetic construct
DIQMTQSPSSLSASLGDRATITCRASKTVSTSSYSYMHWYQQKPGQPPKLLIKYASYLESGVPSRFSGSGSGTDFTLTISSLQPEDAATYYCQHSREFPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC